(19) United States
(12) Patent Application Publication
(10) Pub. No.: US 2023/0180601 A1
(43) Pub. Date: Jun. 8, 2023

(54) COMPOSITION FOR ORGANIC OPTOELECTRONIC ELEMENT, ORGANIC OPTOELECTRONIC ELEMENT, AND DISPLAY DEVICE

(71) Applicant: SAMSUNG SDI CO., LTD., Yongin-si, Gyeonggi-do (KR)

(72) Inventors: Seonyeong GWAK, Suwon-si, Gyeonggi-do (KR); Jongwoo WON, Suwon-si, Gyeonggi-do (KR); Mijin LEE, Suwon-si, Gyeonggi-do (KR); Jonghoon KO, Suwon-si, Gyeonggi-do (KR); Hyung Sun KIM, Suwon-si, Gyeonggi-do (KR); Hayun SONG, Suwon-si, Gyeonggi-do (KR); Chang Ju SHIN, Suwon-si, Gyeonggi-do (KR); Seungjae LEE, Suwon-si, Gyeonggi-do (KR); Sung-Hyun JUNG, Suwon-si, Gyeonggi-do (KR); Ho Kuk JUNG, Suwon-si, Gyeonggi-do (KR)

(21) Appl. No.: 17/924,003

(22) PCT Filed: Apr. 28, 2021
(86) PCT No.: PCT/KR2021/005383

§ 371 Date: Nov. 8, 2022

(30) Foreign Application Priority Data
May 20, 2020 (KR) 10-2020-0060487
Apr. 27, 2021 (KR) 10-2021-0054497

(51) Int. Cl. H10K 85/60

(52) U.S. Cl. CPC H10K 85/657; H10K 85/654; H10K 85/622; H10K 85/6572; H10K 85/6574; H10K 85/6576; H10K 85/615; H10K 2102/301

(57) ABSTRACT

Disclosed are a composition for an organic optoelectronic device including a first compound represented by Chemical Formula 1, and a second compound represented by Chemical Formula 2, and an organic optoelectronic device and a display device including the same.

The contents of Chemical Formula 1 and Chemical Formula 2 are as defined in the specification.

### TECHNICAL FIELD

A composition for an organic optoelectronic device, an organic optoelectronic device, and a display device are disclosed.

### BACKGROUND ART

An organic optoelectronic device (organic optoelectronic diode) is a device that converts electrical energy into photoenergy, and vice versa.

An organic optoelectronic device may be classified as follows in accordance with its driving principles. One is a photoelectric device that generates electrical energy by separating excitons formed by light energy into electrons and holes, and transferring the electrons and holes to different electrodes, respectively and the other is a light emitting device that generates light energy from electrical energy by supplying voltage or current to the electrodes.

Examples of the organic optoelectronic device include an organic photoelectric element, an organic light emitting diode, an organic solar cell, and an organic photo conductor drum.

Among them, organic light emitting diodes (OLEDs) are attracting much attention in recent years due to increasing demands for flat panel display devices. The organic light emitting diode is a device that converts electrical energy into light, and the performance of the organic light emitting diode is greatly influenced by an organic material between electrodes.

### DISCLOSURE

### Technical Problem

An embodiment provides a composition for an organic optoelectronic device capable of implementing a high efficiency and low driving voltage organic optoelectronic device.

Another embodiment provides an organic optoelectronic device including the composition for an organic optoelectronic device.

Another embodiment provides a display device including the organic optoelectronic device.

### Technical Solution

According to an embodiment, a composition for an organic optoelectronic device includes a first compound represented by Chemical Formula 1 and a second compound represented by Chemical Formula 2.

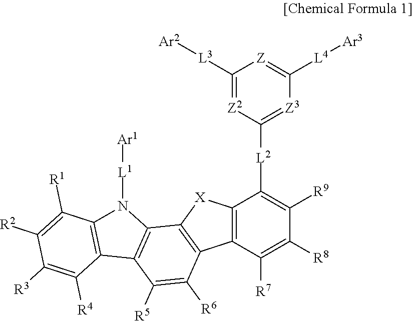

In Chemical Formula 1,

X is O or S,

Z¹ to Z³ are each independently N or CR^{a},

at least two of Z¹ to Z³ are N,

R^{a} and R¹ to R⁹ are each independently hydrogen, deuterium, a substituted or unsubstituted C1 to C30 alkyl group, a substituted or unsubstituted C6 to C30 aryl group, or a substituted or unsubstituted C2 to C30 heterocyclic group,

L¹ to L⁴ are each independently a single bond, or a substituted or unsubstituted C6 to C30 arylene group, and

Ar¹ to Ar³ are each independently a substituted or unsubstituted C6 to C30 aryl group, or a substituted or unsubstituted C2 to C30 heterocyclic group;

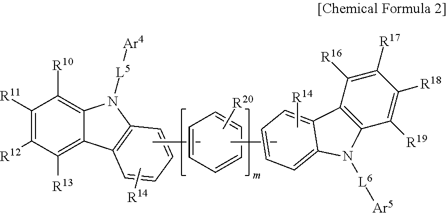

wherein, in Chemical Formula 2,

Ar⁴ and Ar⁵ are each independently a substituted or unsubstituted C6 to C30 aryl group, or a substituted or unsubstituted C2 to C30 heterocyclic group,

L⁵ and L⁶ are each independently a single bond, or a substituted or unsubstituted C6 to C30 arylene group,

R¹⁰ to R²⁰ are each independently hydrogen, deuterium, a substituted or unsubstituted C1 to C30 alkyl group, a substituted or unsubstituted C6 to C30 aryl group, or a substituted or unsubstituted C2 to C30 heterocyclic group, and m is an integer of 0 to 2.

According to another embodiment, an organic optoelectronic device includes an anode and a cathode facing each other, and at least one organic layer between the anode and the cathode, wherein the organic layer includes a light emitting layer and the light emitting layer includes the aforementioned composition for an organic optoelectronic device.

According to another embodiment, a display device including the organic optoelectronic device is provided.

### Advantageous Effects

Low driving voltage organic optoelectronic device may be implemented.

### DESCRIPTION OF THE DRAWINGS

FIGS. 1 and 2 are cross-sectional views each illustrating an organic light emitting diode according to embodiments.

### DESCRIPTION OF SYMBOLS

100, 200: organic light emitting diode
105: organic layer
110: cathode
120: anode
130: light emitting layer
140: hole auxiliary layer

### BEST MODE

Hereinafter, embodiments of the present invention are described in detail. However, these embodiments are exemplary, the present invention is not limited thereto and the present invention is defined by the scope of claims.

In the present specification, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example of the present invention, “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In addition, in specific examples of the present invention, “substituted” refers to replacement of at least one hydrogen of a substituent or a compound by deuterium, a C1 to C20 alkyl group, a C6 to C30 aryl group, or a cyano group. In addition, in specific examples of the present invention, “substituted” refers to replacement of at least one hydrogen of a substituent or a compound by deuterium, a C1 to C5 alkyl group, a C6 to C18 aryl group, or a cyano group. In addition, in specific examples of the present invention, “substituted” refers to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

In the present specification, when a definition is not otherwise provided, “hetero” refers to one including one to three heteroatoms selected from N, O, S, P, and Si, and remaining carbons in one functional group.

In the present specification, “aryl group” refers to a group including at least one hydrocarbon aromatic moiety, and may include a group in which all elements of the hydrocarbon aromatic moiety have p-orbitals which form conjugation, for example a phenyl group, a naphthyl group, and the like, a group in which two or more hydrocarbon aromatic moieties may be linked by a sigma bond, for example a biphenyl group, a terphenyl group, a quarterphenyl group, and the like, and a group in which two or more hydrocarbon aromatic moieties are fused directly or indirectly to provide a non-aromatic fused ring, for example, a fluorenyl group, and the like.

The aryl group may include a monocyclic, polycyclic or fused ring polycyclic (i.e., rings sharing adjacent pairs of carbon atoms) functional group.

In the present specification, “heterocyclic group” is a generic concept of a heteroaryl group, and may include at least one heteroatom selected from N, O, S, P, and Si instead of carbon (C) in a cyclic compound such as an aryl group, a cycloalkyl group, a fused ring thereof, or a combination thereof. When the heterocyclic group is a fused ring, the entire ring or each ring of the heterocyclic group may include one or more heteroatoms.

For example, “heteroaryl group” refers to an aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, a substituted or unsubstituted furanyl group, or a combination thereof, but is not limited thereto.

More specifically, the substituted or unsubstituted C2 to C30 heterocyclic group may be a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, or a substituted or unsubstituted dibenzothiophenyl group, or a combination thereof, but is not limited thereto.

In the present specification, hole characteristics refer to an ability to donate an electron to form a hole when an electric field is applied and that a hole formed in the anode may be easily injected into the light emitting layer and transported in the light emitting layer due to conductive characteristics according to the highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to the lowest unoccupied molecular orbital (LUMO) level.

Hereinafter, a composition for an organic optoelectronic device according to an embodiment is described.

The composition for an organic optoelectronic device according to an embodiment includes a first compound represented by Chemical Formula 1, and a second compound represented by Chemical Formula 2.

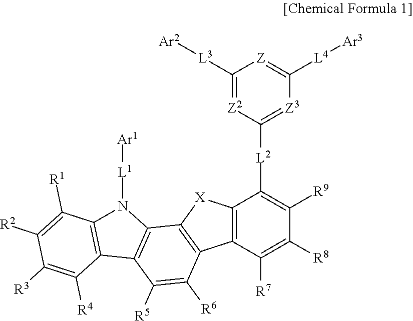

In Chemical Formula 1,

X is O or S,

Z¹ to Z³ are each independently N or CR′,

at least two of Z¹ to Z³ are N,

R^{a} and R¹ to R⁹ are each independently hydrogen, deuterium, a substituted or unsubstituted C1 to C30 alkyl group, a substituted or unsubstituted C6 to C30 aryl group, or a substituted or unsubstituted C2 to C30 heterocyclic group,

L¹ to L⁴ are each independently a single bond, or a substituted or unsubstituted C6 to C30 arylene group, and

Ar¹ to Ar³ are each independently a substituted or unsubstituted C6 to C30 aryl group, or a substituted or unsubstituted C2 to C30 heterocyclic group;

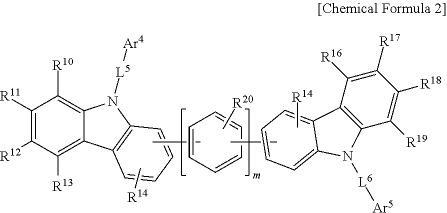

In Chemical Formula 2,

Ar⁴ and Ar⁵ are each independently a substituted or unsubstituted C6 to C30 aryl group, or a substituted or unsubstituted C2 to C30 heterocyclic group,

L⁵ and L⁶ are each independently a single bond, or a substituted or unsubstituted C6 to C30 arylene group,

R¹⁰ to R²⁰ are each independently hydrogen, deuterium, a substituted or unsubstituted C1 to C30 alkyl group, a substituted or unsubstituted C6 to C30 aryl group, or a substituted or unsubstituted C2 to C30 heterocyclic group,

m is an integer of 0 to 2.

The first compound represented by Chemical Formula 1 includes an indolodibenzofuran (or indolodibenzothiophene) backbone, and in the indolodibenzofuran (or indolodibenzothiophene) backbone, dibenzofuran (or dibenzothiophene) has a structure which is substituted with a 6-membered nitrogen-containing ring at the 4th position.

The first compound having such a structure is a compound capable of receiving both holes and electrons when an electric field is applied, that is, a compound having a bipolar property. Among them, the electron cloud is widely spread and thus has a fast and stable electron transfer property. In addition, since it has a high glass transition temperature and is deposited at a relatively low temperature, thermal stability is improved.

Particularly, when applied to an organic light emitting diode together with a second compound having excellent hole transport properties, charge balance is achieved to realize low driving characteristics.

The second compound may include a bicarbazole backbone, and is included together with the aforementioned first compound to increase the balance between holes and electrons, thereby greatly improving driving characteristics of a device including the same.

According to an embodiment, Ar¹ of Chemical Formula 1 may be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted fluorenyl group, a substituted or unsubstituted dibenzofuranyl group, or a substituted or unsubstituted dibenzothiophenyl group.

For example, Ar¹ may be a substituted or unsubstituted phenyl group.

According to an embodiment, Ar² and Ar³ of Chemical Formula 1 may each independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted fluorenyl group, a substituted or unsubstituted carbazolyl group, a substituted or unsubstituted dibenzofuranyl group, a substituted or unsubstituted dibenzothiophenyl group, or a substituted or unsubstituted pyridinyl group, and

L³ and L⁴ may each independently be a single bond, a substituted or unsubstituted phenylene group, or a substituted or unsubstituted biphenylene group.

For example, *-L³-Ar² and *-L⁴-Ar³ of Chemical Formula 1 may each independently be selected from the substituents of Group I.

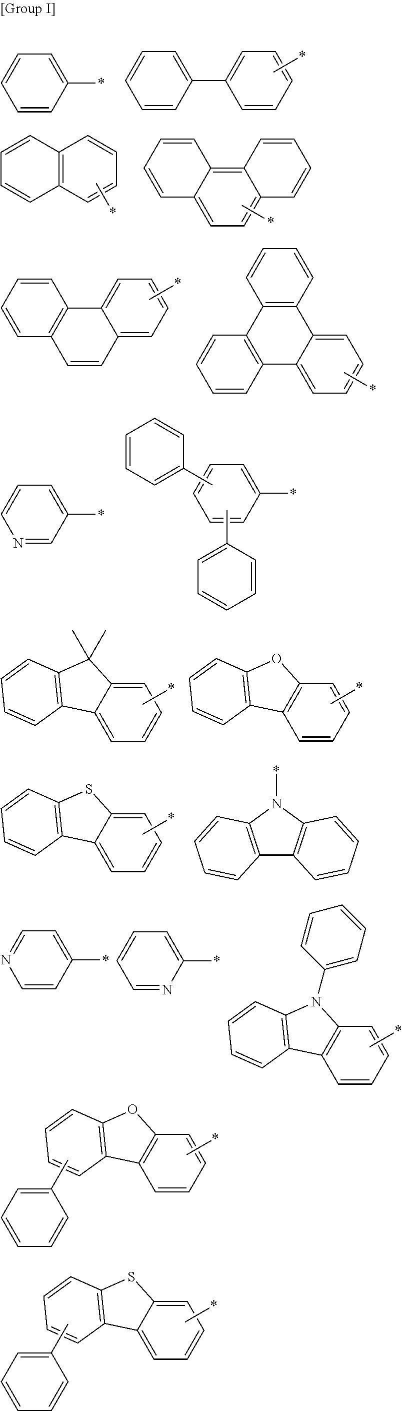

As a specific example, *-L³-Ar² and *-L⁴-Ar³ of Chemical Formula 1 may each independently be selected from the substituents of Group I-1.

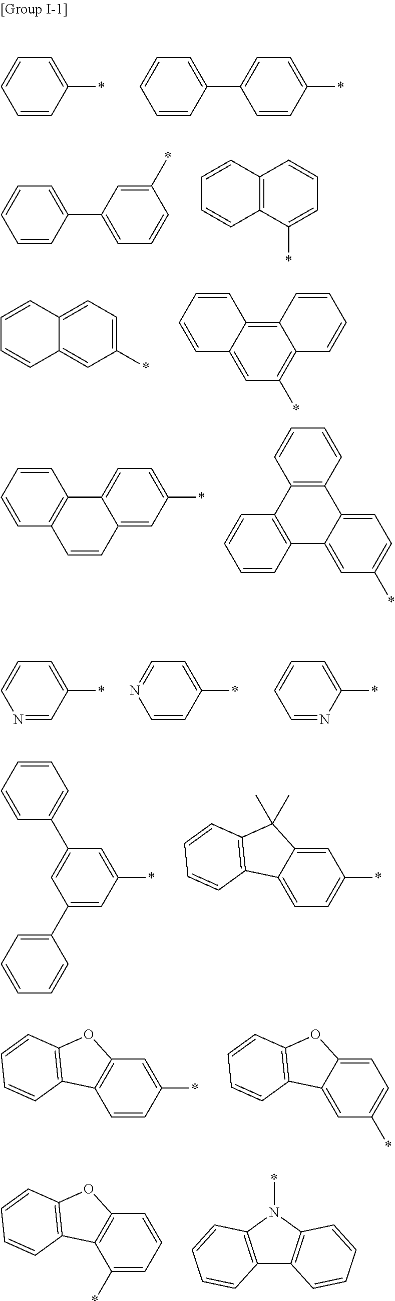

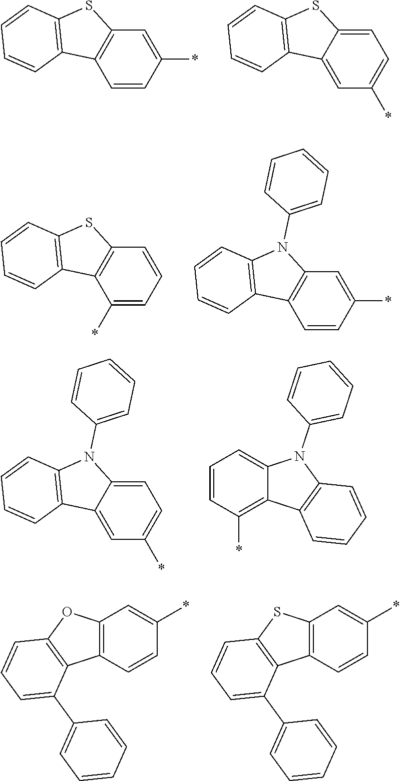

In Groups I and Group I-1, * is a linking point.

As a specific example, Ar² and Ar³ of Chemical Formula 1 may each independently be a substituted or unsubstituted phenyl group, or a substituted or unsubstituted biphenyl group, and L³ and L⁴ may each independently be a single bond, or a substituted or unsubstituted phenylene group.

As a more specific example, *-L³-Ar² and *-L⁴-Ar³ may each independently be a substituted or unsubstituted phenyl group or a substituted or unsubstituted biphenyl group.

In an embodiment, at least one of *-L³-Ar² and *-L⁴-Ar³ may be a substituted or unsubstituted biphenyl group.

For example, the first compound may be one selected from compounds of Group 1, but is not limited thereto.

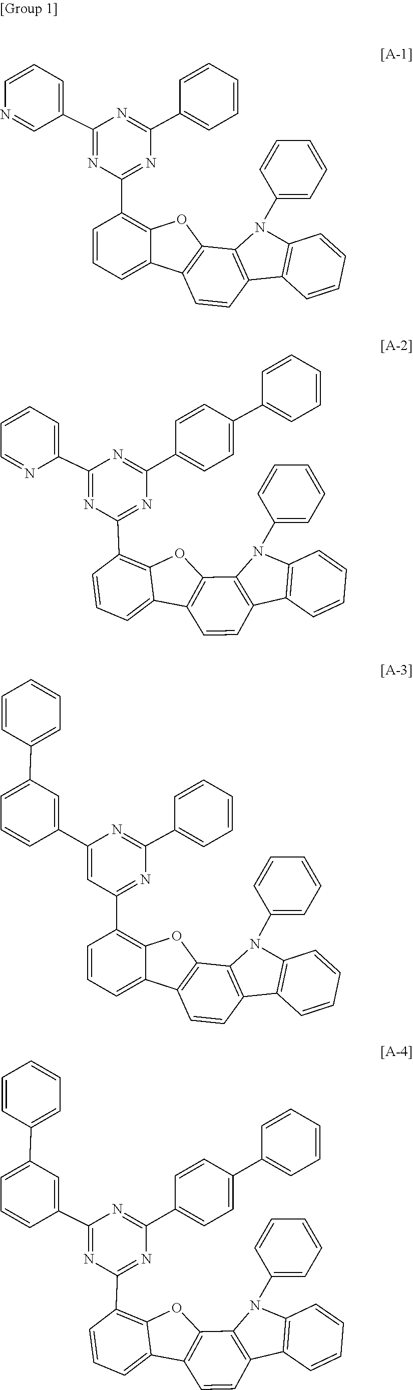 [SMILES: c1ccc(-c2nc(-c3cccnc3)nc(-c3cccc4c3oc3c4ccc4c5ccccc5n(-c5ccccc5)c43)n2)cc1 ; c1ccc(-c2ccc(-c3nc(-c4ccccn4)nc(-c4cccc5c4oc4c5ccc5c6ccccc6n(-c6ccccc6)c54)n3)cc2)cc1 ; c1ccc(-c2cccc(-c3cc(-c4cccc5c4oc4c5ccc5c6ccccc6n(-c6ccccc6)c54)nc(-c4ccccc4)n3)c2)cc1 ; c1ccc(-c2ccc(-c3nc(-c4cccc(-c5ccccc5)c4)nc(-c4cccc5c4oc4c5ccc5c6ccccc6n(-c6ccccc6)c54)n3)cc2)cc1]

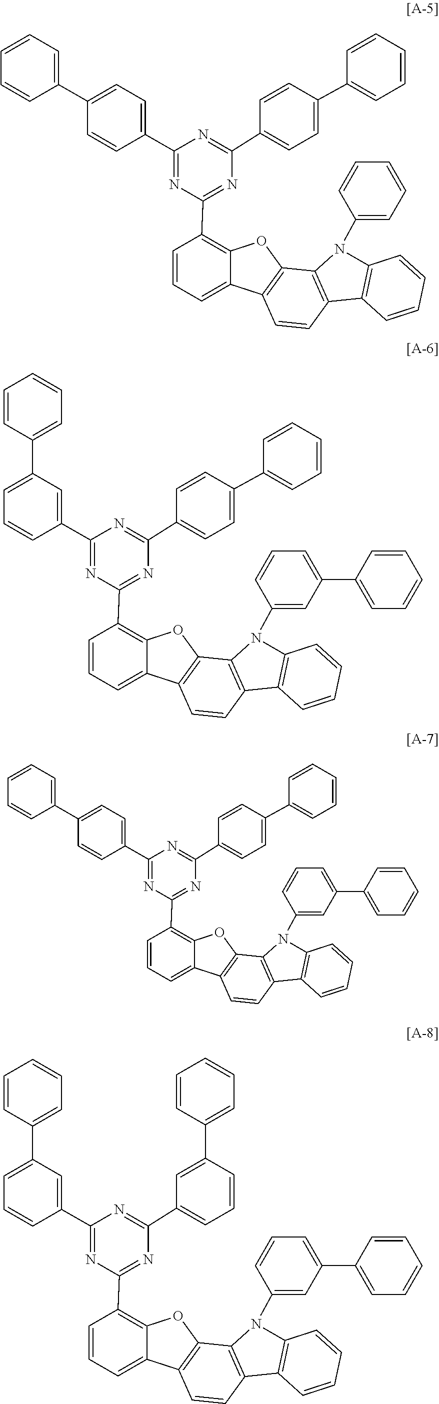 [SMILES: c1ccc(-c2ccc(-c3nc(-c4ccc(-c5ccccc5)cc4)nc(-c4cccc5c4oc4c5ccc5c6ccccc6n(-c6ccccc6)c54)n3)cc2)cc1 ; c1ccc(-c2ccc(-c3nc(-c4cccc(-c5ccccc5)c4)nc(-c4cccc5c4oc4c5ccc5c6ccccc6n(-c6cccc(-c7ccccc7)c6)c54)n3)cc2)cc1 ; c1ccc(-c2ccc(-c3nc(-c4ccc(-c5ccccc5)cc4)nc(-c4cccc5c4oc4c5ccc5c6ccccc6n(-c6cccc(-c7ccccc7)c6)c54)n3)cc2)cc1 ; c1ccc(-c2cccc(-c3nc(-c4cccc(-c5ccccc5)c4)nc(-c4cccc5c4oc4c5ccc5c6ccccc6n(-c6cccc(-c7ccccc7)c6)c54)n3)c2)cc1]

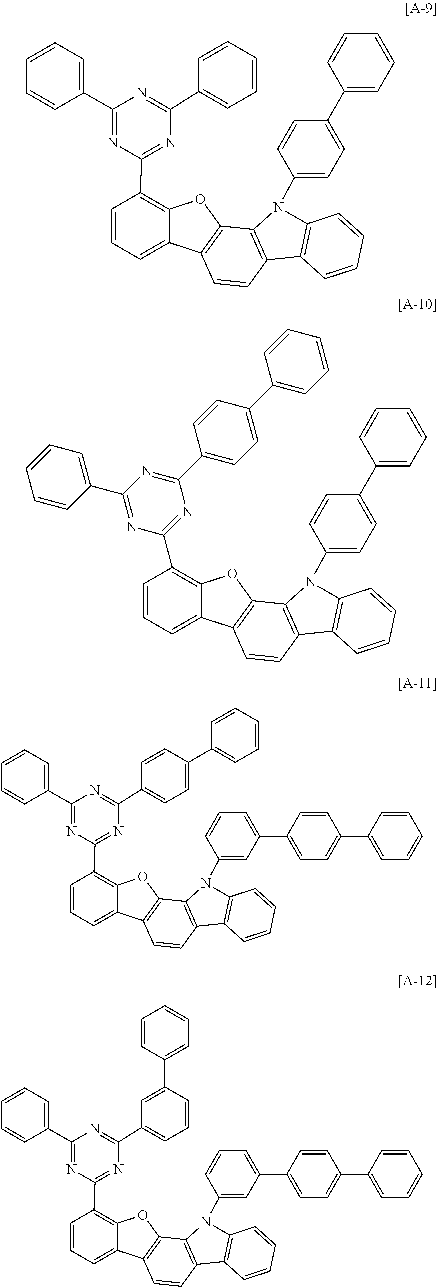 [SMILES: c1ccc(-c2ccc(-n3c4ccccc4c4ccc5c6cccc(-c7nc(-c8ccccc8)nc(-c8ccccc8)n7)c6oc5c43)cc2)cc1 ; c1ccc(-c2ccc(-c3nc(-c4ccccc4)nc(-c4cccc5c4oc4c5ccc5c6ccccc6n(-c6ccc(-c7ccccc7)cc6)c54)n3)cc2)cc1 ; c1ccc(-c2ccc(-c3cccc(-n4c5ccccc5c5ccc6c7cccc(-c8nc(-c9ccccc9)nc(-c9ccc(-c%10ccccc%10)cc9)n8)c7oc6c54)c3)cc2)cc1 ; c1ccc(-c2ccc(-c3cccc(-n4c5ccccc5c5ccc6c7cccc(-c8nc(-c9ccccc9)nc(-c9cccc(-c%10ccccc%10)c9)n8)c7oc6c54)c3)cc2)cc1]

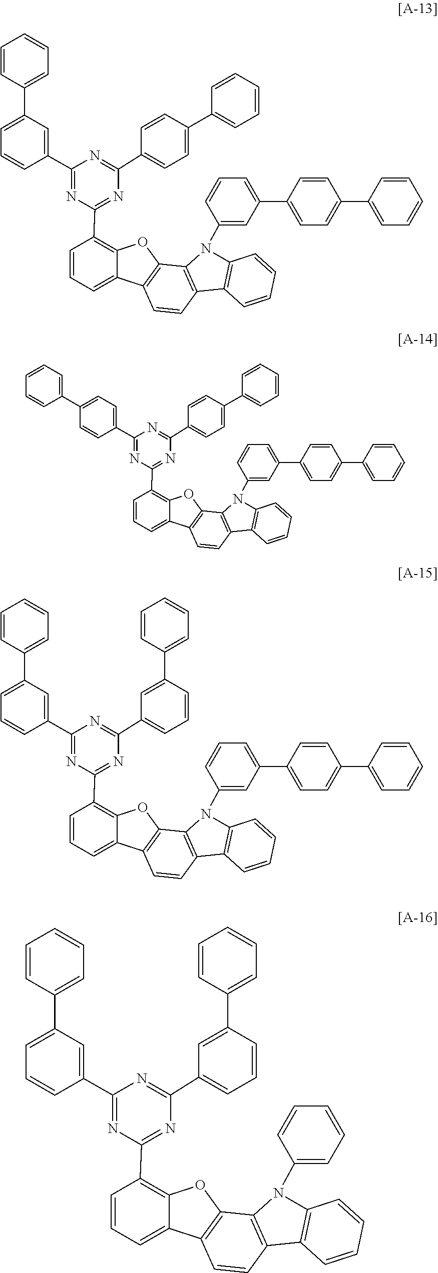 [SMILES: c1ccc(-c2ccc(-c3cccc(-n4c5ccccc5c5ccc6c7cccc(-c8nc(-c9ccc(-c%10ccccc%10)cc9)nc(-c9cccc(-c%10ccccc%10)c9)n8)c7oc6c54)c3)cc2)cc1 ; c1ccc(-c2ccc(-c3cccc(-n4c5ccccc5c5ccc6c7cccc(-c8nc(-c9ccc(-c%10ccccc%10)cc9)nc(-c9ccc(-c%10ccccc%10)cc9)n8)c7oc6c54)c3)cc2)cc1 ; c1ccc(-c2ccc(-c3cccc(-n4c5ccccc5c5ccc6c7cccc(-c8nc(-c9cccc(-c%10ccccc%10)c9)nc(-c9cccc(-c%10ccccc%10)c9)n8)c7oc6c54)c3)cc2)cc1 ; c1ccc(-c2cccc(-c3nc(-c4cccc(-c5ccccc5)c4)nc(-c4cccc5c4oc4c5ccc5c6ccccc6n(-c6ccccc6)c54)n3)c2)cc1]

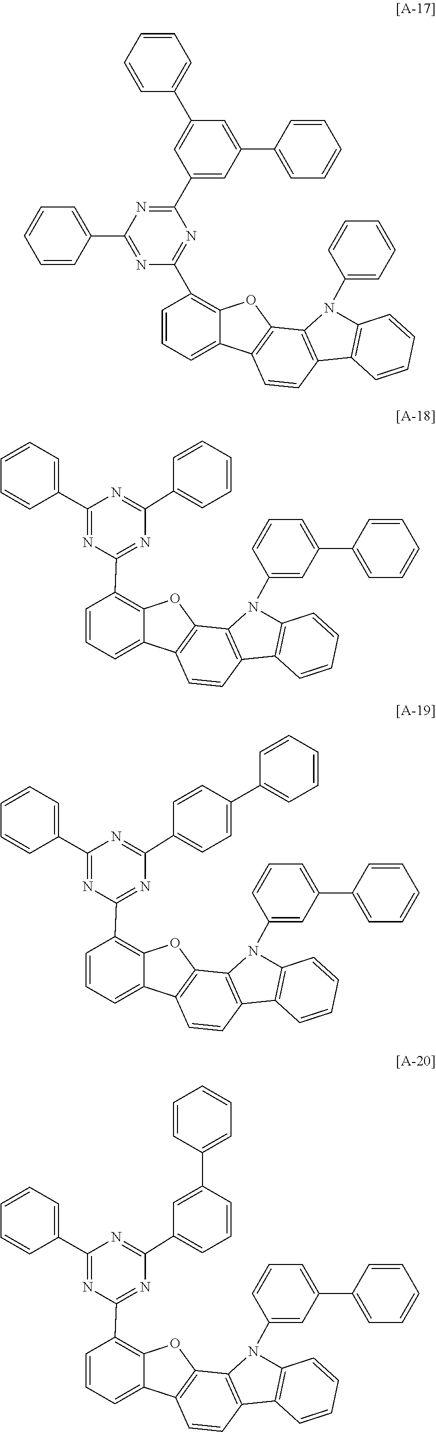 [SMILES: c1ccc(-c2cc(-c3ccccc3)cc(-c3nc(-c4ccccc4)nc(-c4cccc5c4oc4c5ccc5c6ccccc6n(-c6ccccc6)c54)n3)c2)cc1 ; c1ccc(-c2cccc(-n3c4ccccc4c4ccc5c6cccc(-c7nc(-c8ccccc8)nc(-c8ccccc8)n7)c6oc5c43)c2)cc1 ; c1ccc(-c2ccc(-c3nc(-c4ccccc4)nc(-c4cccc5c4oc4c5ccc5c6ccccc6n(-c6cccc(-c7ccccc7)c6)c54)n3)cc2)cc1 ; c1ccc(-c2cccc(-c3nc(-c4ccccc4)nc(-c4cccc5c4oc4c5ccc5c6ccccc6n(-c6cccc(-c7ccccc7)c6)c54)n3)c2)cc1]

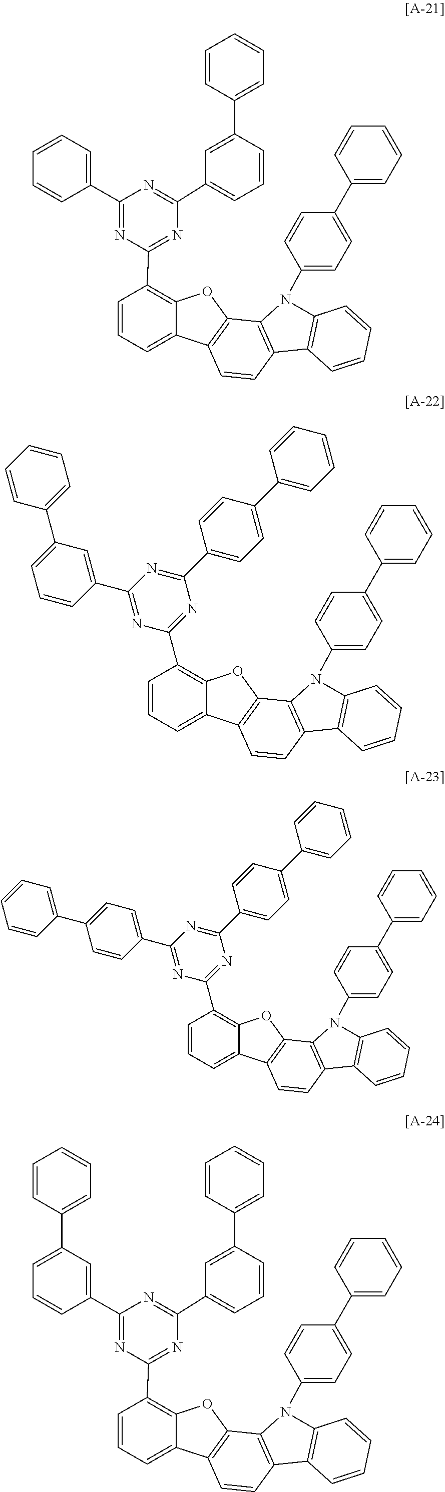 [SMILES: c1ccc(-c2ccc(-n3c4ccccc4c4ccc5c6cccc(-c7nc(-c8ccccc8)nc(-c8cccc(-c9ccccc9)c8)n7)c6oc5c43)cc2)cc1 ; c1ccc(-c2ccc(-c3nc(-c4cccc(-c5ccccc5)c4)nc(-c4cccc5c4oc4c5ccc5c6ccccc6n(-c6ccc(-c7ccccc7)cc6)c54)n3)cc2)cc1 ; c1ccc(-c2ccc(-c3nc(-c4ccc(-c5ccccc5)cc4)nc(-c4cccc5c4oc4c5ccc5c6ccccc6n(-c6ccc(-c7ccccc7)cc6)c54)n3)cc2)cc1 ; c1ccc(-c2ccc(-n3c4ccccc4c4ccc5c6cccc(-c7nc(-c8cccc(-c9ccccc9)c8)nc(-c8cccc(-c9ccccc9)c8)n7)c6oc5c43)cc2)cc1]

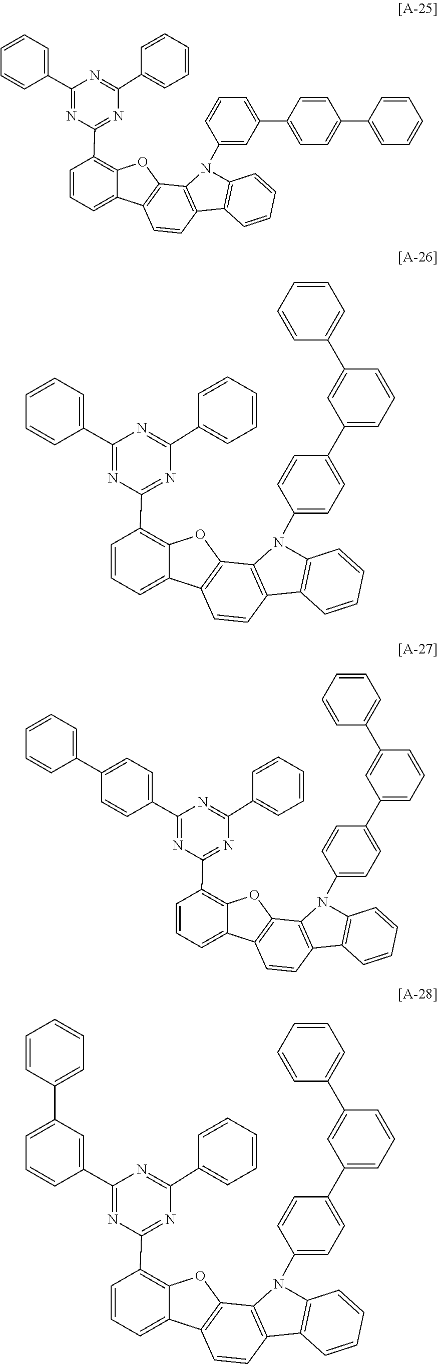 [SMILES: c1ccc(-c2ccc(-c3cccc(-n4c5ccccc5c5ccc6c7cccc(-c8nc(-c9ccccc9)nc(-c9ccccc9)n8)c7oc6c54)c3)cc2)cc1 ; c1ccc(-c2cccc(-c3ccc(-n4c5ccccc5c5ccc6c7cccc(-c8nc(-c9ccccc9)nc(-c9ccccc9)n8)c7oc6c54)cc3)c2)cc1 ; c1ccc(-c2ccc(-c3nc(-c4ccccc4)nc(-c4cccc5c4oc4c5ccc5c6ccccc6n(-c6ccc(-c7cccc(-c8ccccc8)c7)cc6)c54)n3)cc2)cc1 ; c1ccc(-c2cccc(-c3ccc(-n4c5ccccc5c5ccc6c7cccc(-c8nc(-c9ccccc9)nc(-c9cccc(-c%10ccccc%10)c9)n8)c7oc6c54)cc3)c2)cc1]

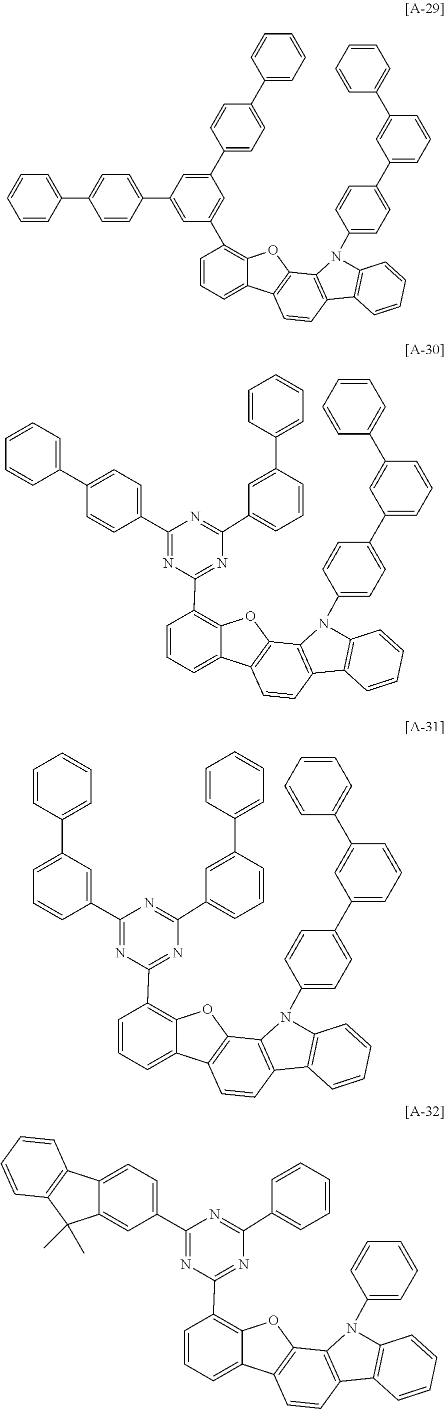 [SMILES: c1ccc(-c2ccc(-c3cc(-c4ccc(-c5ccccc5)cc4)cc(-c4cccc5c4oc4c5ccc5c6ccccc6n(-c6ccc(-c7cccc(-c8ccccc8)c7)cc6)c54)c3)cc2)cc1 ; c1ccc(-c2ccc(-c3nc(-c4cccc(-c5ccccc5)c4)nc(-c4cccc5c4oc4c5ccc5c6ccccc6n(-c6ccc(-c7cccc(-c8ccccc8)c7)cc6)c54)n3)cc2)cc1 ; c1ccc(-c2cccc(-c3ccc(-n4c5ccccc5c5ccc6c7cccc(-c8nc(-c9cccc(-c%10ccccc%10)c9)nc(-c9cccc(-c%10ccccc%10)c9)n8)c7oc6c54)cc3)c2)cc1 ; CC1(C)c2ccccc2-c2ccc(-c3nc(-c4ccccc4)nc(-c4cccc5c4oc4c5ccc5c6ccccc6n(-c6ccccc6)c54)n3)cc21]

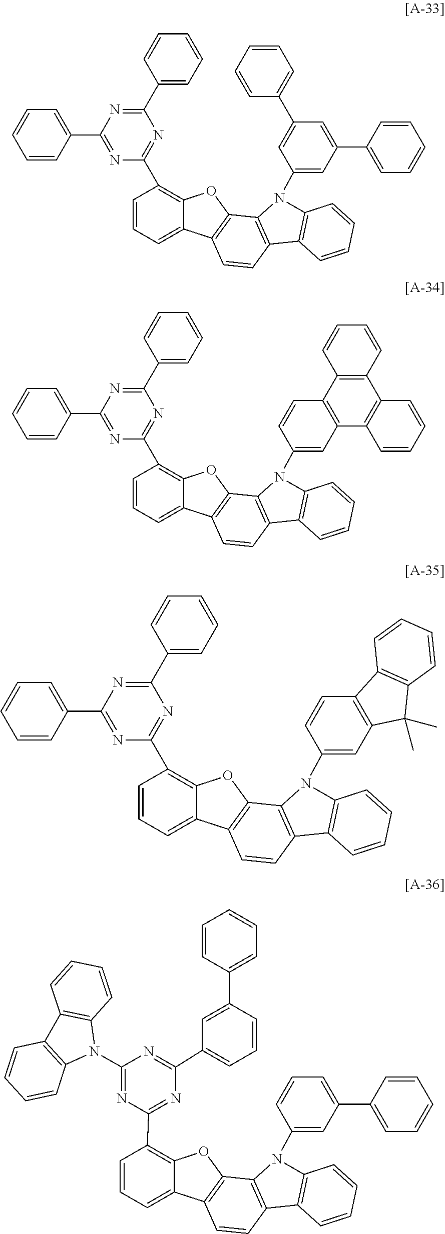 [SMILES: c1ccc(-c2cc(-c3ccccc3)cc(-n3c4ccccc4c4ccc5c6cccc(-c7nc(-c8ccccc8)nc(-c8ccccc8)n7)c6oc5c43)c2)cc1 ; c1ccc(-c2nc(-c3ccccc3)nc(-c3cccc4c3oc3c4ccc4c5ccccc5n(-c5ccc6c7ccccc7c7ccccc7c6c5)c43)n2)cc1 ; CC1(C)c2ccccc2-c2ccc(-n3c4ccccc4c4ccc5c6cccc(-c7nc(-c8ccccc8)nc(-c8ccccc8)n7)c6oc5c43)cc21 ; c1ccc(-c2cccc(-c3nc(-c4cccc5c4oc4c5ccc5c6ccccc6n(-c6cccc(-c7ccccc7)c6)c54)nc(-n4c5ccccc5c5ccccc54)n3)c2)cc1]

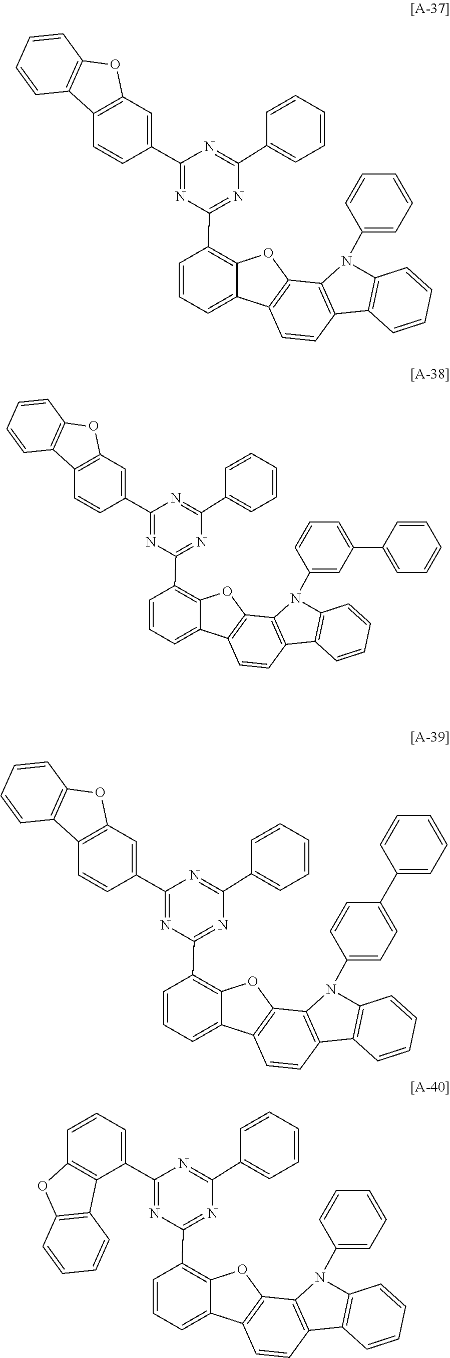

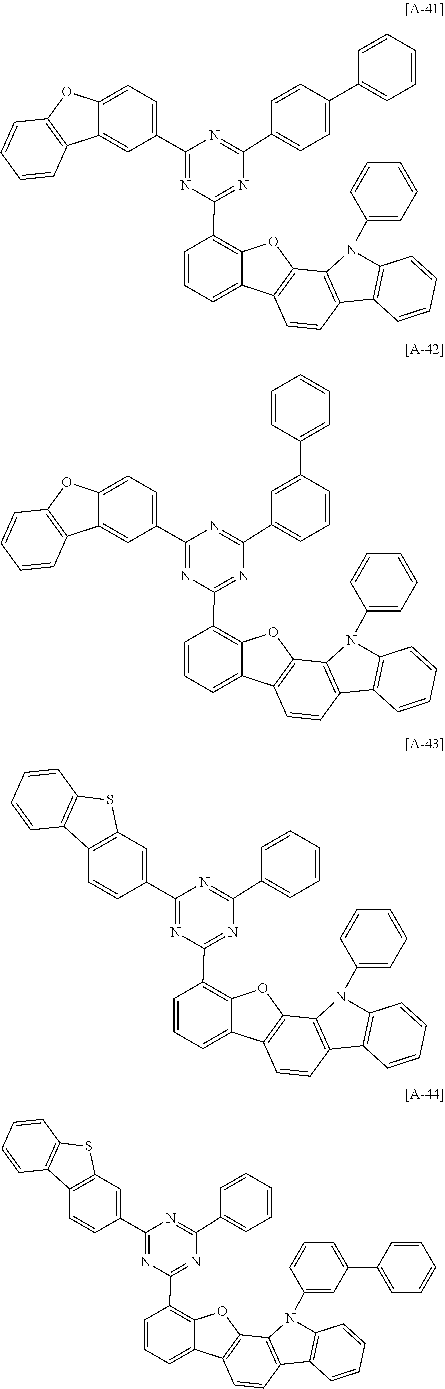 [SMILES: c1ccc(-c2ccc(-c3nc(-c4ccc5oc6ccccc6c5c4)nc(-c4cccc5c4oc4c5ccc5c6ccccc6n(-c6ccccc6)c54)n3)cc2)cc1 ; c1ccc(-c2cccc(-c3nc(-c4ccc5oc6ccccc6c5c4)nc(-c4cccc5c4oc4c5ccc5c6ccccc6n(-c6ccccc6)c54)n3)c2)cc1 ; c1ccc(-c2nc(-c3ccc4c(c3)sc3ccccc34)nc(-c3cccc4c3oc3c4ccc4c5ccccc5n(-c5ccccc5)c43)n2)cc1 ; c1ccc(-c2cccc(-n3c4ccccc4c4ccc5c6cccc(-c7nc(-c8ccccc8)nc(-c8ccc9c(c8)sc8ccccc89)n7)c6oc5c43)c2)cc1]

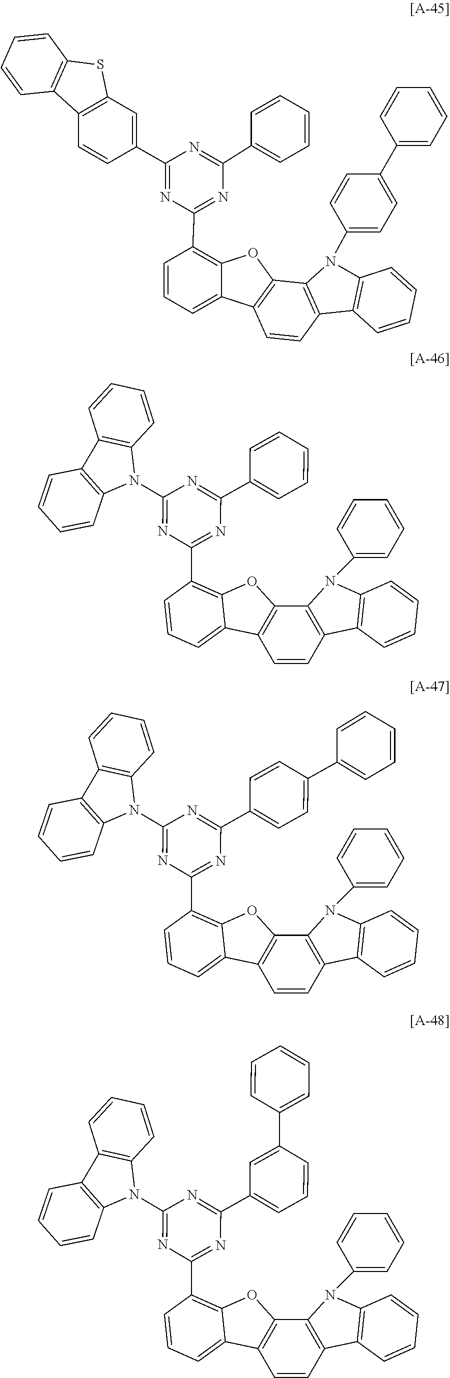 [SMILES: c1ccc(-c2ccc(-n3c4ccccc4c4ccc5c6cccc(-c7nc(-c8ccccc8)nc(-c8ccc9c(c8)sc8ccccc89)n7)c6oc5c43)cc2)cc1 ; c1ccc(-c2nc(-c3cccc4c3oc3c4ccc4c5ccccc5n(-c5ccccc5)c43)nc(-n3c4ccccc4c4ccccc43)n2)cc1 ; c1ccc(-c2ccc(-c3nc(-c4cccc5c4oc4c5ccc5c6ccccc6n(-c6ccccc6)c54)nc(-n4c5ccccc5c5ccccc54)n3)cc2)cc1 ; c1ccc(-c2cccc(-c3nc(-c4cccc5c4oc4c5ccc5c6ccccc6n(-c6ccccc6)c54)nc(-n4c5ccccc5c5ccccc54)n3)c2)cc1]

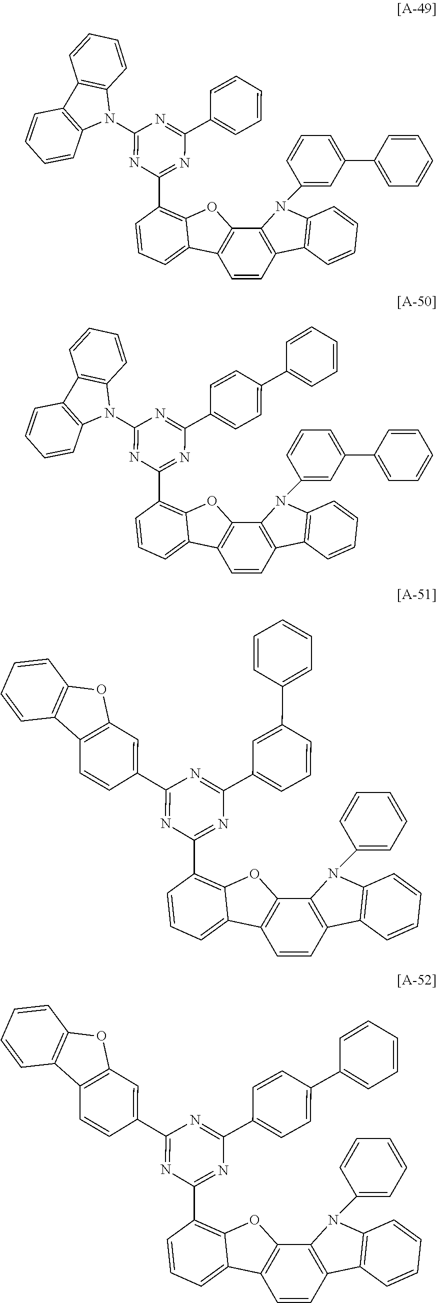 [SMILES: c1ccc(-c2cccc(-n3c4ccccc4c4ccc5c6cccc(-c7nc(-c8ccccc8)nc(-n8c9ccccc9c9ccccc98)n7)c6oc5c43)c2)cc1 ; c1ccc(-c2ccc(-c3nc(-c4cccc5c4oc4c5ccc5c6ccccc6n(-c6cccc(-c7ccccc7)c6)c54)nc(-n4c5ccccc5c5ccccc54)n3)cc2)cc1 ; c1ccc(-c2cccc(-c3nc(-c4ccc5c(c4)oc4ccccc45)nc(-c4cccc5c4oc4c5ccc5c6ccccc6n(-c6ccccc6)c54)n3)c2)cc1 ; c1ccc(-c2ccc(-c3nc(-c4ccc5c(c4)oc4ccccc45)nc(-c4cccc5c4oc4c5ccc5c6ccccc6n(-c6ccccc6)c54)n3)cc2)cc1]

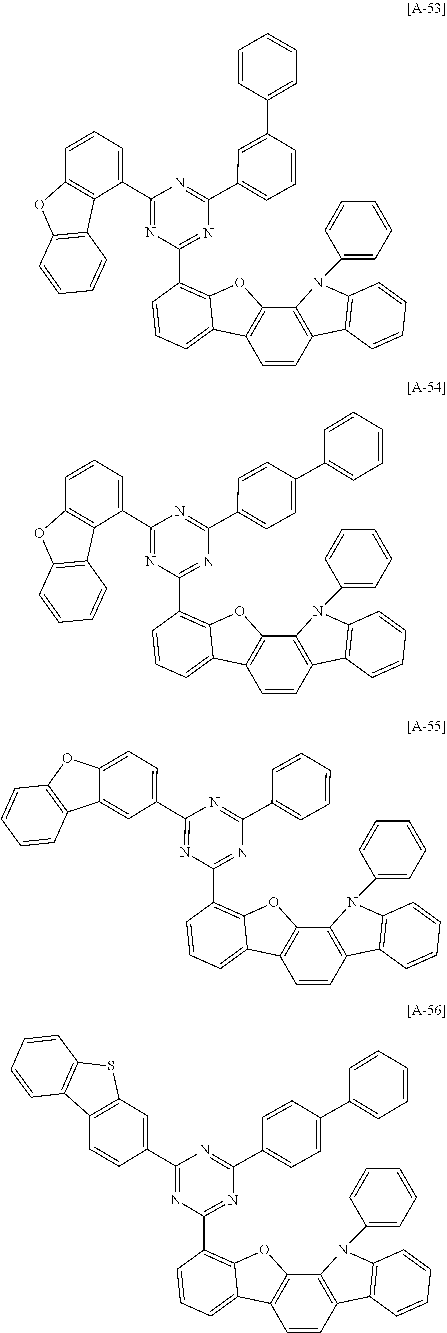 [SMILES: c1ccc(-c2cccc(-c3nc(-c4cccc5c4oc4c5ccc5c6ccccc6n(-c6ccccc6)c54)nc(-c4cccc5oc6ccccc6c45)n3)c2)cc1 ; c1ccc(-c2ccc(-c3nc(-c4cccc5c4oc4c5ccc5c6ccccc6n(-c6ccccc6)c54)nc(-c4cccc5oc6ccccc6c45)n3)cc2)cc1 ; c1ccc(-c2nc(-c3ccc4oc5ccccc5c4c3)nc(-c3cccc4c3oc3c4ccc4c5ccccc5n(-c5ccccc5)c43)n2)cc1 ; c1ccc(-c2ccc(-c3nc(-c4ccc5c(c4)sc4ccccc45)nc(-c4cccc5c4oc4c5ccc5c6ccccc6n(-c6ccccc6)c54)n3)cc2)cc1]

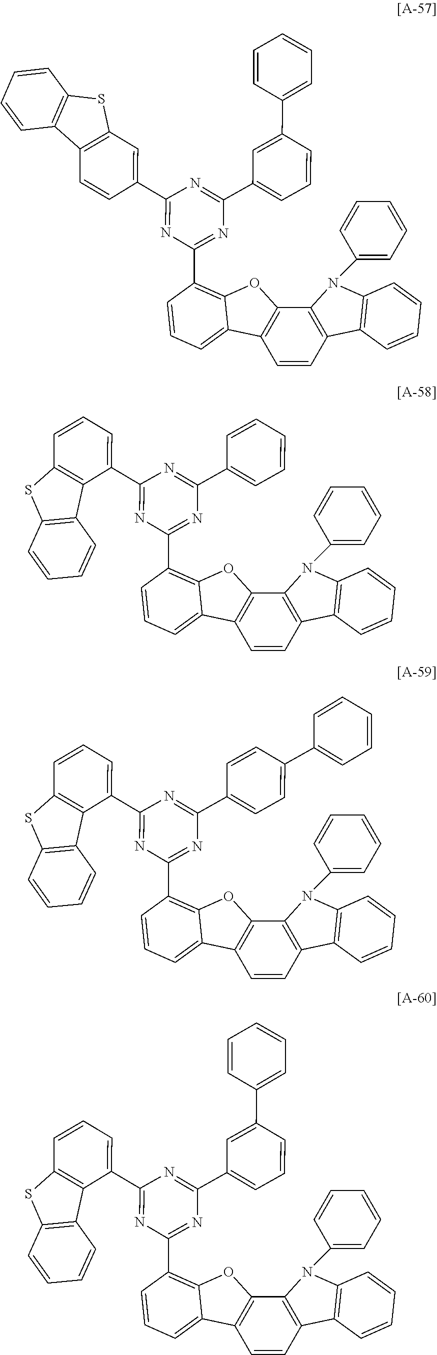

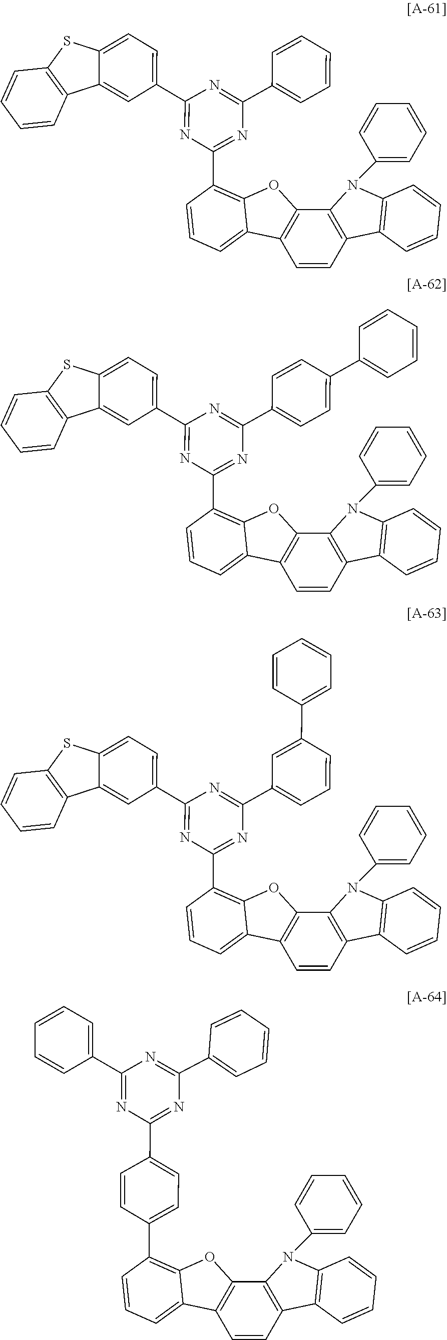 [SMILES: c1ccc(-c2nc(-c3ccc4sc5ccccc5c4c3)nc(-c3cccc4c3oc3c4ccc4c5ccccc5n(-c5ccccc5)c43)n2)cc1 ; c1ccc(-c2ccc(-c3nc(-c4ccc5sc6ccccc6c5c4)nc(-c4cccc5c4oc4c5ccc5c6ccccc6n(-c6ccccc6)c54)n3)cc2)cc1 ; c1ccc(-c2cccc(-c3nc(-c4ccc5sc6ccccc6c5c4)nc(-c4cccc5c4oc4c5ccc5c6ccccc6n(-c6ccccc6)c54)n3)c2)cc1 ; c1ccc(-c2nc(-c3ccccc3)nc(-c3ccc(-c4cccc5c4oc4c5ccc5c6ccccc6n(-c6ccccc6)c54)cc3)n2)cc1]

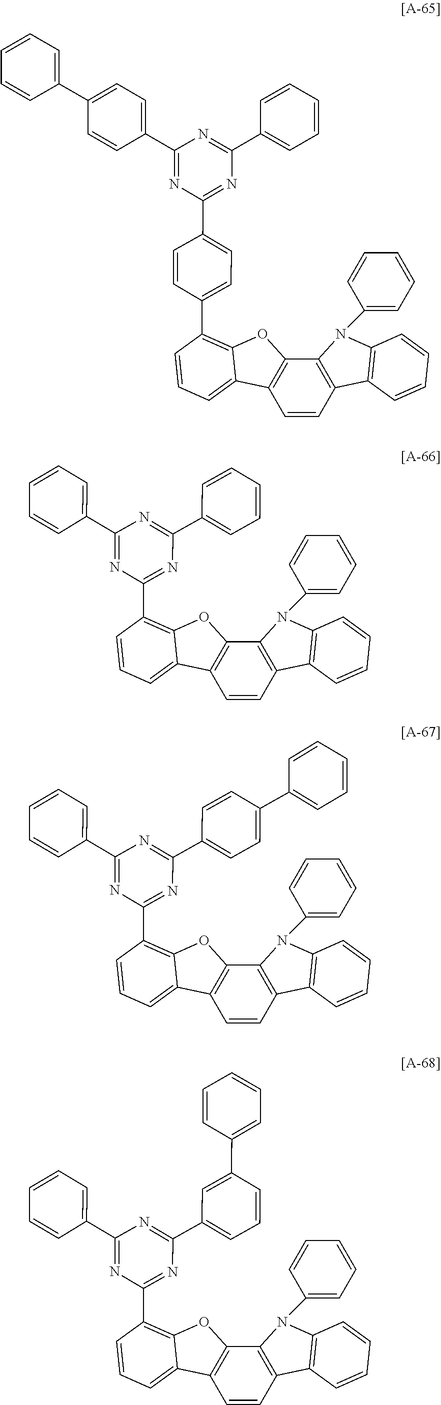 [SMILES: c1ccc(-c2ccc(-c3nc(-c4ccccc4)nc(-c4ccc(-c5cccc6c5oc5c6ccc6c7ccccc7n(-c7ccccc7)c65)cc4)n3)cc2)cc1 ; c1ccc(-c2nc(-c3ccccc3)nc(-c3cccc4c3oc3c4ccc4c5ccccc5n(-c5ccccc5)c43)n2)cc1 ; c1ccc(-c2ccc(-c3nc(-c4ccccc4)nc(-c4cccc5c4oc4c5ccc5c6ccccc6n(-c6ccccc6)c54)n3)cc2)cc1 ; c1ccc(-c2cccc(-c3nc(-c4ccccc4)nc(-c4cccc5c4oc4c5ccc5c6ccccc6n(-c6ccccc6)c54)n3)c2)cc1]

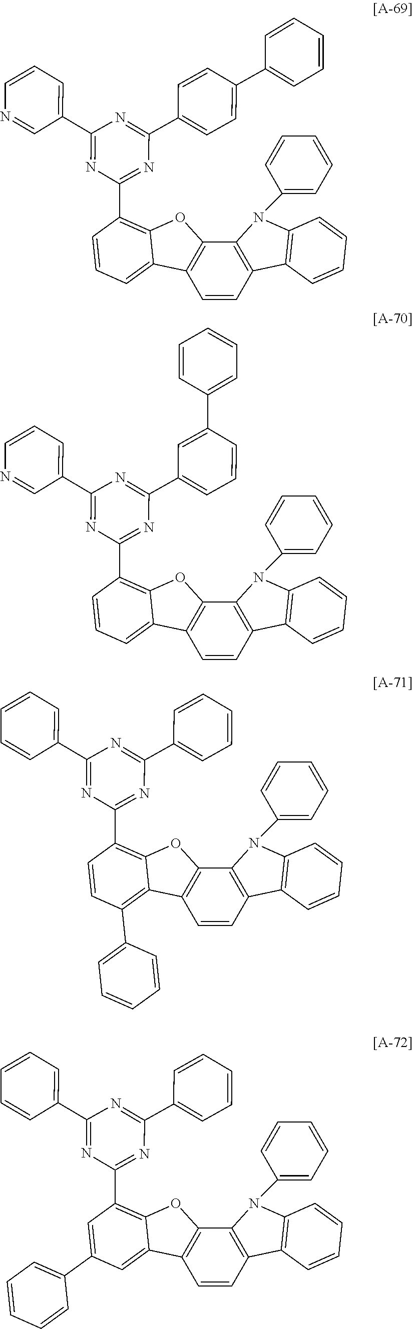

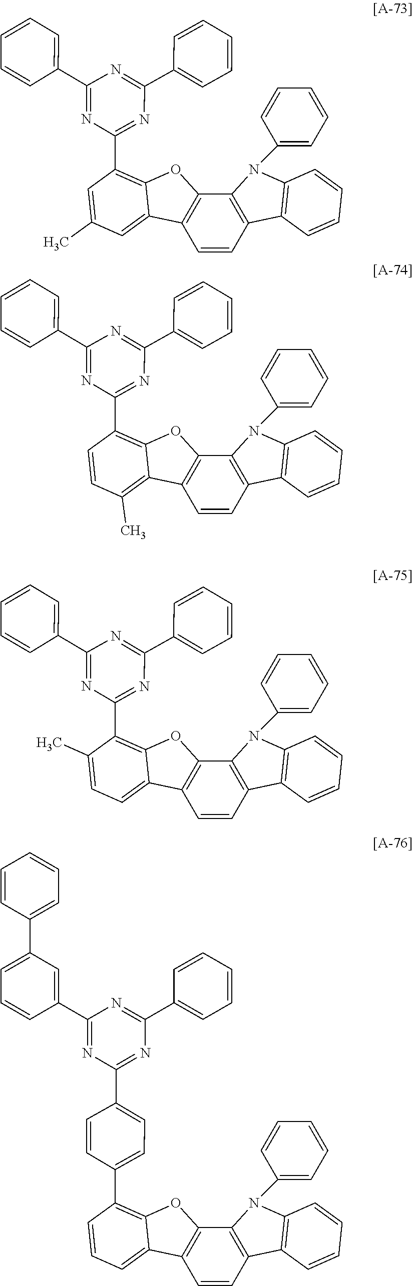

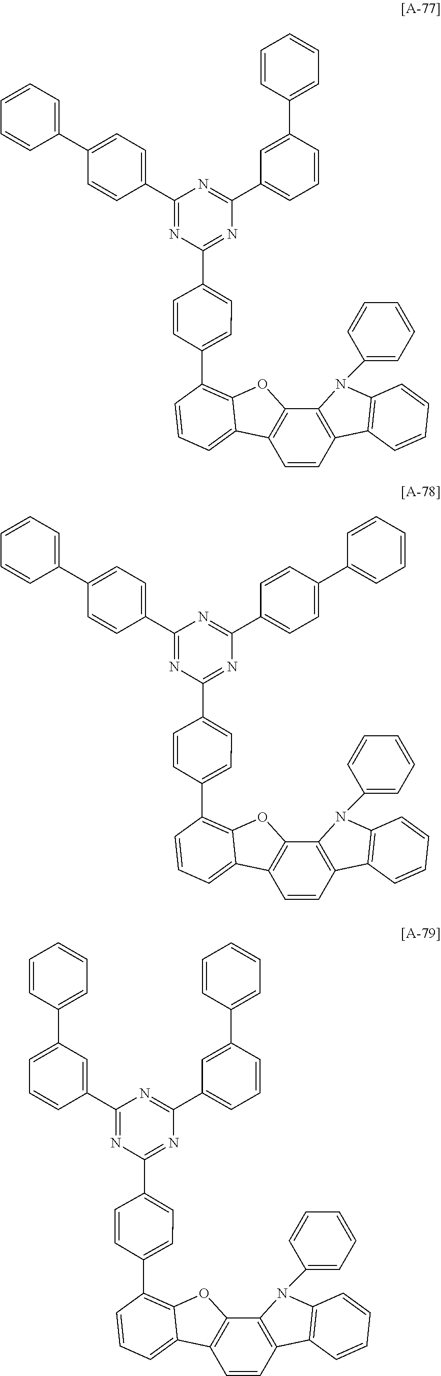 [SMILES: c1ccc(-c2ccc(-c3nc(-c4ccc(-c5cccc6c5oc5c6ccc6c7ccccc7n(-c7ccccc7)c65)cc4)nc(-c4cccc(-c5ccccc5)c4)n3)cc2)cc1 ; c1ccc(-c2ccc(-c3nc(-c4ccc(-c5ccccc5)cc4)nc(-c4ccc(-c5cccc6c5oc5c6ccc6c7ccccc7n(-c7ccccc7)c65)cc4)n3)cc2)cc1 ; c1ccc(-c2cccc(-c3nc(-c4ccc(-c5cccc6c5oc5c6ccc6c7ccccc7n(-c7ccccc7)c65)cc4)nc(-c4cccc(-c5ccccc5)c4)n3)c2)cc1]

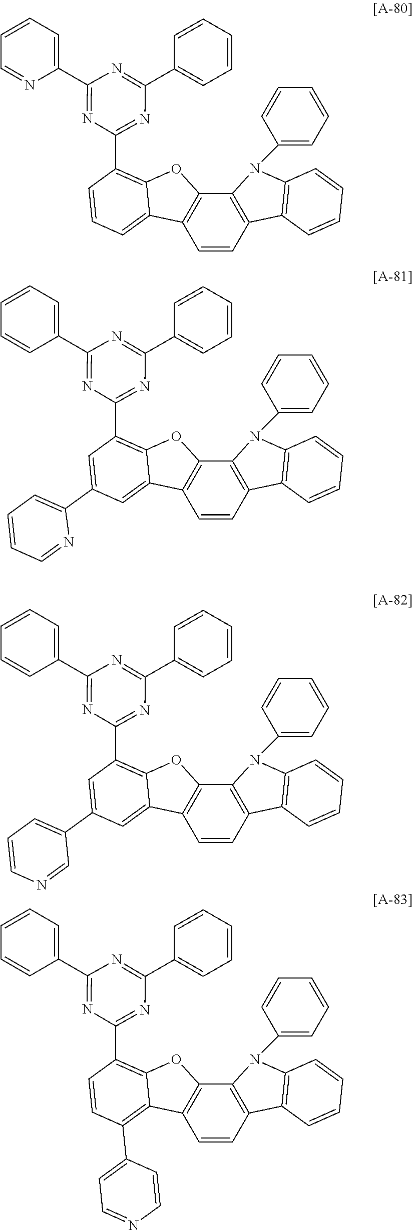

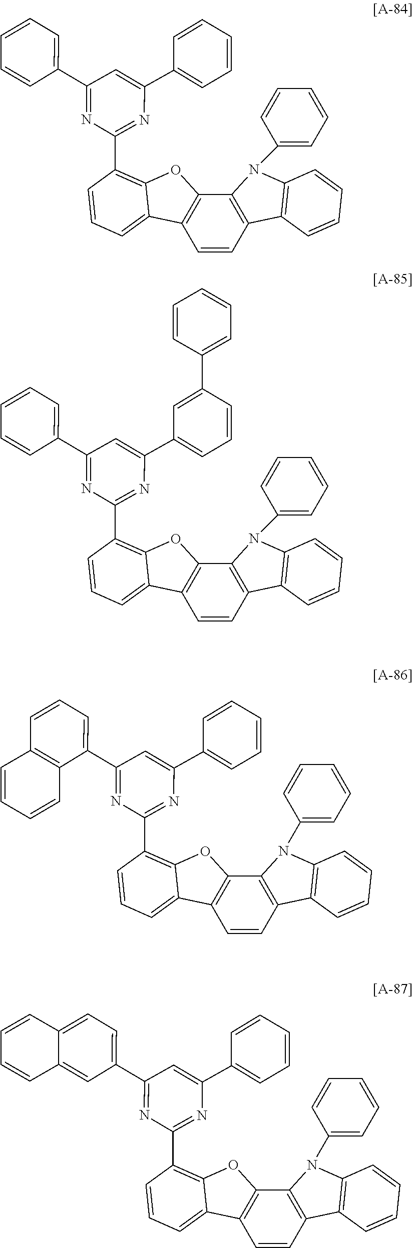

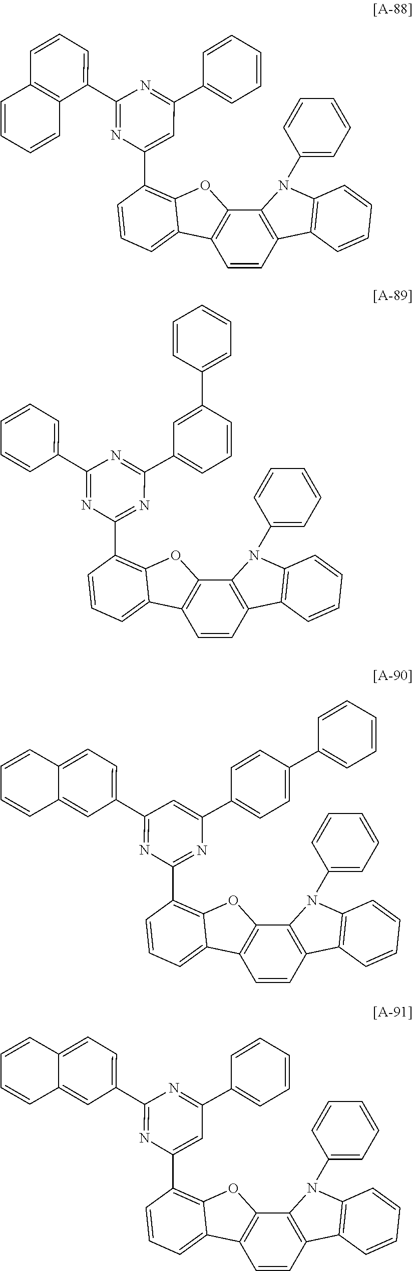

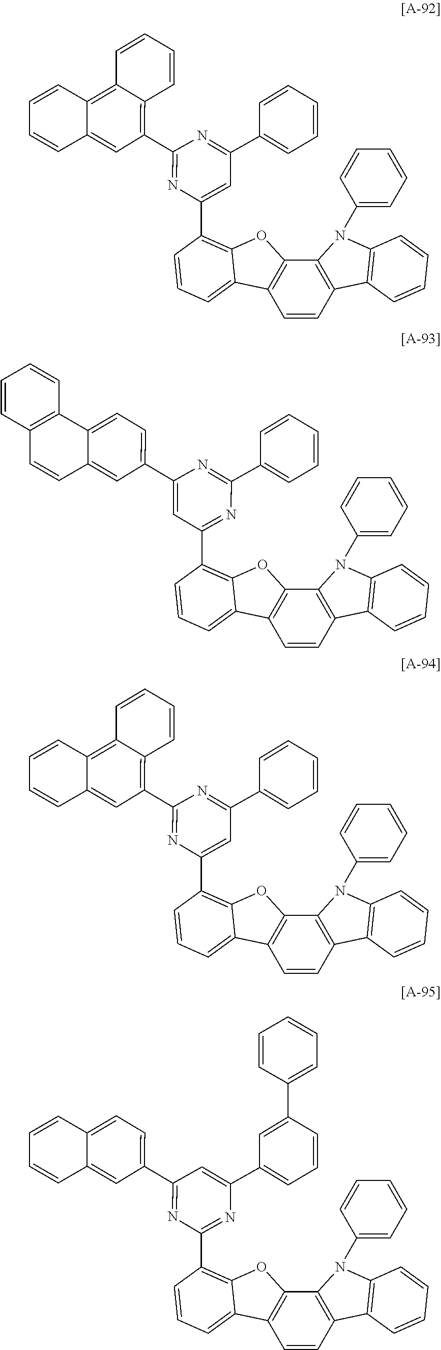

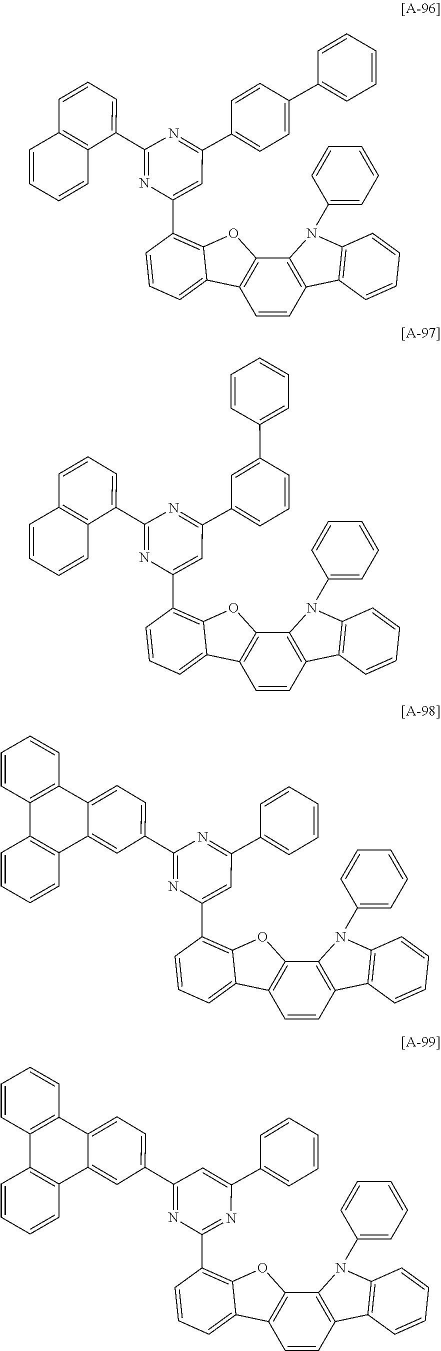

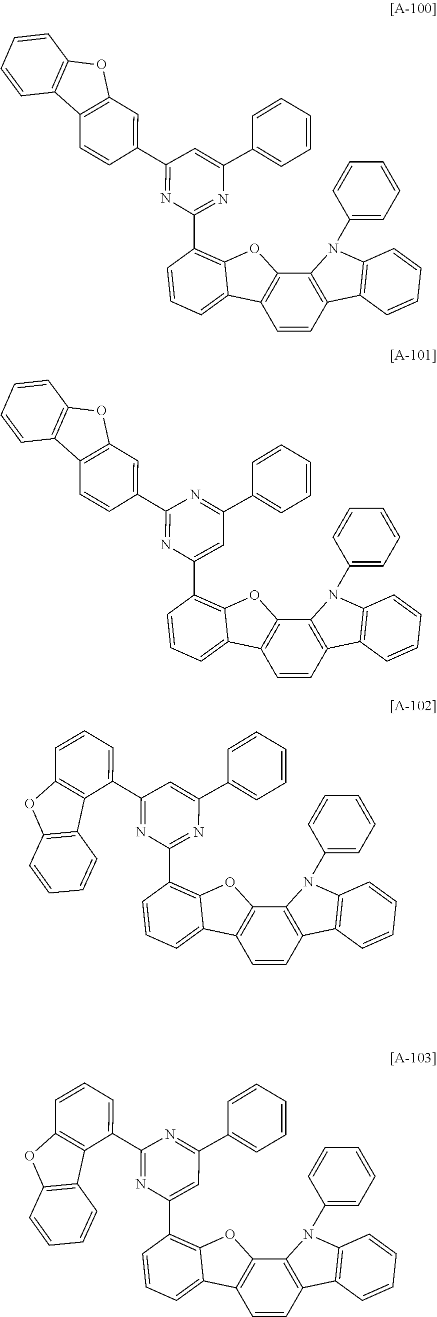 [SMILES: c1ccc(-c2cc(-c3ccc4c(c3)oc3ccccc34)nc(-c3cccc4c3oc3c4ccc4c5ccccc5n(-c5ccccc5)c43)n2)cc1 ; c1ccc(-c2cc(-c3cccc4c3oc3c4ccc4c5ccccc5n(-c5ccccc5)c43)nc(-c3ccc4c(c3)oc3ccccc34)n2)cc1 ; c1ccc(-c2cc(-c3cccc4oc5ccccc5c34)nc(-c3cccc4c3oc3c4ccc4c5ccccc5n(-c5ccccc5)c43)n2)cc1 ; c1ccc(-c2cc(-c3cccc4c3oc3c4ccc4c5ccccc5n(-c5ccccc5)c43)nc(-c3cccc4oc5ccccc5c34)n2)cc1]

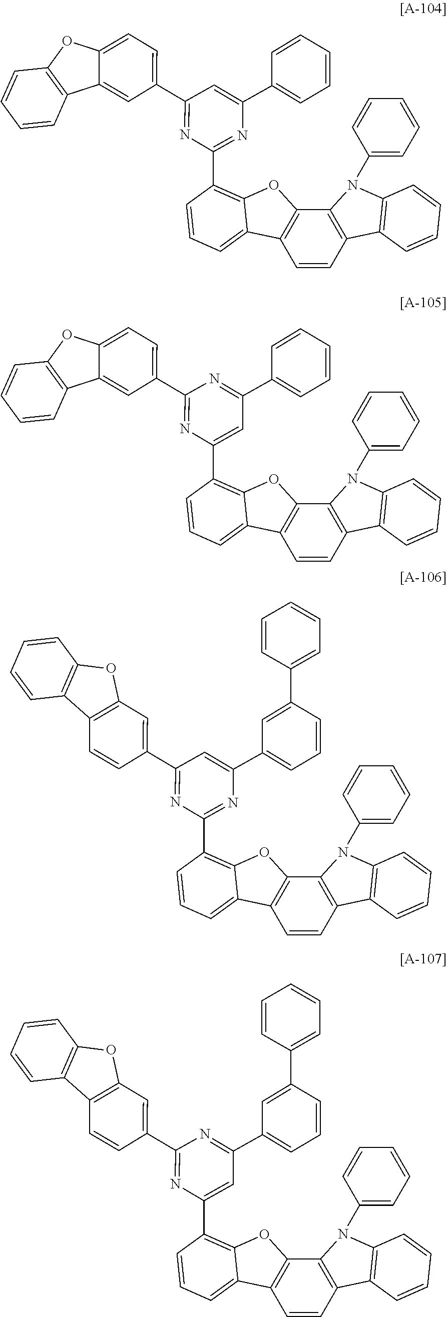

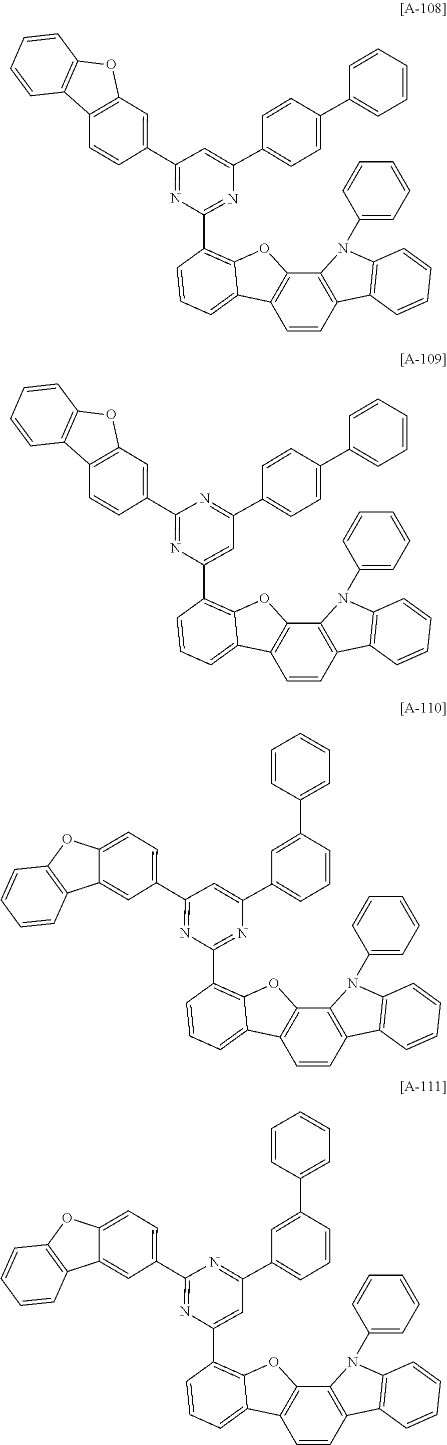

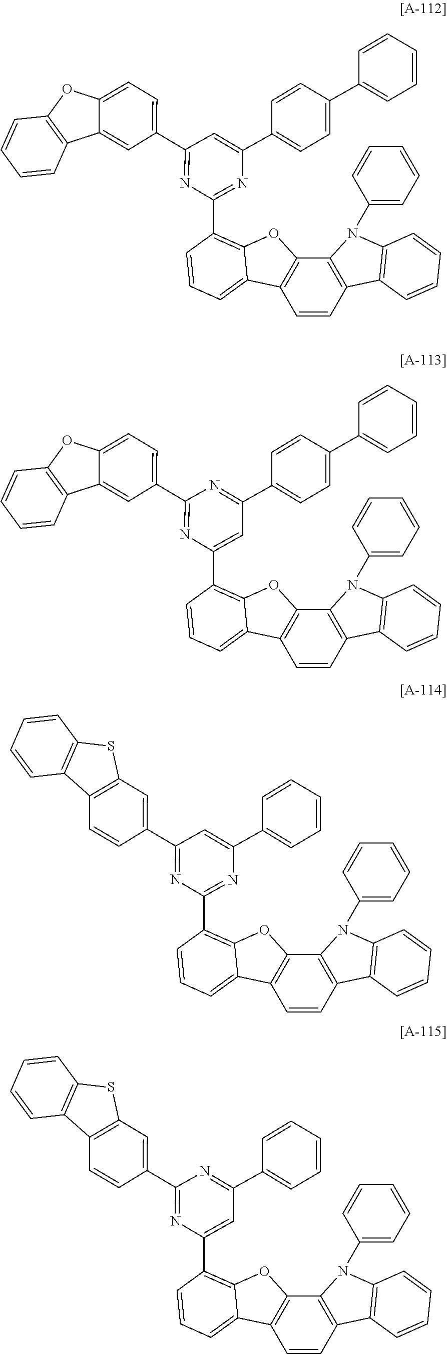 [SMILES: c1ccc(-c2ccc(-c3cc(-c4ccc5oc6ccccc6c5c4)nc(-c4cccc5c4oc4c5ccc5c6ccccc6n(-c6ccccc6)c54)n3)cc2)cc1 ; c1ccc(-c2ccc(-c3cc(-c4cccc5c4oc4c5ccc5c6ccccc6n(-c6ccccc6)c54)nc(-c4ccc5oc6ccccc6c5c4)n3)cc2)cc1 ; c1ccc(-c2cc(-c3ccc4c(c3)sc3ccccc34)nc(-c3cccc4c3oc3c4ccc4c5ccccc5n(-c5ccccc5)c43)n2)cc1 ; c1ccc(-c2cc(-c3cccc4c3oc3c4ccc4c5ccccc5n(-c5ccccc5)c43)nc(-c3ccc4c(c3)sc3ccccc34)n2)cc1]

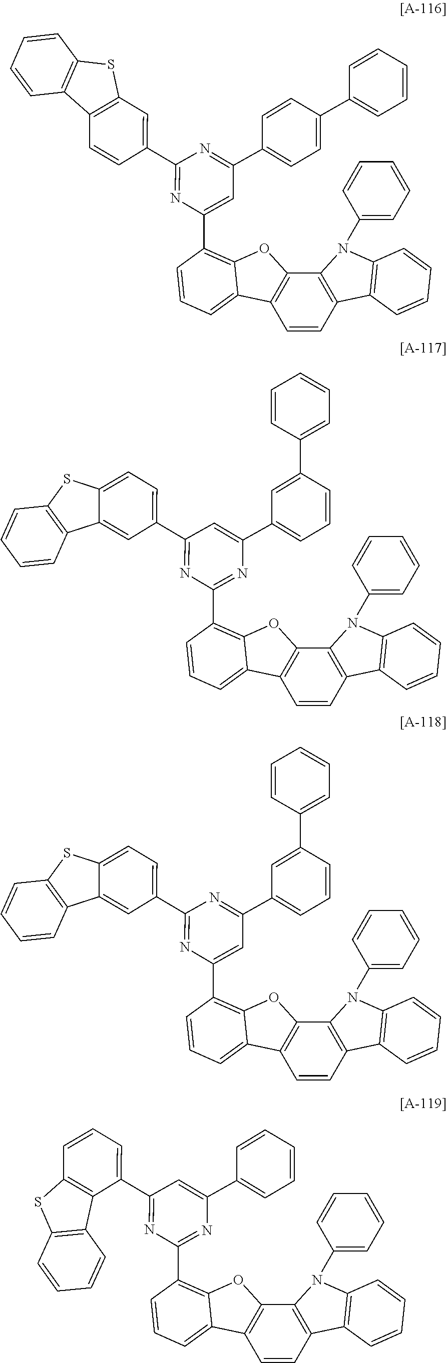

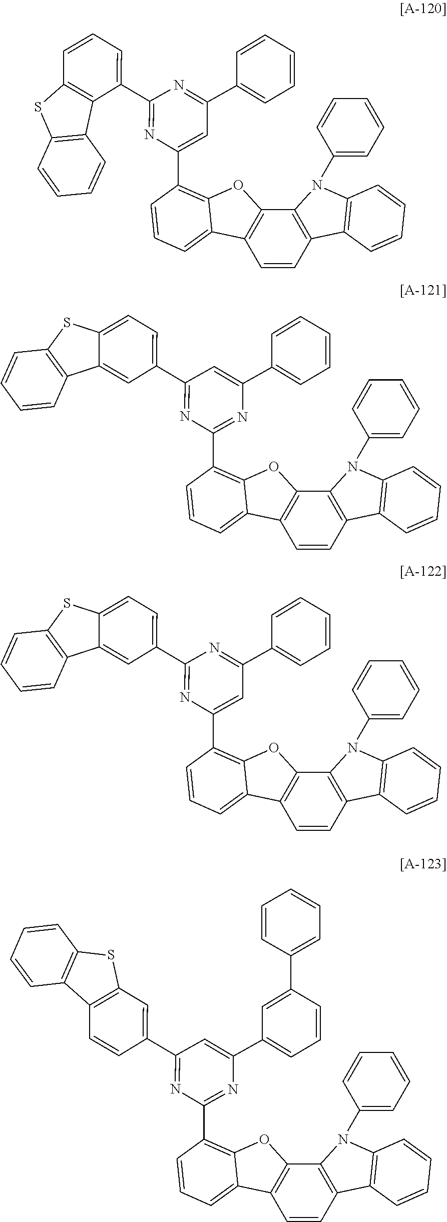 [SMILES: c1ccc(-c2cc(-c3cccc4c3oc3c4ccc4c5ccccc5n(-c5ccccc5)c43)nc(-c3cccc4sc5ccccc5c34)n2)cc1 ; c1ccc(-c2cc(-c3ccc4sc5ccccc5c4c3)nc(-c3cccc4c3oc3c4ccc4c5ccccc5n(-c5ccccc5)c43)n2)cc1 ; c1ccc(-c2cc(-c3cccc4c3oc3c4ccc4c5ccccc5n(-c5ccccc5)c43)nc(-c3ccc4sc5ccccc5c4c3)n2)cc1 ; c1ccc(-c2cccc(-c3cc(-c4ccc5c(c4)sc4ccccc45)nc(-c4cccc5c4oc4c5ccc5c6ccccc6n(-c6ccccc6)c54)n3)c2)cc1]

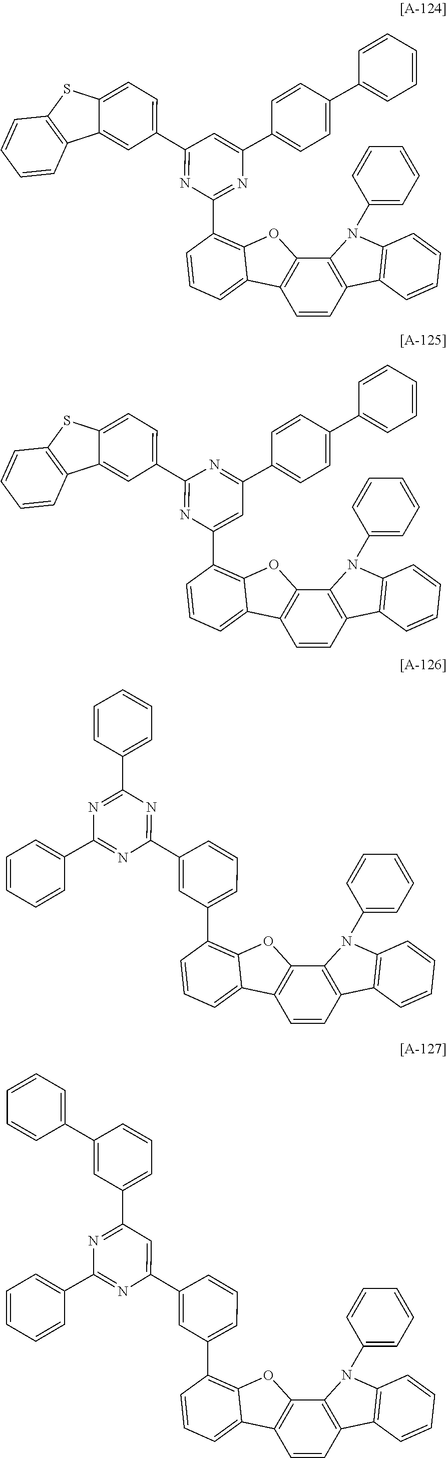 [SMILES: c1ccc(-c2ccc(-c3cc(-c4ccc5sc6ccccc6c5c4)nc(-c4cccc5c4oc4c5ccc5c6ccccc6n(-c6ccccc6)c54)n3)cc2)cc1 ; c1ccc(-c2ccc(-c3cc(-c4cccc5c4oc4c5ccc5c6ccccc6n(-c6ccccc6)c54)nc(-c4ccc5sc6ccccc6c5c4)n3)cc2)cc1 ; c1ccc(-c2nc(-c3ccccc3)nc(-c3cccc(-c4cccc5c4oc4c5ccc5c6ccccc6n(-c6ccccc6)c54)c3)n2)cc1 ; c1ccc(-c2cccc(-c3cc(-c4cccc(-c5cccc6c5oc5c6ccc6c7ccccc7n(-c7ccccc7)c65)c4)nc(-c4ccccc4)n3)c2)cc1]

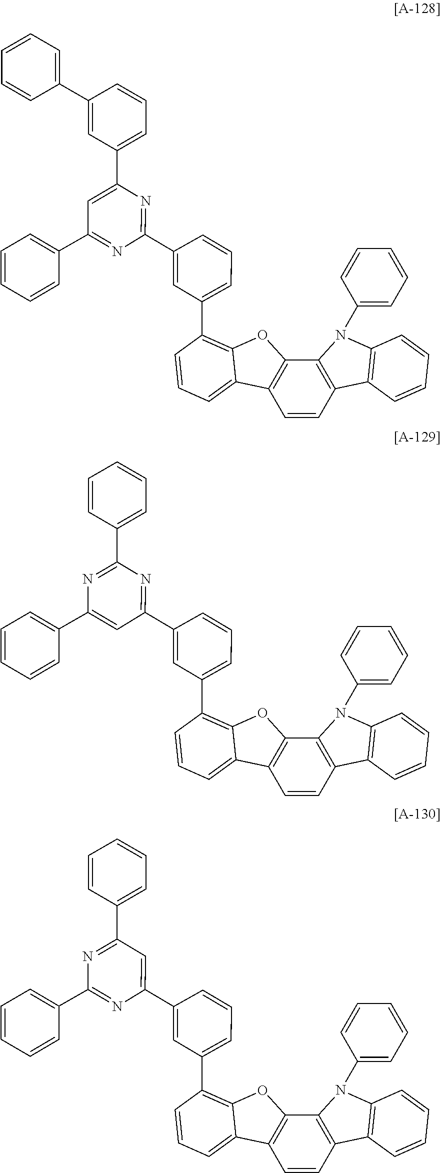 [SMILES: c1ccc(-c2cccc(-c3cc(-c4ccccc4)nc(-c4cccc(-c5cccc6c5oc5c6ccc6c7ccccc7n(-c7ccccc7)c65)c4)n3)c2)cc1 ; c1ccc(-c2cc(-c3cccc(-c4cccc5c4oc4c5ccc5c6ccccc6n(-c6ccccc6)c54)c3)nc(-c3ccccc3)n2)cc1 ; c1ccc(-c2cc(-c3cccc(-c4cccc5c4oc4c5ccc5c6ccccc6n(-c6ccccc6)c54)c3)nc(-c3ccccc3)n2)cc1]

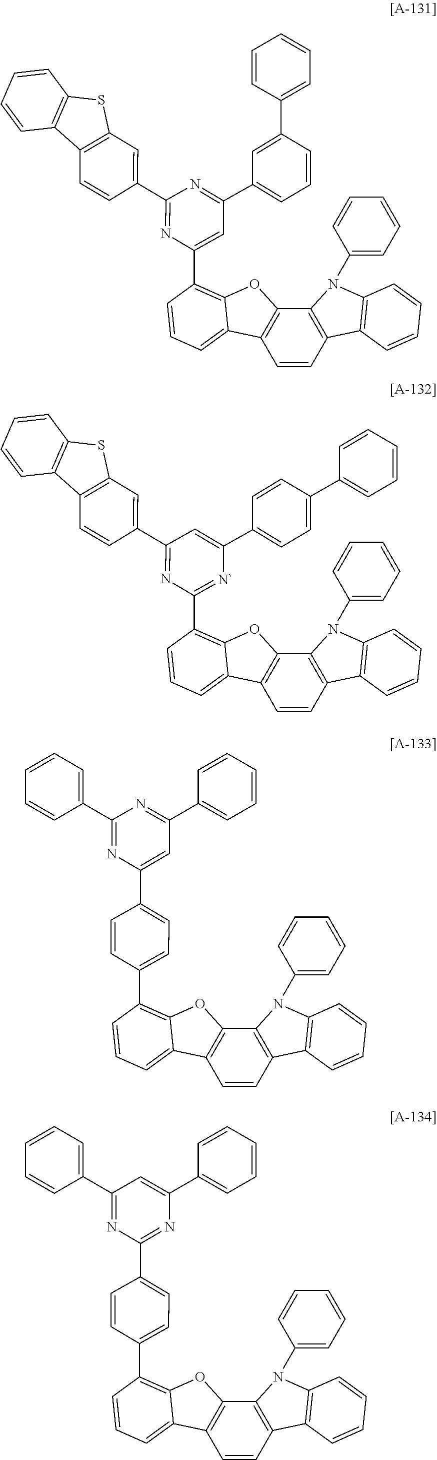

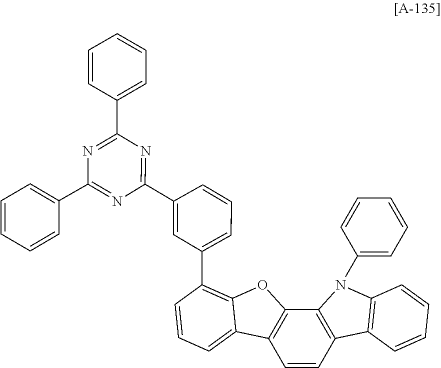 [SMILES: c1ccc(-c2nc(-c3ccccc3)nc(-c3cccc(-c4cccc5c4oc4c5ccc5c6ccccc6n(-c6ccccc6)c54)c3)n2)cc1]

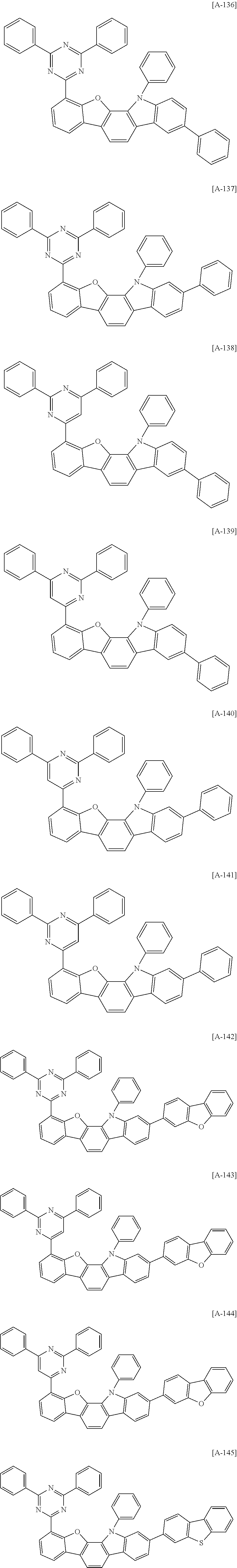

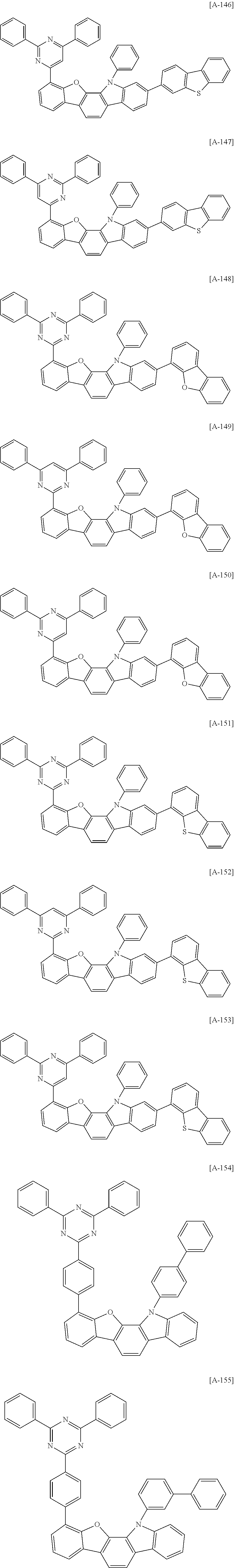

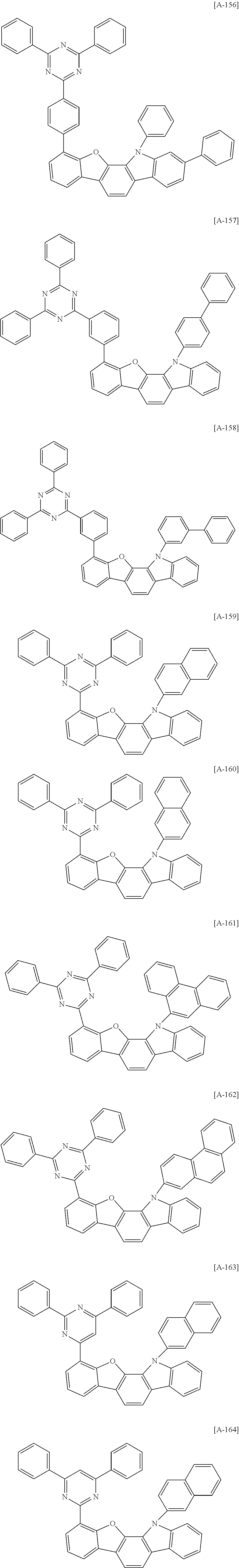

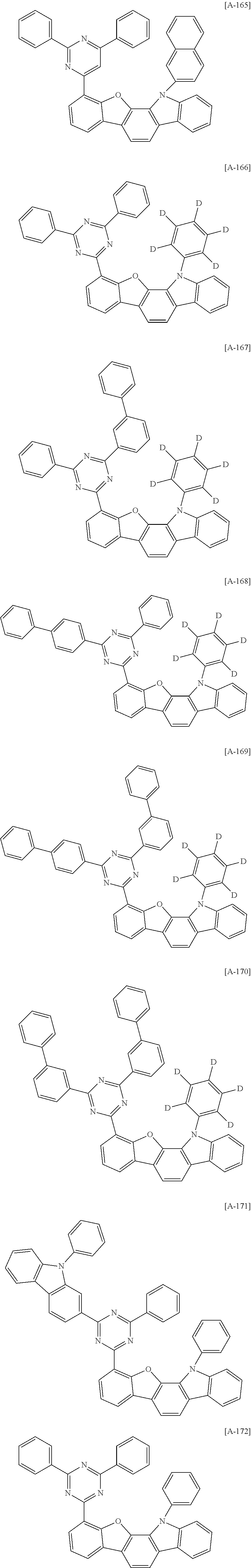

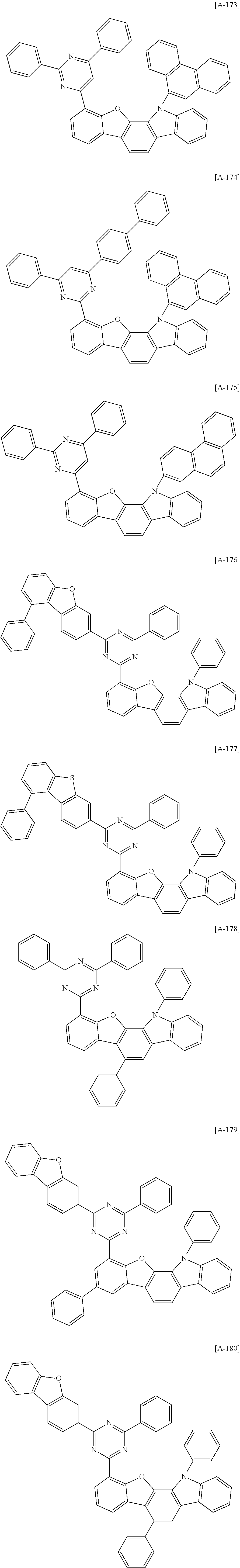

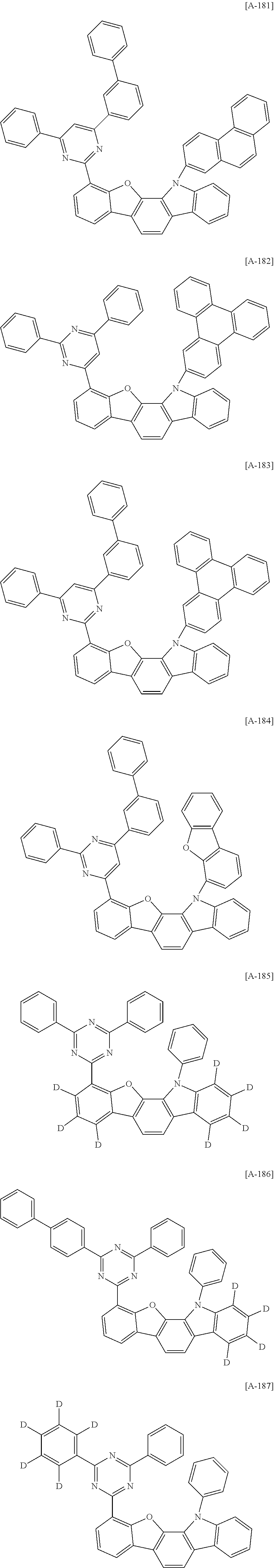

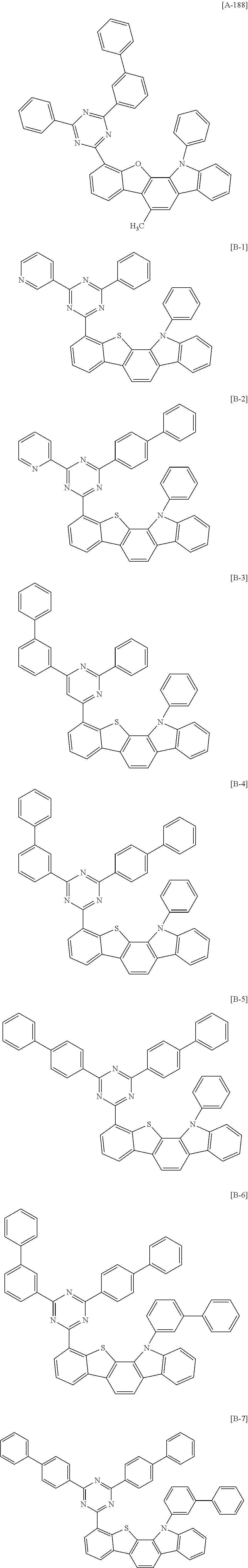

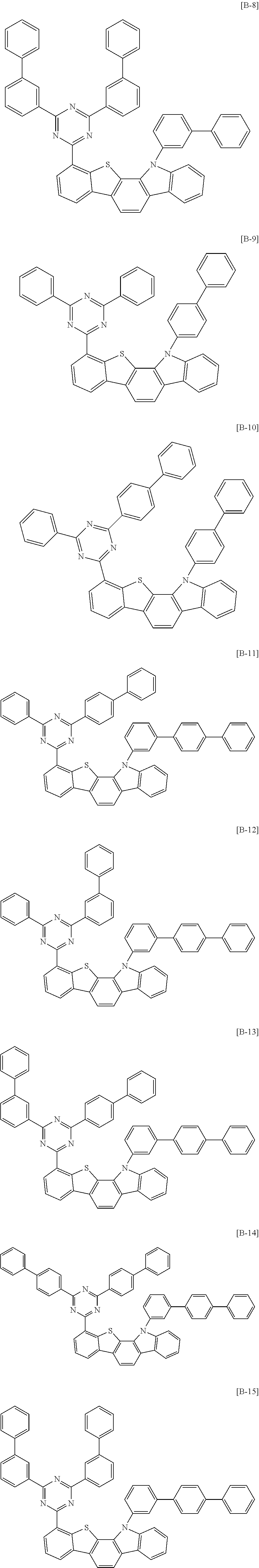

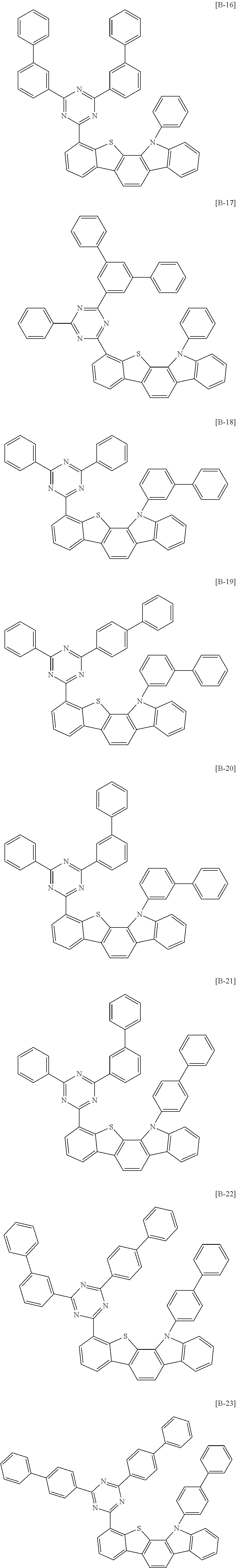

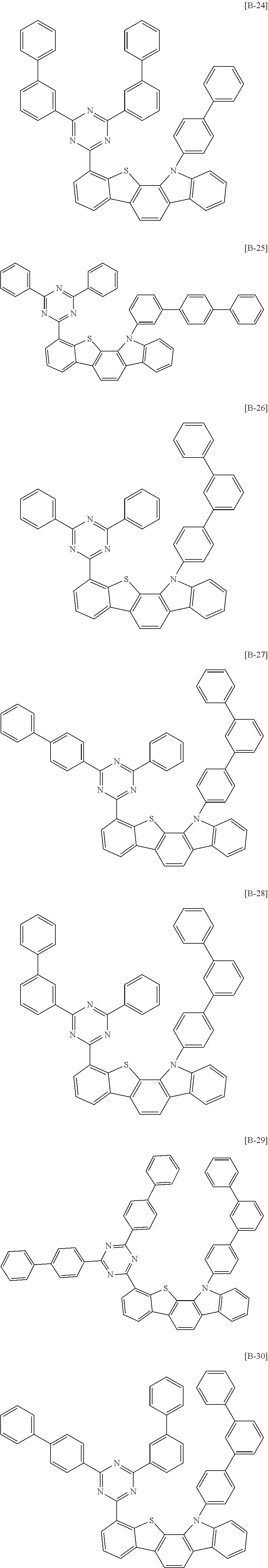

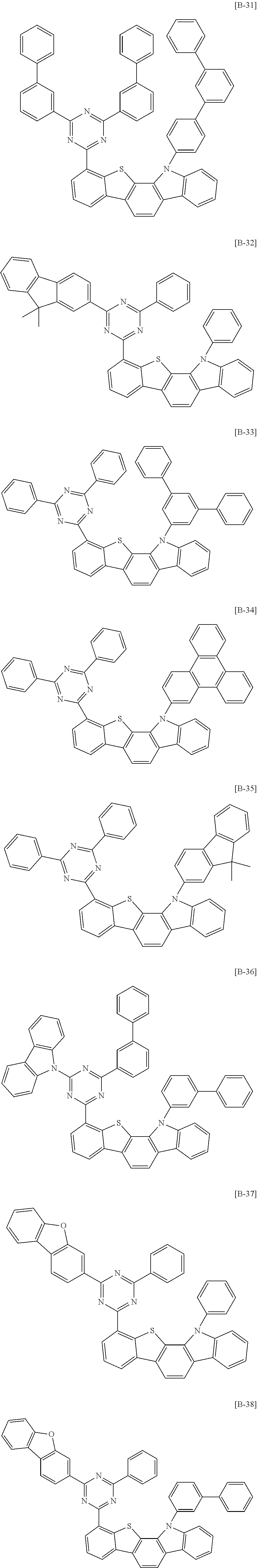

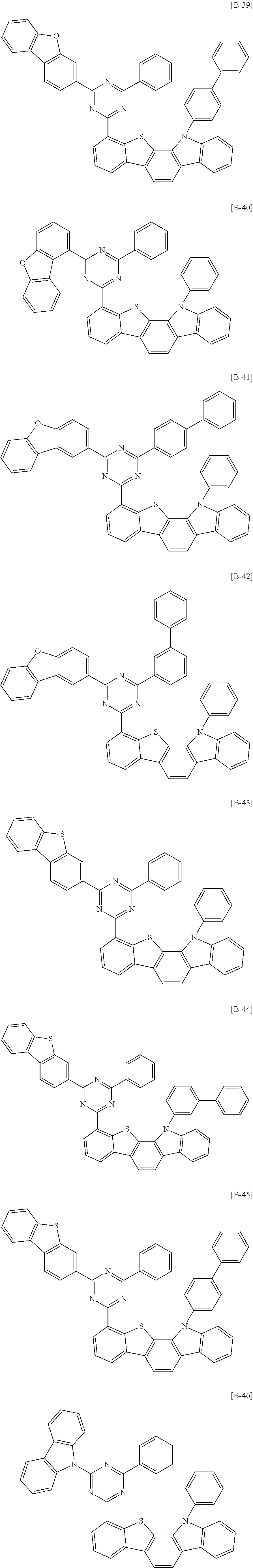

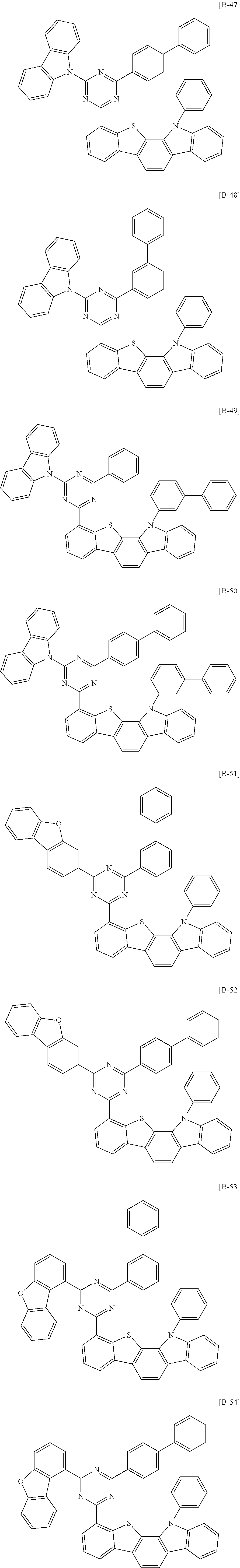

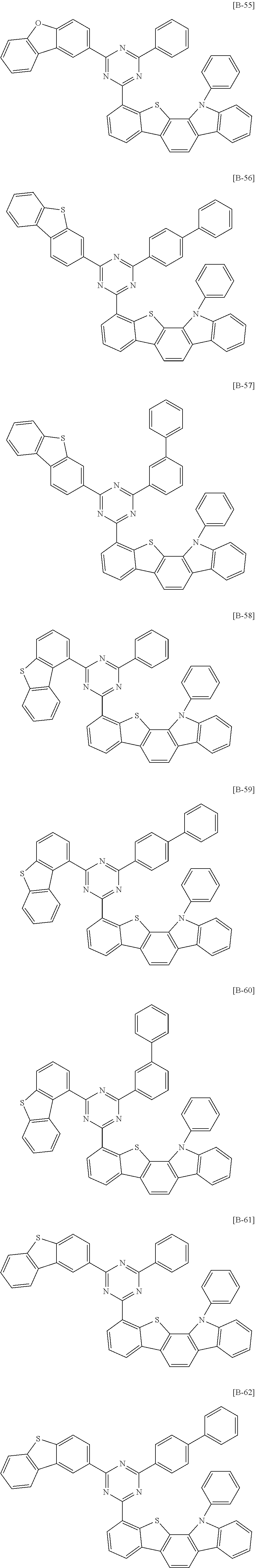

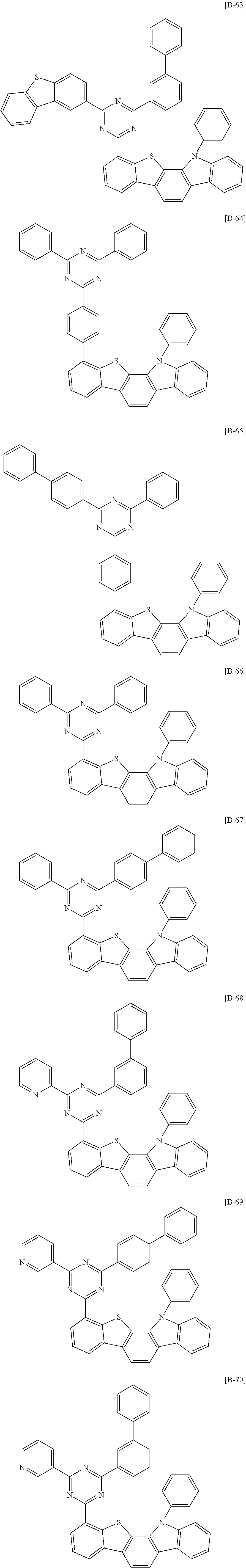

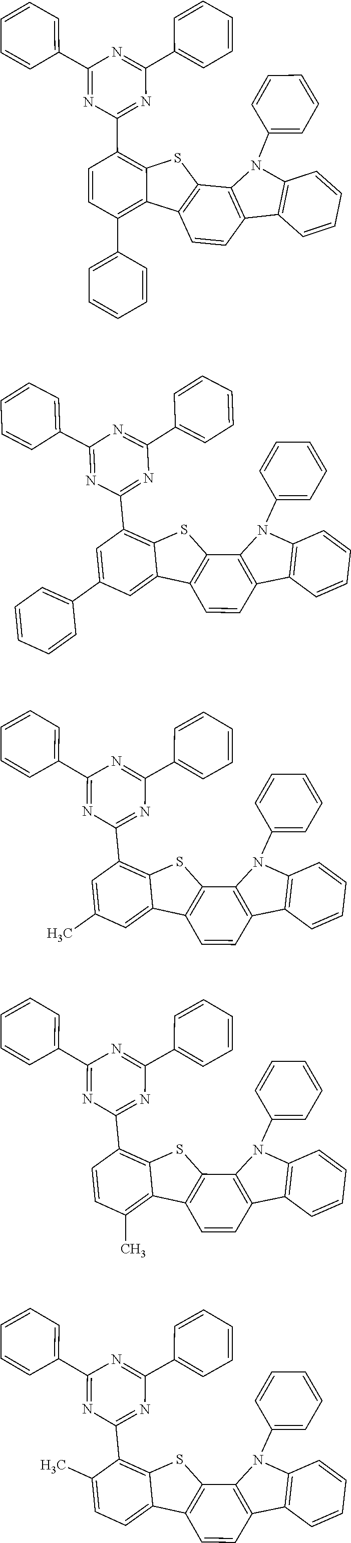 [SMILES: c1ccc(-c2nc(-c3ccccc3)nc(-c3ccc(-c4ccccc4)c4c3sc3c4ccc4c5ccccc5n(-c5ccccc5)c43)n2)cc1 ; c1ccc(-c2cc(-c3nc(-c4ccccc4)nc(-c4ccccc4)n3)c3sc4c(ccc5c6ccccc6n(-c6ccccc6)c54)c3c2)cc1 ; Cc1cc(-c2nc(-c3ccccc3)nc(-c3ccccc3)n2)c2sc3c(ccc4c5ccccc5n(-c5ccccc5)c43)c2c1 ; Cc1ccc(-c2nc(-c3ccccc3)nc(-c3ccccc3)n2)c2sc3c(ccc4c5ccccc5n(-c5ccccc5)c43)c12 ; Cc1ccc2c(sc3c2ccc2c4ccccc4n(-c4ccccc4)c23)c1-c1nc(-c2ccccc2)nc(-c2ccccc2)n1]

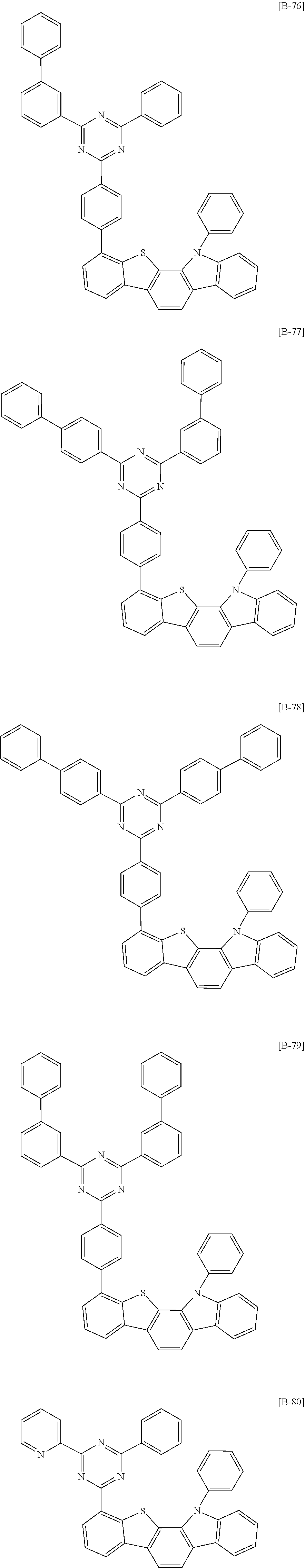

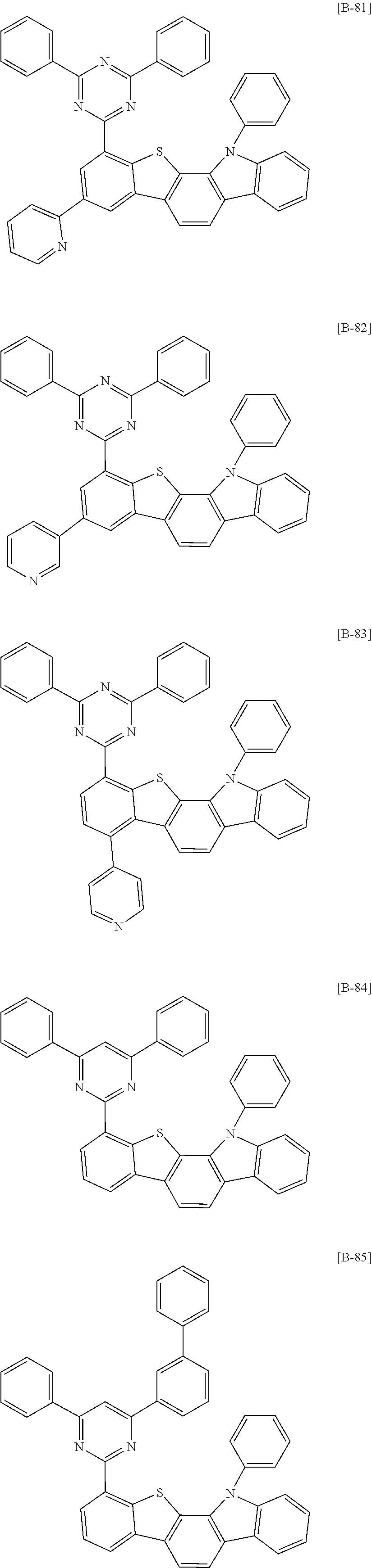 [SMILES: c1ccc(-c2nc(-c3ccccc3)nc(-c3cc(-c4ccccn4)cc4c3sc3c4ccc4c5ccccc5n(-c5ccccc5)c43)n2)cc1 ; c1ccc(-c2nc(-c3ccccc3)nc(-c3cc(-c4cccnc4)cc4c3sc3c4ccc4c5ccccc5n(-c5ccccc5)c43)n2)cc1 ; c1ccc(-c2nc(-c3ccccc3)nc(-c3ccc(-c4ccncc4)c4c3sc3c4ccc4c5ccccc5n(-c5ccccc5)c43)n2)cc1 ; c1ccc(-c2cc(-c3ccccc3)nc(-c3cccc4c3sc3c4ccc4c5ccccc5n(-c5ccccc5)c43)n2)cc1 ; c1ccc(-c2cccc(-c3cc(-c4ccccc4)nc(-c4cccc5c4sc4c5ccc5c6ccccc6n(-c6ccccc6)c54)n3)c2)cc1]

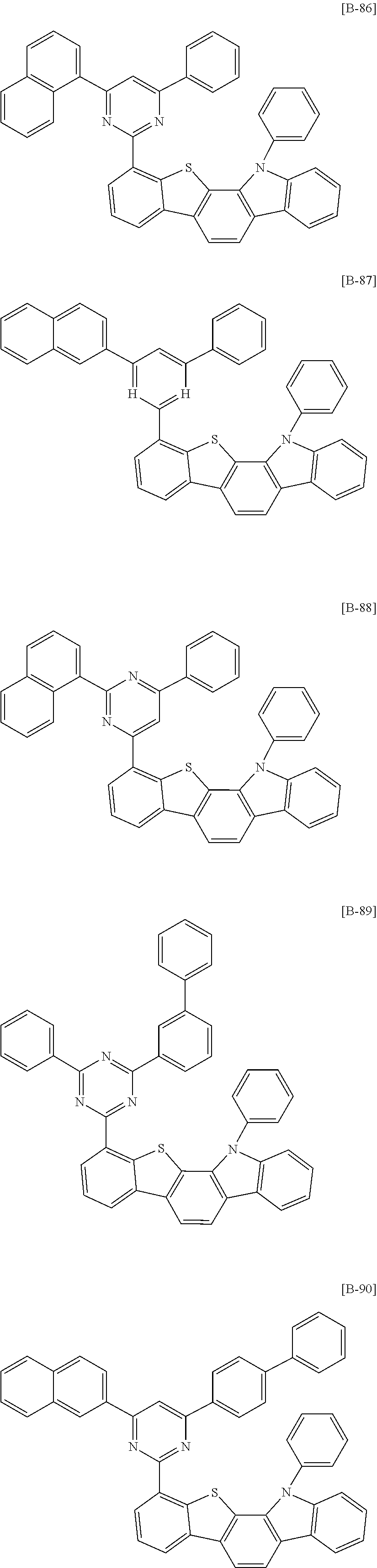

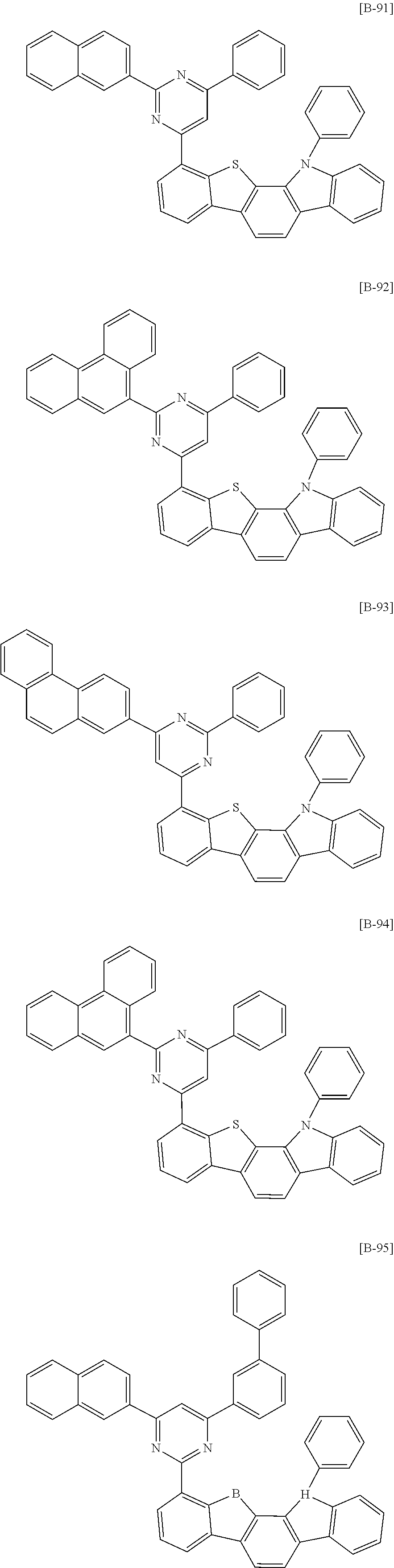

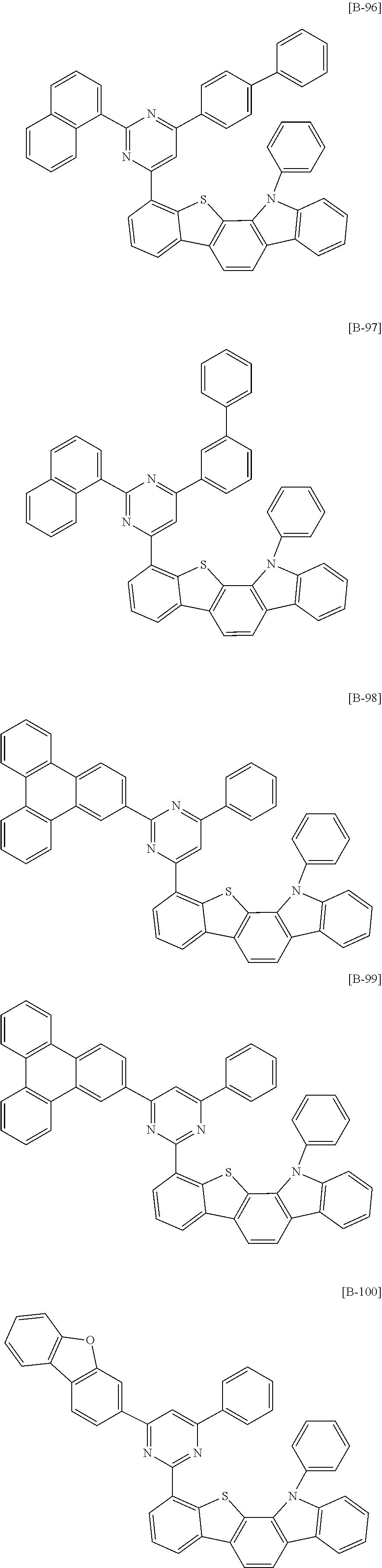 [SMILES: c1ccc(-c2ccc(-c3cc(-c4cccc5c4sc4c5ccc5c6ccccc6n(-c6ccccc6)c54)nc(-c4cccc5ccccc45)n3)cc2)cc1 ; c1ccc(-c2cccc(-c3cc(-c4cccc5c4sc4c5ccc5c6ccccc6n(-c6ccccc6)c54)nc(-c4cccc5ccccc45)n3)c2)cc1 ; c1ccc(-c2cc(-c3cccc4c3sc3c4ccc4c5ccccc5n(-c5ccccc5)c43)nc(-c3ccc4c5ccccc5c5ccccc5c4c3)n2)cc1 ; c1ccc(-c2cc(-c3ccc4c5ccccc5c5ccccc5c4c3)nc(-c3cccc4c3sc3c4ccc4c5ccccc5n(-c5ccccc5)c43)n2)cc1 ; c1ccc(-c2cc(-c3ccc4c(c3)oc3ccccc34)nc(-c3cccc4c3sc3c4ccc4c5ccccc5n(-c5ccccc5)c43)n2)cc1]

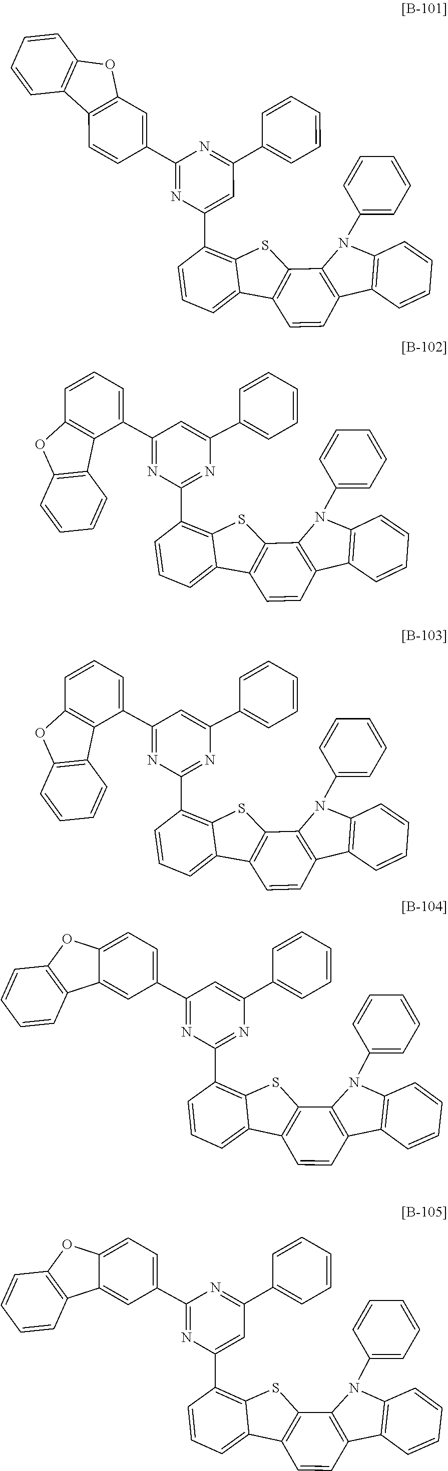

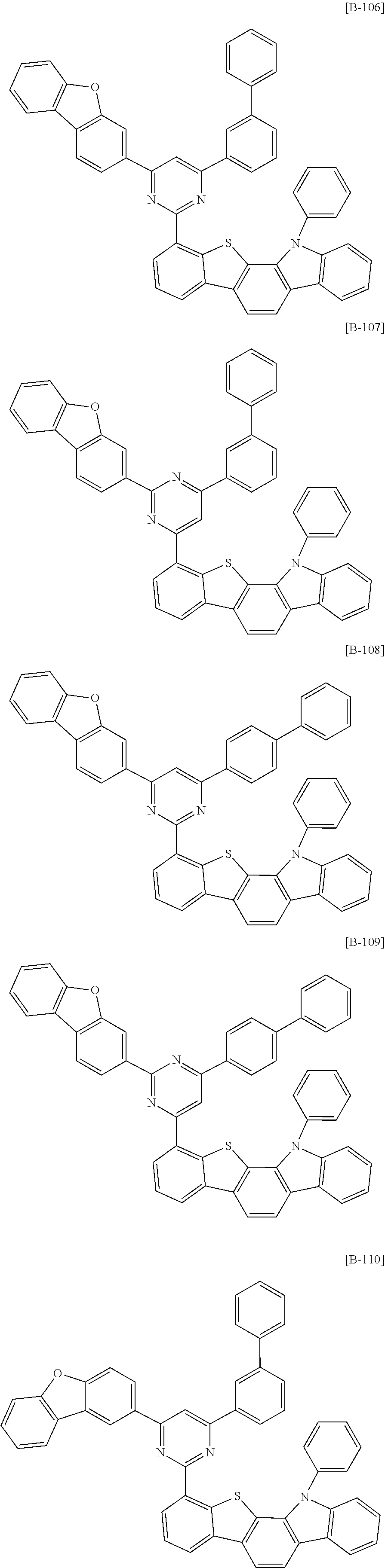

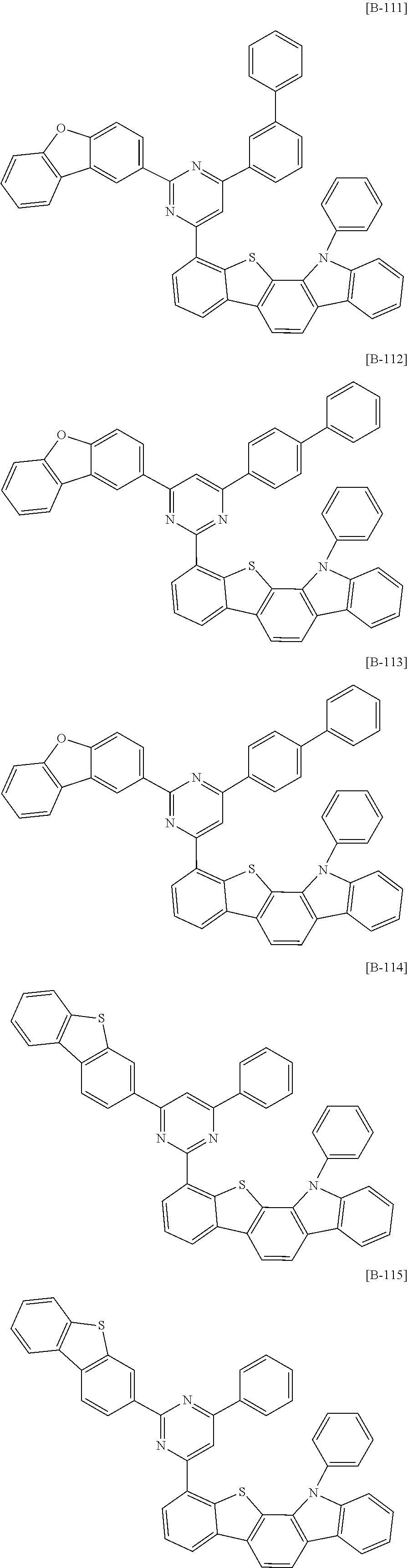 [SMILES: c1ccc(-c2cccc(-c3cc(-c4cccc5c4sc4c5ccc5c6ccccc6n(-c6ccccc6)c54)nc(-c4ccc5oc6ccccc6c5c4)n3)c2)cc1 ; c1ccc(-c2ccc(-c3cc(-c4ccc5oc6ccccc6c5c4)nc(-c4cccc5c4sc4c5ccc5c6ccccc6n(-c6ccccc6)c54)n3)cc2)cc1 ; c1ccc(-c2ccc(-c3cc(-c4cccc5c4sc4c5ccc5c6ccccc6n(-c6ccccc6)c54)nc(-c4ccc5oc6ccccc6c5c4)n3)cc2)cc1 ; c1ccc(-c2cc(-c3ccc4c(c3)sc3ccccc34)nc(-c3cccc4c3sc3c4ccc4c5ccccc5n(-c5ccccc5)c43)n2)cc1 ; c1ccc(-c2cc(-c3cccc4c3sc3c4ccc4c5ccccc5n(-c5ccccc5)c43)nc(-c3ccc4c(c3)sc3ccccc34)n2)cc1]

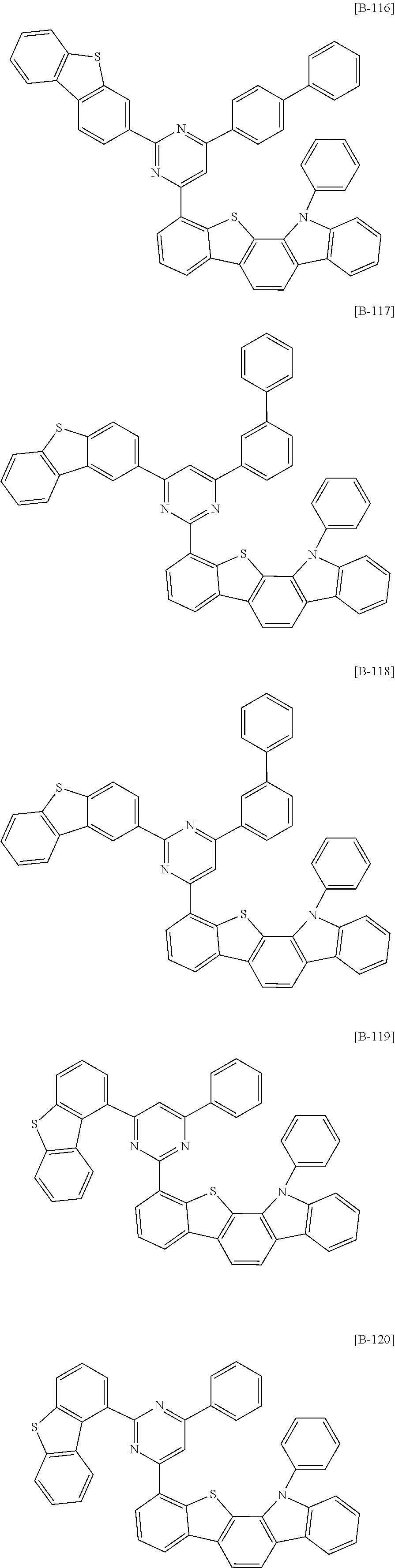 [SMILES: c1ccc(-c2ccc(-c3cc(-c4cccc5c4sc4c5ccc5c6ccccc6n(-c6ccccc6)c54)nc(-c4ccc5c(c4)sc4ccccc45)n3)cc2)cc1 ; c1ccc(-c2cccc(-c3cc(-c4ccc5sc6ccccc6c5c4)nc(-c4cccc5c4sc4c5ccc5c6ccccc6n(-c6ccccc6)c54)n3)c2)cc1 ; c1ccc(-c2cccc(-c3cc(-c4cccc5c4sc4c5ccc5c6ccccc6n(-c6ccccc6)c54)nc(-c4ccc5sc6ccccc6c5c4)n3)c2)cc1 ; c1ccc(-c2cc(-c3cccc4sc5ccccc5c34)nc(-c3cccc4c3sc3c4ccc4c5ccccc5n(-c5ccccc5)c43)n2)cc1 ; c1ccc(-c2cc(-c3cccc4c3sc3c4ccc4c5ccccc5n(-c5ccccc5)c43)nc(-c3cccc4sc5ccccc5c34)n2)cc1]

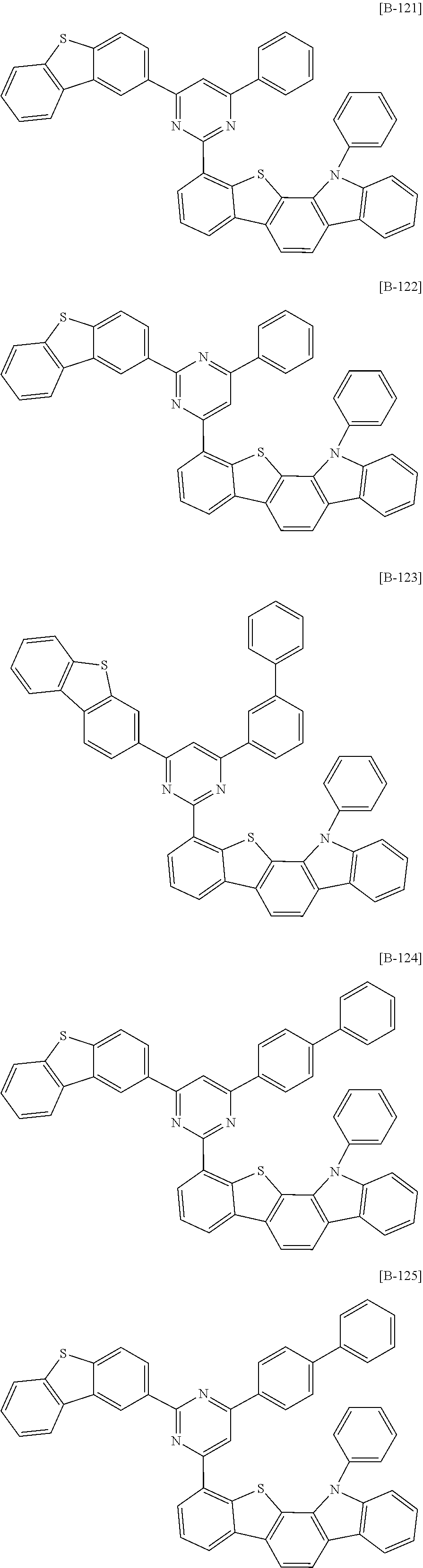 [SMILES: c1ccc(-c2cc(-c3ccc4sc5ccccc5c4c3)nc(-c3cccc4c3sc3c4ccc4c5ccccc5n(-c5ccccc5)c43)n2)cc1 ; c1ccc(-c2cc(-c3cccc4c3sc3c4ccc4c5ccccc5n(-c5ccccc5)c43)nc(-c3ccc4sc5ccccc5c4c3)n2)cc1 ; c1ccc(-c2cccc(-c3cc(-c4ccc5c(c4)sc4ccccc45)nc(-c4cccc5c4sc4c5ccc5c6ccccc6n(-c6ccccc6)c54)n3)c2)cc1 ; c1ccc(-c2ccc(-c3cc(-c4ccc5sc6ccccc6c5c4)nc(-c4cccc5c4sc4c5ccc5c6ccccc6n(-c6ccccc6)c54)n3)cc2)cc1 ; c1ccc(-c2ccc(-c3cc(-c4cccc5c4sc4c5ccc5c6ccccc6n(-c6ccccc6)c54)nc(-c4ccc5sc6ccccc6c5c4)n3)cc2)cc1]

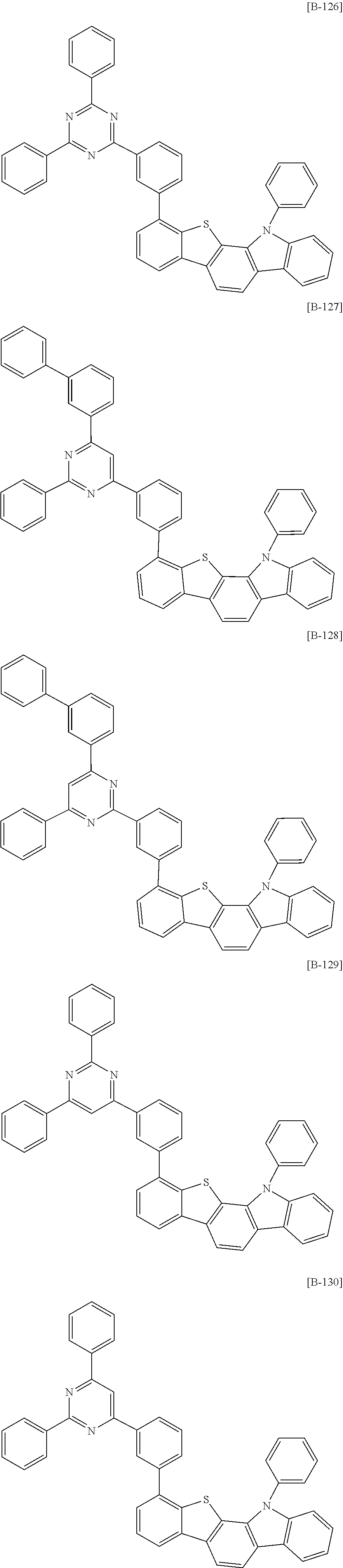 [SMILES: c1ccc(-c2nc(-c3ccccc3)nc(-c3cccc(-c4cccc5c4sc4c5ccc5c6ccccc6n(-c6ccccc6)c54)c3)n2)cc1 ; c1ccc(-c2cccc(-c3cc(-c4cccc(-c5cccc6c5sc5c6ccc6c7ccccc7n(-c7ccccc7)c65)c4)nc(-c4ccccc4)n3)c2)cc1 ; c1ccc(-c2cccc(-c3cc(-c4ccccc4)nc(-c4cccc(-c5cccc6c5sc5c6ccc6c7ccccc7n(-c7ccccc7)c65)c4)n3)c2)cc1 ; c1ccc(-c2cc(-c3cccc(-c4cccc5c4sc4c5ccc5c6ccccc6n(-c6ccccc6)c54)c3)nc(-c3ccccc3)n2)cc1 ; c1ccc(-c2cc(-c3cccc(-c4cccc5c4sc4c5ccc5c6ccccc6n(-c6ccccc6)c54)c3)nc(-c3ccccc3)n2)cc1]

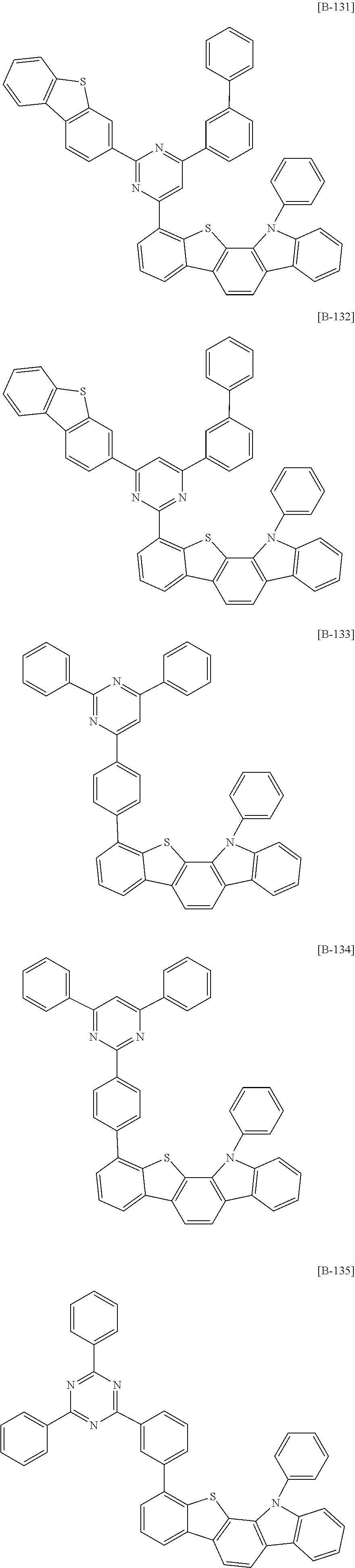 [SMILES: c1ccc(-c2cccc(-c3cc(-c4cccc5c4sc4c5ccc5c6ccccc6n(-c6ccccc6)c54)nc(-c4ccc5c(c4)sc4ccccc45)n3)c2)cc1 ; c1ccc(-c2cccc(-c3cc(-c4ccc5c(c4)sc4ccccc45)nc(-c4cccc5c4sc4c5ccc5c6ccccc6n(-c6ccccc6)c54)n3)c2)cc1 ; c1ccc(-c2cc(-c3ccc(-c4cccc5c4sc4c5ccc5c6ccccc6n(-c6ccccc6)c54)cc3)nc(-c3ccccc3)n2)cc1 ; c1ccc(-c2cc(-c3ccccc3)nc(-c3ccc(-c4cccc5c4sc4c5ccc5c6ccccc6n(-c6ccccc6)c54)cc3)n2)cc1 ; c1ccc(-c2nc(-c3ccccc3)nc(-c3cccc(-c4cccc5c4sc4c5ccc5c6ccccc6n(-c6ccccc6)c54)c3)n2)cc1]

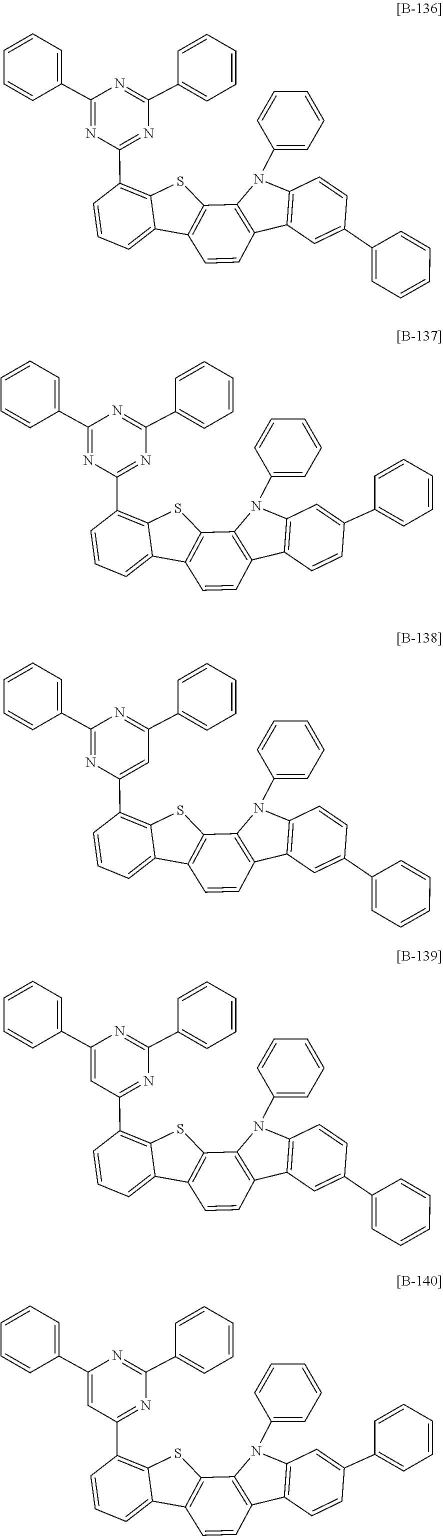 [SMILES: c1ccc(-c2ccc3c(c2)c2ccc4c5cccc(-c6nc(-c7ccccc7)nc(-c7ccccc7)n6)c5sc4c2n3-c2ccccc2)cc1 ; c1ccc(-c2ccc3c4ccc5c6cccc(-c7nc(-c8ccccc8)nc(-c8ccccc8)n7)c6sc5c4n(-c4ccccc4)c3c2)cc1 ; c1ccc(-c2ccc3c(c2)c2ccc4c5cccc(-c6cc(-c7ccccc7)nc(-c7ccccc7)n6)c5sc4c2n3-c2ccccc2)cc1 ; c1ccc(-c2ccc3c(c2)c2ccc4c5cccc(-c6cc(-c7ccccc7)nc(-c7ccccc7)n6)c5sc4c2n3-c2ccccc2)cc1 ; c1ccc(-c2ccc3c4ccc5c6cccc(-c7cc(-c8ccccc8)nc(-c8ccccc8)n7)c6sc5c4n(-c4ccccc4)c3c2)cc1]

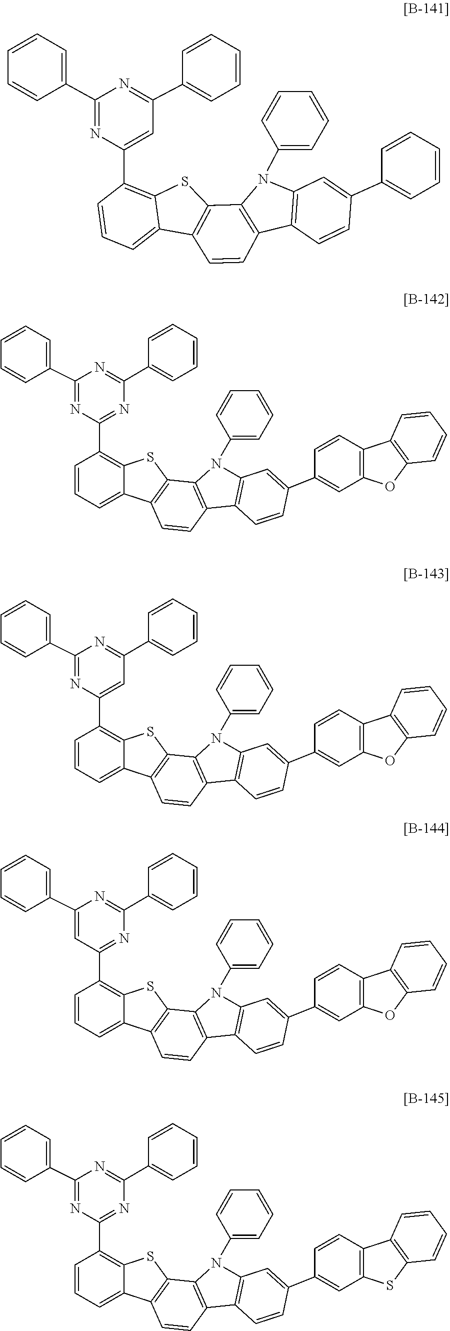 [SMILES: c1ccc(-c2ccc3c4ccc5c6cccc(-c7cc(-c8ccccc8)nc(-c8ccccc8)n7)c6sc5c4n(-c4ccccc4)c3c2)cc1 ; c1ccc(-c2nc(-c3ccccc3)nc(-c3cccc4c3sc3c4ccc4c5ccc(-c6ccc7c(c6)oc6ccccc67)cc5n(-c5ccccc5)c43)n2)cc1 ; c1ccc(-c2cc(-c3cccc4c3sc3c4ccc4c5ccc(-c6ccc7c(c6)oc6ccccc67)cc5n(-c5ccccc5)c43)nc(-c3ccccc3)n2)cc1 ; c1ccc(-c2cc(-c3cccc4c3sc3c4ccc4c5ccc(-c6ccc7c(c6)oc6ccccc67)cc5n(-c5ccccc5)c43)nc(-c3ccccc3)n2)cc1 ; c1ccc(-c2nc(-c3ccccc3)nc(-c3cccc4c3sc3c4ccc4c5ccc(-c6ccc7c(c6)sc6ccccc67)cc5n(-c5ccccc5)c43)n2)cc1]

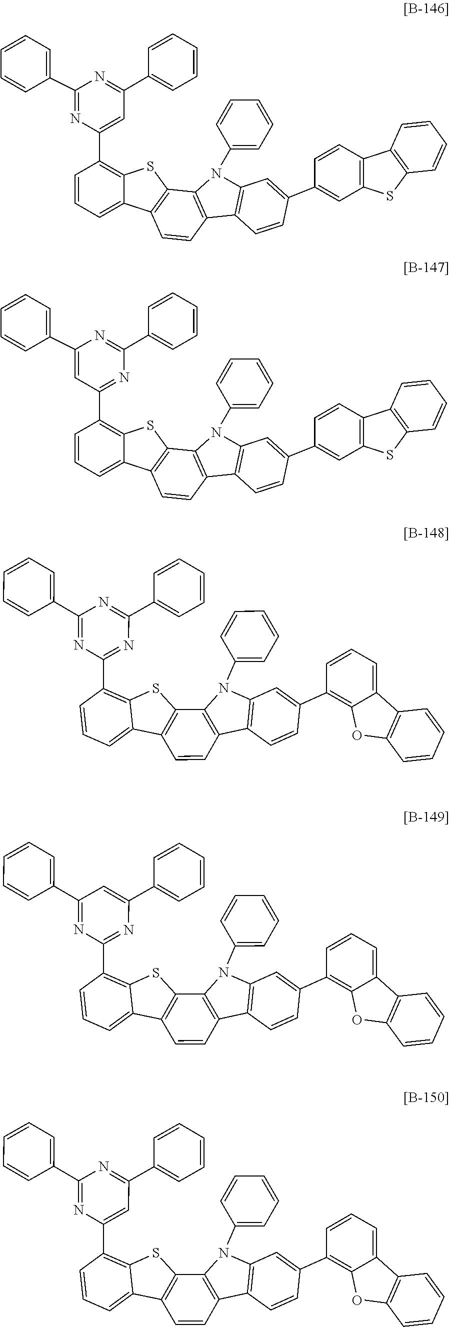 [SMILES: c1ccc(-c2cc(-c3cccc4c3sc3c4ccc4c5ccc(-c6ccc7c(c6)sc6ccccc67)cc5n(-c5ccccc5)c43)nc(-c3ccccc3)n2)cc1 ; c1ccc(-c2cc(-c3cccc4c3sc3c4ccc4c5ccc(-c6ccc7c(c6)sc6ccccc67)cc5n(-c5ccccc5)c43)nc(-c3ccccc3)n2)cc1 ; c1ccc(-c2nc(-c3ccccc3)nc(-c3cccc4c3sc3c4ccc4c5ccc(-c6cccc7c6oc6ccccc67)cc5n(-c5ccccc5)c43)n2)cc1 ; c1ccc(-c2cc(-c3ccccc3)nc(-c3cccc4c3sc3c4ccc4c5ccc(-c6cccc7c6oc6ccccc67)cc5n(-c5ccccc5)c43)n2)cc1 ; c1ccc(-c2cc(-c3cccc4c3sc3c4ccc4c5ccc(-c6cccc7c6oc6ccccc67)cc5n(-c5ccccc5)c43)nc(-c3ccccc3)n2)cc1]

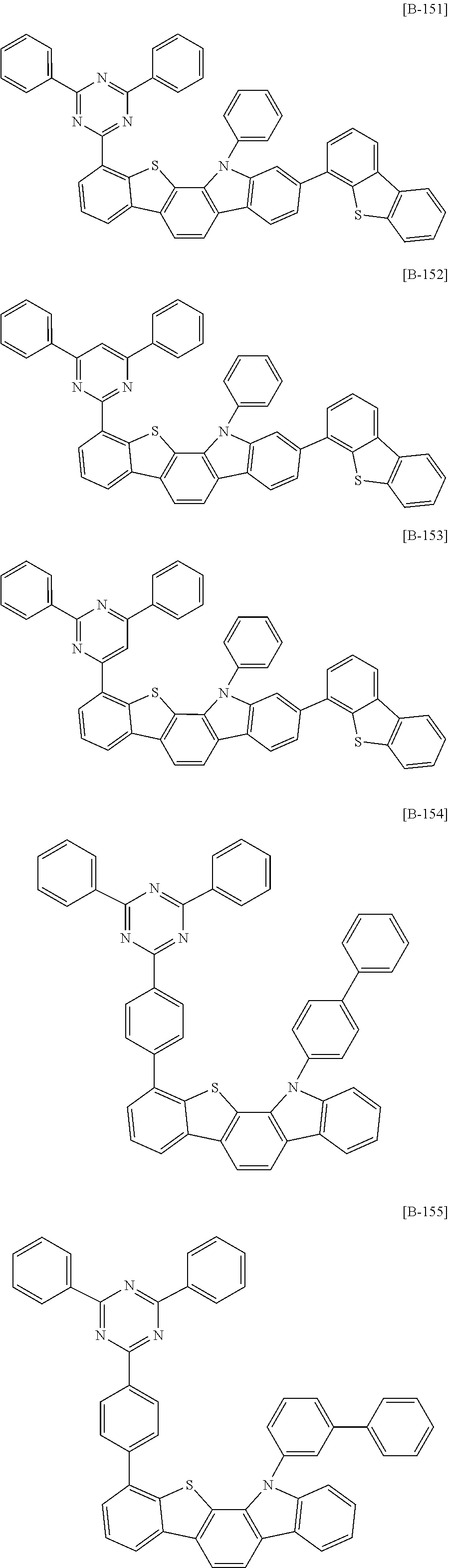 [SMILES: c1ccc(-c2nc(-c3ccccc3)nc(-c3cccc4c3sc3c4ccc4c5ccc(-c6cccc7c6sc6ccccc67)cc5n(-c5ccccc5)c43)n2)cc1 ; c1ccc(-c2cc(-c3ccccc3)nc(-c3cccc4c3sc3c4ccc4c5ccc(-c6cccc7c6sc6ccccc67)cc5n(-c5ccccc5)c43)n2)cc1 ; c1ccc(-c2cc(-c3cccc4c3sc3c4ccc4c5ccc(-c6cccc7c6sc6ccccc67)cc5n(-c5ccccc5)c43)nc(-c3ccccc3)n2)cc1 ; c1ccc(-c2ccc(-n3c4ccccc4c4ccc5c6cccc(-c7ccc(-c8nc(-c9ccccc9)nc(-c9ccccc9)n8)cc7)c6sc5c43)cc2)cc1 ; c1ccc(-c2cccc(-n3c4ccccc4c4ccc5c6cccc(-c7ccc(-c8nc(-c9ccccc9)nc(-c9ccccc9)n8)cc7)c6sc5c43)c2)cc1]

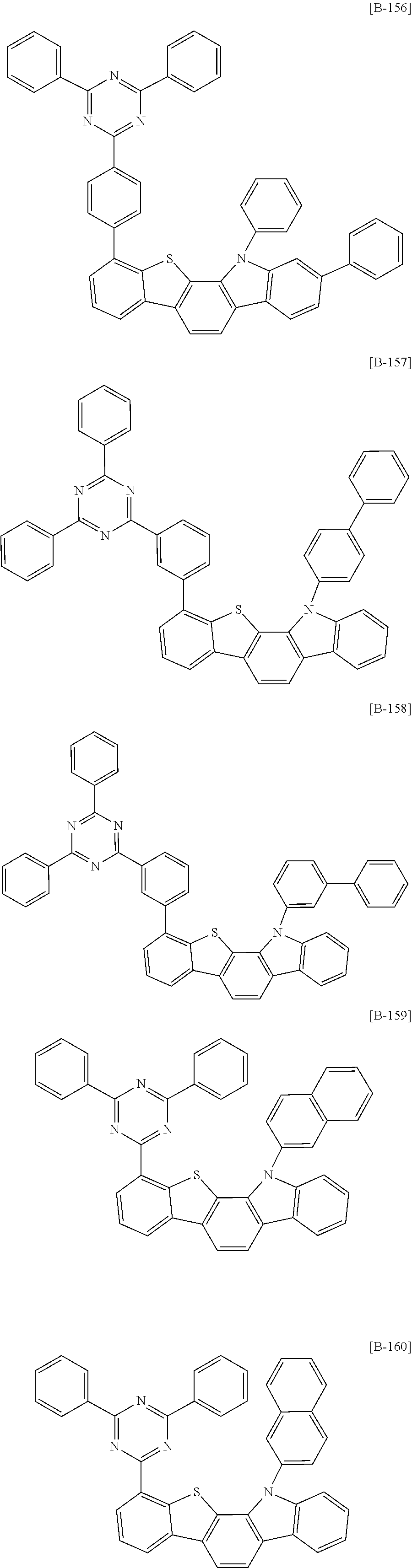

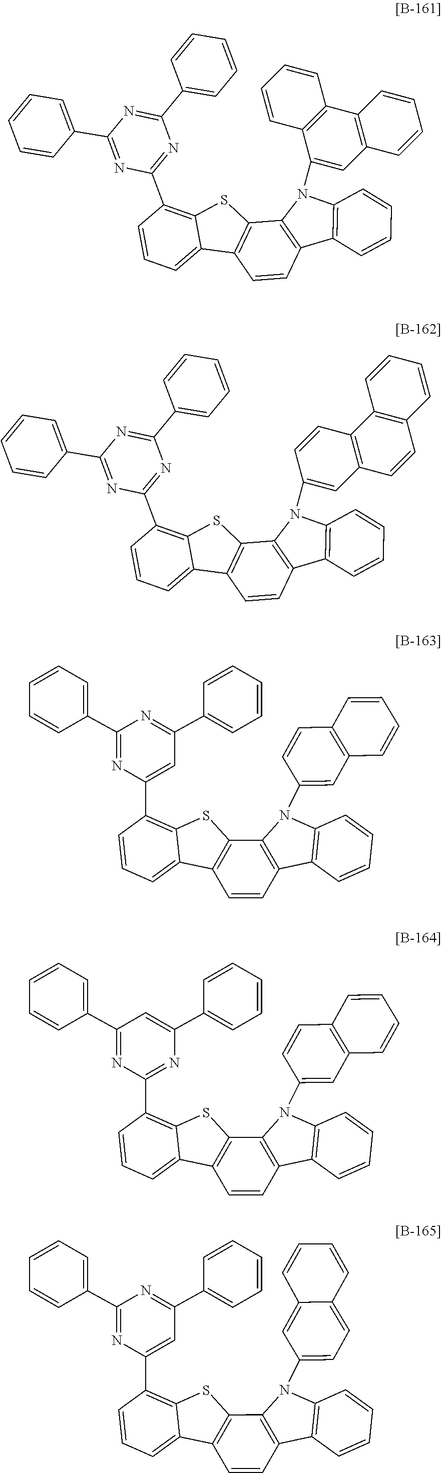

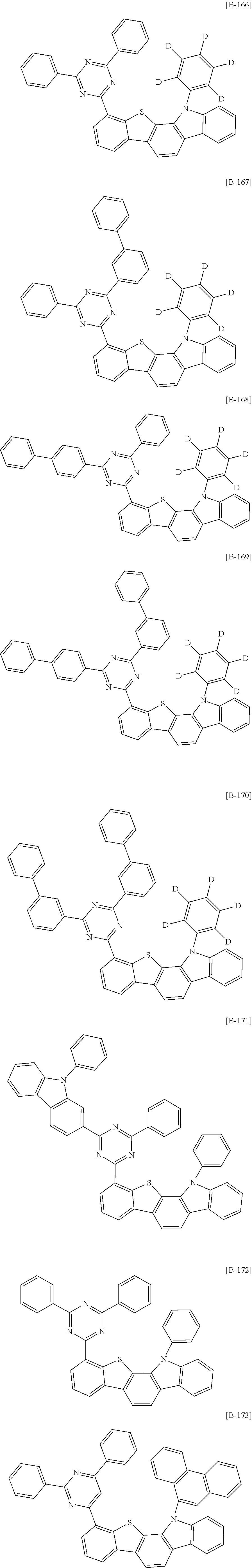

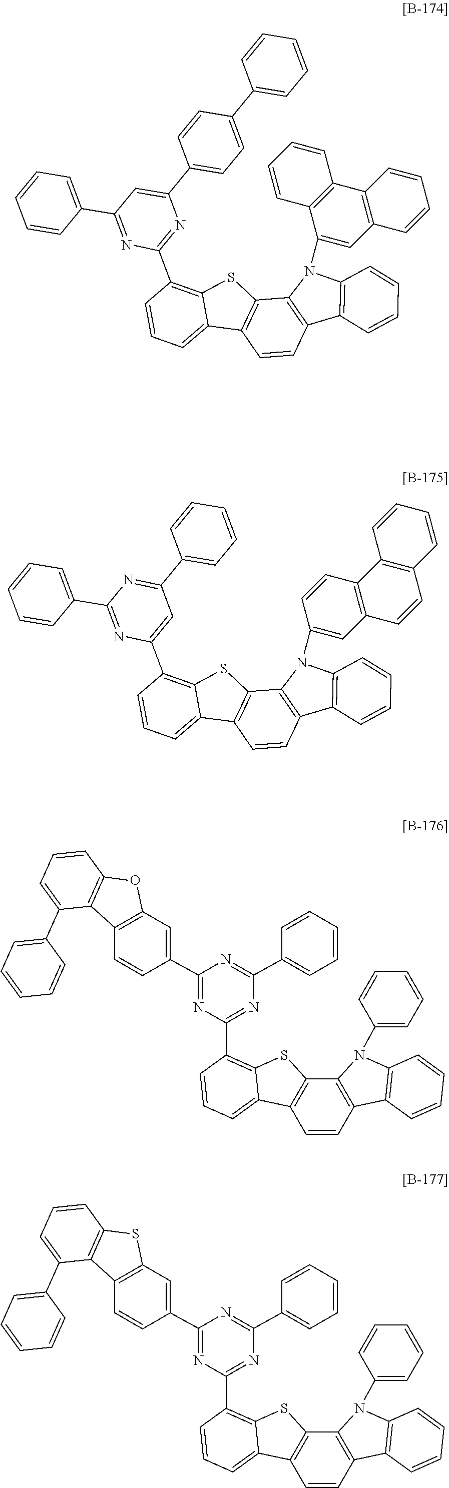 [SMILES: c1ccc(-c2ccc(-c3cc(-c4ccccc4)nc(-c4cccc5c4sc4c5ccc5c6ccccc6n(-c6cc7ccccc7c7ccccc67)c54)n3)cc2)cc1 ; c1ccc(-c2cc(-c3cccc4c3sc3c4ccc4c5ccccc5n(-c5ccc6c(ccc7ccccc76)c5)c43)nc(-c3ccccc3)n2)cc1 ; c1ccc(-c2nc(-c3ccc4c(c3)oc3cccc(-c5ccccc5)c34)nc(-c3cccc4c3sc3c4ccc4c5ccccc5n(-c5ccccc5)c43)n2)cc1 ; c1ccc(-c2nc(-c3ccc4c(c3)sc3cccc(-c5ccccc5)c34)nc(-c3cccc4c3sc3c4ccc4c5ccccc5n(-c5ccccc5)c43)n2)cc1]

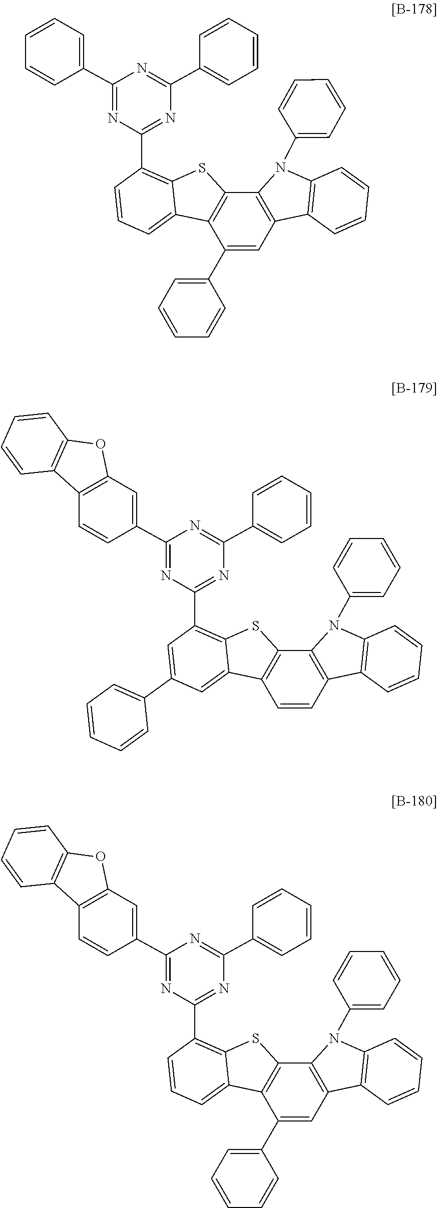 [SMILES: c1ccc(-c2nc(-c3ccccc3)nc(-c3cccc4c3sc3c4c(-c4ccccc4)cc4c5ccccc5n(-c5ccccc5)c43)n2)cc1 ; c1ccc(-c2cc(-c3nc(-c4ccccc4)nc(-c4ccc5c(c4)oc4ccccc45)n3)c3sc4c(ccc5c6ccccc6n(-c6ccccc6)c54)c3c2)cc1 ; c1ccc(-c2nc(-c3ccc4c(c3)oc3ccccc34)nc(-c3cccc4c3sc3c4c(-c4ccccc4)cc4c5ccccc5n(-c5ccccc5)c43)n2)cc1]

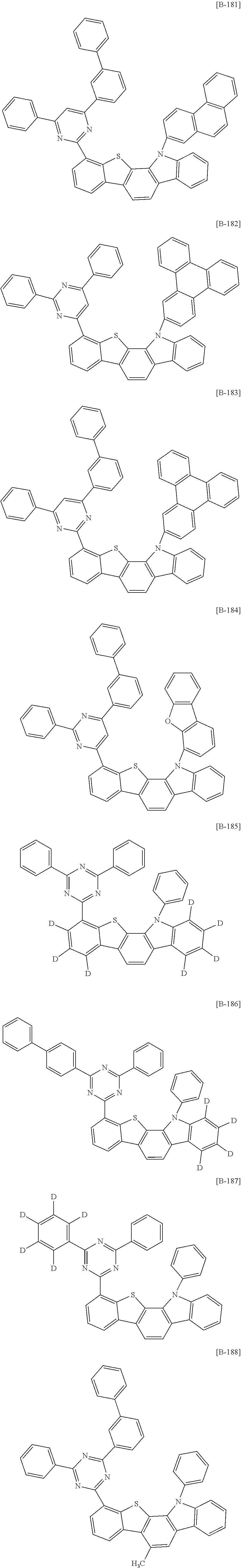

Meanwhile, the second compound may be represented by any one of Chemical Formula 2-1 to Chemical Formula 2-15, depending on the presence or absence of a linking group and/or a linking position.

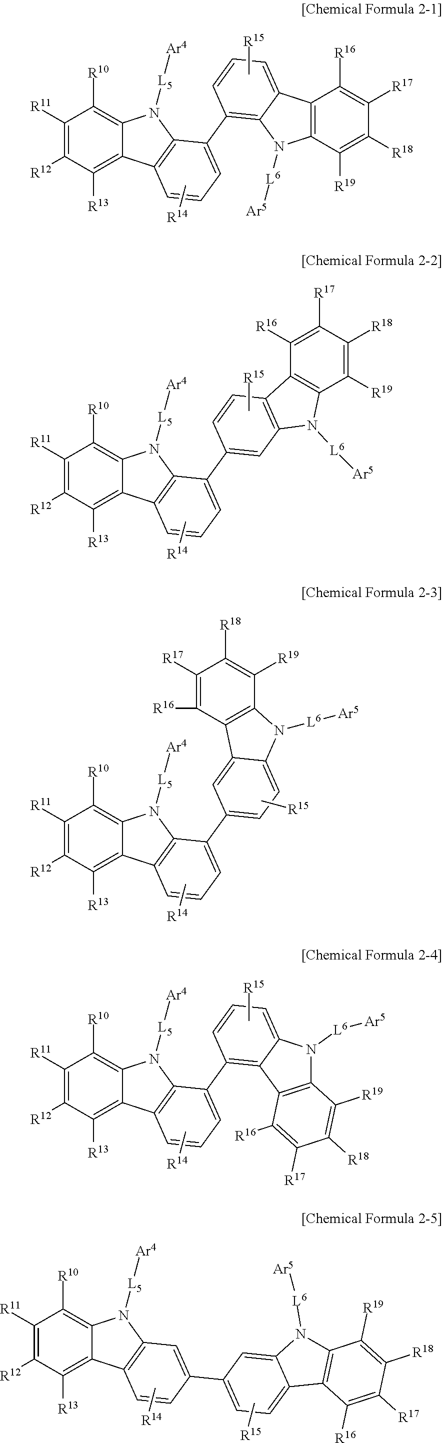

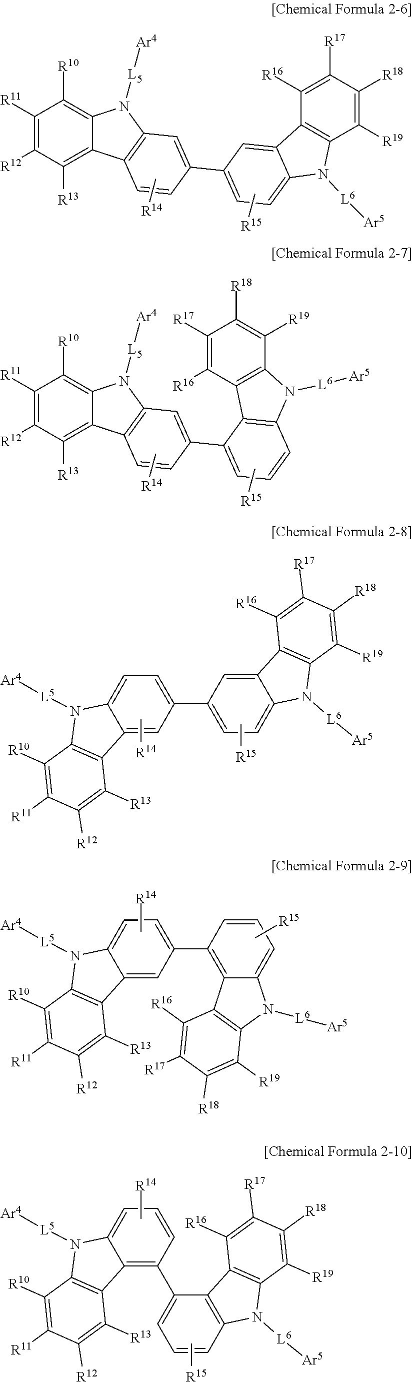

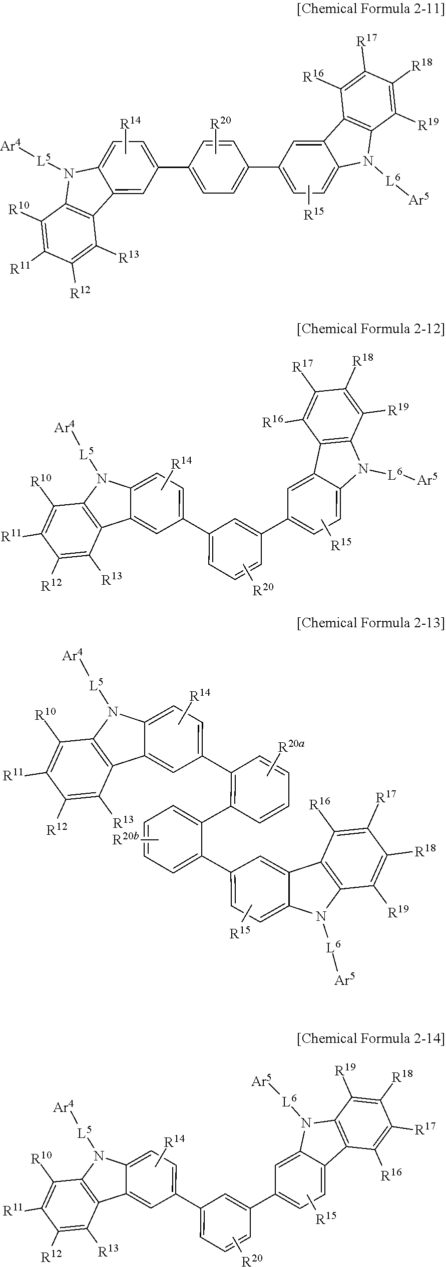

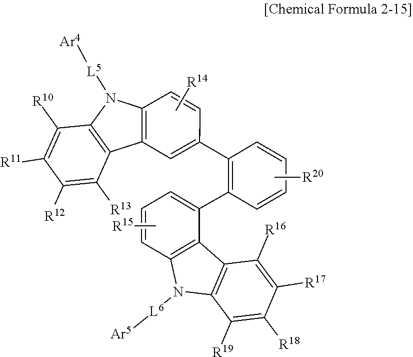

In Chemical Formula 2-1 to Chemical Formula 2-15, Ar⁴, Ar⁵, L⁵, L⁶, and R¹⁰ to R²⁰ are the same as described above, and

R^{20a} and R^{20b} are each independently the same as the aforementioned definition of R.

In an embodiment, the second compound may be represented by Chemical Formula 2-8.

For example, in Chemical Formula 2-8, L⁵ and L⁶ may each independently be a single bond, a substituted or unsubstituted phenylene group, or a substituted or unsubstituted biphenylene group,

Ar⁴ and Ar⁵ may each independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, a substituted or unsubstituted fluorenyl group, or a substituted or unsubstituted pyridinyl group, and

R¹⁰ to R¹⁹ may each independently be hydrogen, deuterium, a substituted or unsubstituted C1 to C10 alkyl group, a substituted or unsubstituted C6 to C12 aryl group, a substituted or unsubstituted dibenzofuranyl group, or a substituted or unsubstituted dibenzothiophenyl group.

For example, in Chemical Formula 2-8, *-L⁵-Ar⁴ and *-L⁶-Ar⁵ may each independently be selected from substituted or unsubstituted groups of Group II.

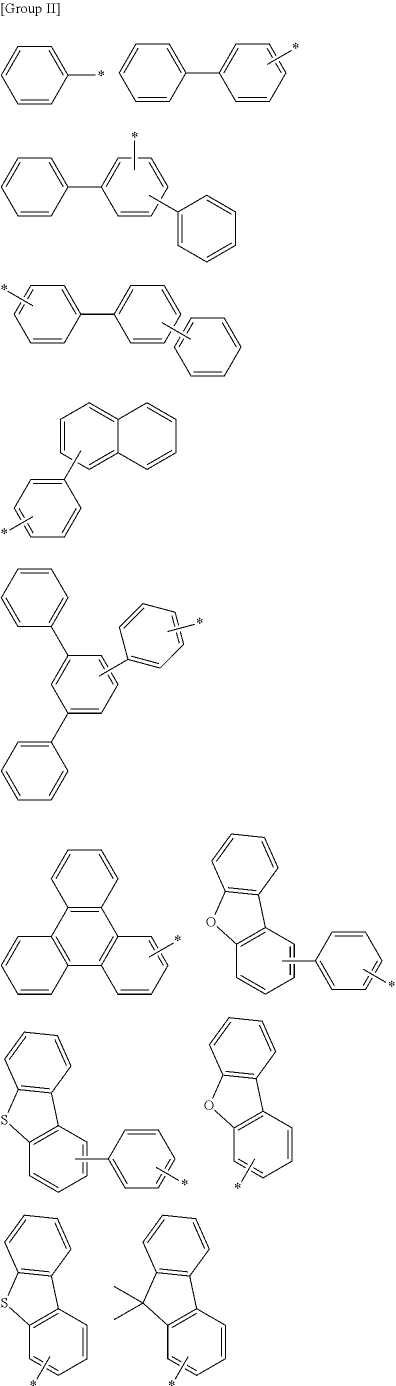

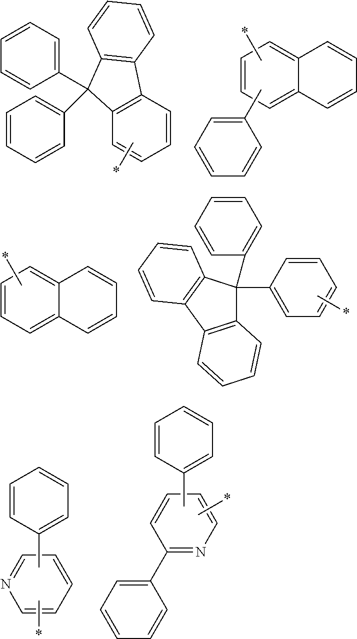

For example, in Chemical Formula 2-8, *-L⁵-Ar⁴ and *-L⁶-Ar⁵ may each independently be selected from substituted or unsubstituted groups of Group II-1.

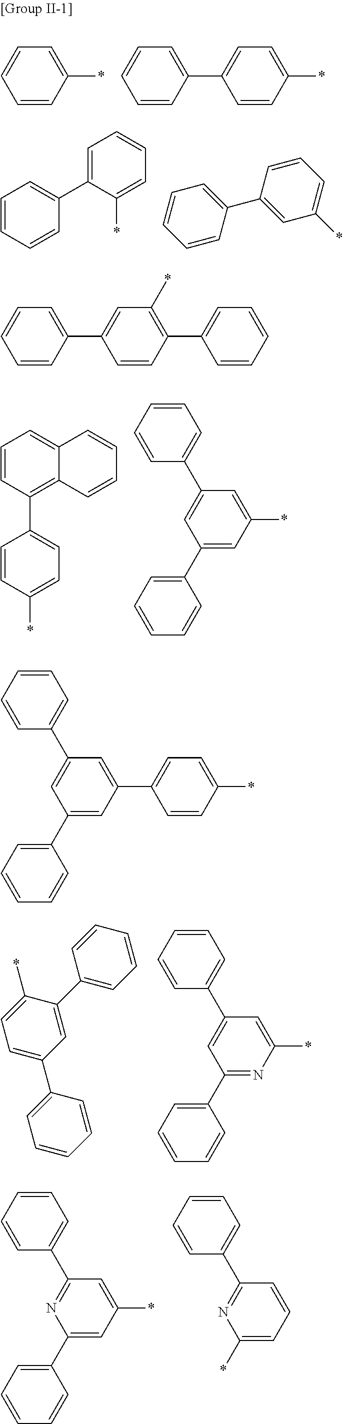

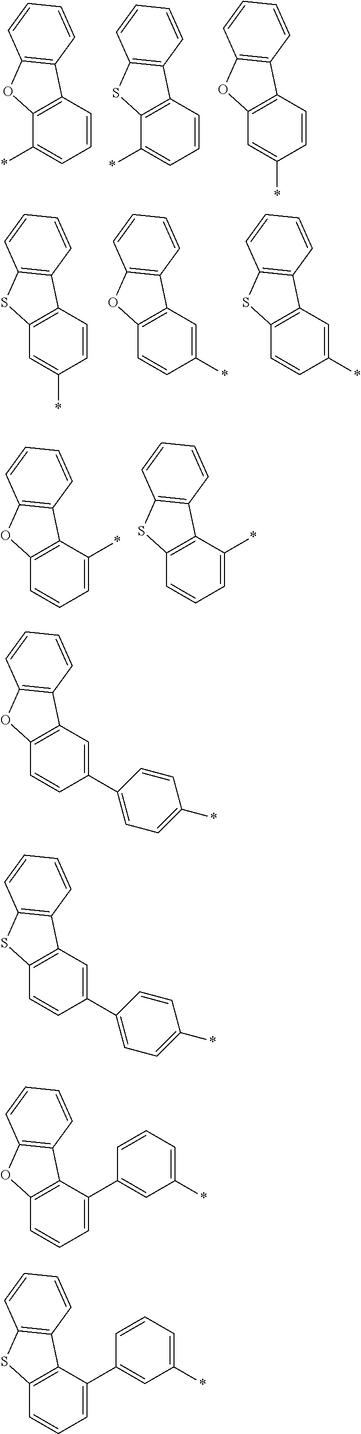

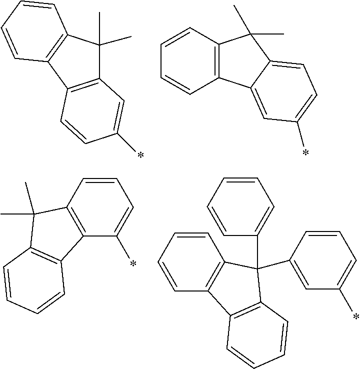

In Group II and Group II-1, “substituted” means substitution with deuterium, a fluoro group, a C1 to C5 alkyl group, or a C6 to C12 aryl group, *and

* is a linking point.

As a specific example, L⁵ and L⁶ may each independently be a single bond or a substituted or unsubstituted phenylene group, and

Ar⁴ and Ar⁵ may each independently be a substituted or unsubstituted phenyl group or a substituted or unsubstituted biphenyl group.

For example, the second compound may be one selected from compounds of Group 2, but is not limited thereto.

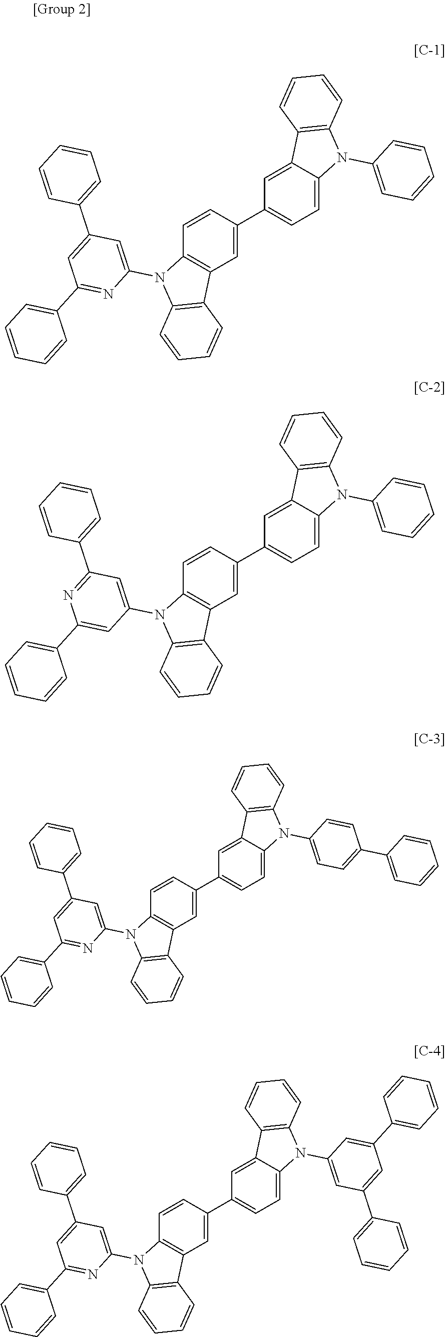 [SMILES: c1ccc(-c2cc(-c3ccccc3)nc(-n3c4ccccc4c4cc(-c5ccc6c(c5)c5ccccc5n6-c5ccccc5)ccc43)c2)cc1 ; c1ccc(-c2cc(-n3c4ccccc4c4cc(-c5ccc6c(c5)c5ccccc5n6-c5ccccc5)ccc43)cc(-c3ccccc3)n2)cc1 ; c1ccc(-c2ccc(-n3c4ccccc4c4cc(-c5ccc6c(c5)c5ccccc5n6-c5cc(-c6ccccc6)cc(-c6ccccc6)n5)ccc43)cc2)cc1 ; c1ccc(-c2cc(-c3ccccc3)cc(-n3c4ccccc4c4cc(-c5ccc6c(c5)c5ccccc5n6-c5cc(-c6ccccc6)cc(-c6ccccc6)n5)ccc43)c2)cc1]

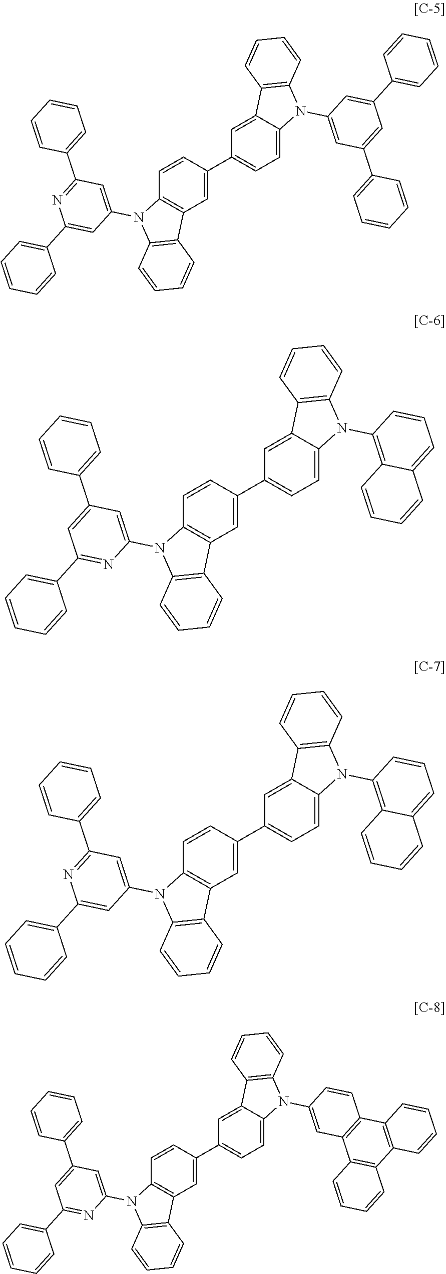 [SMILES: c1ccc(-c2cc(-c3ccccc3)cc(-n3c4ccccc4c4cc(-c5ccc6c(c5)c5ccccc5n6-c5cc(-c6ccccc6)nc(-c6ccccc6)c5)ccc43)c2)cc1 ; c1ccc(-c2cc(-c3ccccc3)nc(-n3c4ccccc4c4cc(-c5ccc6c(c5)c5ccccc5n6-c5cccc6ccccc56)ccc43)c2)cc1 ; c1ccc(-c2cc(-n3c4ccccc4c4cc(-c5ccc6c(c5)c5ccccc5n6-c5cccc6ccccc56)ccc43)cc(-c3ccccc3)n2)cc1 ; c1ccc(-c2cc(-c3ccccc3)nc(-n3c4ccccc4c4cc(-c5ccc6c(c5)c5ccccc5n6-c5ccc6c7ccccc7c7ccccc7c6c5)ccc43)c2)cc1]

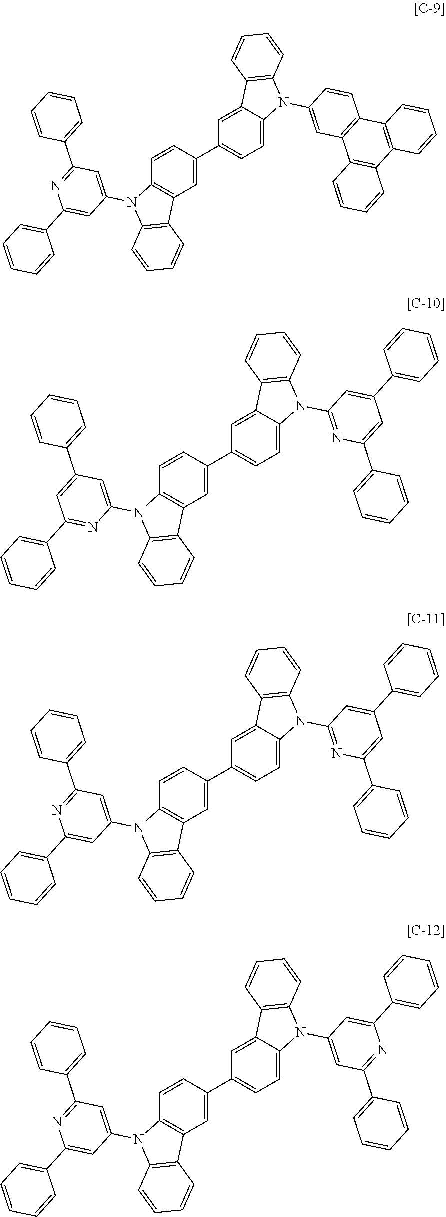 [SMILES: c1ccc(-c2cc(-n3c4ccccc4c4cc(-c5ccc6c(c5)c5ccccc5n6-c5ccc6c7ccccc7c7ccccc7c6c5)ccc43)cc(-c3ccccc3)n2)cc1 ; c1ccc(-c2cc(-c3ccccc3)nc(-n3c4ccccc4c4cc(-c5ccc6c(c5)c5ccccc5n6-c5cc(-c6ccccc6)cc(-c6ccccc6)n5)ccc43)c2)cc1 ; c1ccc(-c2cc(-c3ccccc3)nc(-n3c4ccccc4c4cc(-c5ccc6c(c5)c5ccccc5n6-c5cc(-c6ccccc6)nc(-c6ccccc6)c5)ccc43)c2)cc1 ; c1ccc(-c2cc(-n3c4ccccc4c4cc(-c5ccc6c(c5)c5ccccc5n6-c5cc(-c6ccccc6)nc(-c6ccccc6)c5)ccc43)cc(-c3ccccc3)n2)cc1]

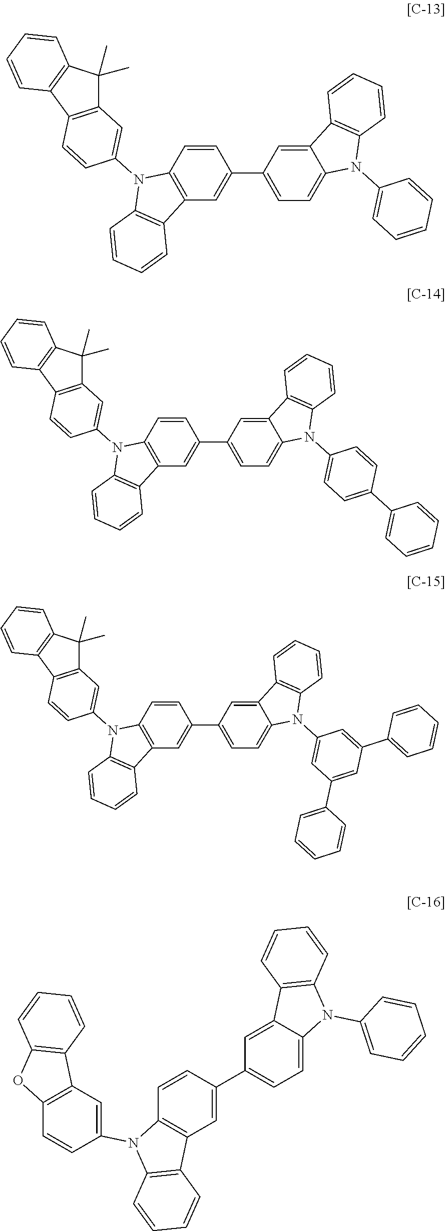 [SMILES: CC1(C)c2ccccc2-c2ccc(-n3c4ccccc4c4cc(-c5ccc6c(c5)c5ccccc5n6-c5ccccc5)ccc43)cc21 ; CC1(C)c2ccccc2-c2ccc(-n3c4ccccc4c4cc(-c5ccc6c(c5)c5ccccc5n6-c5ccc(-c6ccccc6)cc5)ccc43)cc21 ; CC1(C)c2ccccc2-c2ccc(-n3c4ccccc4c4cc(-c5ccc6c(c5)c5ccccc5n6-c5cc(-c6ccccc6)cc(-c6ccccc6)c5)ccc43)cc21 ; c1ccc(-n2c3ccccc3c3cc(-c4ccc5c(c4)c4ccccc4n5-c4ccc5oc6ccccc6c5c4)ccc32)cc1]

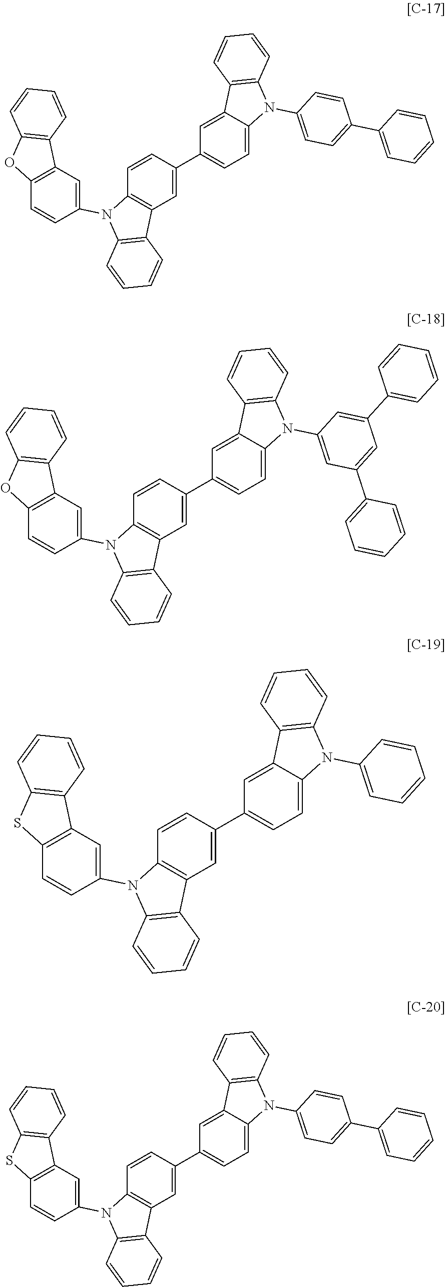 [SMILES: c1ccc(-c2ccc(-n3c4ccccc4c4cc(-c5ccc6c(c5)c5ccccc5n6-c5ccc6oc7ccccc7c6c5)ccc43)cc2)cc1 ; c1ccc(-c2cc(-c3ccccc3)cc(-n3c4ccccc4c4cc(-c5ccc6c(c5)c5ccccc5n6-c5ccc6oc7ccccc7c6c5)ccc43)c2)cc1 ; c1ccc(-n2c3ccccc3c3cc(-c4ccc5c(c4)c4ccccc4n5-c4ccc5sc6ccccc6c5c4)ccc32)cc1 ; c1ccc(-c2ccc(-n3c4ccccc4c4cc(-c5ccc6c(c5)c5ccccc5n6-c5ccc6sc7ccccc7c6c5)ccc43)cc2)cc1]

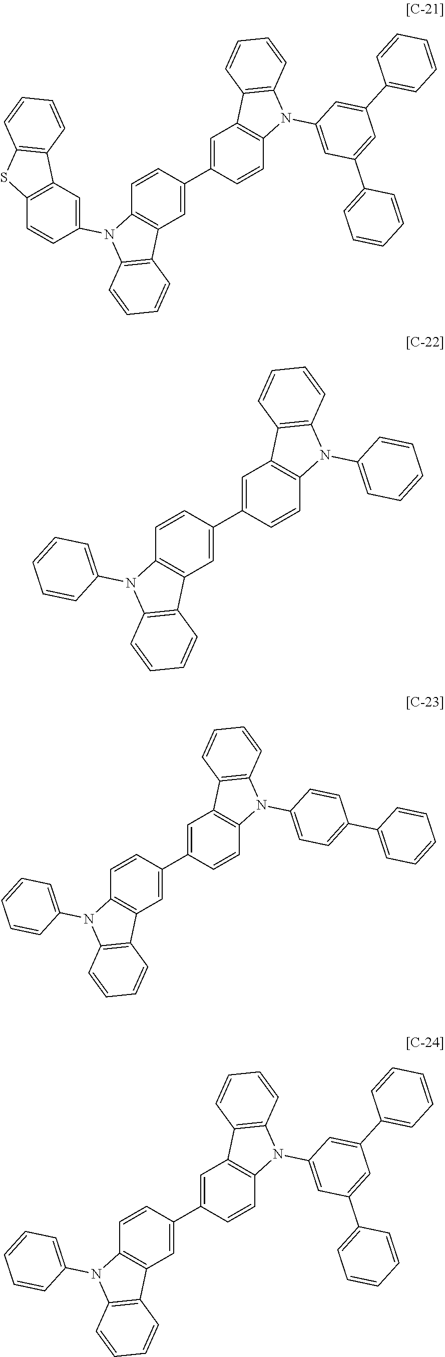 [SMILES: c1ccc(-c2cc(-c3ccccc3)cc(-n3c4ccccc4c4cc(-c5ccc6c(c5)c5ccccc5n6-c5ccc6sc7ccccc7c6c5)ccc43)c2)cc1 ; c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-c3ccccc3)cc(-n3c4ccccc4c4cc(-c5ccc6c(c5)c5ccccc5n6-c5ccccc5)ccc43)c2)cc1]

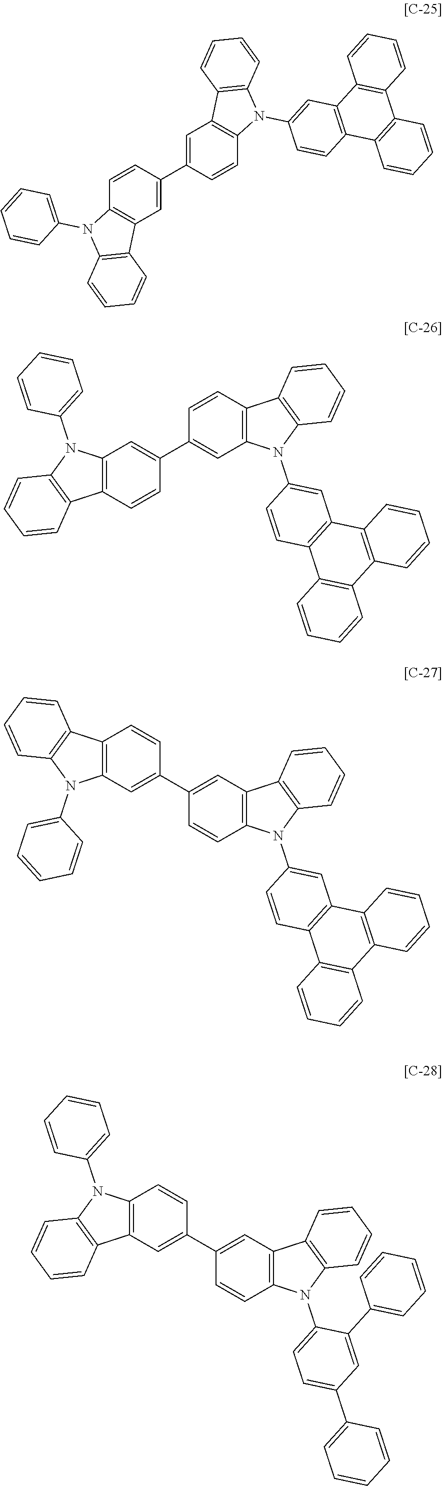 [SMILES: c1ccc(-n2c3ccccc3c3cc(-c4ccc5c(c4)c4ccccc4n5-c4ccc5c6ccccc6c6ccccc6c5c4)ccc32)cc1 ; c1ccc(-n2c3ccccc3c3ccc(-c4ccc5c6ccccc6n(-c6ccc7c8ccccc8c8ccccc8c7c6)c5c4)cc32)cc1 ; c1ccc(-n2c3ccccc3c3ccc(-c4ccc5c(c4)c4ccccc4n5-c4ccc5c6ccccc6c6ccccc6c5c4)cc32)cc1 ; c1ccc(-c2ccc(-n3c4ccccc4c4cc(-c5ccc6c(c5)c5ccccc5n6-c5ccccc5)ccc43)c(-c3ccccc3)c2)cc1]

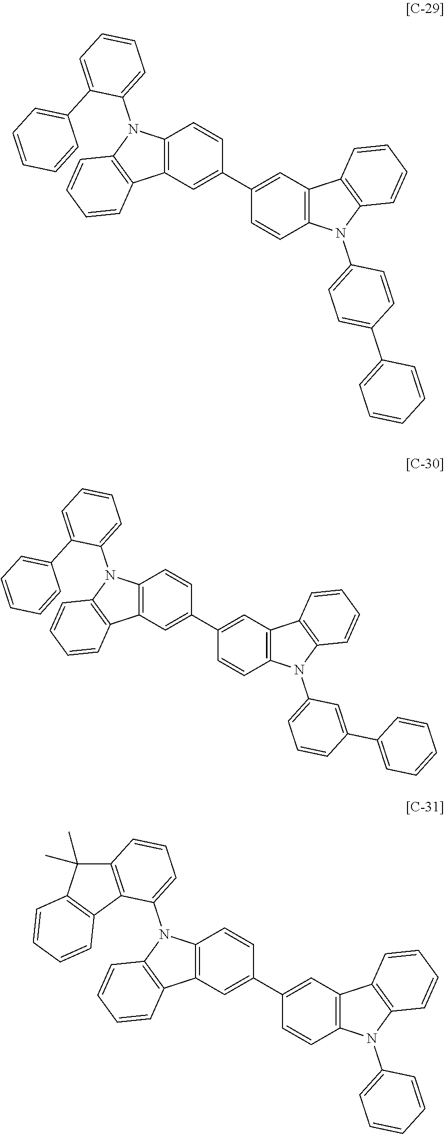 [SMILES: c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2cccc(-n3c4ccccc4c4cc(-c5ccc6c(c5)c5ccccc5n6-c5ccccc5-c5ccccc5)ccc43)c2)cc1 ; CC1(C)c2ccccc2-c2c(-n3c4ccccc4c4cc(-c5ccc6c(c5)c5ccccc5n6-c5ccccc5)ccc43)cccc21]

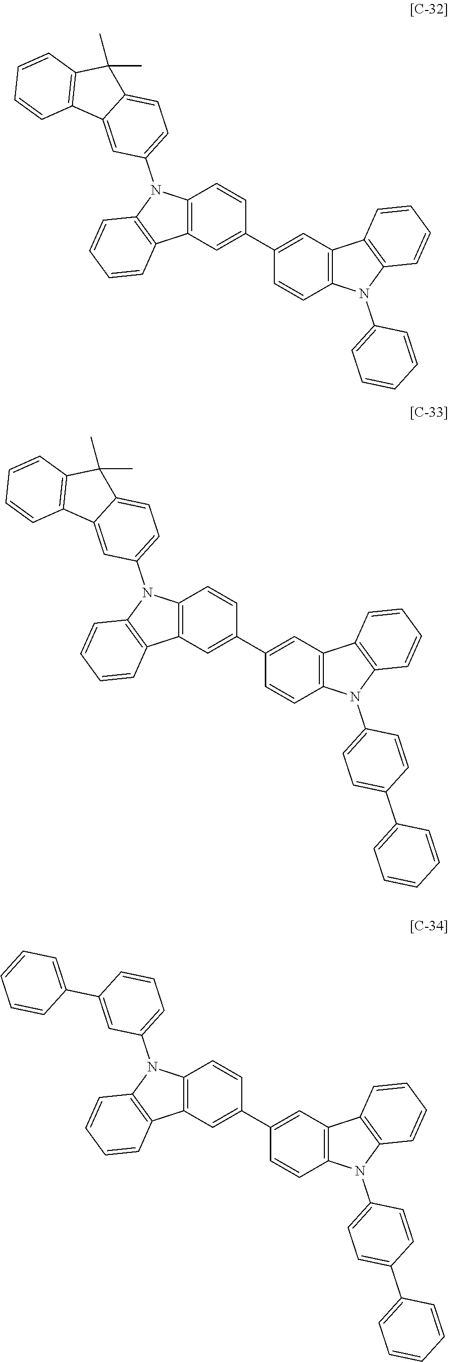 [SMILES: CC1(C)c2ccccc2-c2cc(-n3c4ccccc4c4cc(-c5ccc6c(c5)c5ccccc5n6-c5ccccc5)ccc43)ccc21 ; CC1(C)c2ccccc2-c2cc(-n3c4ccccc4c4cc(-c5ccc6c(c5)c5ccccc5n6-c5ccc(-c6ccccc6)cc5)ccc43)ccc21 ; c1ccc(-c2ccc(-n3c4ccccc4c4cc(-c5ccc6c(c5)c5ccccc5n6-c5cccc(-c6ccccc6)c5)ccc43)cc2)cc1]

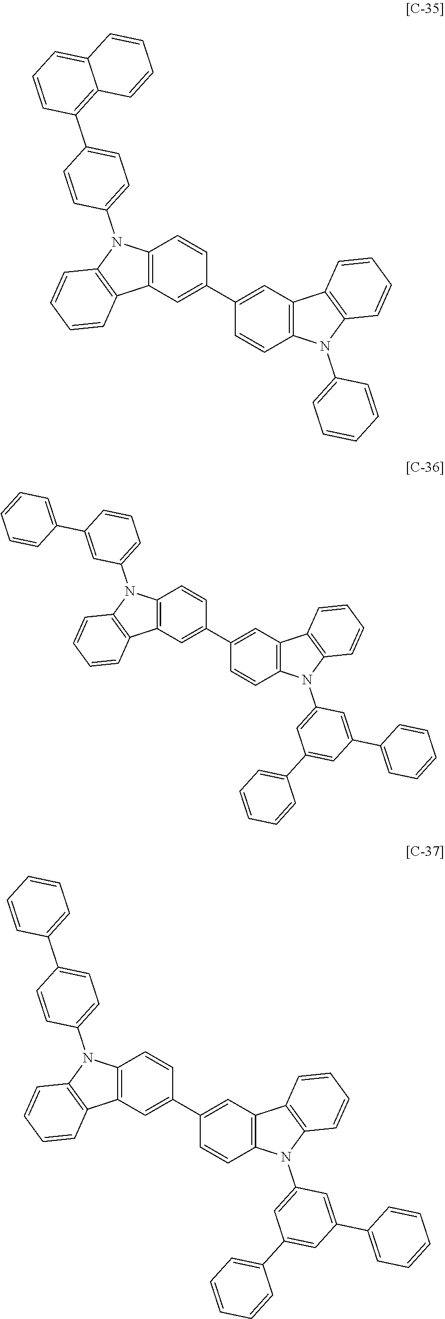 [SMILES: c1ccc(-n2c3ccccc3c3cc(-c4ccc5c(c4)c4ccccc4n5-c4ccc(-c5cccc6ccccc56)cc4)ccc32)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1]

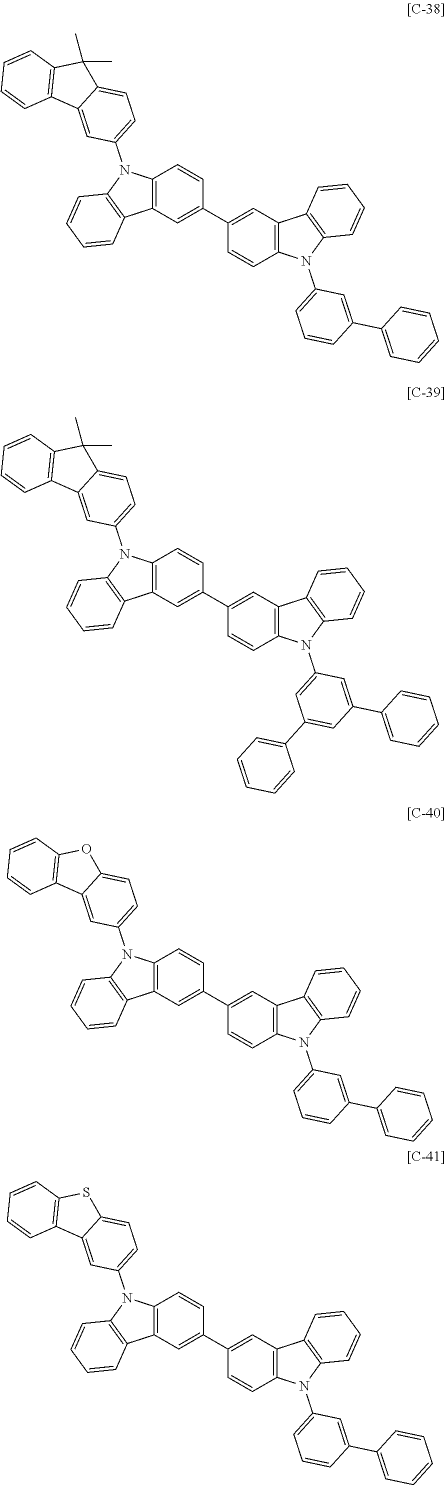 [SMILES: CC1(C)c2ccccc2-c2cc(-n3c4ccccc4c4cc(-c5ccc6c(c5)c5ccccc5n6-c5cccc(-c6ccccc6)c5)ccc43)ccc21 ; CC1(C)c2ccccc2-c2cc(-n3c4ccccc4c4cc(-c5ccc6c(c5)c5ccccc5n6-c5cc(-c6ccccc6)cc(-c6ccccc6)c5)ccc43)ccc21 ; c1ccc(-c2cccc(-n3c4ccccc4c4cc(-c5ccc6c(c5)c5ccccc5n6-c5ccc6oc7ccccc7c6c5)ccc43)c2)cc1 ; c1ccc(-c2cccc(-n3c4ccccc4c4cc(-c5ccc6c(c5)c5ccccc5n6-c5ccc6sc7ccccc7c6c5)ccc43)c2)cc1]

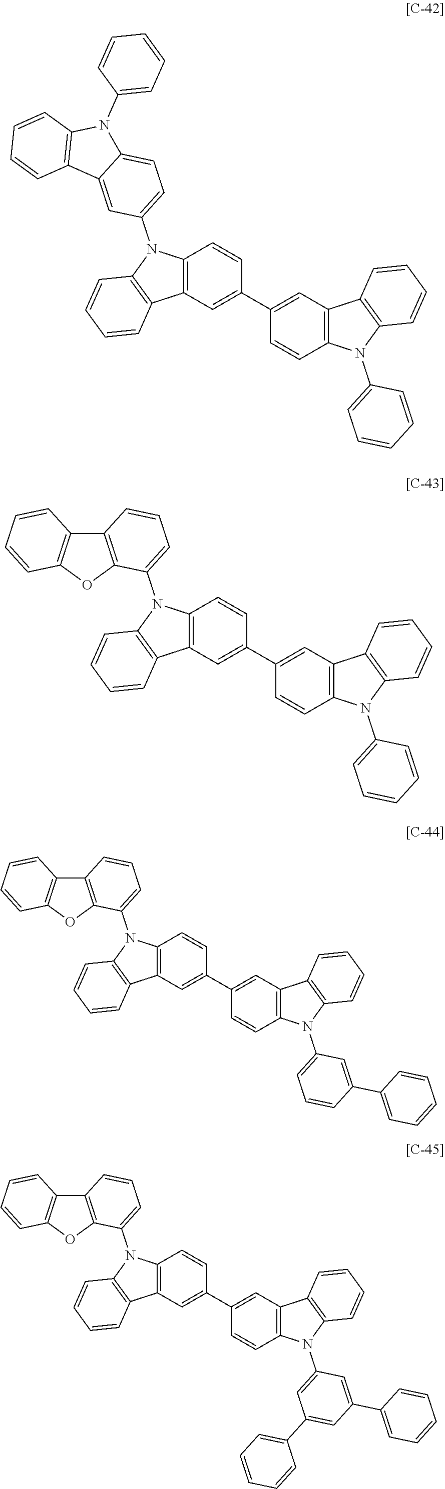 [SMILES: c1ccc(-n2c3ccccc3c3cc(-c4ccc5c(c4)c4ccccc4n5-c4ccc5c(c4)c4ccccc4n5-c4ccccc4)ccc32)cc1 ; c1ccc(-n2c3ccccc3c3cc(-c4ccc5c(c4)c4ccccc4n5-c4cccc5c4oc4ccccc45)ccc32)cc1 ; c1ccc(-c2cccc(-n3c4ccccc4c4cc(-c5ccc6c(c5)c5ccccc5n6-c5cccc6c5oc5ccccc56)ccc43)c2)cc1 ; c1ccc(-c2cc(-c3ccccc3)cc(-n3c4ccccc4c4cc(-c5ccc6c(c5)c5ccccc5n6-c5cccc6c5oc5ccccc56)ccc43)c2)cc1]

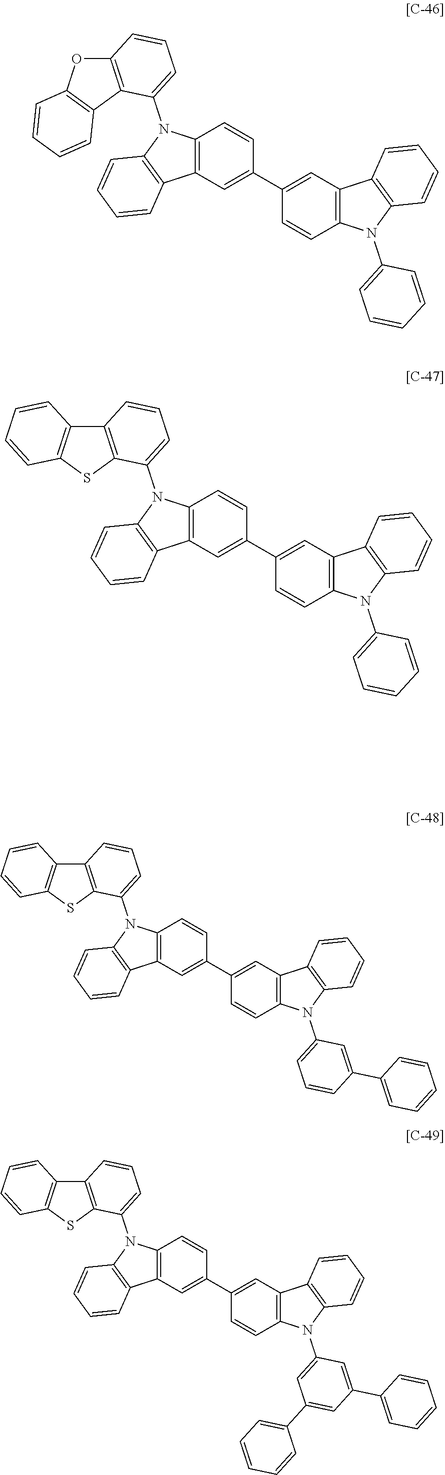 [SMILES: c1ccc(-n2c3ccccc3c3cc(-c4ccc5c(c4)c4ccccc4n5-c4cccc5oc6ccccc6c45)ccc32)cc1 ; c1ccc(-n2c3ccccc3c3cc(-c4ccc5c(c4)c4ccccc4n5-c4cccc5c4sc4ccccc45)ccc32)cc1 ; c1ccc(-c2cccc(-n3c4ccccc4c4cc(-c5ccc6c(c5)c5ccccc5n6-c5cccc6c5sc5ccccc56)ccc43)c2)cc1 ; c1ccc(-c2cc(-c3ccccc3)cc(-n3c4ccccc4c4cc(-c5ccc6c(c5)c5ccccc5n6-c5cccc6c5sc5ccccc56)ccc43)c2)cc1]

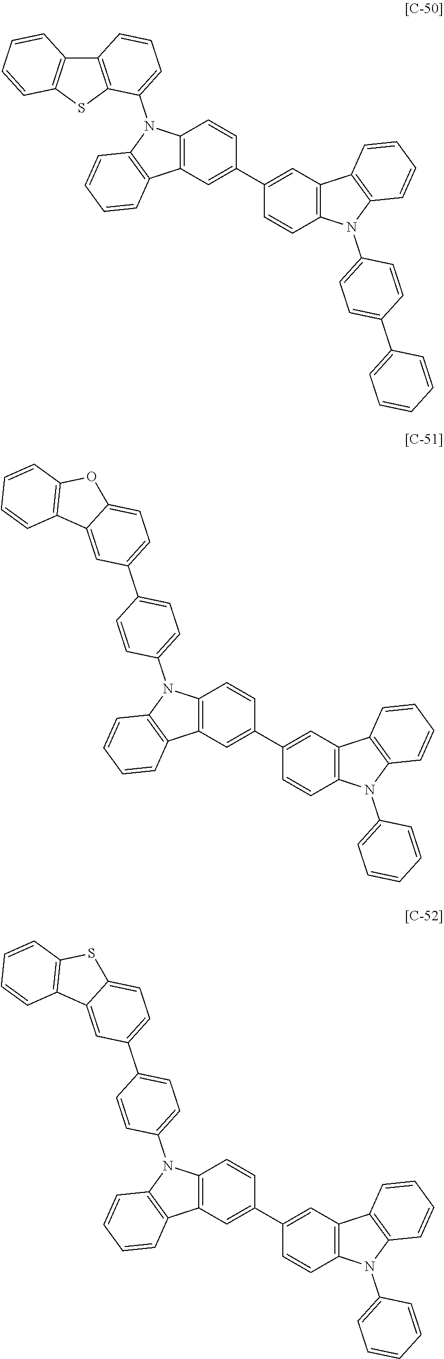 [SMILES: c1ccc(-c2ccc(-n3c4ccccc4c4cc(-c5ccc6c(c5)c5ccccc5n6-c5cccc6c5sc5ccccc56)ccc43)cc2)cc1 ; c1ccc(-n2c3ccccc3c3cc(-c4ccc5c(c4)c4ccccc4n5-c4ccc(-c5ccc6oc7ccccc7c6c5)cc4)ccc32)cc1 ; c1ccc(-n2c3ccccc3c3cc(-c4ccc5c(c4)c4ccccc4n5-c4ccc(-c5ccc6sc7ccccc7c6c5)cc4)ccc32)cc1]

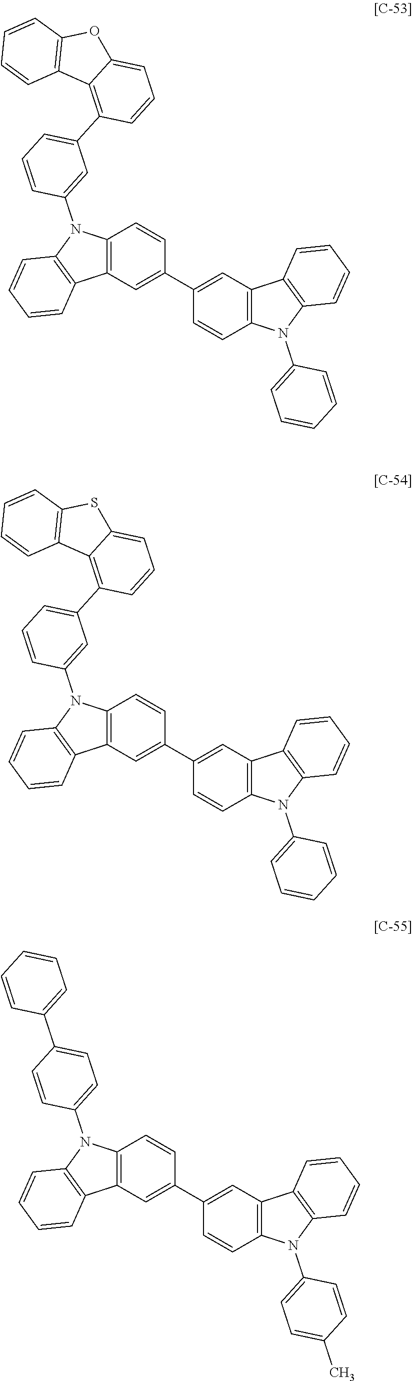 [SMILES: c1ccc(-n2c3ccccc3c3cc(-c4ccc5c(c4)c4ccccc4n5-c4cccc(-c5cccc6oc7ccccc7c56)c4)ccc32)cc1 ; c1ccc(-n2c3ccccc3c3cc(-c4ccc5c(c4)c4ccccc4n5-c4cccc(-c5cccc6sc7ccccc7c56)c4)ccc32)cc1 ; Cc1ccc(-n2c3ccccc3c3cc(-c4ccc5c(c4)c4ccccc4n5-c4ccc(-c5ccccc5)cc4)ccc32)cc1]

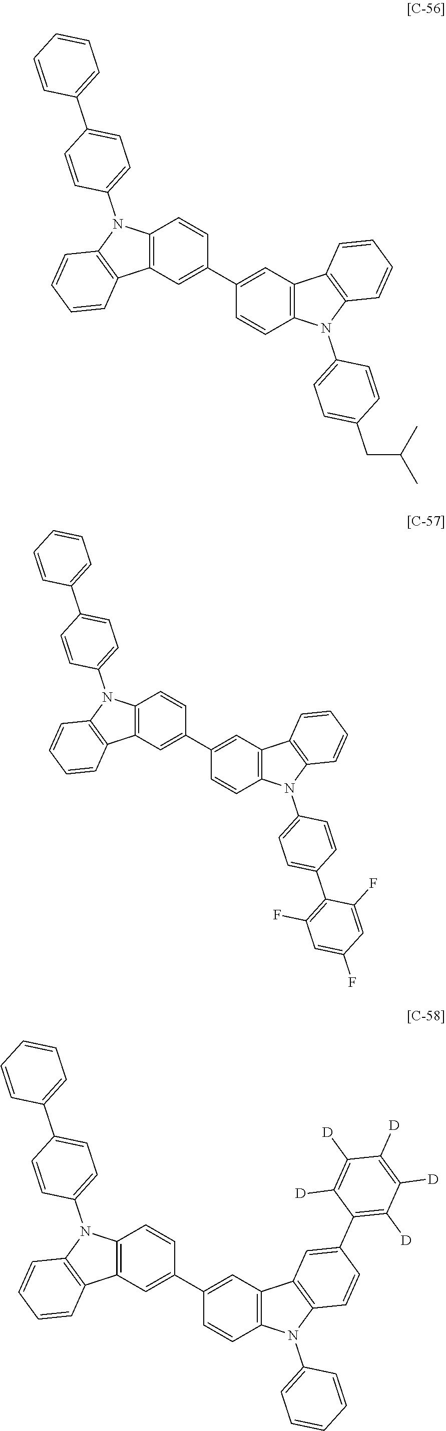 [SMILES: CC(C)Cc1ccc(-n2c3ccccc3c3cc(-c4ccc5c(c4)c4ccccc4n5-c4ccc(-c5ccccc5)cc4)ccc32)cc1 ; Fc1cc(F)c(-c2ccc(-n3c4ccccc4c4cc(-c5ccc6c(c5)c5ccccc5n6-c5ccc(-c6ccccc6)cc5)ccc43)cc2)c(F)c1 ; [2H]c1c([2H])c([2H])c(-c2ccc3c(c2)c2cc(-c4ccc5c(c4)c4ccccc4n5-c4ccc(-c5ccccc5)cc4)ccc2n3-c2ccccc2)c([2H])c1[2H]]

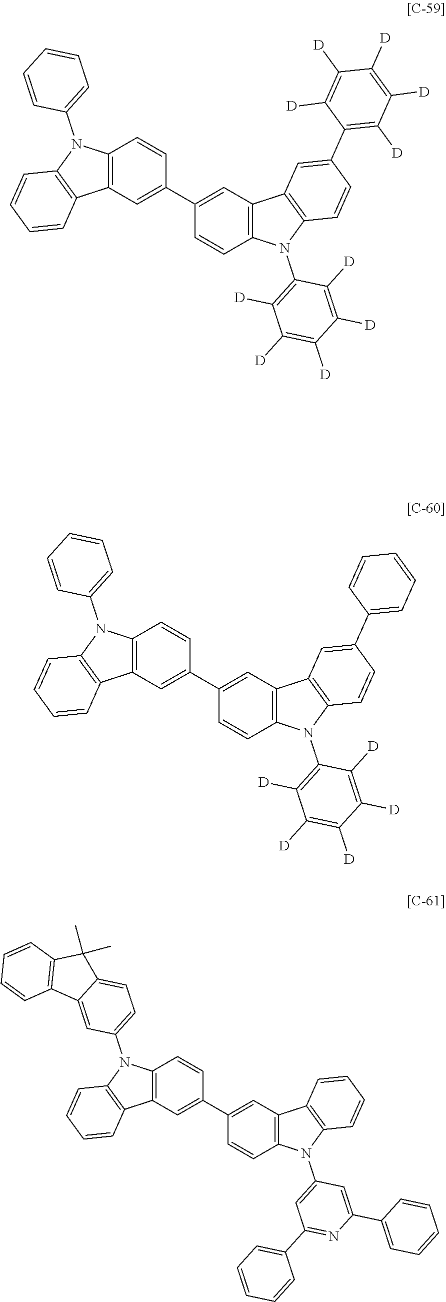 [SMILES: [2H]c1c([2H])c([2H])c(-c2ccc3c(c2)c2cc(-c4ccc5c(c4)c4ccccc4n5-c4ccccc4)ccc2n3-c2c([2H])c([2H])c([2H])c([2H])c2[2H])c([2H])c1[2H] ; [2H]c1c([2H])c([2H])c(-n2c3ccc(-c4ccccc4)cc3c3cc(-c4ccc5c(c4)c4ccccc4n5-c4ccccc4)ccc32)c([2H])c1[2H] ; CC1(C)c2ccccc2-c2cc(-n3c4ccccc4c4cc(-c5ccc6c(c5)c5ccccc5n6-c5cc(-c6ccccc6)nc(-c6ccccc6)c5)ccc43)ccc21]

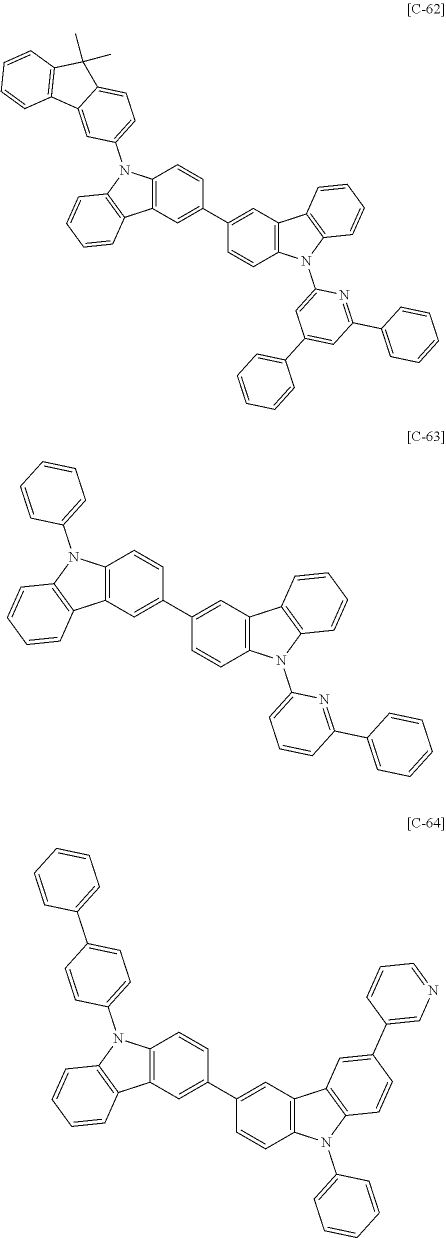 [SMILES: CC1(C)c2ccccc2-c2cc(-n3c4ccccc4c4cc(-c5ccc6c(c5)c5ccccc5n6-c5cc(-c6ccccc6)cc(-c6ccccc6)n5)ccc43)ccc21 ; c1ccc(-c2cccc(-n3c4ccccc4c4cc(-c5ccc6c(c5)c5ccccc5n6-c5ccccc5)ccc43)n2)cc1 ; c1ccc(-c2ccc(-n3c4ccccc4c4cc(-c5ccc6c(c5)c5cc(-c7cccnc7)ccc5n6-c5ccccc5)ccc43)cc2)cc1]

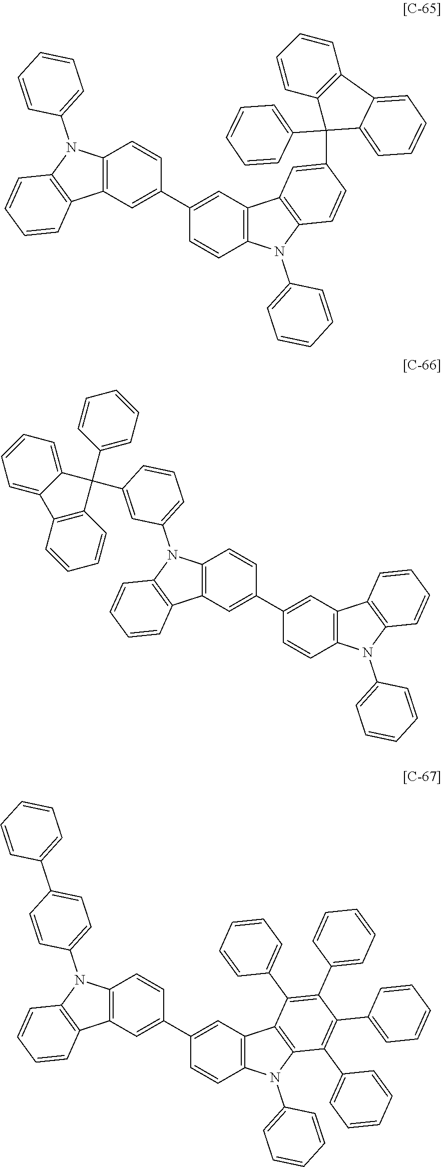 [SMILES: c1ccc(-n2c3ccccc3c3cc(-c4ccc5c(c4)c4cc(C6(c7ccccc7)c7ccccc7-c7ccccc76)ccc4n5-c4ccccc4)ccc32)cc1 ; c1ccc(-n2c3ccccc3c3cc(-c4ccc5c(c4)c4ccccc4n5-c4cccc(C5(c6ccccc6)c6ccccc6-c6ccccc65)c4)ccc32)cc1 ; c1ccc(-c2ccc(-n3c4ccccc4c4cc(-c5ccc6c(c5)c5c(-c7ccccc7)c(-c7ccccc7)c(-c7ccccc7)c(-c7ccccc7)c5n6-c5ccccc5)ccc43)cc2)cc1]

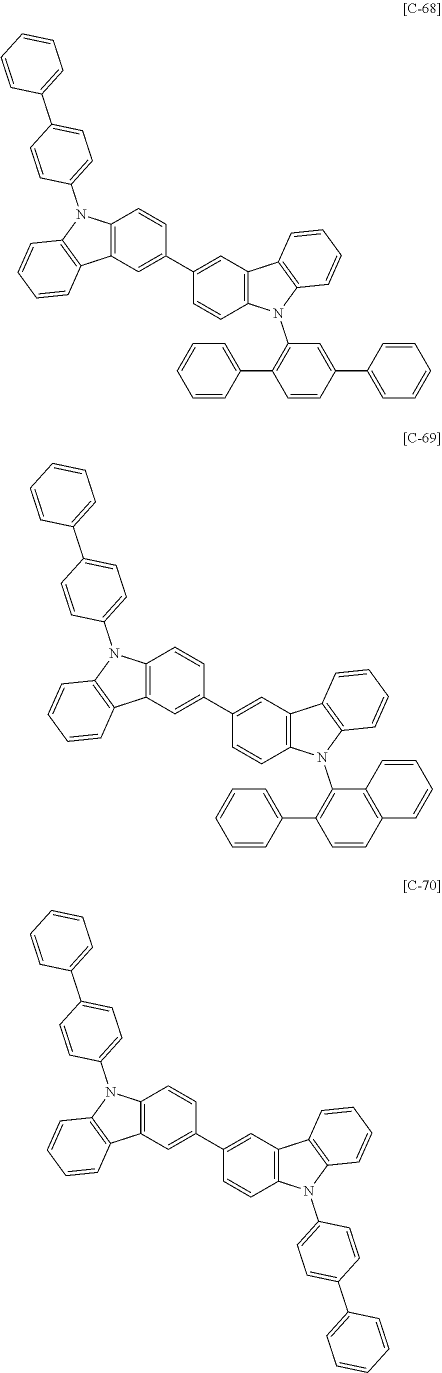 [SMILES: c1ccc(-c2ccc(-n3c4ccccc4c4cc(-c5ccc6c(c5)c5ccccc5n6-c5cc(-c6ccccc6)ccc5-c5ccccc5)ccc43)cc2)cc1 ; c1ccc(-c2ccc(-n3c4ccccc4c4cc(-c5ccc6c(c5)c5ccccc5n6-c5c(-c6ccccc6)ccc6ccccc56)ccc43)cc2)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

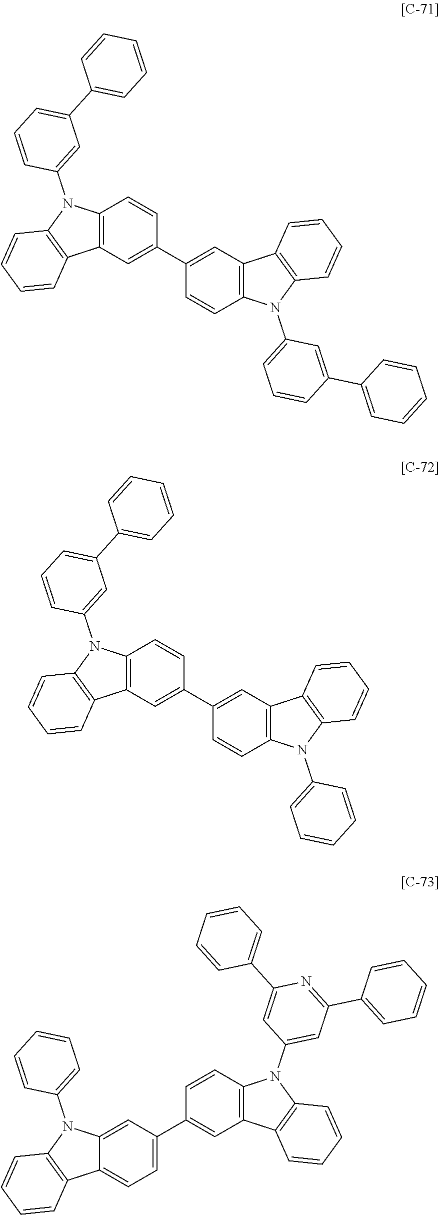 [SMILES: c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2cccc(-n3c4ccccc4c4cc(-c5ccc6c(c5)c5ccccc5n6-c5ccccc5)ccc43)c2)cc1 ; c1ccc(-c2cc(-n3c4ccccc4c4cc(-c5ccc6c7ccccc7n(-c7ccccc7)c6c5)ccc43)cc(-c3ccccc3)n2)cc1]

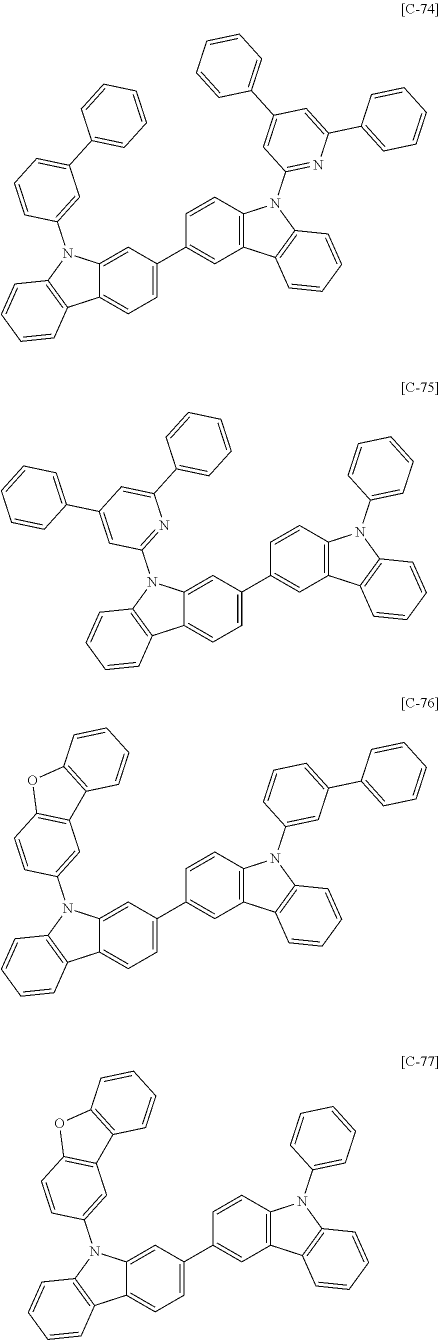 [SMILES: c1ccc(-c2cccc(-n3c4ccccc4c4ccc(-c5ccc6c(c5)c5ccccc5n6-c5cc(-c6ccccc6)cc(-c6ccccc6)n5)cc43)c2)cc1 ; c1ccc(-c2cc(-c3ccccc3)nc(-n3c4ccccc4c4ccc(-c5ccc6c(c5)c5ccccc5n6-c5ccccc5)cc43)c2)cc1 ; c1ccc(-c2cccc(-n3c4ccccc4c4cc(-c5ccc6c7ccccc7n(-c7ccc8oc9ccccc9c8c7)c6c5)ccc43)c2)cc1 ; c1ccc(-n2c3ccccc3c3cc(-c4ccc5c6ccccc6n(-c6ccc7oc8ccccc8c7c6)c5c4)ccc32)cc1]

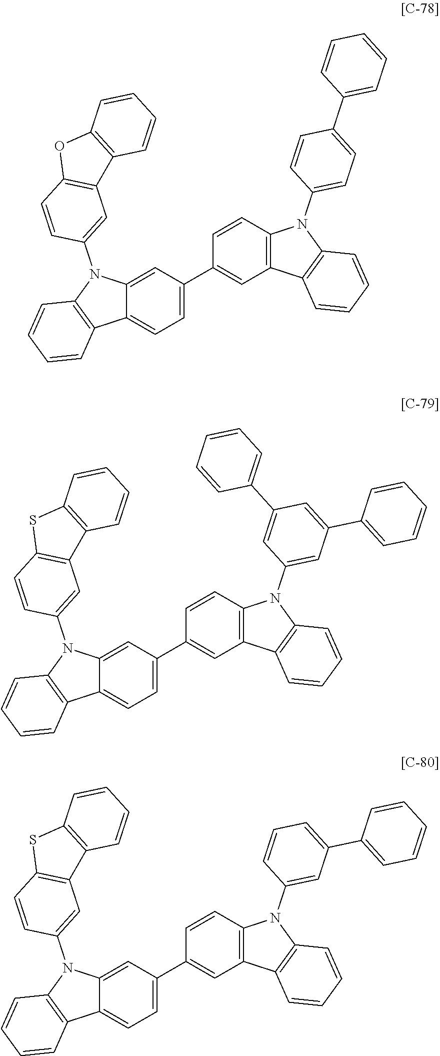 [SMILES: c1ccc(-c2ccc(-n3c4ccccc4c4cc(-c5ccc6c7ccccc7n(-c7ccc8oc9ccccc9c8c7)c6c5)ccc43)cc2)cc1 ; c1ccc(-c2cc(-c3ccccc3)cc(-n3c4ccccc4c4cc(-c5ccc6c7ccccc7n(-c7ccc8sc9ccccc9c8c7)c6c5)ccc43)c2)cc1 ; c1ccc(-c2cccc(-n3c4ccccc4c4cc(-c5ccc6c7ccccc7n(-c7ccc8sc9ccccc9c8c7)c6c5)ccc43)c2)cc1]

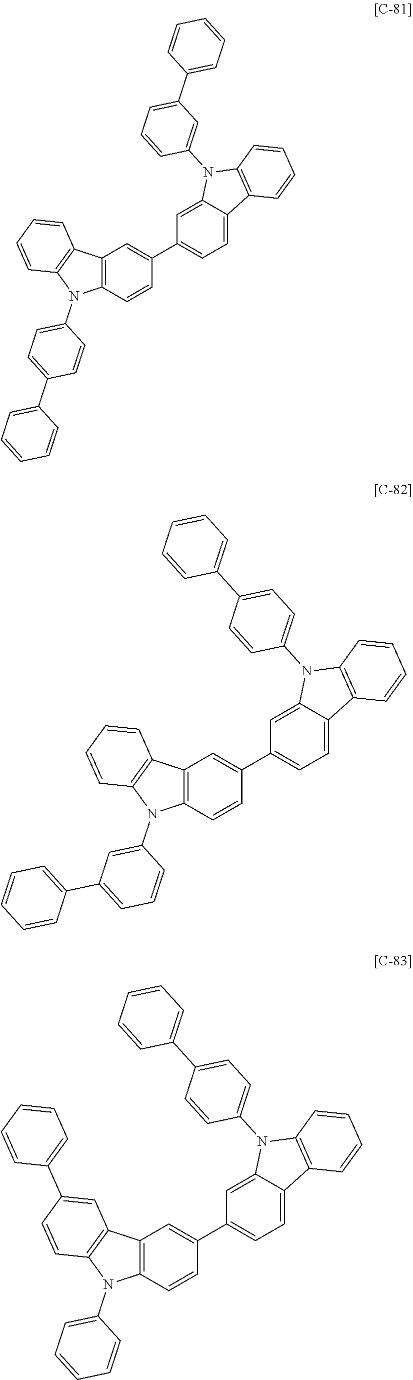 [SMILES: c1ccc(-c2ccc(-n3c4ccccc4c4cc(-c5ccc6c7ccccc7n(-c7cccc(-c8ccccc8)c7)c6c5)ccc43)cc2)cc1 ; c1ccc(-c2ccc(-n3c4ccccc4c4ccc(-c5ccc6c(c5)c5ccccc5n6-c5cccc(-c6ccccc6)c5)cc43)cc2)cc1 ; c1ccc(-c2ccc(-n3c4ccccc4c4ccc(-c5ccc6c(c5)c5cc(-c7ccccc7)ccc5n6-c5ccccc5)cc43)cc2)cc1]

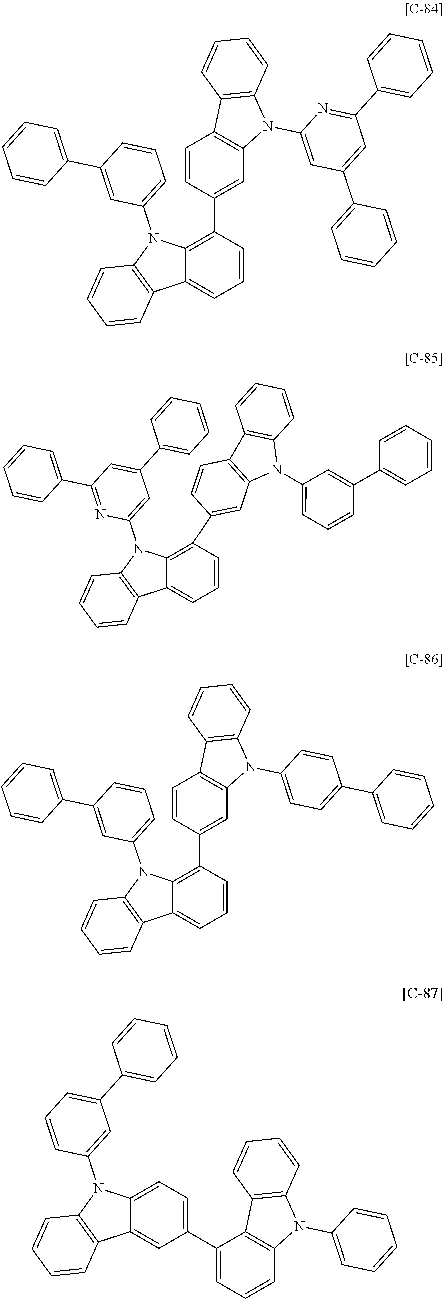 [SMILES: c1ccc(-c2cccc(-n3c4ccccc4c4cccc(-c5ccc6c7ccccc7n(-c7cc(-c8ccccc8)cc(-c8ccccc8)n7)c6c5)c43)c2)cc1 ; c1ccc(-c2cccc(-n3c4ccccc4c4ccc(-c5cccc6c7ccccc7n(-c7cc(-c8ccccc8)cc(-c8ccccc8)n7)c56)cc43)c2)cc1 ; c1ccc(-c2ccc(-n3c4ccccc4c4ccc(-c5cccc6c7ccccc7n(-c7cccc(-c8ccccc8)c7)c56)cc43)cc2)cc1 ; c1ccc(-c2cccc(-n3c4ccccc4c4cc(-c5cccc6c5c5ccccc5n6-c5ccccc5)ccc43)c2)cc1]

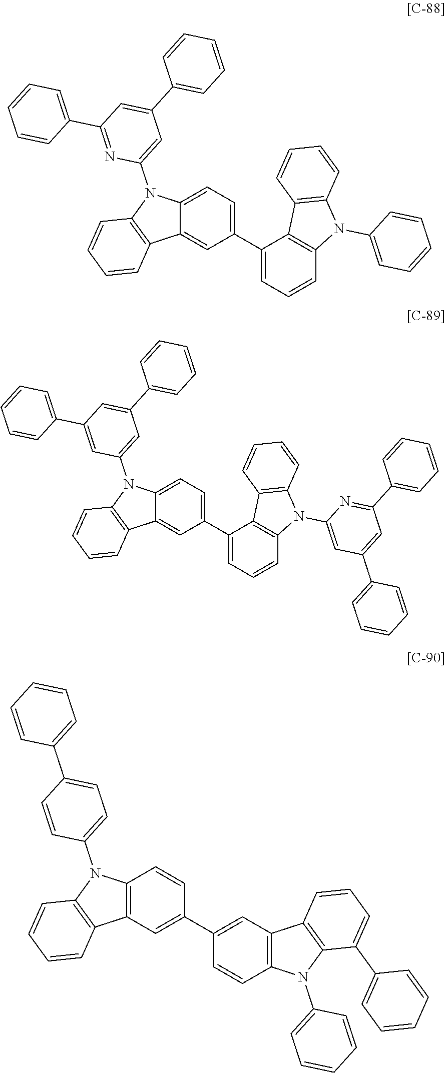 [SMILES: c1ccc(-c2cc(-c3ccccc3)nc(-n3c4ccccc4c4cc(-c5cccc6c5c5ccccc5n6-c5ccccc5)ccc43)c2)cc1 ; c1ccc(-c2cc(-c3ccccc3)cc(-n3c4ccccc4c4cc(-c5cccc6c5c5ccccc5n6-c5cc(-c6ccccc6)cc(-c6ccccc6)n5)ccc43)c2)cc1 ; c1ccc(-c2ccc(-n3c4ccccc4c4cc(-c5ccc6c(c5)c5cccc(-c7ccccc7)c5n6-c5ccccc5)ccc43)cc2)cc1]

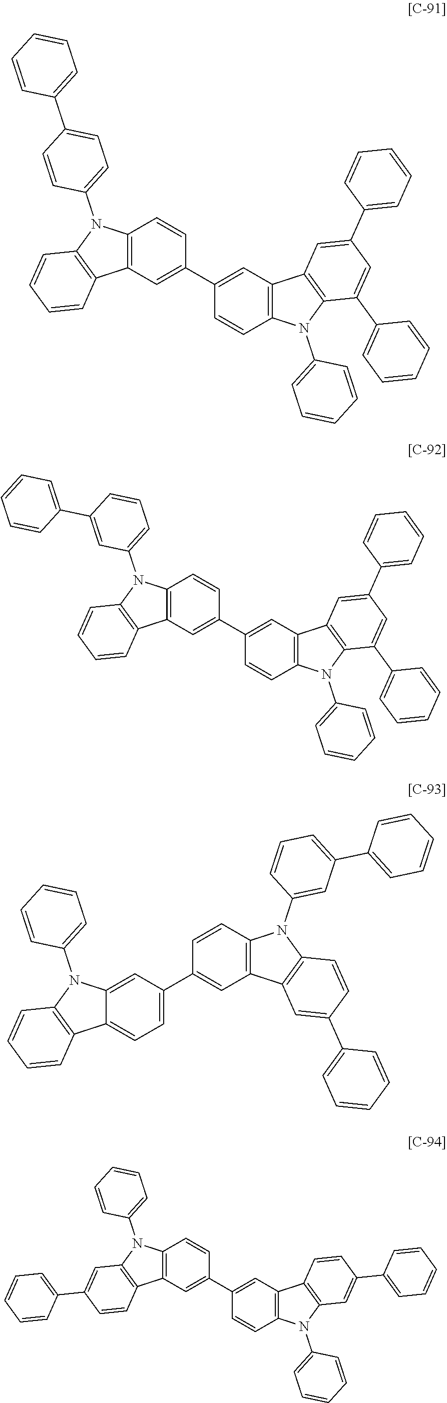 [SMILES: c1ccc(-c2ccc(-n3c4ccccc4c4cc(-c5ccc6c(c5)c5cc(-c7ccccc7)cc(-c7ccccc7)c5n6-c5ccccc5)ccc43)cc2)cc1 ; c1ccc(-c2cccc(-n3c4ccccc4c4cc(-c5ccc6c(c5)c5cc(-c7ccccc7)cc(-c7ccccc7)c5n6-c5ccccc5)ccc43)c2)cc1 ; c1ccc(-c2cccc(-n3c4ccc(-c5ccccc5)cc4c4cc(-c5ccc6c7ccccc7n(-c7ccccc7)c6c5)ccc43)c2)cc1 ; c1ccc(-c2ccc3c4cc(-c5ccc6c(c5)c5ccc(-c7ccccc7)cc5n6-c5ccccc5)ccc4n(-c4ccccc4)c3c2)cc1]

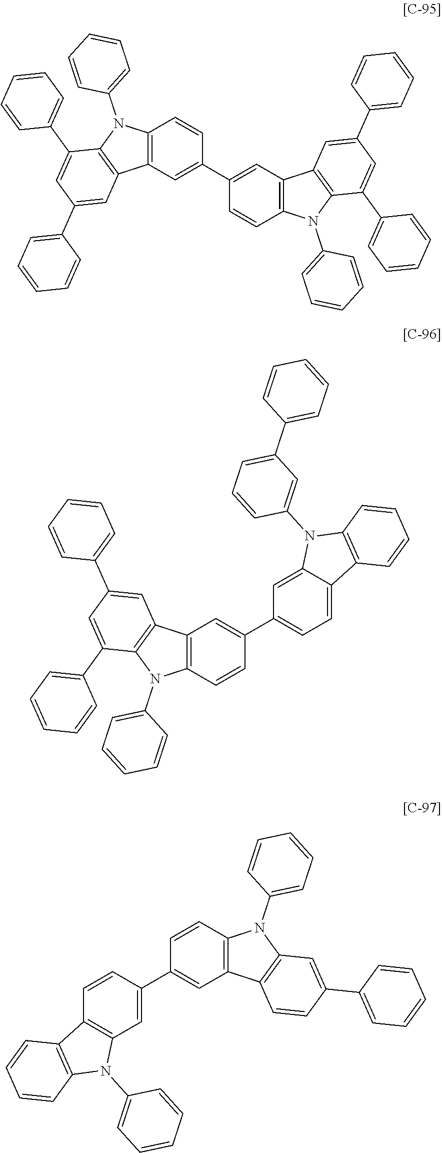 [SMILES: c1ccc(-c2cc(-c3ccccc3)c3c(c2)c2cc(-c4ccc5c(c4)c4cc(-c6ccccc6)cc(-c6ccccc6)c4n5-c4ccccc4)ccc2n3-c2ccccc2)cc1 ; c1ccc(-c2cccc(-n3c4ccccc4c4ccc(-c5ccc6c(c5)c5cc(-c7ccccc7)cc(-c7ccccc7)c5n6-c5ccccc5)cc43)c2)cc1 ; c1ccc(-c2ccc3c4cc(-c5ccc6c7ccccc7n(-c7ccccc7)c6c5)ccc4n(-c4ccccc4)c3c2)cc1]

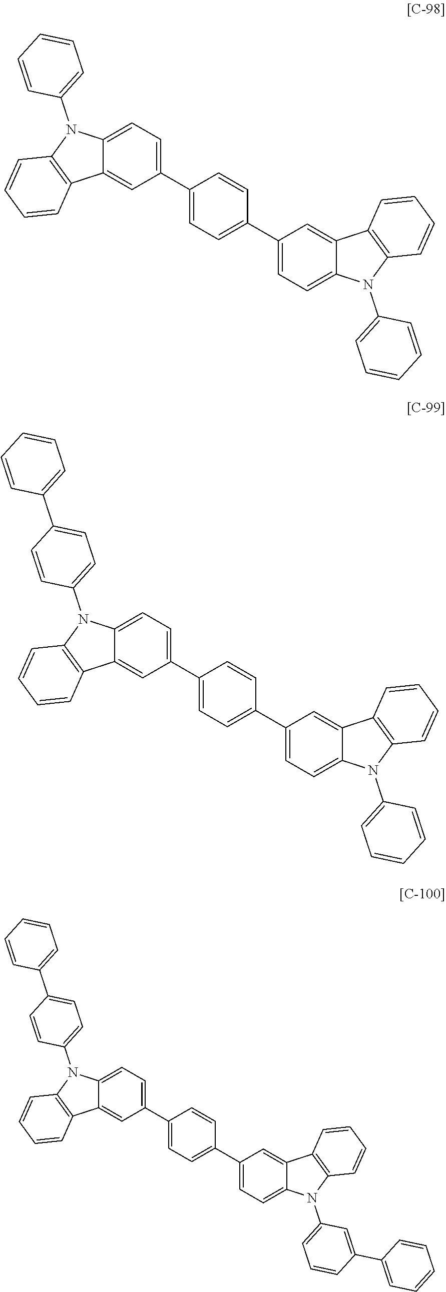 [SMILES: c1ccc(-n2c3ccccc3c3cc(-c4ccc(-c5ccc6c(c5)c5ccccc5n6-c5ccccc5)cc4)ccc32)cc1 ; c1ccc(-c2ccc(-n3c4ccccc4c4cc(-c5ccc(-c6ccc7c(c6)c6ccccc6n7-c6ccccc6)cc5)ccc43)cc2)cc1 ; c1ccc(-c2ccc(-n3c4ccccc4c4cc(-c5ccc(-c6ccc7c(c6)c6ccccc6n7-c6cccc(-c7ccccc7)c6)cc5)ccc43)cc2)cc1]

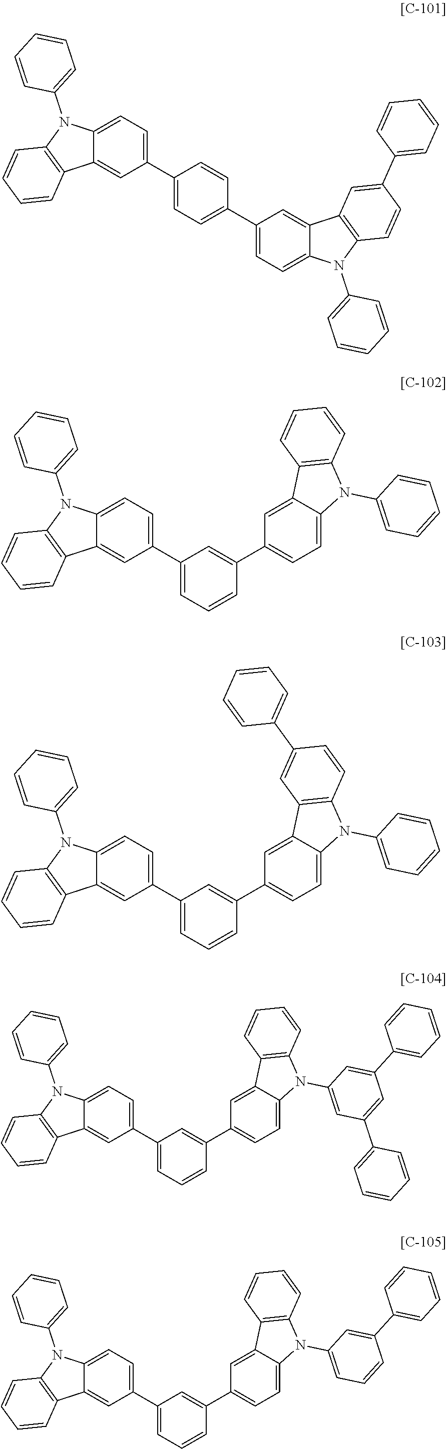 [SMILES: c1ccc(-c2ccc3c(c2)c2cc(-c4ccc(-c5ccc6c(c5)c5ccccc5n6-c5ccccc5)cc4)ccc2n3-c2ccccc2)cc1 ; c1ccc(-n2c3ccccc3c3cc(-c4cccc(-c5ccc6c(c5)c5ccccc5n6-c5ccccc5)c4)ccc32)cc1 ; c1ccc(-c2ccc3c(c2)c2cc(-c4cccc(-c5ccc6c(c5)c5ccccc5n6-c5ccccc5)c4)ccc2n3-c2ccccc2)cc1 ; c1ccc(-c2cc(-c3ccccc3)cc(-n3c4ccccc4c4cc(-c5cccc(-c6ccc7c(c6)c6ccccc6n7-c6ccccc6)c5)ccc43)c2)cc1 ; c1ccc(-c2cccc(-n3c4ccccc4c4cc(-c5cccc(-c6ccc7c(c6)c6ccccc6n7-c6ccccc6)c5)ccc43)c2)cc1]

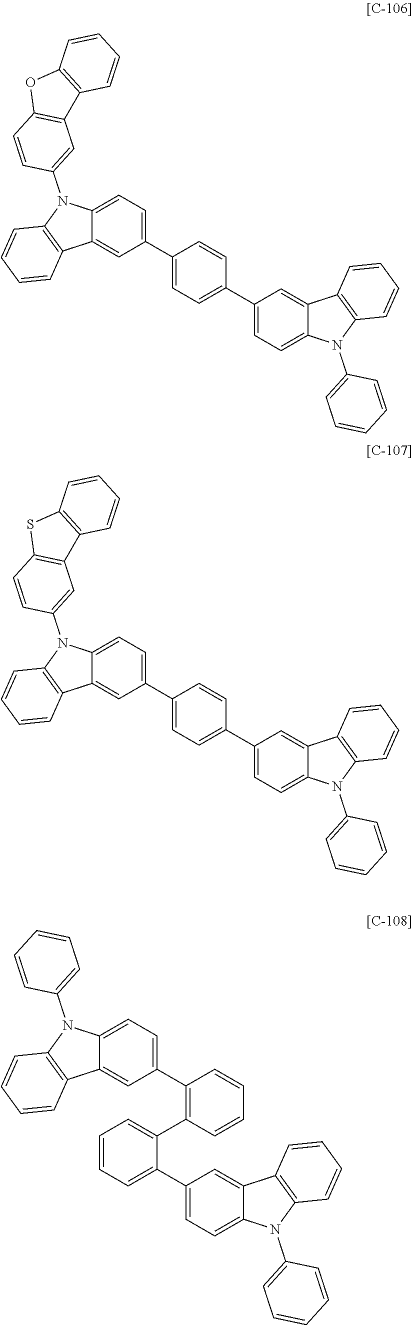 [SMILES: c1ccc(-n2c3ccccc3c3cc(-c4ccc(-c5ccc6c(c5)c5ccccc5n6-c5ccc6oc7ccccc7c6c5)cc4)ccc32)cc1 ; c1ccc(-n2c3ccccc3c3cc(-c4ccc(-c5ccc6c(c5)c5ccccc5n6-c5ccc6sc7ccccc7c6c5)cc4)ccc32)cc1 ; c1ccc(-n2c3ccccc3c3cc(-c4ccccc4-c4ccccc4-c4ccc5c(c4)c4ccccc4n5-c4ccccc4)ccc32)cc1]

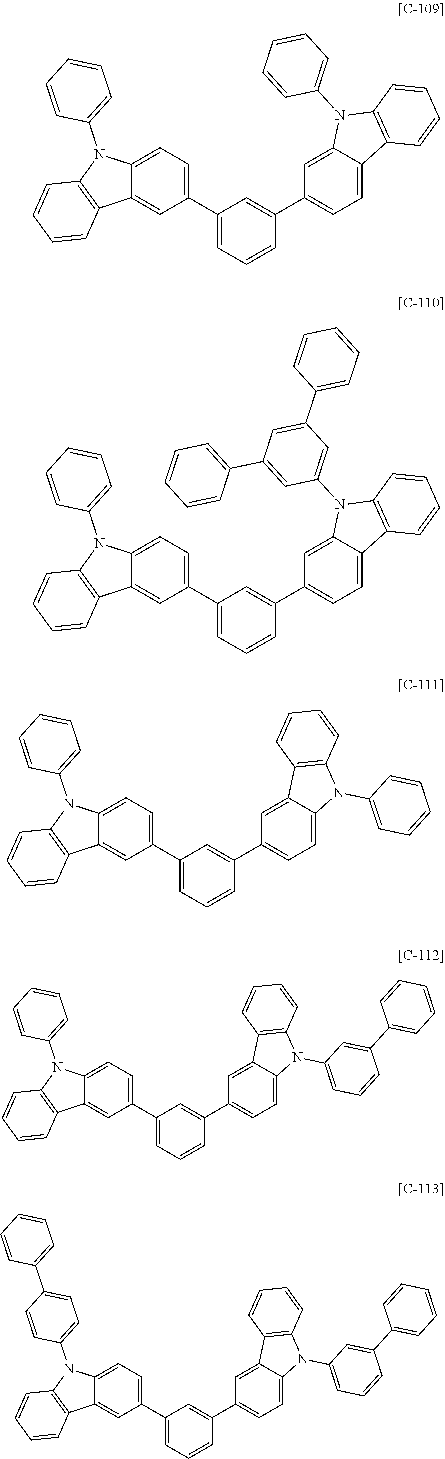 [SMILES: c1ccc(-n2c3ccccc3c3cc(-c4cccc(-c5ccc6c7ccccc7n(-c7ccccc7)c6c5)c4)ccc32)cc1 ; c1ccc(-c2cc(-c3ccccc3)cc(-n3c4ccccc4c4ccc(-c5cccc(-c6ccc7c(c6)c6ccccc6n7-c6ccccc6)c5)cc43)c2)cc1 ; c1ccc(-n2c3ccccc3c3cc(-c4cccc(-c5ccc6c(c5)c5ccccc5n6-c5ccccc5)c4)ccc32)cc1 ; c1ccc(-c2cccc(-n3c4ccccc4c4cc(-c5cccc(-c6ccc7c(c6)c6ccccc6n7-c6ccccc6)c5)ccc43)c2)cc1 ; c1ccc(-c2ccc(-n3c4ccccc4c4cc(-c5cccc(-c6ccc7c(c6)c6ccccc6n7-c6cccc(-c7ccccc7)c6)c5)ccc43)cc2)cc1]

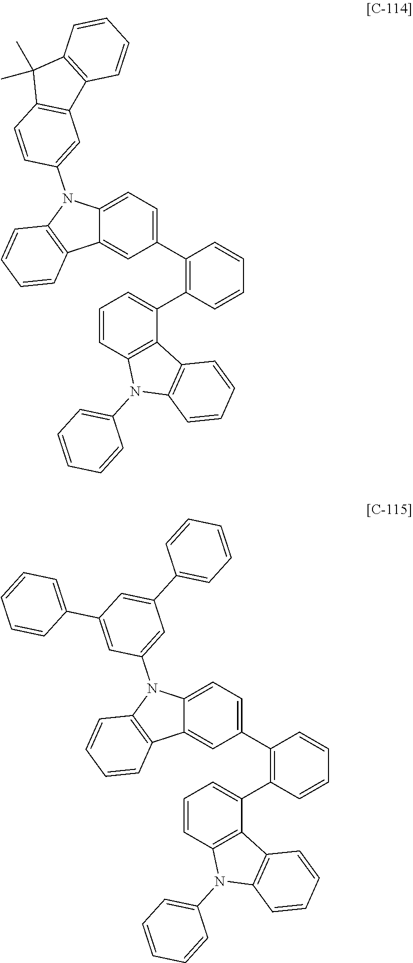 [SMILES: CC1(C)c2ccccc2-c2cc(-n3c4ccccc4c4cc(-c5ccccc5-c5cccc6c5c5ccccc5n6-c5ccccc5)ccc43)ccc21 ; c1ccc(-c2cc(-c3ccccc3)cc(-n3c4ccccc4c4cc(-c5ccccc5-c5cccc6c5c5ccccc5n6-c5ccccc5)ccc43)c2)cc1]

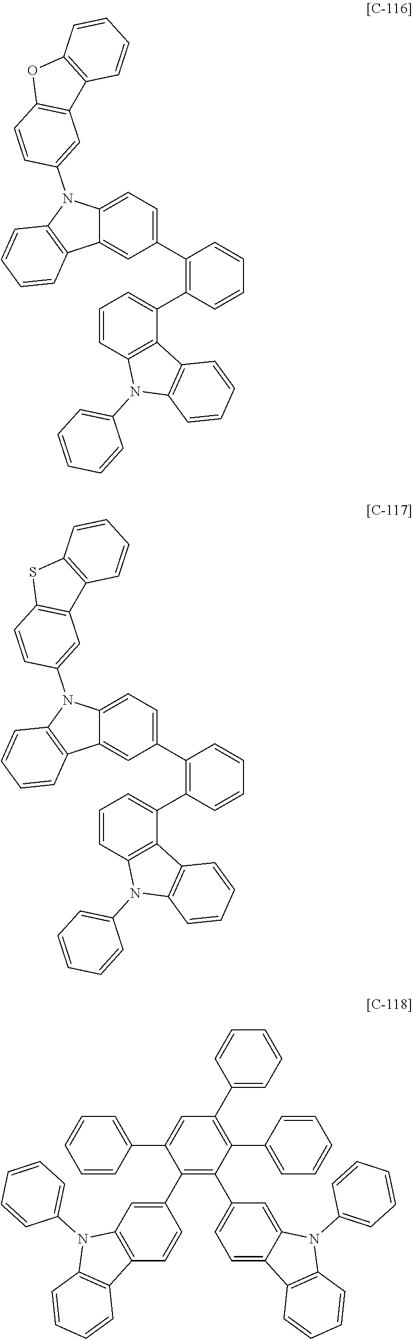 [SMILES: c1ccc(-n2c3ccccc3c3c(-c4ccccc4-c4ccc5c(c4)c4ccccc4n5-c4ccc5oc6ccccc6c5c4)cccc32)cc1 ; c1ccc(-n2c3ccccc3c3c(-c4ccccc4-c4ccc5c(c4)c4ccccc4n5-c4ccc5sc6ccccc6c5c4)cccc32)cc1 ; c1ccc(-c2cc(-c3ccccc3)c(-c3ccc4c5ccccc5n(-c5ccccc5)c4c3)c(-c3ccc4c5ccccc5n(-c5ccccc5)c4c3)c2-c2ccccc2)cc1]

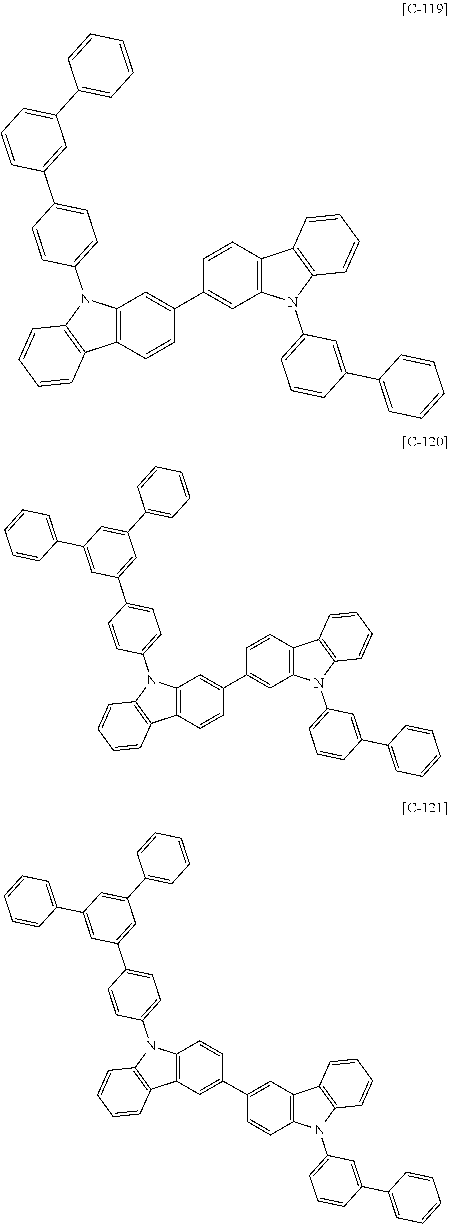 [SMILES: c1ccc(-c2cccc(-c3ccc(-n4c5ccccc5c5ccc(-c6ccc7c8ccccc8n(-c8cccc(-c9ccccc9)c8)c7c6)cc54)cc3)c2)cc1 ; c1ccc(-c2cc(-c3ccccc3)cc(-c3ccc(-n4c5ccccc5c5ccc(-c6ccc7c8ccccc8n(-c8cccc(-c9ccccc9)c8)c7c6)cc54)cc3)c2)cc1 ; c1ccc(-c2cc(-c3ccccc3)cc(-c3ccc(-n4c5ccccc5c5cc(-c6ccc7c(c6)c6ccccc6n7-c6cccc(-c7ccccc7)c6)ccc54)cc3)c2)cc1]

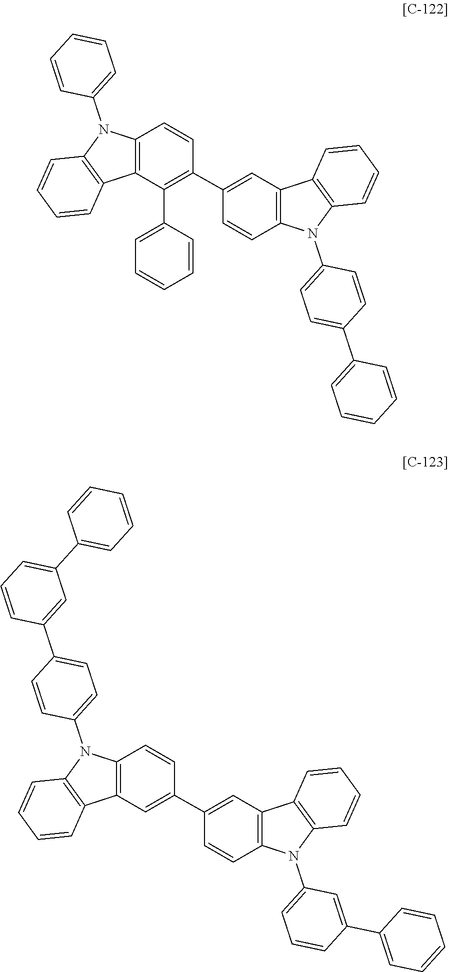 [SMILES: c1ccc(-c2ccc(-n3c4ccccc4c4cc(-c5ccc6c(c5-c5ccccc5)c5ccccc5n6-c5ccccc5)ccc43)cc2)cc1 ; c1ccc(-c2cccc(-c3ccc(-n4c5ccccc5c5cc(-c6ccc7c(c6)c6ccccc6n7-c6cccc(-c7ccccc7)c6)ccc54)cc3)c2)cc1]

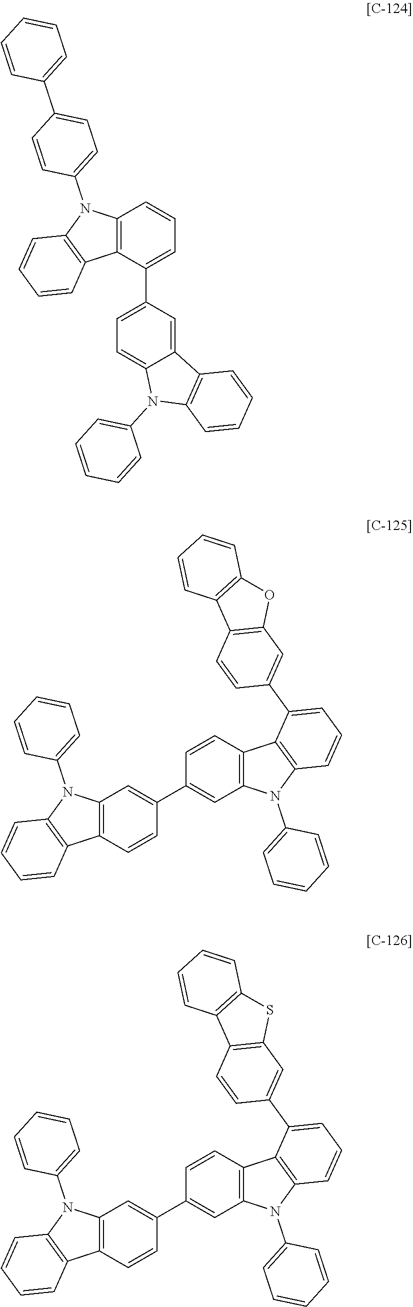 [SMILES: c1ccc(-c2ccc(-n3c4ccccc4c4c(-c5ccc6c(c5)c5ccccc5n6-c5ccccc5)cccc43)cc2)cc1 ; c1ccc(-n2c3ccccc3c3ccc(-c4ccc5c6c(-c7ccc8c(c7)oc7ccccc78)cccc6n(-c6ccccc6)c5c4)cc32)cc1 ; c1ccc(-n2c3ccccc3c3ccc(-c4ccc5c6c(-c7ccc8c(c7)sc7ccccc78)cccc6n(-c6ccccc6)c5c4)cc32)cc1]

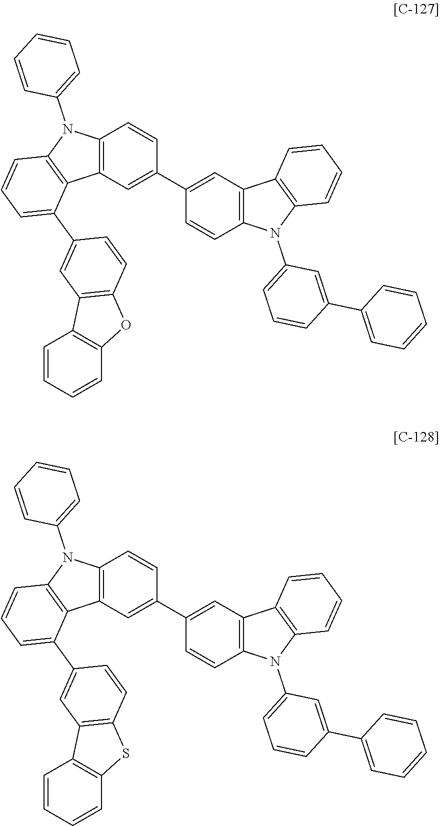 [SMILES: c1ccc(-c2cccc(-n3c4ccccc4c4cc(-c5ccc6c(c5)c5c(-c7ccc8oc9ccccc9c8c7)cccc5n6-c5ccccc5)ccc43)c2)cc1 ; c1ccc(-c2cccc(-n3c4ccccc4c4cc(-c5ccc6c(c5)c5c(-c7ccc8sc9ccccc9c8c7)cccc5n6-c5ccccc5)ccc43)c2)cc1]

The composition for an organic optoelectronic device according to the specific embodiment of the present invention may include a first compound selected from compounds of Group 1-1 and a second compound selected from compounds of Group 2-1.

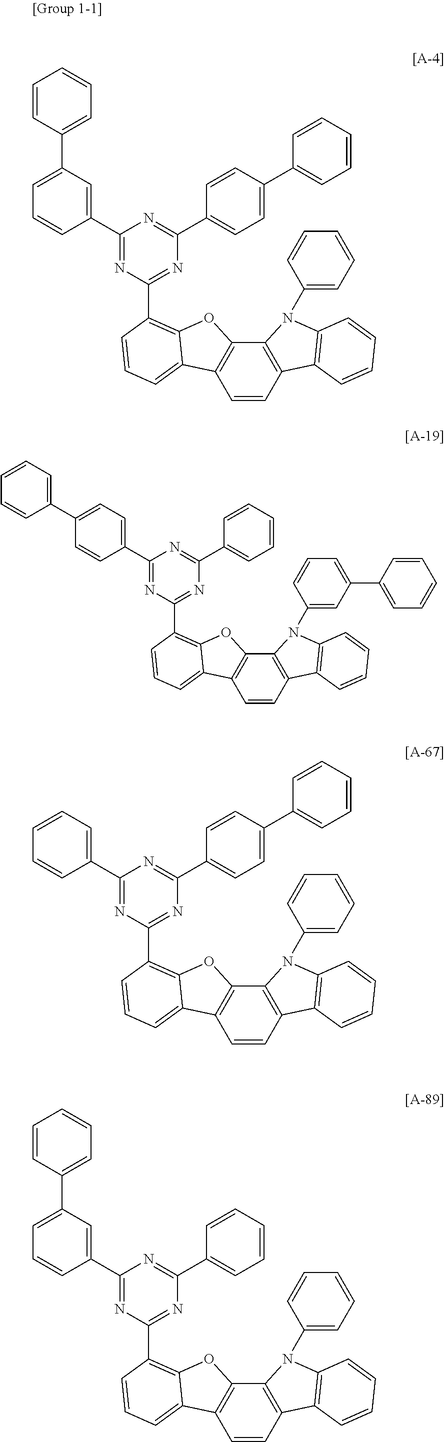 [SMILES: c1ccc(-c2ccc(-c3nc(-c4cccc(-c5ccccc5)c4)nc(-c4cccc5c4oc4c5ccc5c6ccccc6n(-c6ccccc6)c54)n3)cc2)cc1 ; c1ccc(-c2ccc(-c3nc(-c4ccccc4)nc(-c4cccc5c4oc4c5ccc5c6ccccc6n(-c6cccc(-c7ccccc7)c6)c54)n3)cc2)cc1 ; c1ccc(-c2ccc(-c3nc(-c4ccccc4)nc(-c4cccc5c4oc4c5ccc5c6ccccc6n(-c6ccccc6)c54)n3)cc2)cc1 ; c1ccc(-c2cccc(-c3nc(-c4ccccc4)nc(-c4cccc5c4oc4c5ccc5c6ccccc6n(-c6ccccc6)c54)n3)c2)cc1]

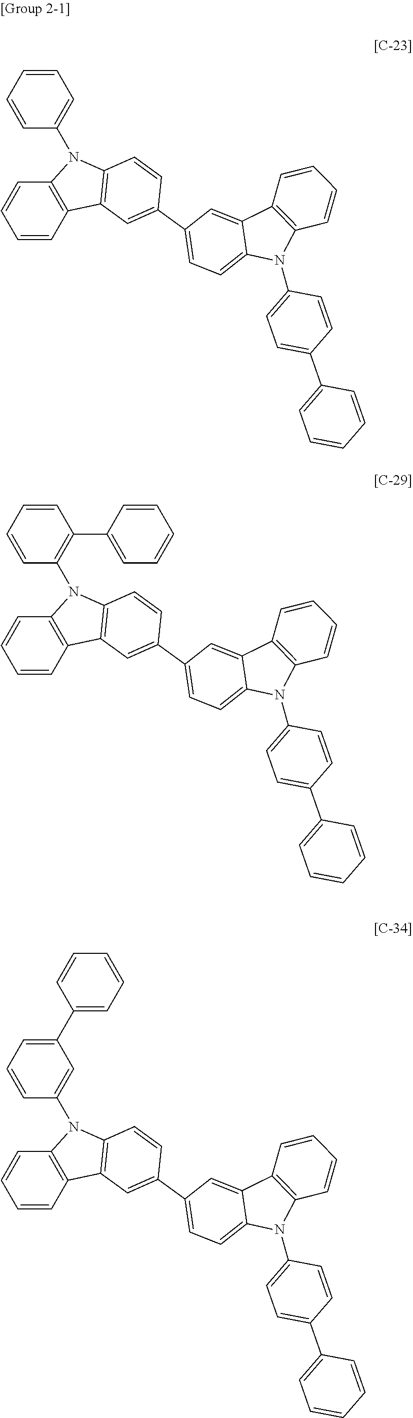 [SMILES: c1ccc(-c2ccc(-n3c4ccccc4c4cc(-c5ccc6c(c5)c5ccccc5n6-c5ccccc5)ccc43)cc2)cc1 ; c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2ccc(-n3c4ccccc4c4cc(-c5ccc6c(c5)c5ccccc5n6-c5cccc(-c6ccccc6)c5)ccc43)cc2)cc1]

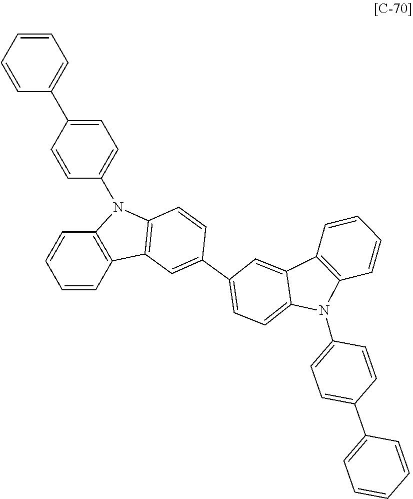 [SMILES: c1ccc(-c2ccc(-n3c4ccccc4c4cc(-c5ccc6c(c5)c5ccccc5n6-c5ccc(-c6ccccc6)cc5)ccc43)cc2)cc1]

The first compound and the second compound may be included in a weight ratio of 1:99 to 99:1. Within the above range, by adjusting an appropriate weight ratio using the electron transport capability of the first compound and the hole transport capability of the second compound, bipolar properties may be implemented to improve efficiency and driving. Within the range, they may be for example included in a weight ratio of about 10:90 to 90:10, about 20:80 to 80:20, about 20:80 to 70:30, about 20:80 to 60:40, or about 20:80 to about 50:50. For example, they may be included in a weight ratio of 30:70 to 50:50, for example, 30:70.

In an embodiment of the present invention, the first compound and the second compound may each be included as a host of a light emitting layer, for example, a phosphorescent host.

The aforementioned composition for an organic optoelectronic device may be formed by a dry film formation method such as chemical vapor deposition (CVD).

Hereinafter, an organic optoelectronic device including the aforementioned composition for an organic optoelectronic device is described.

The organic optoelectronic device may be any device to convert electrical energy into photoenergy and vice versa without particular limitation, and may be, for example an organic photoelectric device, an organic light emitting diode, an organic solar cell, and an organic photo-conductor drum.

Herein, an organic light emitting diode as one example of an organic optoelectronic device is described referring to drawings.

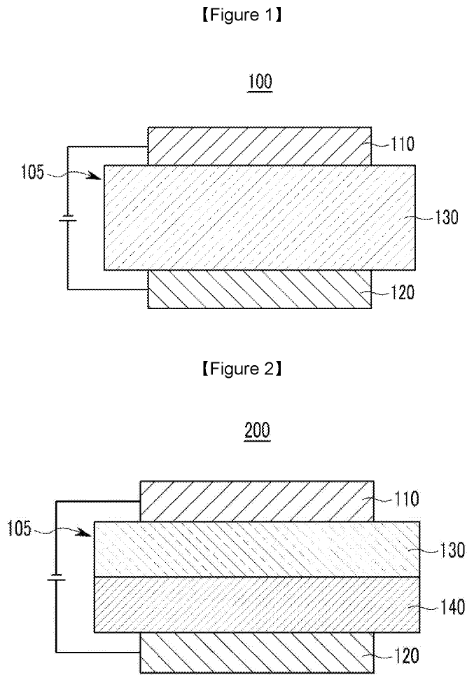

FIGS. 1 and 2 are cross-sectional views showing organic light emitting diodes according to embodiments.

Referring to FIG. 1, an organic light emitting diode 100 according to an embodiment includes an anode 120 and a cathode 110 facing each other and an organic layer 105 disposed between the anode 120 and cathode 110.

The anode 120 may be made of a conductor having a large work function to help hole injection, and may be for example a metal, a metal oxide and/or a conductive polymer. The anode 120 may be, for example a metal such as nickel, platinum, vanadium, chromium, copper, zinc, gold, and the like or an alloy thereof; a metal oxide such as zinc oxide, indium oxide, indium tin oxide (ITO), indium zinc oxide (IZO), and the like; a combination of a metal and an oxide such as ZnO and Al or SnO₂ and Sb; a conductive polymer such as poly(3-methylthiophene), poly(3,4-(ethylene-1,2-dioxy)thiophene) (PEDOT), polypyrrole, and polyaniline, but is not limited thereto.

The cathode 110 may be made of a conductor having a small work function to help electron injection, and may be for example a metal, a metal oxide, and/or a conductive polymer. The cathode 110 may be for example a metal such as magnesium, calcium, sodium, potassium, titanium, indium, yttrium, lithium, gadolinium, aluminum silver, tin, lead, cesium, barium, and the like, or an alloy thereof; a multi-layer structure material such as LiF/Al, LiO₂/Al, LiF/Ca, LiF/Al, and BaF₂/Ca, but is not limited thereto.

The organic layer 105 includes the aforementioned composition for an organic optoelectronic device.

The organic layer 105 may include the light emitting layer 130, and the light emitting layer 130 may include the aforementioned composition for an organic optoelectronic device.

The light emitting layer 130 may include, for example, the aforementioned composition for an organic optoelectronic device as a phosphorescent host.

In addition to the aforementioned host, the light emitting layer may further include one or more compounds.

The light emitting layer may further include a dopant. The dopant may be, for example, a phosphorescent dopant, and may be, for example, a red, green or blue phosphorescent dopant. The composition for an organic optoelectronic device further including a dopant may be, for example, a green or red light emitting composition.

The dopant is a material mixed with a composition for an organic optoelectronic device in a small amount to cause light emission and may be generally a material such as a metal complex that emits light by multiple excitation into a triplet or more. The dopant may be, for example an inorganic, organic, or organic-inorganic compound, and one or more types thereof may be used.

Examples of the dopant may be a phosphorescent dopant and examples of the phosphorescent dopant may be an organic metal compound including Ir, Pt, Os, Ti, Zr, Hf, Eu, Tb, Tm, Fe, Co, Ni, Ru, Rh, Pd, or a combination thereof. The phosphorescent dopant may be, for example, a compound represented by Chemical Formula Z, but is not limited thereto.

L⁷MX² [Chemical Formula Z]

In Chemical Formula Z, M is a metal, and L⁷ and X² are the same or different, and are a ligand to form a complex compound with M.

The M may be, for example, Ir, Pt, Os, Ti, Zr, Hf, Eu, Tb, Tm, Fe, Co, Ni, Ru, Rh, Pd, or a combination thereof and L⁷ and X² may be, for example, a bidentate ligand.

The organic layer may further include an auxiliary layer in addition to the light emitting layer.

The auxiliary layer may be, for example, a hole auxiliary layer 140.

Referring to FIG. 2, the organic light emitting diode 200 further includes a hole auxiliary layer 140 in addition to the light emitting layer 130. The hole auxiliary layer 140 may further increase hole injection and/or hole mobility and block electrons between the anode 120 and the light emitting layer 130.

The hole auxiliary layer 140 may include, for example, at least one of the compounds of Group A.

Specifically, the hole auxiliary layer 140 may include a hole transport layer between the anode 120 and the light emitting layer 130, and a hole transport auxiliary layer between the light emitting layer 130 and the hole transport layer and at least one of the compounds of Group A may be included in the hole transport auxiliary layer.

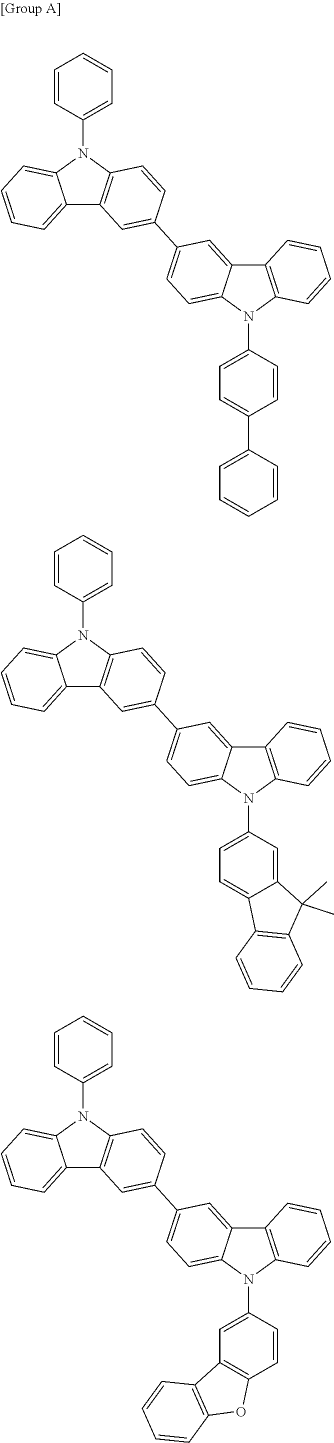 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4ccc5oc6ccccc6c5c4)ccc32)cc1]

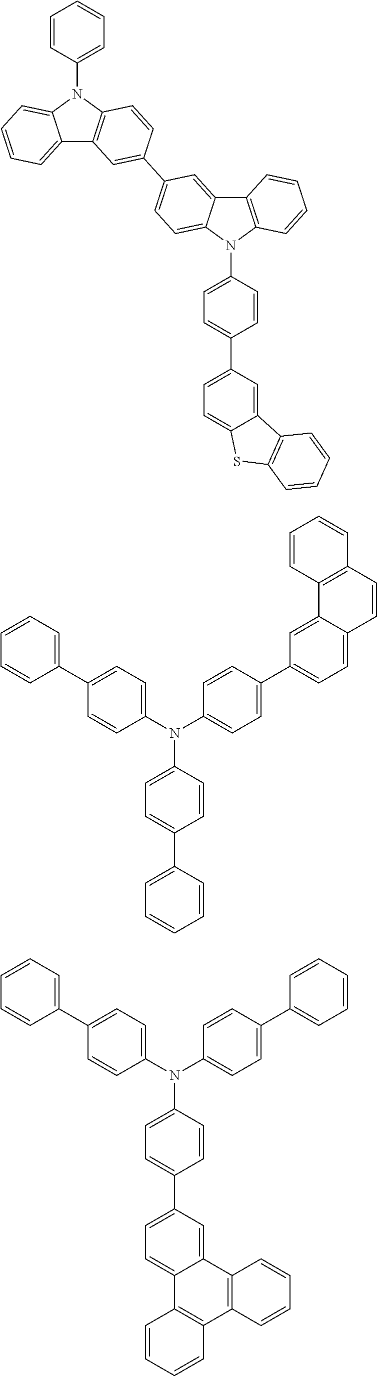

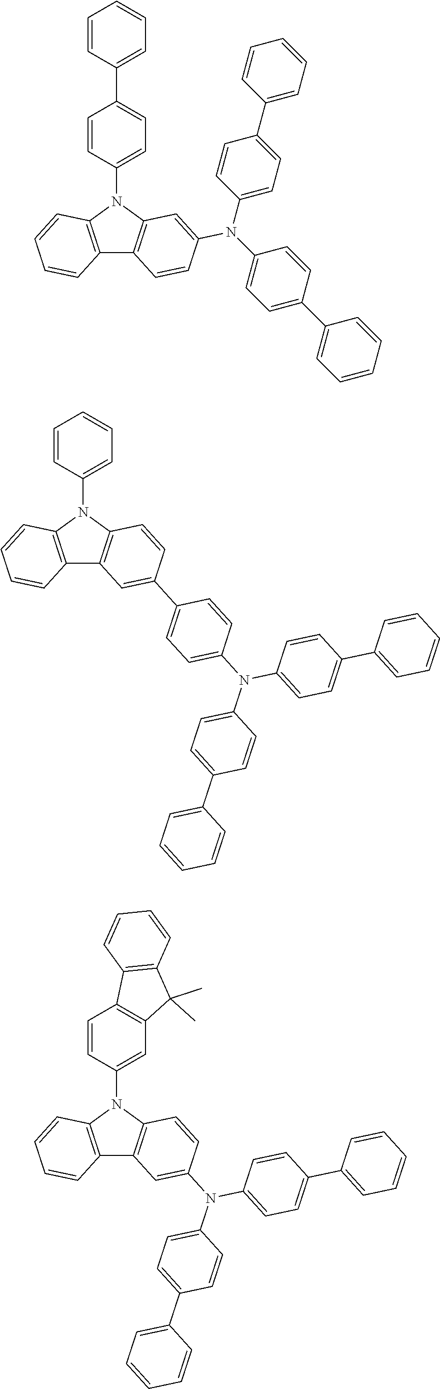 [SMILES: c1ccc(-c2ccc(N(c3ccc(-c4ccccc4)cc3)c3ccc4c5ccccc5n(-c5ccc(-c6ccccc6)cc5)c4c3)cc2)cc1 ; c1ccc(-c2ccc(N(c3ccc(-c4ccccc4)cc3)c3ccc(-c4ccc5c(c4)c4ccccc4n5-c4ccccc4)cc3)cc2)cc1 ; CC1(C)c2ccccc2-c2ccc(-n3c4ccccc4c4cc(N(c5ccc(-c6ccccc6)cc5)c5ccc(-c6ccccc6)cc5)ccc43)cc21]

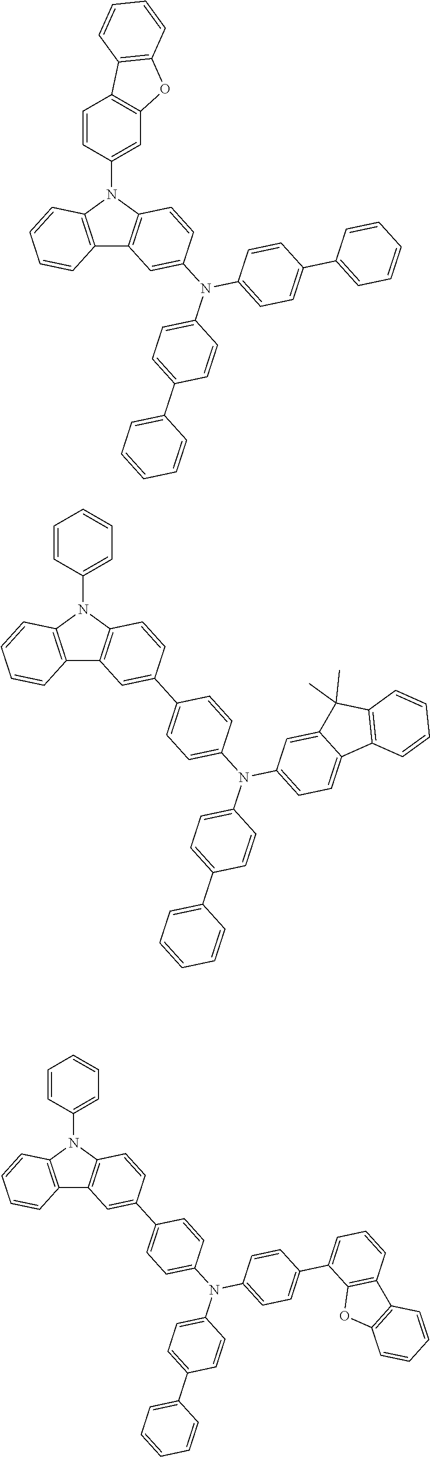 [SMILES: c1ccc(-c2ccc(N(c3ccc(-c4ccccc4)cc3)c3ccc4c(c3)c3ccccc3n4-c3ccc4c(c3)oc3ccccc34)cc2)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccc5c(c4)c4ccccc4n5-c4ccccc4)cc3)c3ccc(-c4cccc5c4oc4ccccc45)cc3)cc2)cc1]

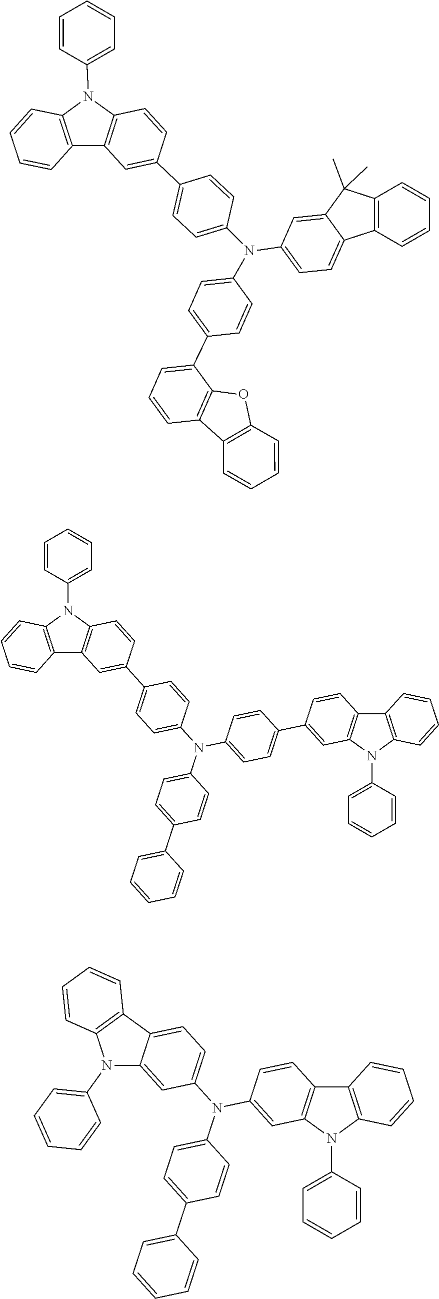 [SMILES: CC1(C)c2ccccc2-c2ccc(N(c3ccc(-c4ccc5c(c4)c4ccccc4n5-c4ccccc4)cc3)c3ccc(-c4cccc5c4oc4ccccc45)cc3)cc21 ; c1ccc(-c2ccc(N(c3ccc(-c4ccc5c(c4)c4ccccc4n5-c4ccccc4)cc3)c3ccc(-c4ccc5c6ccccc6n(-c6ccccc6)c5c4)cc3)cc2)cc1 ; c1ccc(-c2ccc(N(c3ccc4c5ccccc5n(-c5ccccc5)c4c3)c3ccc4c5ccccc5n(-c5ccccc5)c4c3)cc2)cc1]

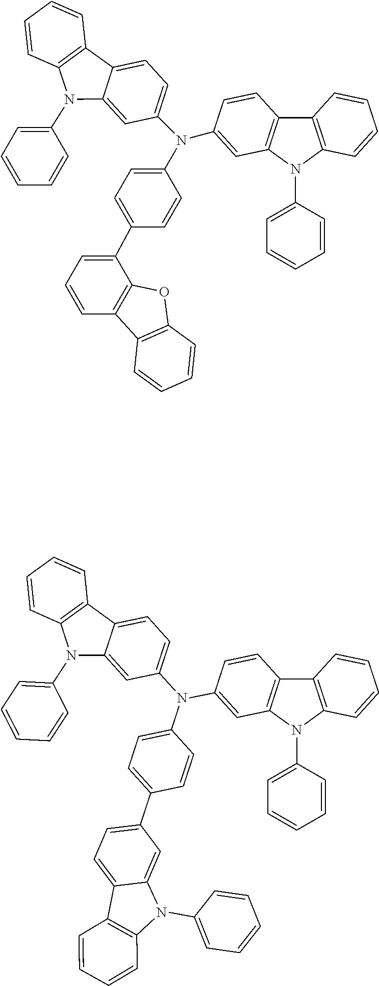 [SMILES: c1ccc(-n2c3ccccc3c3ccc(N(c4ccc(-c5cccc6c5oc5ccccc56)cc4)c4ccc5c6ccccc6n(-c6ccccc6)c5c4)cc32)cc1 ; c1ccc(-n2c3ccccc3c3ccc(-c4ccc(N(c5ccc6c7ccccc7n(-c7ccccc7)c6c5)c5ccc6c7ccccc7n(-c7ccccc7)c6c5)cc4)cc32)cc1]

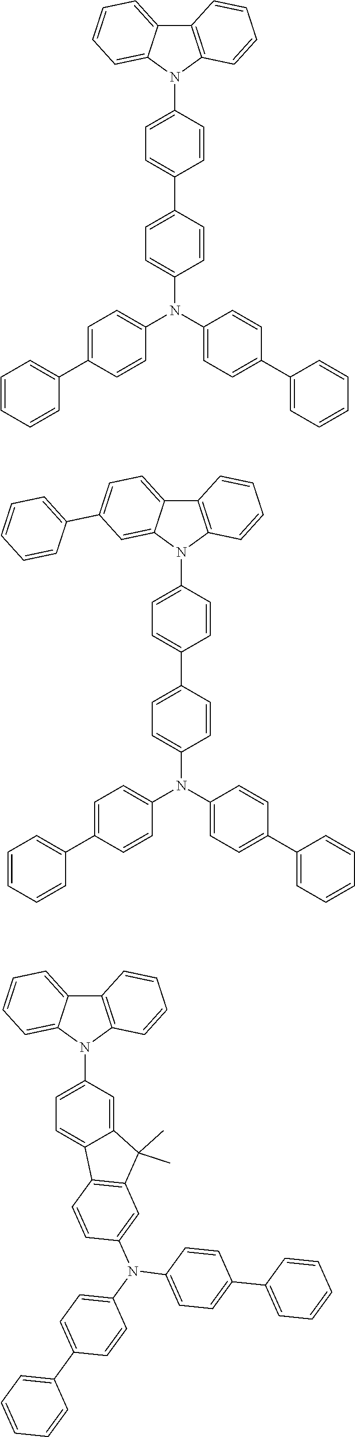 [SMILES: c1ccc(-c2ccc(N(c3ccc(-c4ccccc4)cc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccc(-c7ccccc7)cc65)cc4)cc3)cc2)cc1 ; CC1(C)c2cc(N(c3ccc(-c4ccccc4)cc3)c3ccc(-c4ccccc4)cc3)ccc2-c2ccc(-n3c4ccccc4c4ccccc43)cc21]

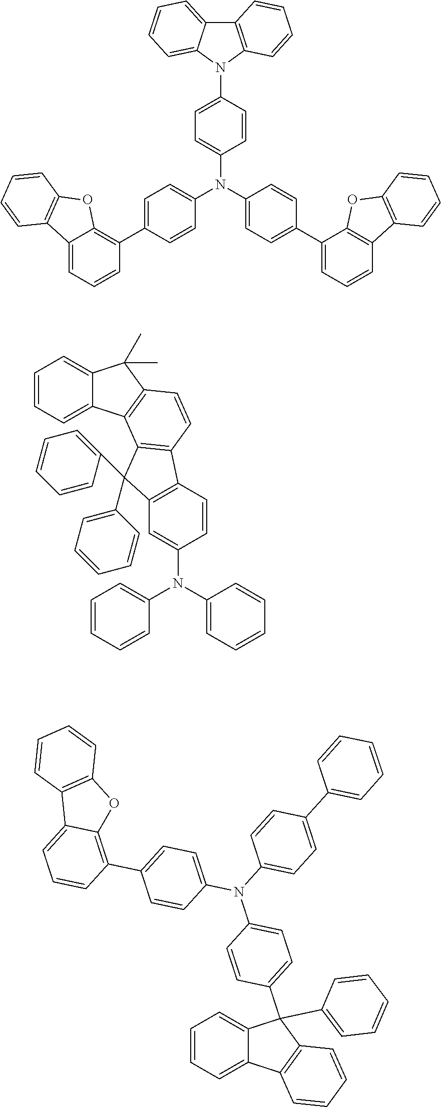 [SMILES: c1ccc2c(c1)oc1c(-c3ccc(N(c4ccc(-c5cccc6c5oc5ccccc56)cc4)c4ccc(-n5c6ccccc6c6ccccc65)cc4)cc3)cccc12 ; CC1(C)c2ccccc2-c2c1ccc1c2C(c2ccccc2)(c2ccccc2)c2cc(N(c3ccccc3)c3ccccc3)ccc2-1 ; c1ccc(-c2ccc(N(c3ccc(-c4cccc5c4oc4ccccc45)cc3)c3ccc(C4(c5ccccc5)c5ccccc5-c5ccccc54)cc3)cc2)cc1]

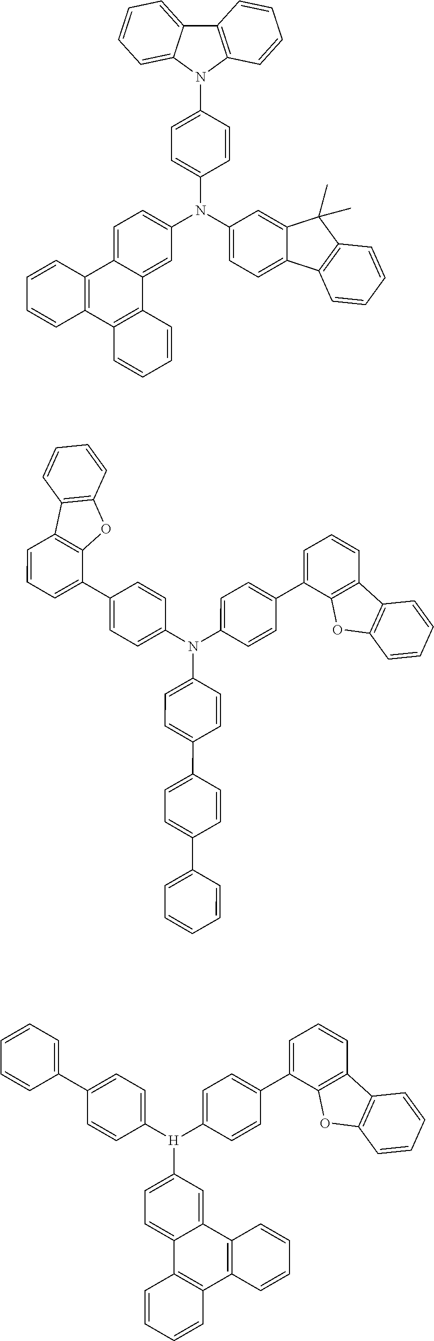

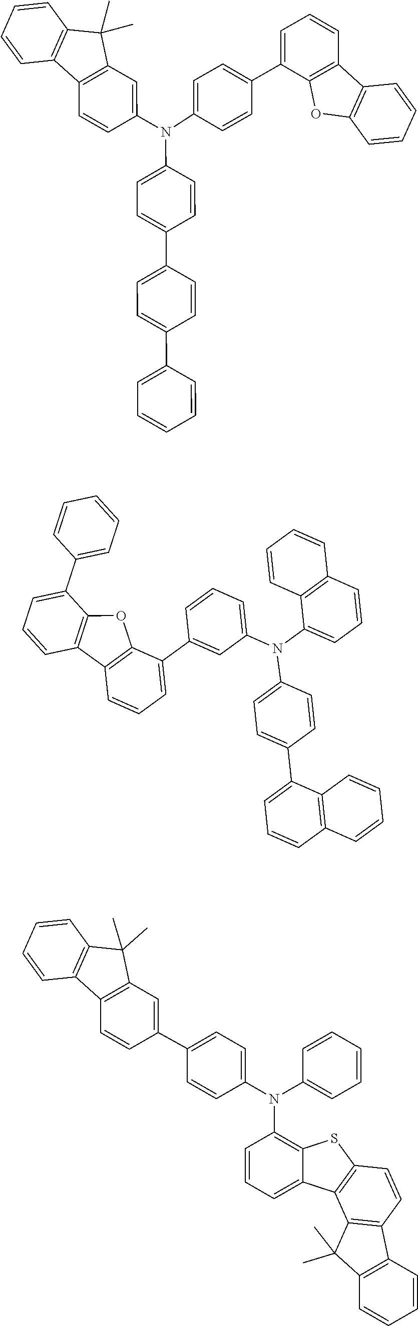 [SMILES: CC1(C)c2ccccc2-c2ccc(N(c3ccc(-c4ccc(-c5ccccc5)cc4)cc3)c3ccc(-c4cccc5c4oc4ccccc45)cc3)cc21 ; c1ccc(-c2cccc3c2oc2c(-c4cccc(N(c5ccc(-c6cccc7ccccc67)cc5)c5cccc6ccccc56)c4)cccc23)cc1 ; CC1(C)c2ccccc2-c2ccc(-c3ccc(N(c4ccccc4)c4cccc5c4sc4ccc6c(c45)C(C)(C)c4ccccc4-6)cc3)cc21]

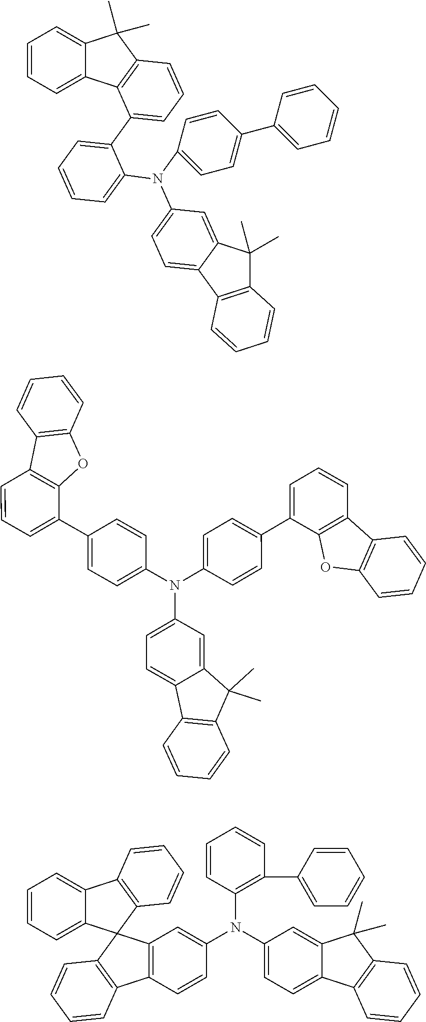 [SMILES: CC1(C)c2ccccc2-c2ccc(N(c3ccc(-c4ccccc4)cc3)c3ccccc3-c3cccc4c3-c3ccccc3C4(C)C)cc21 ; CC1(C)c2ccccc2-c2ccc(N(c3ccc(-c4cccc5c4oc4ccccc45)cc3)c3ccc(-c4cccc5c4oc4ccccc45)cc3)cc21 ; CC1(C)c2ccccc2-c2ccc(N(c3ccc4c(c3)C3(c5ccccc5-c5ccccc53)c3ccccc3-4)c3ccccc3-c3ccccc3)cc21]

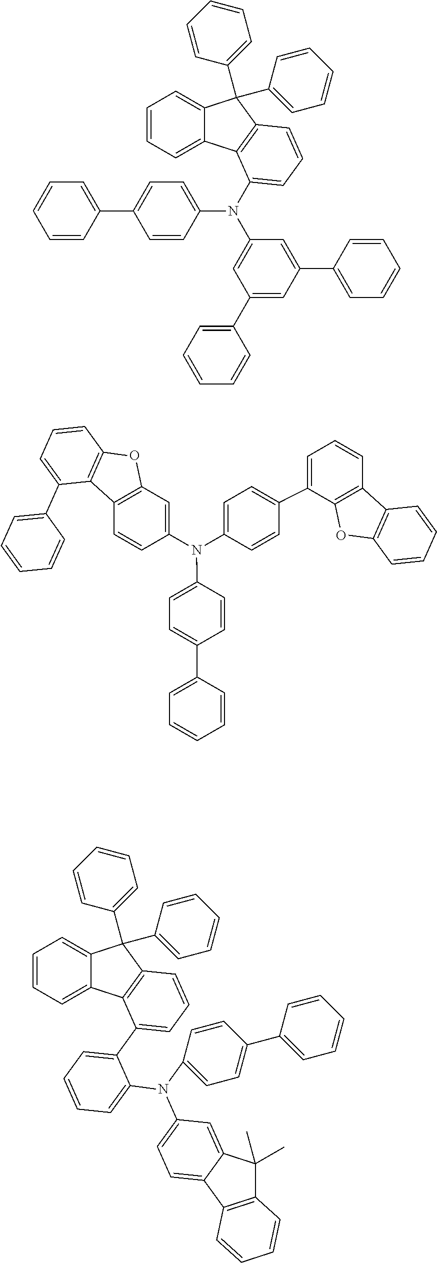 [SMILES: c1ccc(-c2ccc(N(c3cc(-c4ccccc4)cc(-c4ccccc4)c3)c3cccc4c3-c3ccccc3C4(c3ccccc3)c3ccccc3)cc2)cc1 ; c1ccc(-c2ccc(N(c3ccc(-c4cccc5c4oc4ccccc45)cc3)c3ccc4c(c3)oc3cccc(-c5ccccc5)c34)cc2)cc1 ; CC1(C)c2ccccc2-c2ccc(N(c3ccc(-c4ccccc4)cc3)c3ccccc3-c3cccc4c3-c3ccccc3C4(c3ccccc3)c3ccccc3)cc21]

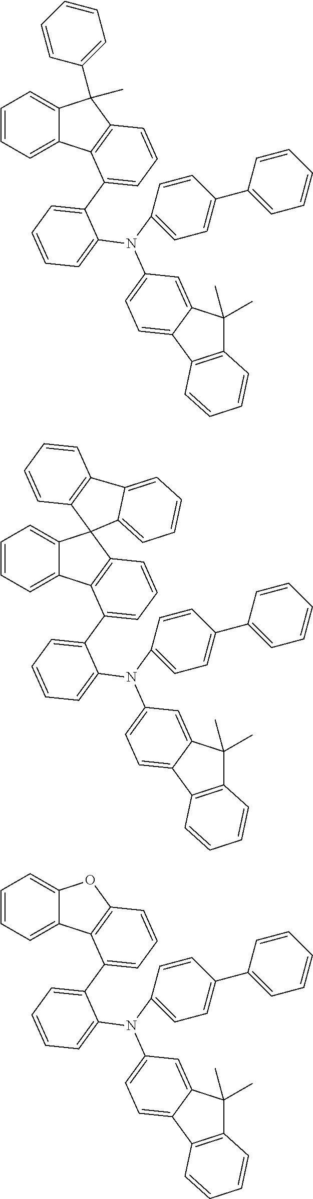 [SMILES: CC1(C)c2ccccc2-c2ccc(N(c3ccc(-c4ccccc4)cc3)c3ccccc3-c3cccc4c3-c3ccccc3C4(C)c3ccccc3)cc21 ; CC1(C)c2ccccc2-c2ccc(N(c3ccc(-c4ccccc4)cc3)c3ccccc3-c3cccc4c3-c3ccccc3C43c4ccccc4-c4ccccc43)cc21 ; CC1(C)c2ccccc2-c2ccc(N(c3ccc(-c4ccccc4)cc3)c3ccccc3-c3cccc4oc5ccccc5c34)cc21]

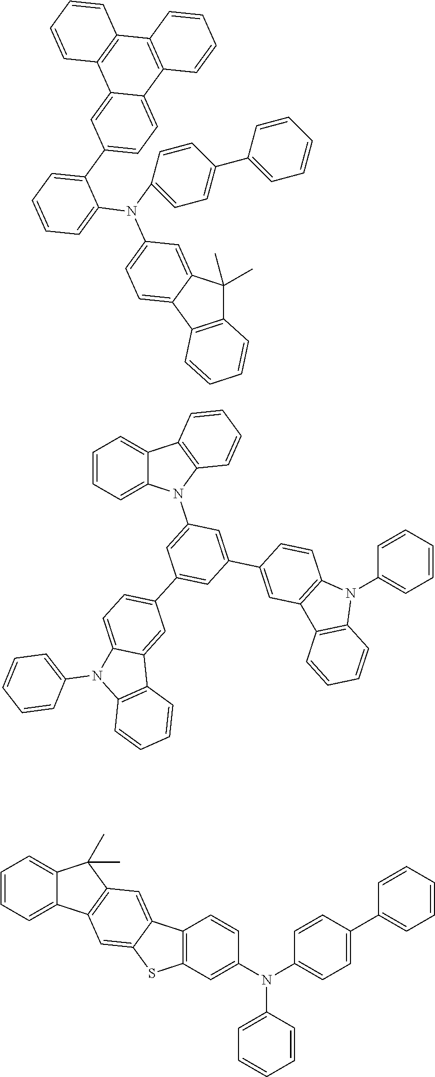

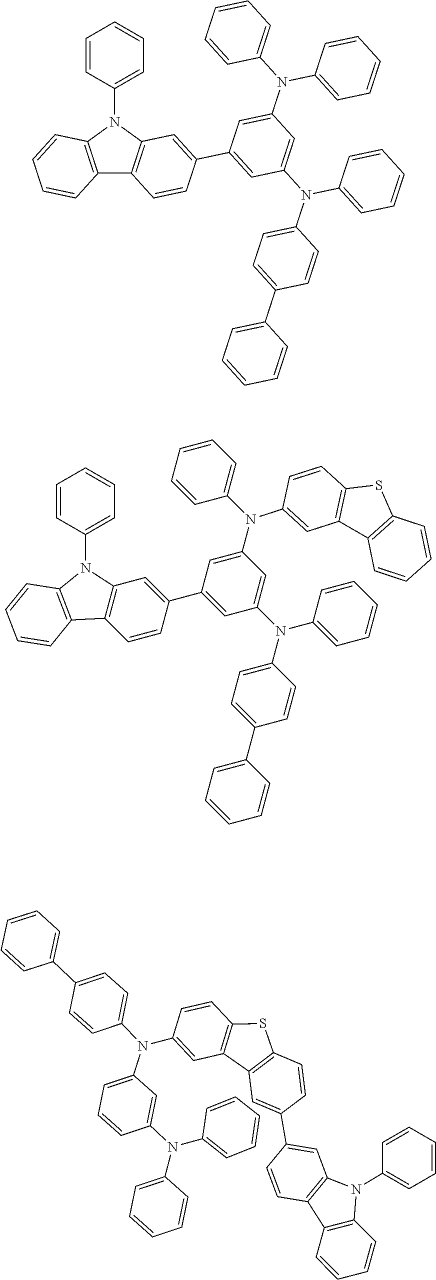 [SMILES: c1ccc(-c2ccc(N(c3ccccc3)c3cc(-c4ccc5c6ccccc6n(-c6ccccc6)c5c4)cc(N(c4ccccc4)c4ccccc4)c3)cc2)cc1 ; c1ccc(-c2ccc(N(c3ccccc3)c3cc(-c4ccc5c6ccccc6n(-c6ccccc6)c5c4)cc(N(c4ccccc4)c4ccc5sc6ccccc6c5c4)c3)cc2)cc1 ; c1ccc(-c2ccc(N(c3cccc(N(c4ccccc4)c4ccccc4)c3)c3ccc4sc5ccc(-c6ccc7c8ccccc8n(-c8ccccc8)c7c6)cc5c4c3)cc2)cc1]

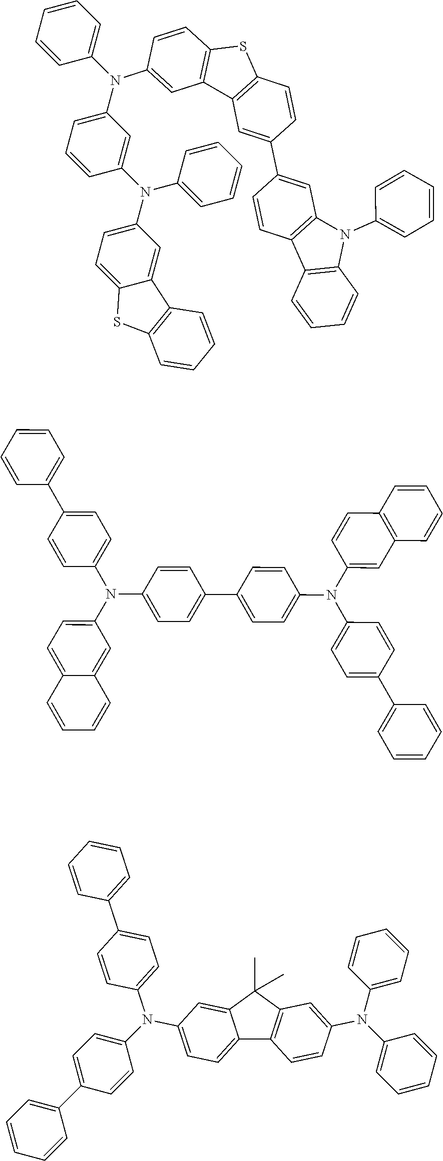 [SMILES: c1ccc(N(c2cccc(N(c3ccccc3)c3ccc4sc5ccc(-c6ccc7c8ccccc8n(-c8ccccc8)c7c6)cc5c4c3)c2)c2ccc3sc4ccccc4c3c2)cc1 ; c1ccc(-c2ccc(N(c3ccc(-c4ccc(N(c5ccc(-c6ccccc6)cc5)c5ccc6ccccc6c5)cc4)cc3)c3ccc4ccccc4c3)cc2)cc1 ; CC1(C)c2cc(N(c3ccccc3)c3ccccc3)ccc2-c2ccc(N(c3ccc(-c4ccccc4)cc3)c3ccc(-c4ccccc4)cc3)cc21]

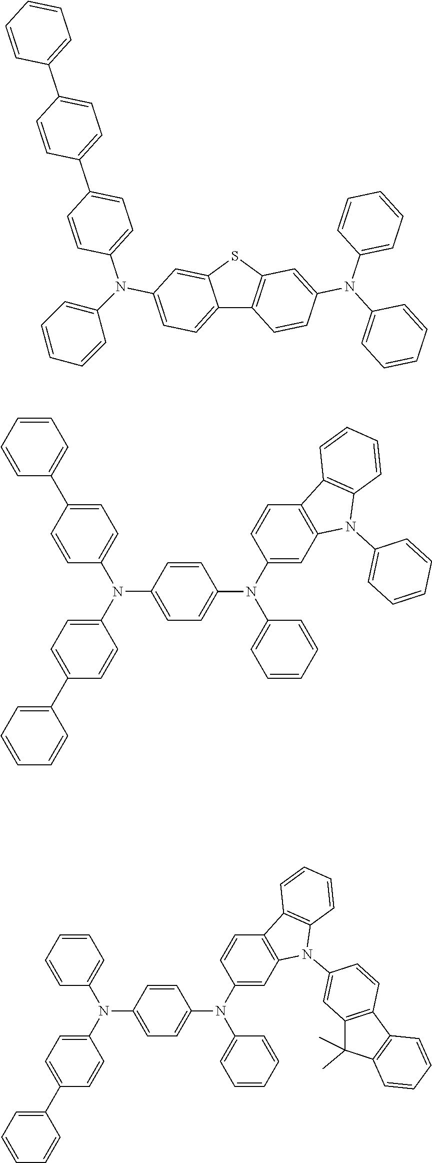 [SMILES: c1ccc(-c2ccc(-c3ccc(N(c4ccccc4)c4ccc5c(c4)sc4cc(N(c6ccccc6)c6ccccc6)ccc45)cc3)cc2)cc1 ; c1ccc(-c2ccc(N(c3ccc(-c4ccccc4)cc3)c3ccc(N(c4ccccc4)c4ccc5c6ccccc6n(-c6ccccc6)c5c4)cc3)cc2)cc1 ; CC1(C)c2ccccc2-c2ccc(-n3c4ccccc4c4ccc(N(c5ccccc5)c5ccc(N(c6ccccc6)c6ccc(-c7ccccc7)cc6)cc5)cc43)cc21]

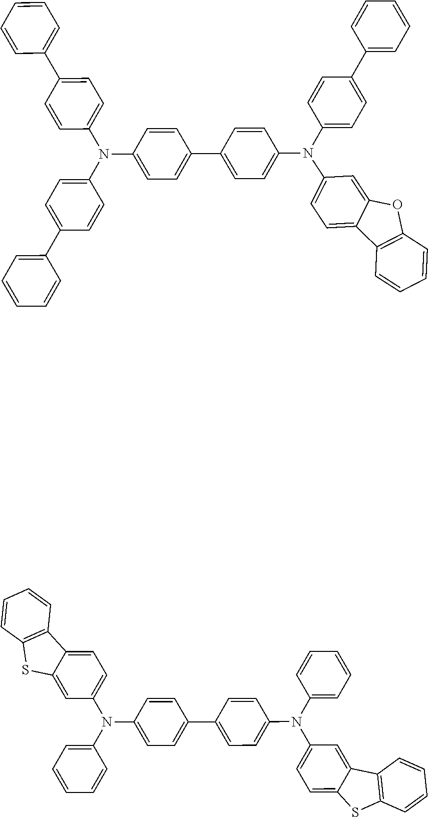 [SMILES: c1ccc(-c2ccc(N(c3ccc(-c4ccccc4)cc3)c3ccc(-c4ccc(N(c5ccc(-c6ccccc6)cc5)c5ccc6c(c5)oc5ccccc56)cc4)cc3)cc2)cc1 ; c1ccc(N(c2ccc(-c3ccc(N(c4ccccc4)c4ccc5sc6ccccc6c5c4)cc3)cc2)c2ccc3c(c2)sc2ccccc23)cc1]

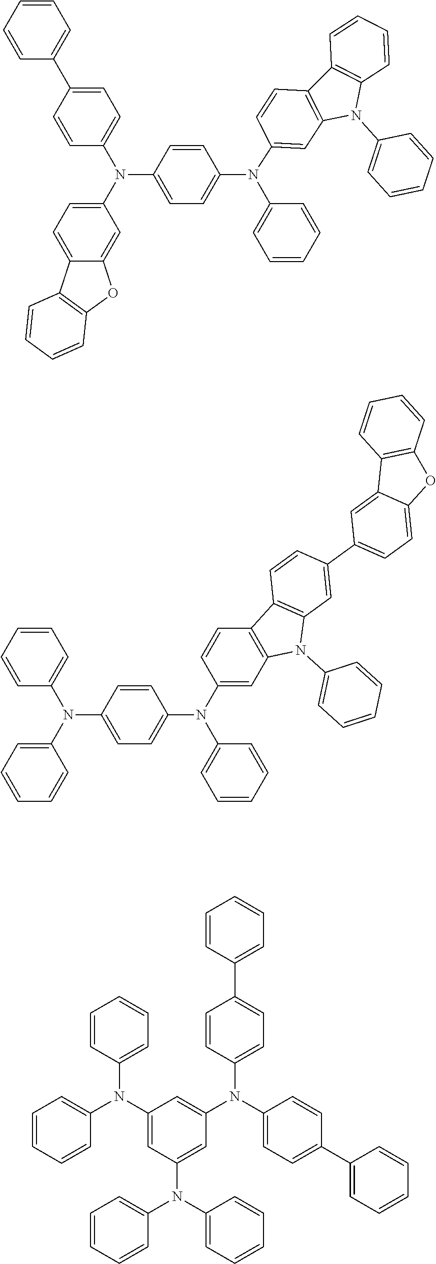 [SMILES: c1ccc(-c2ccc(N(c3ccc(N(c4ccccc4)c4ccc5c6ccccc6n(-c6ccccc6)c5c4)cc3)c3ccc4c(c3)oc3ccccc34)cc2)cc1 ; c1ccc(N(c2ccccc2)c2ccc(N(c3ccccc3)c3ccc4c5ccc(-c6ccc7oc8ccccc8c7c6)cc5n(-c5ccccc5)c4c3)cc2)cc1 ; c1ccc(-c2ccc(N(c3ccc(-c4ccccc4)cc3)c3cc(N(c4ccccc4)c4ccccc4)cc(N(c4ccccc4)c4ccccc4)c3)cc2)cc1]

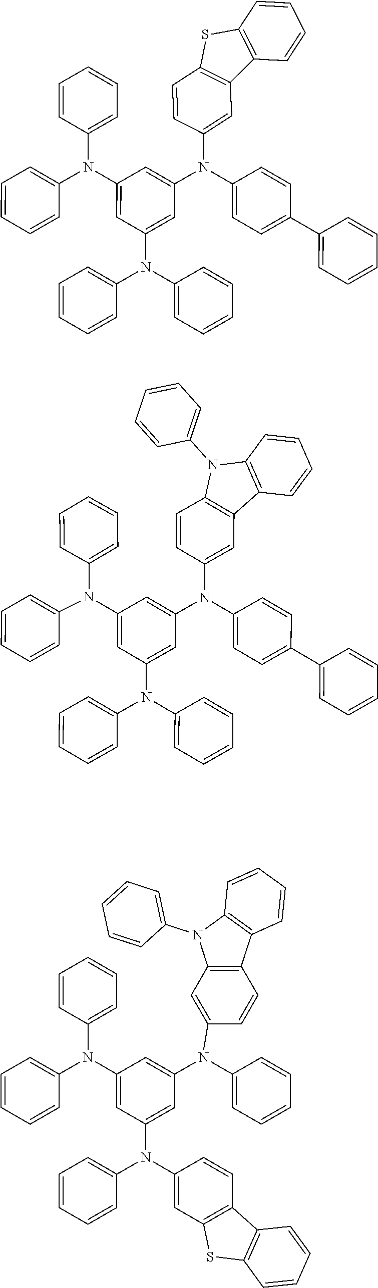 [SMILES: c1ccc(-c2ccc(N(c3cc(N(c4ccccc4)c4ccccc4)cc(N(c4ccccc4)c4ccccc4)c3)c3ccc4sc5ccccc5c4c3)cc2)cc1 ; c1ccc(-c2ccc(N(c3cc(N(c4ccccc4)c4ccccc4)cc(N(c4ccccc4)c4ccccc4)c3)c3ccc4c(c3)c3ccccc3n4-c3ccccc3)cc2)cc1 ; c1ccc(N(c2ccccc2)c2cc(N(c3ccccc3)c3ccc4c(c3)sc3ccccc34)cc(N(c3ccccc3)c3ccc4c5ccccc5n(-c5ccccc5)c4c3)c2)cc1]

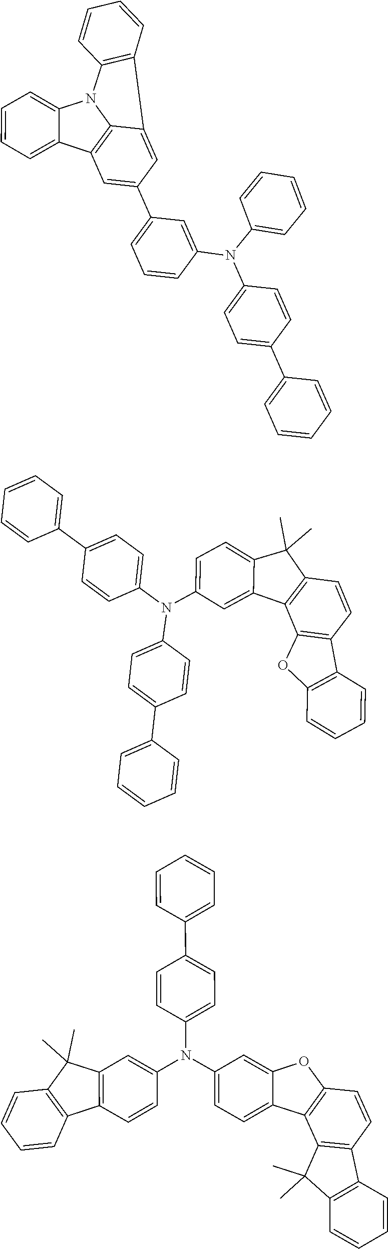 [SMILES: c1ccc(-c2ccc(N(c3ccccc3)c3cccc(-c4cc5c6ccccc6n6c7ccccc7c(c4)c56)c3)cc2)cc1 ; CC1(C)c2ccc(N(c3ccc(-c4ccccc4)cc3)c3ccc(-c4ccccc4)cc3)cc2-c2c1ccc1c2oc2ccccc21 ; CC1(C)c2ccccc2-c2ccc(N(c3ccc(-c4ccccc4)cc3)c3ccc4c(c3)oc3ccc5c(c34)C(C)(C)c3ccccc3-5)cc21]

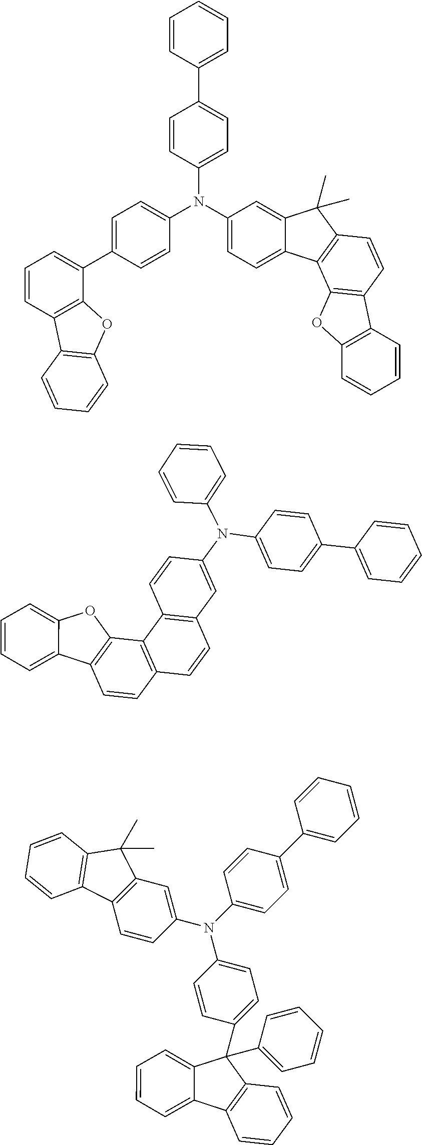 [SMILES: CC1(C)c2cc(N(c3ccc(-c4ccccc4)cc3)c3ccc(-c4cccc5c4oc4ccccc45)cc3)ccc2-c2c1ccc1c2oc2ccccc21 ; c1ccc(-c2ccc(N(c3ccccc3)c3ccc4c(ccc5ccc6c7ccccc7oc6c54)c3)cc2)cc1 ; CC1(C)c2ccccc2-c2ccc(N(c3ccc(-c4ccccc4)cc3)c3ccc(C4(c5ccccc5)c5ccccc5-c5ccccc54)cc3)cc21]

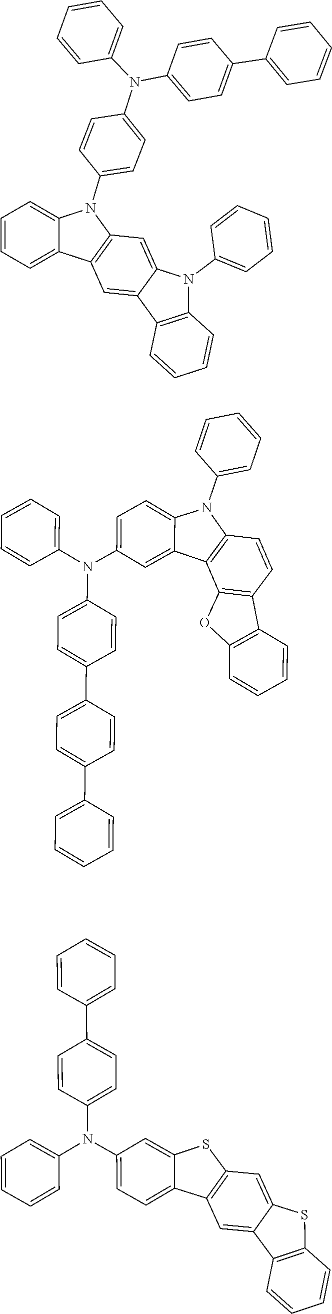 [SMILES: c1ccc(-c2ccc(N(c3ccccc3)c3ccc(-n4c5ccccc5c5cc6c7ccccc7n(-c7ccccc7)c6cc54)cc3)cc2)cc1 ; c1ccc(-c2ccc(-c3ccc(N(c4ccccc4)c4ccc5c(c4)c4c6oc7ccccc7c6ccc4n5-c4ccccc4)cc3)cc2)cc1 ; c1ccc(-c2ccc(N(c3ccccc3)c3ccc4c(c3)sc3cc5sc6ccccc6c5cc34)cc2)cc1]

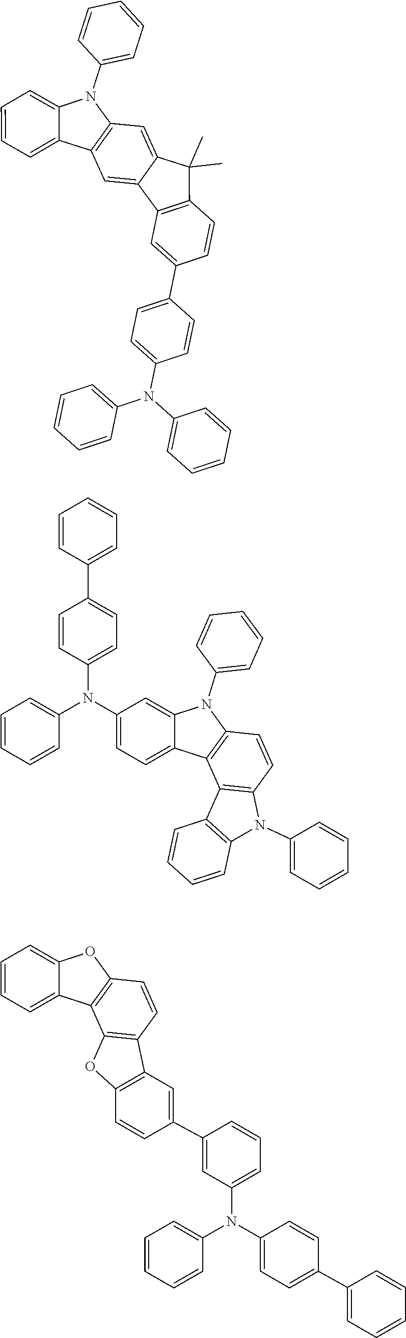 [SMILES: CC1(C)c2ccc(-c3ccc(N(c4ccccc4)c4ccccc4)cc3)cc2-c2cc3c4ccccc4n(-c4ccccc4)c3cc21 ; c1ccc(-c2ccc(N(c3ccccc3)c3ccc4c5c6c7ccccc7n(-c7ccccc7)c6ccc5n(-c5ccccc5)c4c3)cc2)cc1 ; c1ccc(-c2ccc(N(c3ccccc3)c3cccc(-c4ccc5oc6c(ccc7oc8ccccc8c76)c5c4)c3)cc2)cc1]

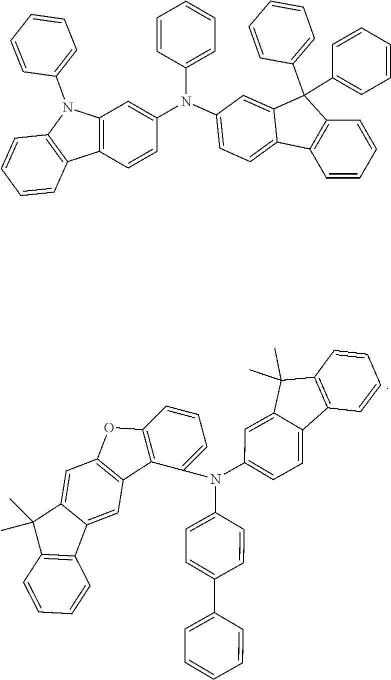

In addition to the aforementioned compounds, known compounds described in U.S. Pat. No. 5,061,569A, JP1993-009471A, WO1995-009147A1, JP1995-126615A, JP1998-095973A, and the like, and compounds having similar structures may be used for the hole transport auxiliary layer.

In addition, in an embodiment of the present invention, the organic light emitting diode may further include an electron transport layer, an electron injection layer, and a hole injection layer as the organic layer 105 in FIG. 1 or 2.

The organic light emitting diodes 100 and 200 may be manufactured by forming an anode or a cathode on a substrate, forming an organic layer by a dry film method such as evaporation, sputtering, plasma plating, and ion plating, and forming a cathode or an anode thereon.

The organic light emitting diode may be applied to an organic light emitting display device.

### MODE FOR INVENTION

Hereinafter, the embodiments are illustrated in more detail with reference to examples. However, these examples are exemplary, and the present scope is not limited thereto.

Hereinafter, starting materials and reactants used in Examples and Synthesis Examples were purchased from Sigma-Aldrich Co. Ltd., TCI Inc., Tokyo chemical industry or P&H tech as far as there is no particular comment or were synthesized by known methods.

### (Preparation of Compound for Organic Optoelectronic Device)

The compounds as more specific examples of the compounds of the present invention were synthesized through the following steps.

### Synthesis of First Compound

### Synthesis Example 1: Synthesis of Intermediate M-1

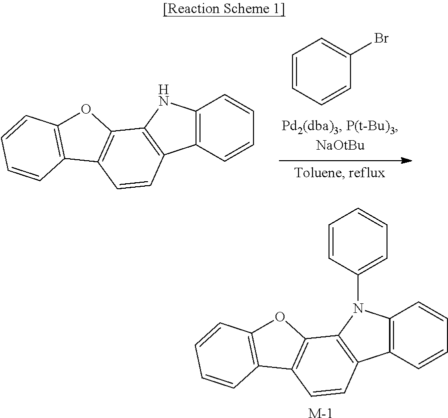

12H-benzofuro[2,3-a]carbazole (20 g, 77.73 mmol), bromobenzene (30.5 g, 194.33 mmol), NaOtBu (11.21 g, 116.6 mmol), and Pd₂(dba)₃ (3.56 g, 3.89 mmol) were added to 130 ml of toluene and suspend therein, and P(t-Bu)₃ (4.72 ml, 11.66 mmol) was added thereto and then, refluxed and stirred under an nitrogen atmosphere for 12 hours. Subsequently, distilled water was added to the reaction solution, and a solid produced therein was filtered and separated under a reduced pressure. The solid was recrystallized with toluene to obtain Intermediate M-1 (29.8 g, Yield: 80%).

LC-MS M+H: 334.12 g/mol,

### Synthesis Example 2: Synthesis of Intermediate M-2

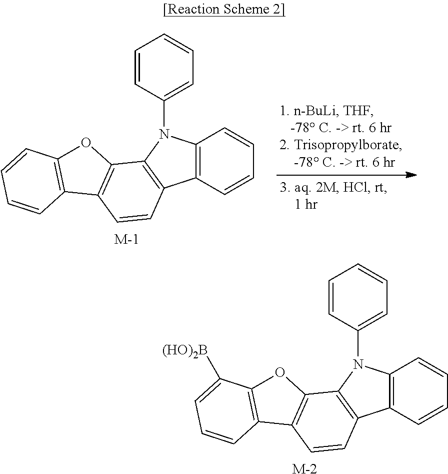

29.8 g (89.39 mmol) of Intermediate M-1 was dissolved by adding 224 ml of tetrahydrofuran thereto and then, cooled down to −78° C. and stirred under a nitrogen atmosphere. Subsequently, 42.9 ml (107.26 mmol) of a 2.5 M n-BuLi (in n-Hexane) solution was slowly added thereto and then, stirred at room temperature under a nitrogen atmosphere for 6 hours. The reaction solution was cooled down to −78° C., and 25.22 g (134.08 mmol) of triisopropylborate was slowly added thereto and then, stirred at room temperature under a nitrogen atmosphere for 6 hours. Subsequently, 223.5 ml of a 2.0 M hydrochloric acid aqueous solution was added thereto and then, stirred for 1 hour, and a solid produced therein was filtered and separated under a reduced pressure. The obtained solid was dissolved in methyl chloride and then, recrystallized to obtain Intermediate M-2 (50 g, Yield: 55%).

LC-MS M+H: 378.12 g/mol

### Synthesis Example 3: Synthesis of Compound A-67

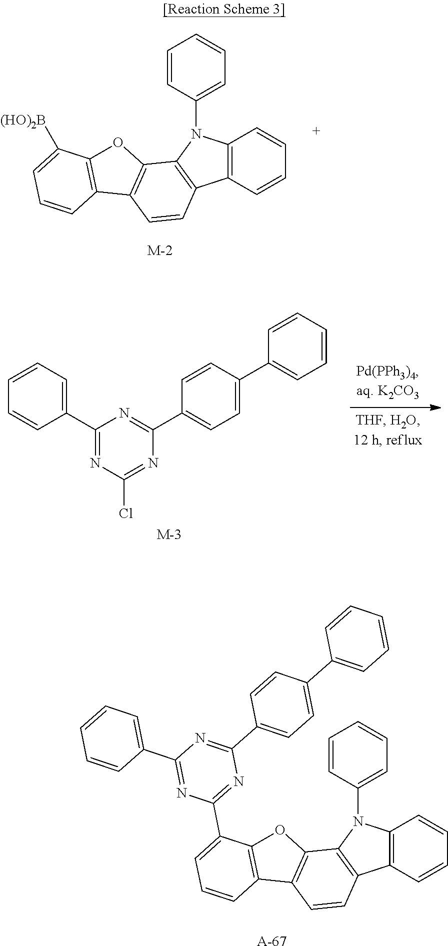

12 g (31.9 mmol) of Intermediate M-2 and 10 g (29.1 mmol) of Intermediate M-3 (2-chloro-4-(biphenyl-4-yl)-6-phenyl-1,3,5-triazine) were dissolved by adding 193 ml of tetrahydrofuran thereto, and then, 97 ml of an aqueous solution in which 10.1 g (72.7 mmol) of K₂CO₃ was dissolved was added thereto and then, stirred. Subsequently, 1.68 g (1.45 mmol) of Pd(PPh₃)₄ was added thereto and then, refluxed and stirred under a nitrogen atmosphere for 12 hours. When a reaction was completed, a solid produced therein was filtered and separated under a reduced pressure and then, dissolved in toluene and recrystallized to obtain Compound A-67 (14 g, Yield: 75%).

LC-MS M+H: 641.23 g/mol

### Synthesis Example 4: Synthesis of Compound A-4

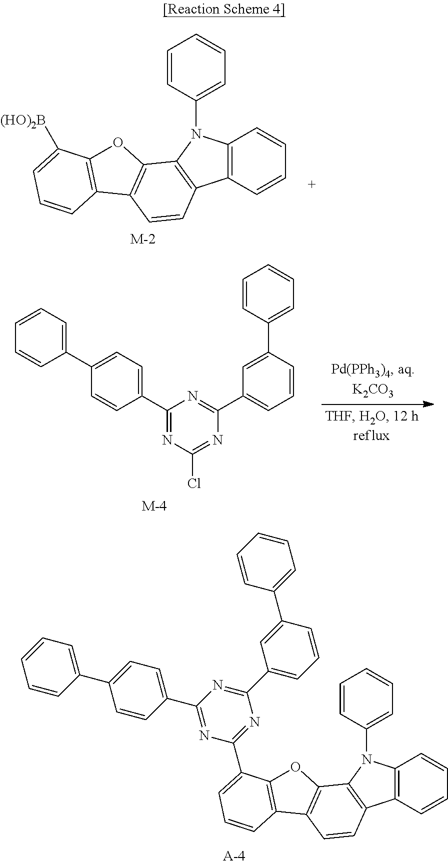

Compound A-4 (20 g, Yield: 79%) was obtained according to the same method as Synthesis Example 3 except that 10 g (23.81 mmol) of Intermediate M-4 (2-{[1,1′-biphenyl]yl}-4-{[1,1′-biphenyl]-4-yl}-6-chloro-1,3,5-triazine) was used instead of Intermediate M-3.

LC-MS M+H: 717.26 g/mol

### Synthesis Example 5: Synthesis of Compound A-89

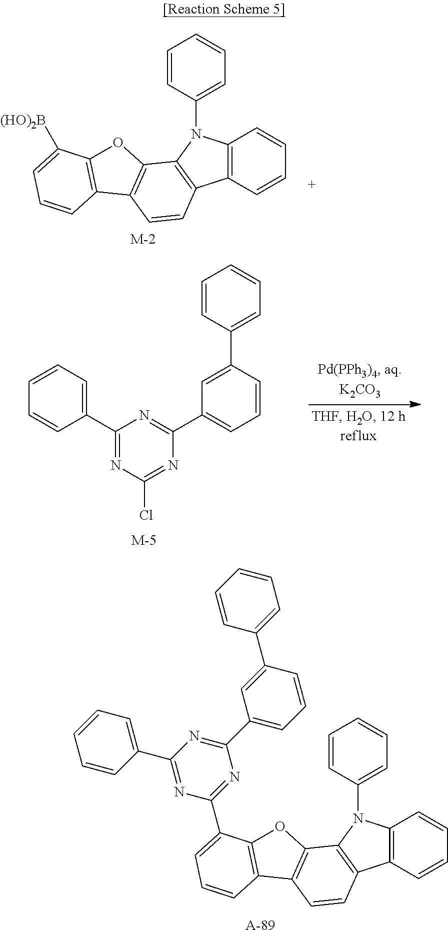

Compound A-89 (14.9 g, Yield: 80%) was obtained according to the same method as Synthesis Example 3 except that 10 g (29.1 mmol) of Intermediate M-5 (2-chloro-4-(biphenyl-3-yl)-6-phenyl-1,3,5-triazine) was used instead of Intermediate M-3.

LC-MS M+H: 641.75 g/mol

### Synthesis Example 6: Comparative Compound R-1

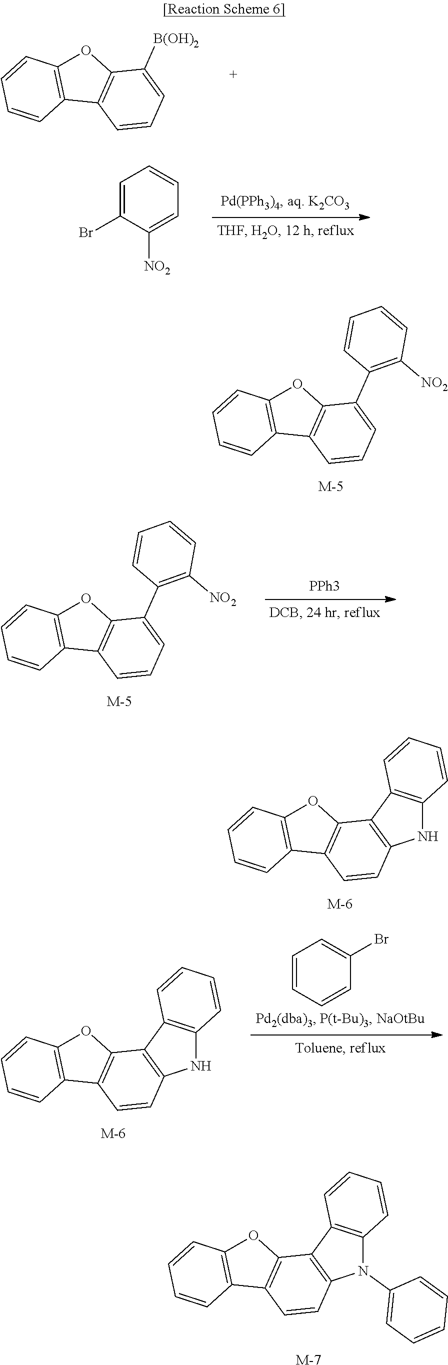

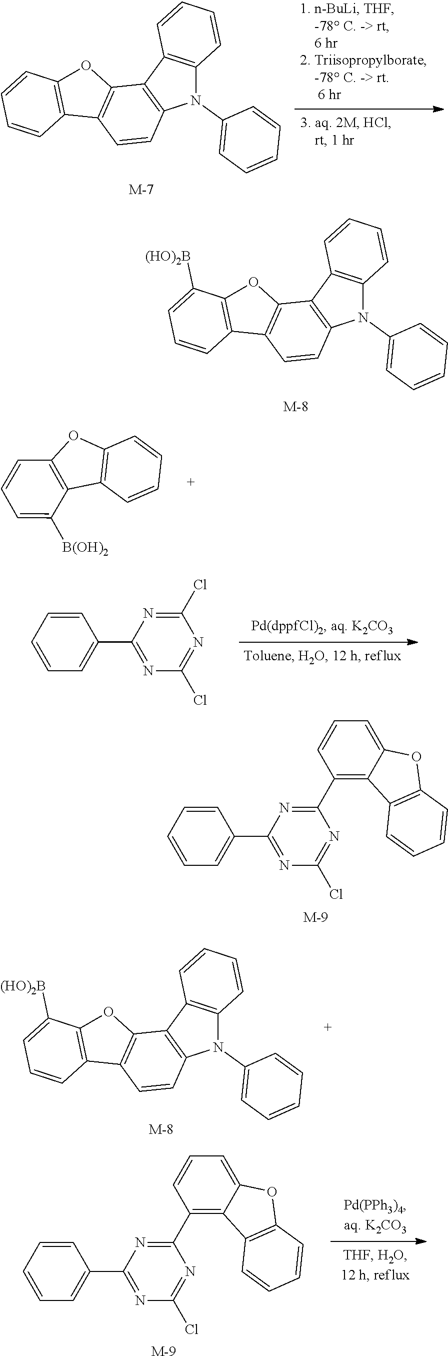

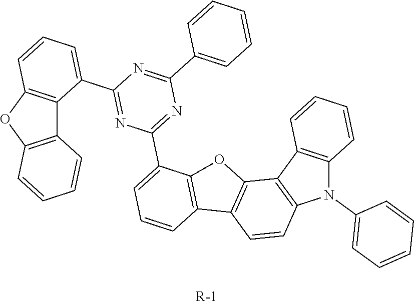 [SMILES: c1ccc(-c2nc(-c3cccc4c3oc3c4ccc4c3c3ccccc3n4-c3ccccc3)nc(-c3cccc4oc5ccccc5c34)n2)cc1 ; "R-1"]

### 1^{st} Step: Synthesis of Intermediate M-5

Intermediate M-5 (50 g, Yield: 92%) was obtained according to the same method as Synthesis Example 3 except that 40 g (188.7 mmol) of dibenzo[b,d]furan-4-ylboronic acid instead of Intermediate M-2 and 42 g (207.54 mmol) of 1-bromo-2-nitrobenzene instead of Intermediate M-3 were used.

LC-MS M+H: 290.07 g/mol

### 2^{nd} Step: Synthesis of Intermediate M-6

25 g (86.4 mmol) of Intermediate M-5 and 45.3 g (173 mmol) of triphenylposphine were dissolved by adding 260 ml of dichlorobenzene thereto and then, stirred under a nitrogen atmosphere for 24 hours at 170° C. When a reaction was completed, the resultant was extracted with toluene and DIW, and an extract therefrom was concentrated under a reduced pressure. The obtained product was purified with n-Hexane/dichloromethane through silica gel column chromatography to obtain Intermediate M-6 (16.7 g, Yield: 75%).

LC-MS M+H: 258.08 g/mol

### 3^{rd} Step: Synthesis of Intermediate M-7

Intermediate M-7 (17.6 g, Yield: 85%) was obtained according to the same method as Synthesis Example 1 except that 16 g (62.2 mmol) of Intermediate M-6 was used instead of 12H-benzofuro[2,3-a]carbazole

LC-MS M+H: 334.16 g/mol

### 4^{th} Step: Synthesis of Intermediate M-8

Intermediate M-8 (12 g, Yield: 71%) was obtained according to the same method as Synthesis Example 2 except that 15 g (45.0 mmol) of Intermediate M-7 was used instead of Intermediate M-1.

### 5^{th} Step: Synthesis of Intermediate M-9

20 g (67.99 mmol) of dibenzo[b,d]furan-1-ylboronic acid and 18.5 g (81.6 mmol) of 2,4-dichloro-6-phenyl-1,3,5-triazine were dissolved by adding 314 ml of toluene thereto, and 118 ml of an aqueous solution in which 32.6 g (235.84 mmol) of K₂CO₃ was dissolved was added thereto and then, stirred. Subsequently, 3.85 g (4.72 mmol) of Pd(dppfCl)₂ was added thereto and then, refluxed and stirred under a nitrogen atmosphere for 12 hours. When a reaction was completed, the resultant was extracted with toluene and DIW, and an extract obtained therefrom was concentrated under a reduced pressure. The obtained product was recrystallized with dimethylchloride and acetone to obtain Intermediate M-9 (24 g, Yield: 71%).

LC-MS M+H: 358.07 g/mol

### 6^{th} Step: Comparative Compound R-1

Comparative Compound R-1 (12.8 g, Yield: 92%) was obtained according to the same method as Synthesis Example 3 except that 8 g (21.21 mmol) of Intermediate M-8 instead of Intermediate M-2 and 8.35 g (23.33 mmol) of Intermediate M-9 instead of Intermediate M-3 were used.

LC-MS M+H: 655.21 g/mol

### Synthesis Example 7: Comparative Compound R-2

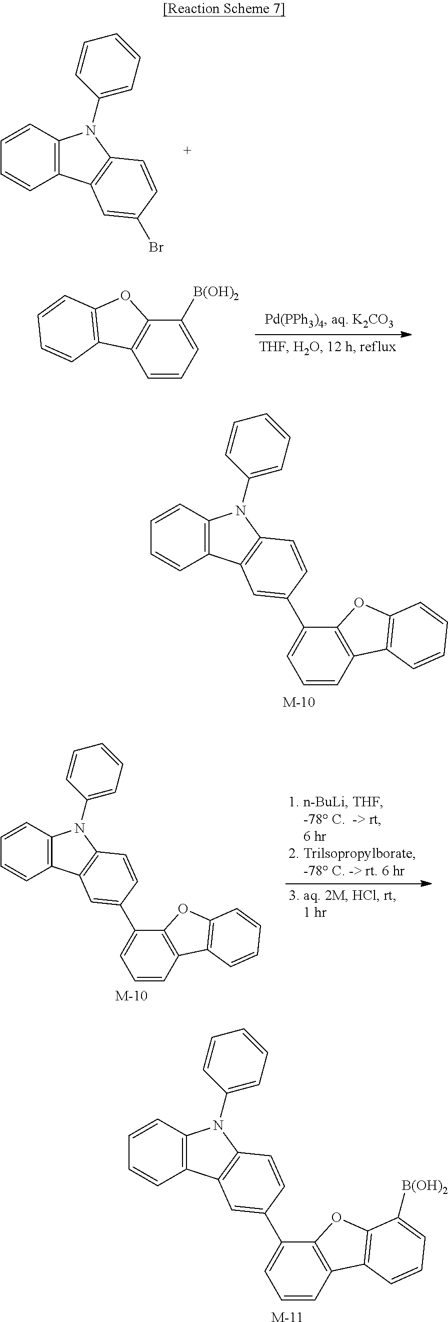

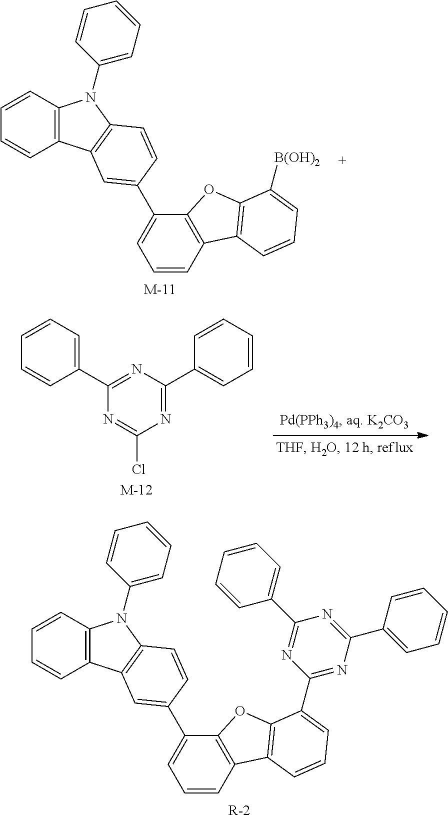

### 1^{st} Step: Synthesis of Intermediate M-10

Intermediate M-10 (15 g, Yield: 59%) was obtained according to the same method as Synthesis Example 3 except that 20 g (62.07 mmol) of 3-bromo-9-phenyl-9H-carbazole instead of Intermediate M-2 and 13.82 g (65.18 mmol) of dibenzo[b,d]furan-4-ylboronic acid instead of Intermediate M-3 were used.

LC-MS M+H: 410.15 g/mol

### 2^{nd} Step: Synthesis of Intermediate M-6

Intermediate M-11 (12 g, Yield: 54%) was obtained according to the same method as Synthesis Example 2 except that 20 g (48.84 mmol) of Intermediate M-10 instead of Intermediate M-1 were used.

LC-MS M+H: 454.15 g/mol

### 3^{rd} Step: Comparative Compound R-2

Comparative Compound R-2 (9 g, Yield: 83%) was obtained according to the same method as Synthesis Example 3 except that 12 g (26.47 mmol) of Intermediate M-11 instead of Intermediate M-2 and 5.9 g (22.06 mmol) of Intermediate M-12 instead of Intermediate M-3 were used.

LC-MS M+H 641.23 g/mol

### Synthesis Example 8: Comparative Compound R-3

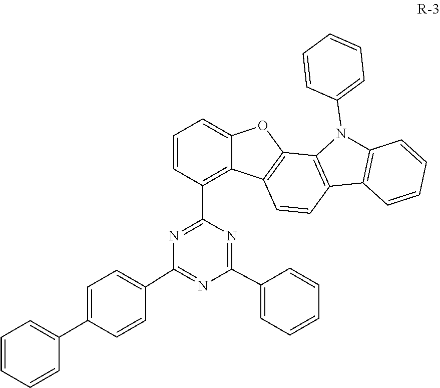 [SMILES: c1ccc(-c2ccc(-c3nc(-c4ccccc4)nc(-c4cccc5oc6c(ccc7c8ccccc8n(-c8ccccc8)c76)c45)n3)cc2)cc1]

Comparative Compound R-3 was synthesized referring to a method known in patent publication No. KR 10-1959047 B1.

LC-MS M+H 641.75 g/mol

### Synthesis Example 9: Comparative Compound R-4

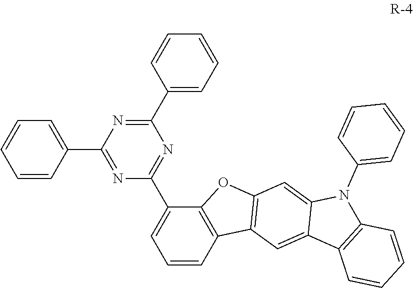 [SMILES: c1ccc(-c2nc(-c3ccccc3)nc(-c3cccc4c3oc3cc5c(cc34)c3ccccc3n5-c3ccccc3)n2)cc1]

Comparative Compound R-4 was synthesized referring to a method known in patent publication No. KR 10-1877678 B1.

LC-MS M+H 565.65 g/mol

### Synthesis of Second Compound

### Synthesis Example 10: Synthesis of Compound C-23

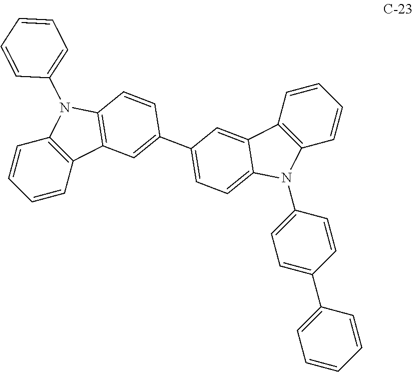 [SMILES: c1ccc(-c2ccc(-n3c4ccccc4c4cc(-c5ccc6c(c5)c5ccccc5n6-c5ccccc5)ccc43)cc2)cc1]

Compound C-23 was synthesized referring to a method known in patent publication No. KR 10-2019-0007968 A.

### Synthesis Example 11: Synthesis of Compound C-34

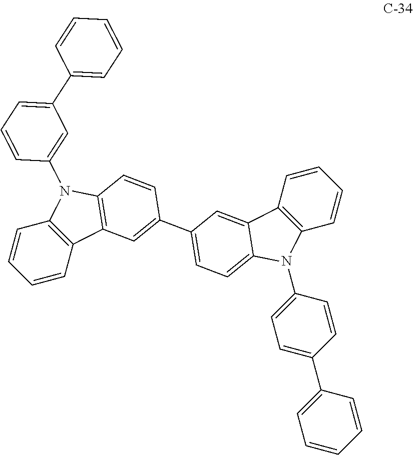 [SMILES: c1ccc(-c2ccc(-n3c4ccccc4c4cc(-c5ccc6c(c5)c5ccccc5n6-c5cccc(-c6ccccc6)c5)ccc43)cc2)cc1]

Compound C-34 was synthesized referring to a method known in patent publication No. KR 10-2019-0007968 A.

### (Manufacture of Organic Light Emitting Diode)

### Example 1

The glass substrate coated with ITO (Indium tin oxide) was washed with distilled water and ultrasonic waves. After washing with the distilled water, the glass substrate was washed with a solvent such as isopropyl alcohol, acetone, methanol, and the like ultrasonically and dried and then, moved to a plasma cleaner, cleaned by using oxygen plasma for 10 minutes, and moved to a vacuum depositor. This obtained ITO transparent electrode was used as an anode, Compound A doped with 1% NDP-9 (available from Novaled) was vacuum-deposited on the ITO substrate to form a 1400 Å-thick hole transport layer, and Compound B was deposited on the hole transport layer to form a 350 Å-thick hole transport auxiliary layer. On the hole transport auxiliary layer, 400 Å-thick light emitting layer was formed by simultaneously vacuum-depositing Compound A-67 of Synthesis Example 3 and Compound C-23 of Synthesis Example 7 as a host and doped with 10 wt % of PhGD as a dopant and the ratios were separately described for the following examples and comparative examples. Compound A-67 and Compound C-23 were used in a weight ratio of 3:7. Subsequently, Compound C was deposited on the light emitting layer to form a 50 Å-thick electron transport auxiliary layer, and Compound D and LiQ were simultaneously vacuum-deposited at a weight ratio of 1:1 to form a 300 Å-thick electron transport layer. LiQ (15 Å) and Al (1200 Å) were sequentially vacuum-deposited on the electron transport layer to form a cathode, thereby manufacturing an organic light emitting diode.

ITO/Compound A (1% NDP-9 doping, 1400 A)/Compound B (350 A)/EML [a mixture of Compound A-6 and Compound C-23 in a weight ratio of 3:7: PhGD=90 wt %: 10 wt %] (400 A)/Compound C (50 A)/Compound D: LiQ (300 A)/LiQ (15 A)/Al (1200 A).

Compound A: N-(biphenyl-4-yl)-9,9-dimethyl-N-(4-(9-phenyl-9H-carbazol-3-yl)phenyl)-9H-fluoren-2-amine

Compound B: N,N-bis(9,9-dimethyl-9H-fluoren-4-yl)-9,9-spirobi(fluorene)-2-amine Compound C: 2-(3-(3-(9,9-dimethyl-9H-fluoren-2-yl)phenyl)phenyl)-4,6-diphenyl-1,3,5-triazine

Compound D: 8-(4-(4,6-di(naphthalen-2-yl)-1,3,5-triazin-2-yl)phenyl)quinolone [PhGD]

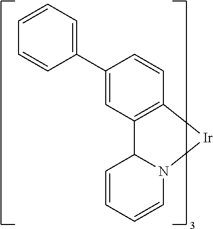

### Comparative Examples 1 and 2

The diodes of Comparative Examples 1 and 2 were manufactured in the same manner as in Example 1 except that the first host was changed as shown in Table 1.

### Example 2

The diode of Example 2 was manufactured in the same manner as in Example 1, except that Compound A-4 and Compound C-23 were changed to a mixed host at a weight ratio of 4:6.

### Comparative Example 3

The diode of Comparative Example 3 was manufactured in the same manner as in Example 2 except that Compound A-4 was changed to Compound R-1.

### Example 3 and Comparative Examples 4 and 5

The diodes of Example 3 and Comparative Examples 4 and 5 were manufactured in the same manner as in Example 2 except that the first host was changed as described in Table 3.

### Evaluation: Decrease of Driving Voltage and Increase of Life-Span

The driving voltages and life-span characteristics of the organic light emitting diodes according to Examples 1 to 3 and Comparative Examples 1 to 5 were evaluated. Specific measurement methods are as follows, and the results are shown in Tables 1 to 3.

(1) Measurement of Life-Span

T95 life-spans of the organic light emitting diodes according to Example 1 to Example 3, and Comparative Example 1 to Comparative Example 5 were measured as a time when their luminance decreased down to 95% relative to the initial luminance (cd/m²) after emitting light with 24,000 cd/m² as the initial luminance (cd/m²) and measuring their luminance decrease depending on a time with a Polanonix life-span measurement system.

(2) Measurement of Driving Voltage

Using a current-voltmeter (Keithley 2400), the driving voltage of each diode was measured at 15 mA/cm² to obtain a result.

(3) Calculation of T95 Life-span Ratio (%)

In Table 2, it was evaluated based on the T95 life-span of Comparative Example 3.

In Table 3, it was evaluated based on the T95 life-span of Comparative Example 4.

(4) Calculation of Driving Voltage Ratio (%)

In Table 1, it was evaluated based on the driving voltage value of Comparative Example

In Table 2, it was evaluated based on the driving voltage value of Comparative Example 3.

In Table 3, it was evaluated based on the driving voltage value of Comparative Example 4.

TABLE 1

| | | | Driving |
|---|---|---|---|
| | First | Second | voltage |
| | host | host | ratio (%) |
| Example 1 | A-67 | C-23 | 94 |
| Comparative Example 1 | R-1 | C-23 | 100 |
| Comparative Example 2 | R-2 | C-23 | 99 |

TABLE 2

| | | | Driving | T95 |
|---|---|---|---|---|
| | First | Second | voltage | life-span |
| | host | host | ratio (%) | ratio (%) |
| Example 2 | A-4 | C-23 | 95 | 227 |
| Comparative Example 3 | R-1 | C-23 | 100 | 100 |

TABLE 3

| | | | Driving | T95 |
|---|---|---|---|---|
| | First | Second | voltage | life-span |
| | host | host | ratio (%) | ratio (%) |
| Example 3 | A-89 | C-23 | 96 | 193 |
| Comparative Example 4 | R-3 | C-23 | 100 | 100 |
| Comparative Example 5 | R-4 | C-23 | 105 | 33 |

Referring to Tables 1 to 3, the driving voltages and life-spans of the compounds according to the present invention are significantly improved compared with the comparative compounds, and the driving voltages and life-spans of the compositions including the compounds according to the present invention are significantly improved compared with the compositions including the comparative compounds.

While this invention has been described in connection with what is presently considered to be practical embodiments, it is to be understood that the invention is not limited to the disclosed embodiments, but, on the contrary, is intended to cover various modifications and equivalent arrangements included within the spirit and scope of the appended claims.

1. A composition for an organic optoelectronic device, comprising:
a first compound represented by Chemical Formula 1; and
a second compound represented by Chemical Formula 2: 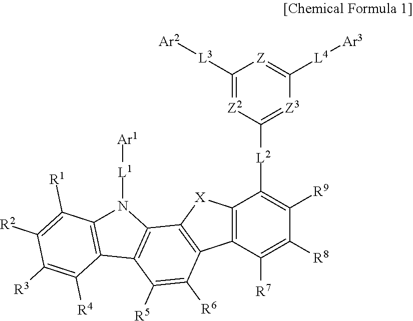
wherein, in Chemical Formula 1,
X is O or S,
Z¹ to Z³ are each independently N or CR^{a},
at least two of Z¹ to Z³ are N,
R^{a} and R¹ to R⁹ are each independently hydrogen, deuterium, a substituted or unsubstituted C1 to C30 alkyl group, a substituted or unsubstituted C6 to C30 aryl group, or a substituted or unsubstituted C2 to C30 heterocyclic group,
L¹ to L⁴ are each independently a single bond, or a substituted or unsubstituted C6 to C30 arylene group, and
Ar¹ to AP are each independently a substituted or unsubstituted C6 to C30 aryl group, or a substituted or unsubstituted C2 to C30 heterocyclic group, 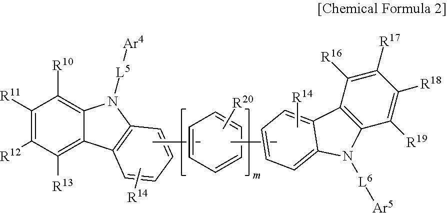
wherein, in Chemical Formula 2,
Ar⁴ and Ar⁵ are each independently a substituted or unsubstituted C6 to C30 aryl group, or a substituted or unsubstituted C2 to C30 heterocyclic group,
L⁵ and L⁶ are each independently a single bond, or a substituted or unsubstituted C6 to C30 arylene group,
R¹⁰ to R²⁰ are each independently hydrogen, deuterium, a substituted or unsubstituted C1 to C30 alkyl group, a substituted or unsubstituted C6 to C30 aryl group, or a substituted or unsubstituted C2 to C30 heterocyclic group, and
m is an integer of 0 to 2.

2. The composition of claim 1, wherein Ar¹ of Chemical Formula 1 is a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted fluorenyl group, a substituted or unsubstituted dibenzofuranyl group, or a substituted or unsubstituted dibenzothiophenyl group.

3. The composition of claim 1, wherein
Ar² and Ar³ of Chemical Formula 1 are each independently a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted fluorenyl group, a substituted or unsubstituted carbazolyl group, a substituted or unsubstituted dibenzofuranyl group, a substituted or unsubstituted dibenzothiophenyl group, or a substituted or unsubstituted pyridinyl group, and
L³ and L⁴ are each independently a single bond, a substituted or unsubstituted phenylene group, or a substituted or unsubstituted biphenylene group.

4. The composition of claim 1, wherein *-L³-Ar² and *-L⁴-Ar³ of Chemical Formula 1 are each independently selected from the substituents of Group I: 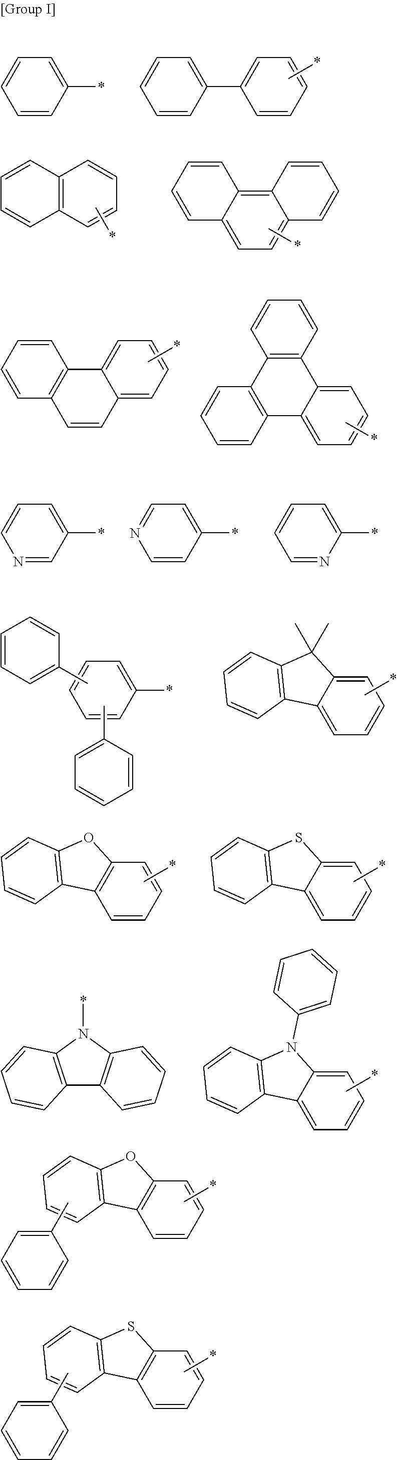
wherein, in Group I, * is a linking point.

5. The composition of claim 1, wherein *—C—Ar² and *-L⁴-Ar³ of Chemical Formula 1 are each independently selected from the substituents of Group I-1: 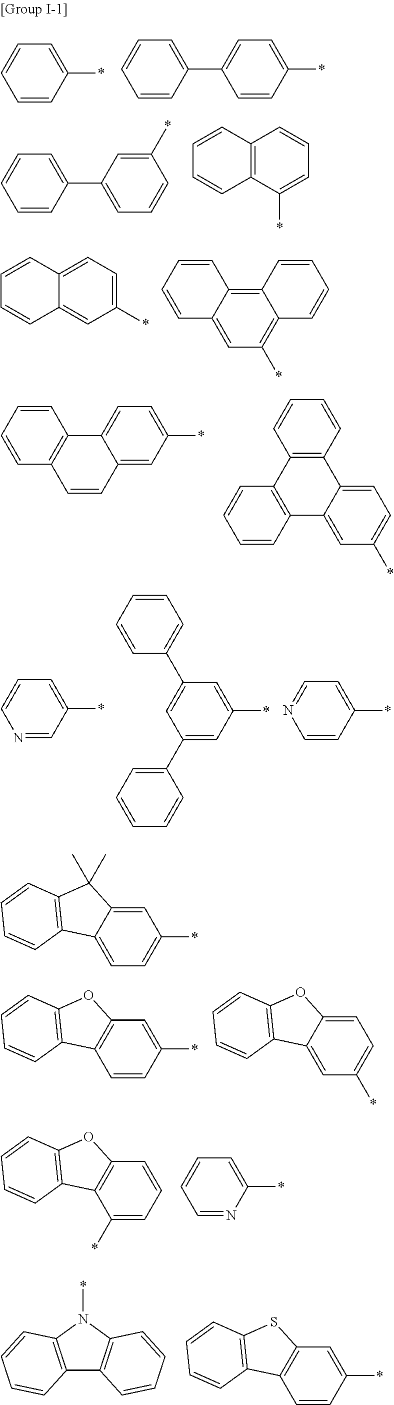 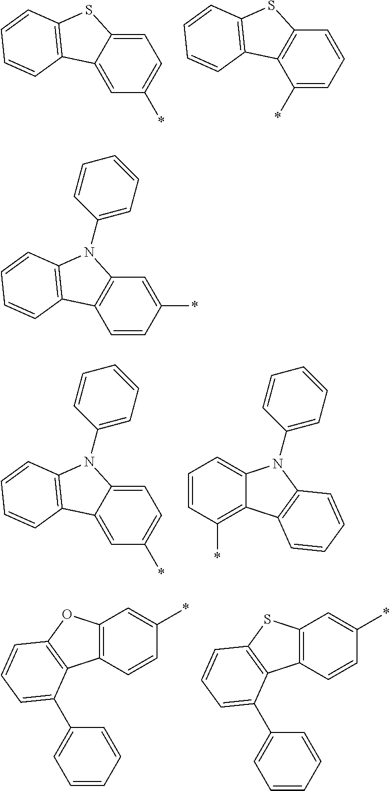
wherein, in Group I-1, * is a linking point.

6. The composition of claim 1, wherein the first compound is selected from compounds of Group 1:

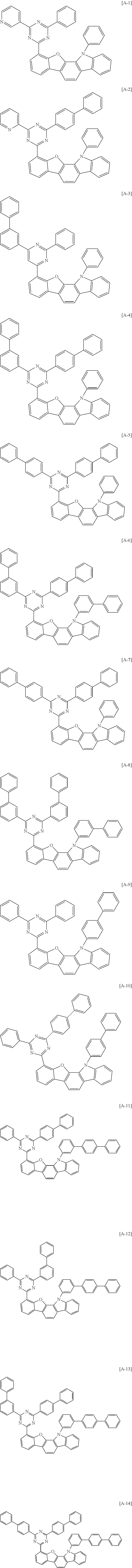

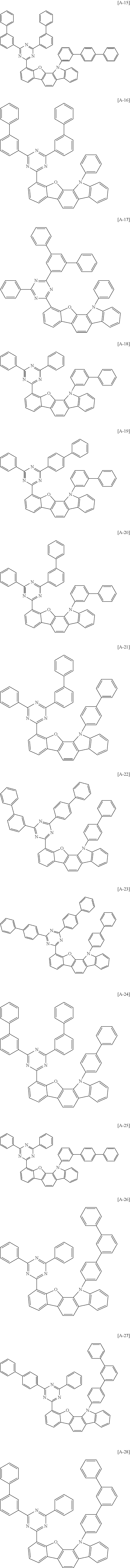

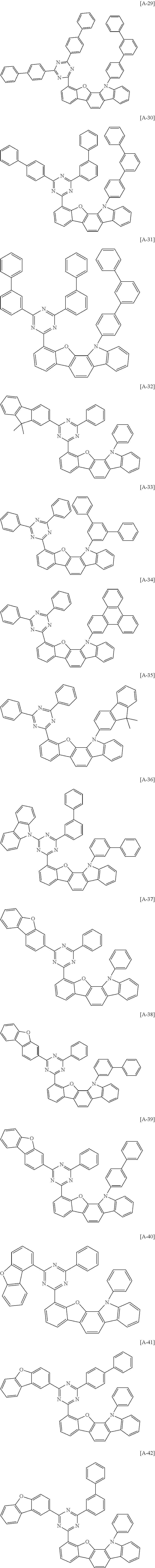

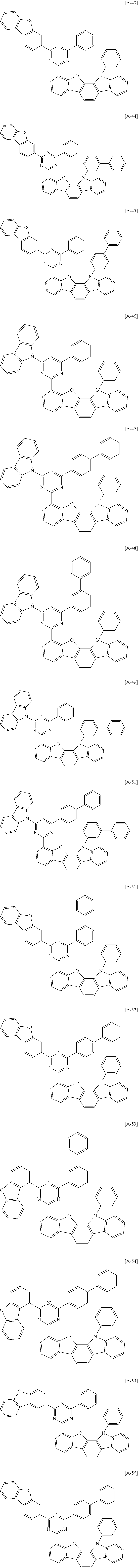

[chemical structure]

[chemical structure]

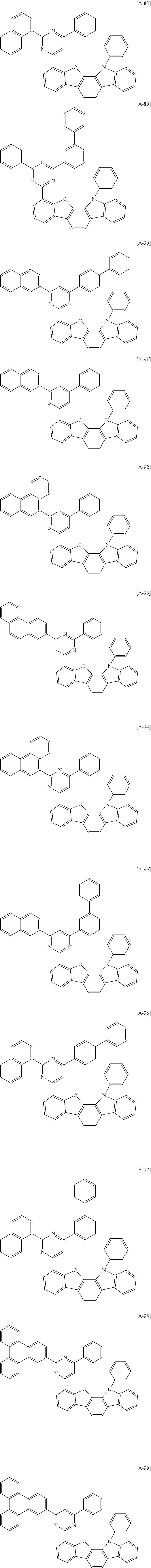

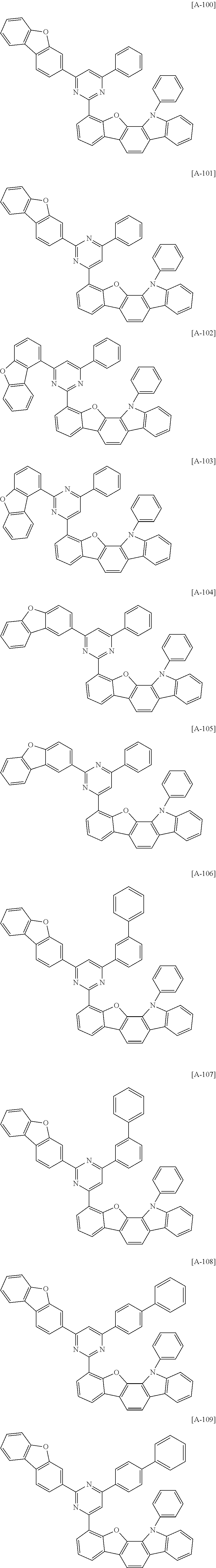

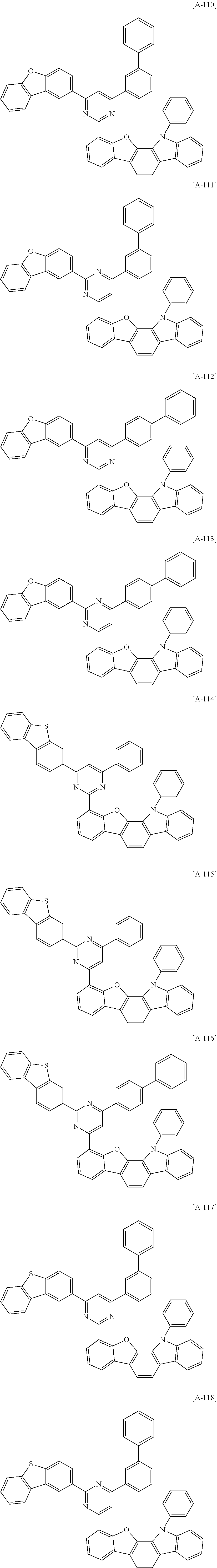

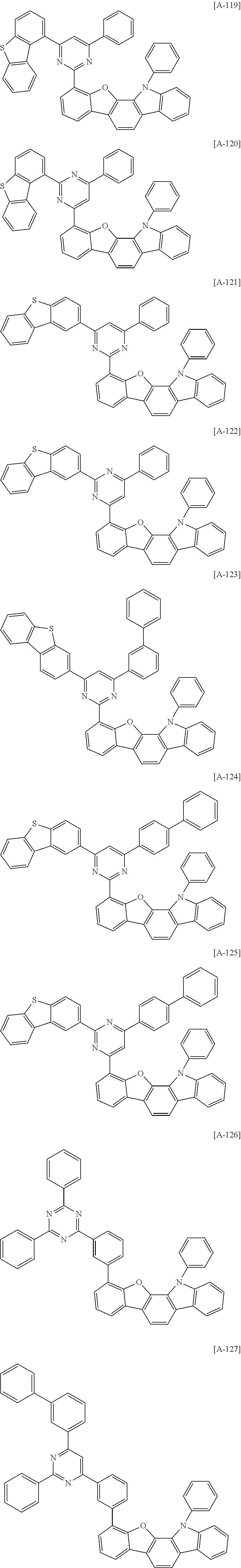

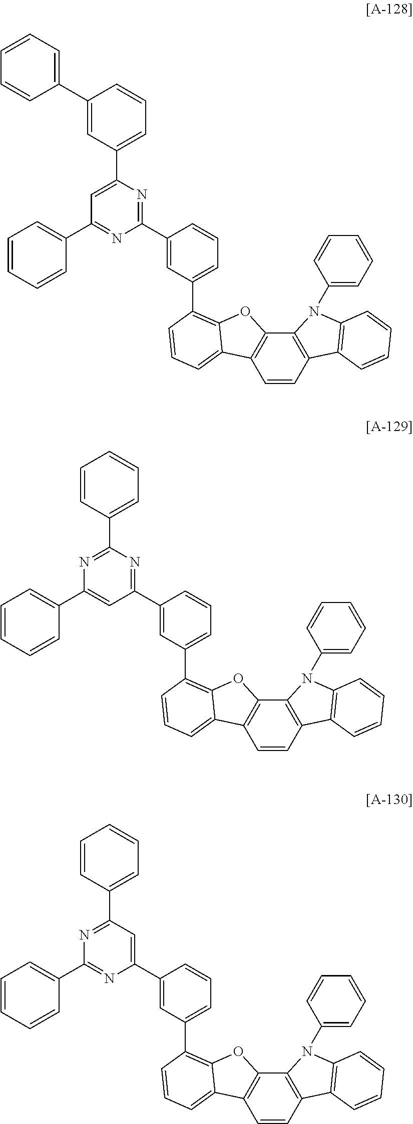 [SMILES: c1ccc(-c2cccc(-c3cc(-c4ccccc4)nc(-c4cccc(-c5cccc6c5oc5c6ccc6c7ccccc7n(-c7ccccc7)c65)c4)n3)c2)cc1 ; c1ccc(-c2cc(-c3cccc(-c4cccc5c4oc4c5ccc5c6ccccc6n(-c6ccccc6)c54)c3)nc(-c3ccccc3)n2)cc1 ; c1ccc(-c2cc(-c3cccc(-c4cccc5c4oc4c5ccc5c6ccccc6n(-c6ccccc6)c54)c3)nc(-c3ccccc3)n2)cc1]

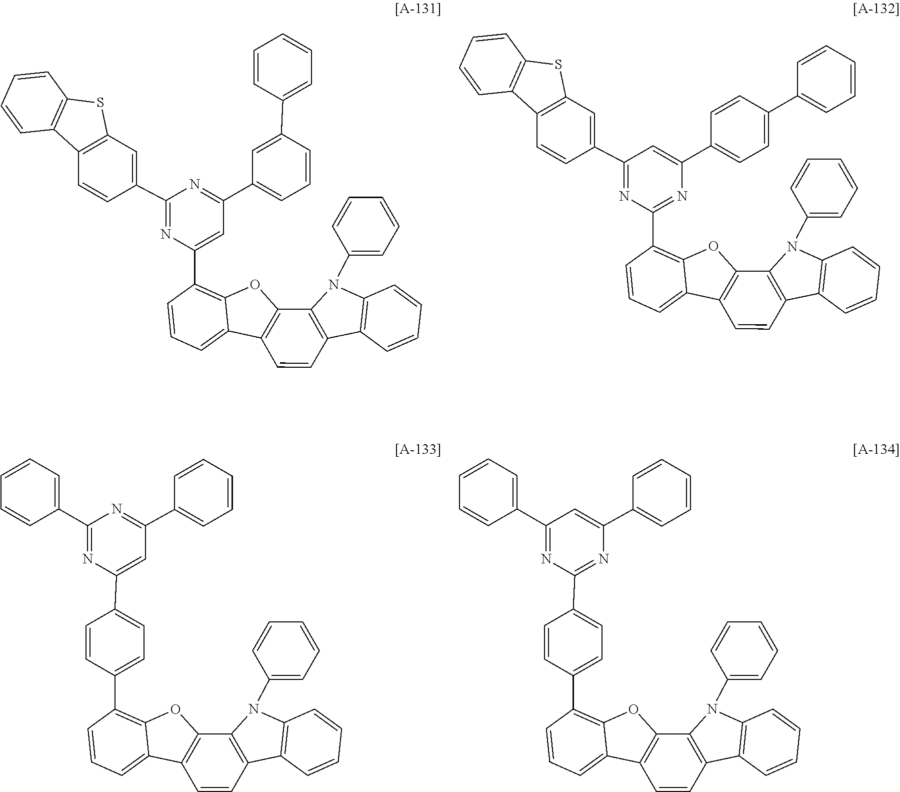 [SMILES: c1ccc(-c2cccc(-c3cc(-c4cccc5c4oc4c5ccc5c6ccccc6n(-c6ccccc6)c54)nc(-c4ccc5c(c4)sc4ccccc45)n3)c2)cc1 ; c1ccc(-c2ccc(-c3cc(-c4ccc5c(c4)sc4ccccc45)nc(-c4cccc5c4oc4c5ccc5c6ccccc6n(-c6ccccc6)c54)n3)cc2)cc1 ; c1ccc(-c2cc(-c3ccc(-c4cccc5c4oc4c5ccc5c6ccccc6n(-c6ccccc6)c54)cc3)nc(-c3ccccc3)n2)cc1 ; c1ccc(-c2cc(-c3ccccc3)nc(-c3ccc(-c4cccc5c4oc4c5ccc5c6ccccc6n(-c6ccccc6)c54)cc3)n2)cc1]

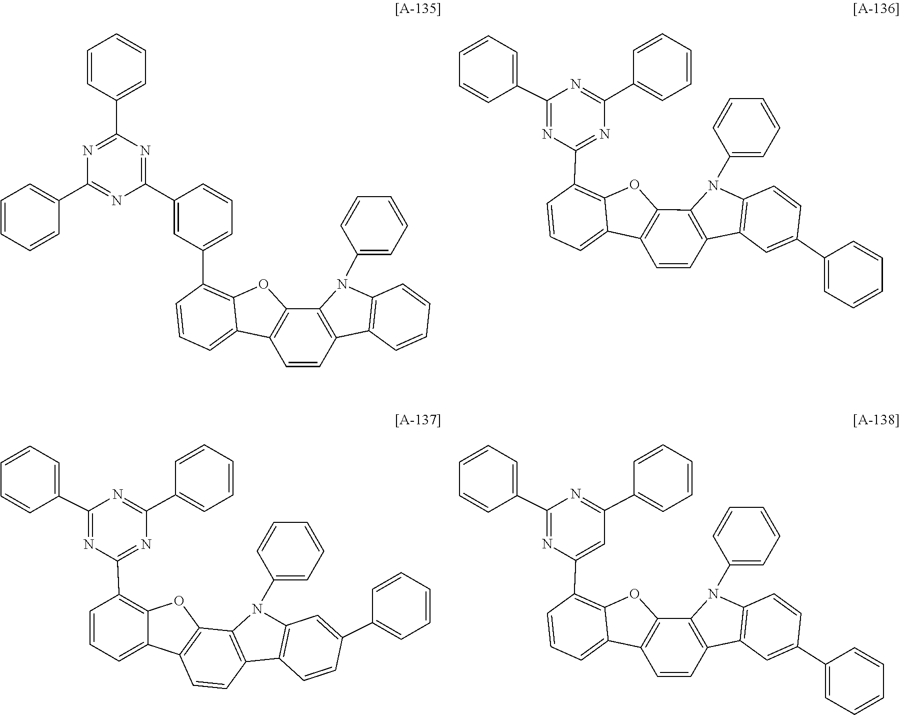 [SMILES: c1ccc(-c2nc(-c3ccccc3)nc(-c3cccc(-c4cccc5c4oc4c5ccc5c6ccccc6n(-c6ccccc6)c54)c3)n2)cc1 ; c1ccc(-c2ccc3c(c2)c2ccc4c5cccc(-c6nc(-c7ccccc7)nc(-c7ccccc7)n6)c5oc4c2n3-c2ccccc2)cc1 ; c1ccc(-c2ccc3c4ccc5c6cccc(-c7nc(-c8ccccc8)nc(-c8ccccc8)n7)c6oc5c4n(-c4ccccc4)c3c2)cc1 ; c1ccc(-c2ccc3c(c2)c2ccc4c5cccc(-c6cc(-c7ccccc7)nc(-c7ccccc7)n6)c5oc4c2n3-c2ccccc2)cc1]

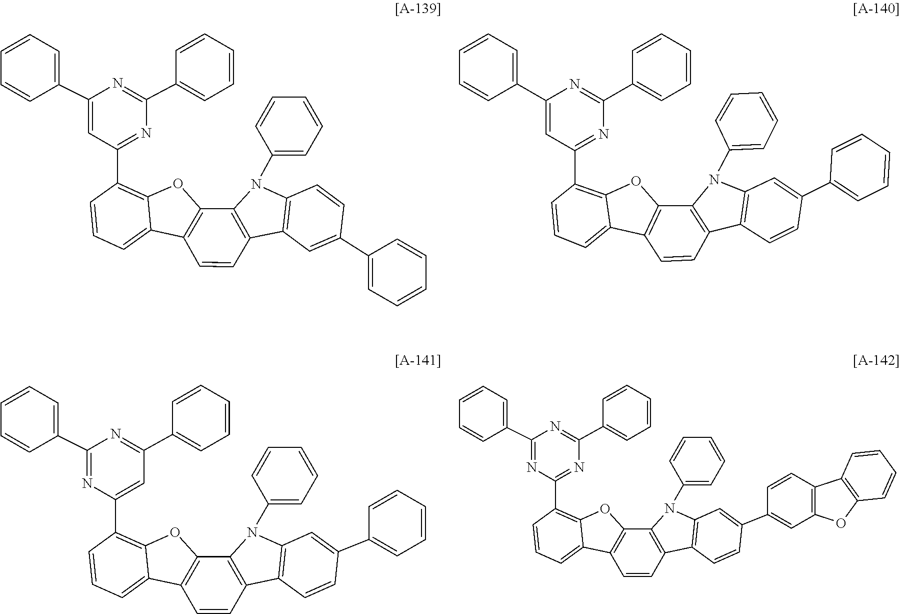 [SMILES: c1ccc(-c2ccc3c(c2)c2ccc4c5cccc(-c6cc(-c7ccccc7)nc(-c7ccccc7)n6)c5oc4c2n3-c2ccccc2)cc1 ; c1ccc(-c2ccc3c4ccc5c6cccc(-c7cc(-c8ccccc8)nc(-c8ccccc8)n7)c6oc5c4n(-c4ccccc4)c3c2)cc1 ; c1ccc(-c2ccc3c4ccc5c6cccc(-c7cc(-c8ccccc8)nc(-c8ccccc8)n7)c6oc5c4n(-c4ccccc4)c3c2)cc1 ; c1ccc(-c2nc(-c3ccccc3)nc(-c3cccc4c3oc3c4ccc4c5ccc(-c6ccc7c(c6)oc6ccccc67)cc5n(-c5ccccc5)c43)n2)cc1]

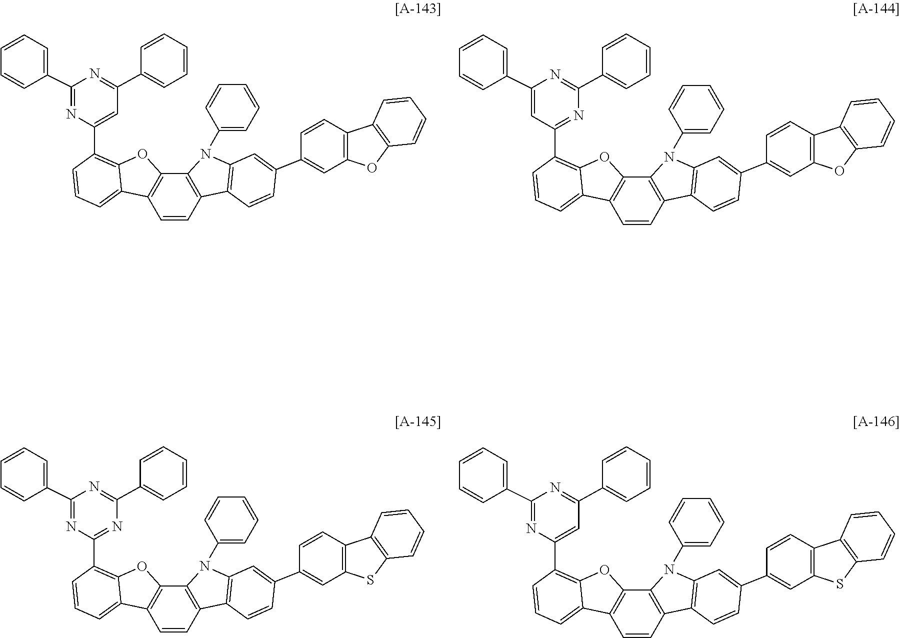 [SMILES: c1ccc(-c2cc(-c3cccc4c3oc3c4ccc4c5ccc(-c6ccc7c(c6)oc6ccccc67)cc5n(-c5ccccc5)c43)nc(-c3ccccc3)n2)cc1 ; c1ccc(-c2cc(-c3cccc4c3oc3c4ccc4c5ccc(-c6ccc7c(c6)oc6ccccc67)cc5n(-c5ccccc5)c43)nc(-c3ccccc3)n2)cc1 ; c1ccc(-c2nc(-c3ccccc3)nc(-c3cccc4c3oc3c4ccc4c5ccc(-c6ccc7c(c6)sc6ccccc67)cc5n(-c5ccccc5)c43)n2)cc1 ; c1ccc(-c2cc(-c3cccc4c3oc3c4ccc4c5ccc(-c6ccc7c(c6)sc6ccccc67)cc5n(-c5ccccc5)c43)nc(-c3ccccc3)n2)cc1]

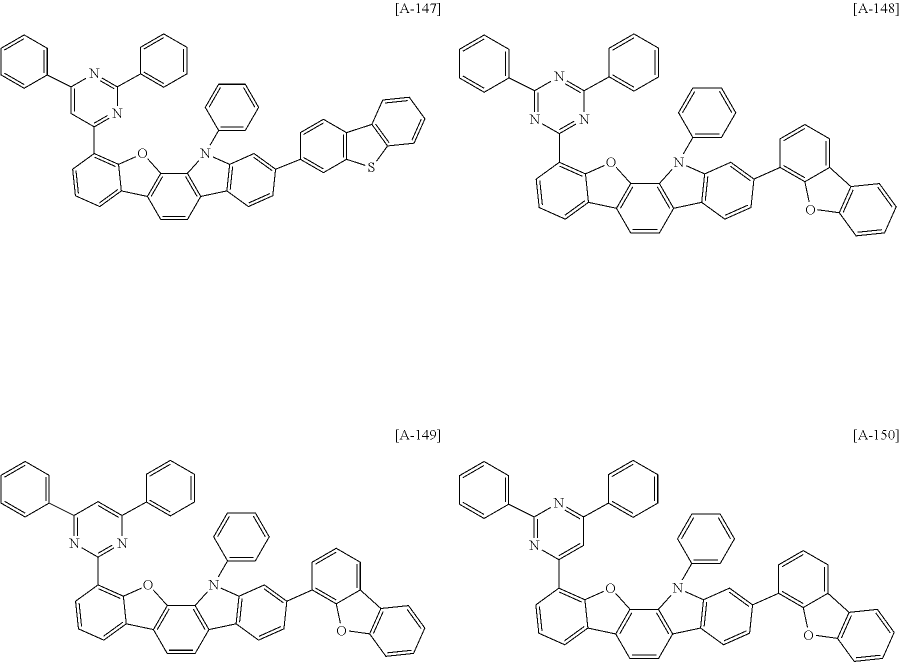 [SMILES: c1ccc(-c2cc(-c3cccc4c3oc3c4ccc4c5ccc(-c6ccc7c(c6)sc6ccccc67)cc5n(-c5ccccc5)c43)nc(-c3ccccc3)n2)cc1 ; c1ccc(-c2nc(-c3ccccc3)nc(-c3cccc4c3oc3c4ccc4c5ccc(-c6cccc7c6oc6ccccc67)cc5n(-c5ccccc5)c43)n2)cc1 ; c1ccc(-c2cc(-c3ccccc3)nc(-c3cccc4c3oc3c4ccc4c5ccc(-c6cccc7c6oc6ccccc67)cc5n(-c5ccccc5)c43)n2)cc1 ; c1ccc(-c2cc(-c3cccc4c3oc3c4ccc4c5ccc(-c6cccc7c6oc6ccccc67)cc5n(-c5ccccc5)c43)nc(-c3ccccc3)n2)cc1]

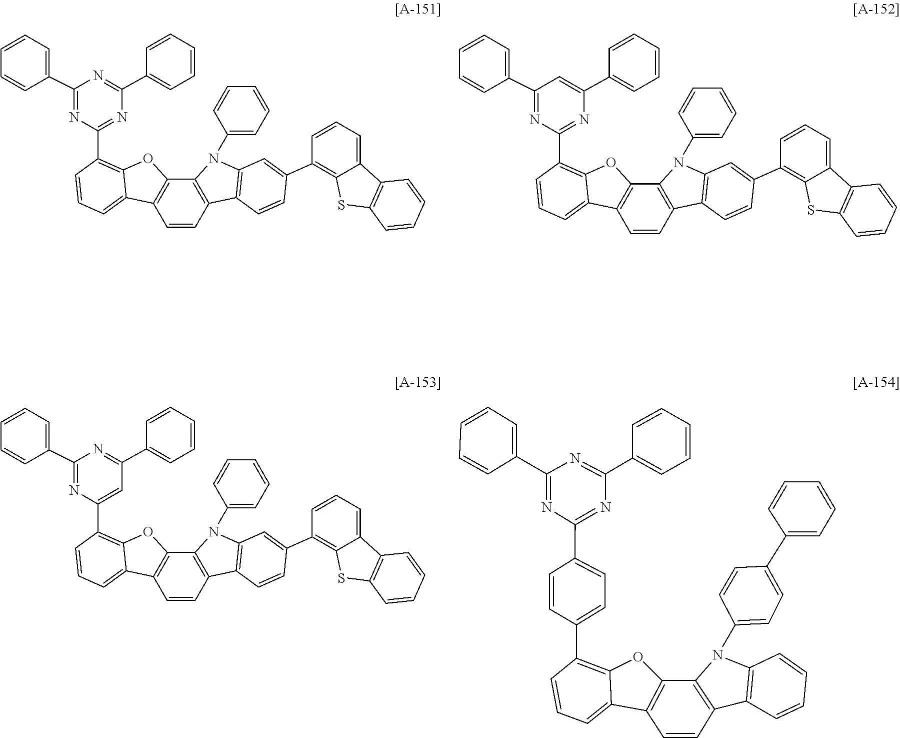 [SMILES: c1ccc(-c2nc(-c3ccccc3)nc(-c3cccc4c3oc3c4ccc4c5ccc(-c6cccc7c6sc6ccccc67)cc5n(-c5ccccc5)c43)n2)cc1 ; c1ccc(-c2cc(-c3ccccc3)nc(-c3cccc4c3oc3c4ccc4c5ccc(-c6cccc7c6sc6ccccc67)cc5n(-c5ccccc5)c43)n2)cc1 ; c1ccc(-c2cc(-c3cccc4c3oc3c4ccc4c5ccc(-c6cccc7c6sc6ccccc67)cc5n(-c5ccccc5)c43)nc(-c3ccccc3)n2)cc1 ; c1ccc(-c2ccc(-n3c4ccccc4c4ccc5c6cccc(-c7ccc(-c8nc(-c9ccccc9)nc(-c9ccccc9)n8)cc7)c6oc5c43)cc2)cc1]

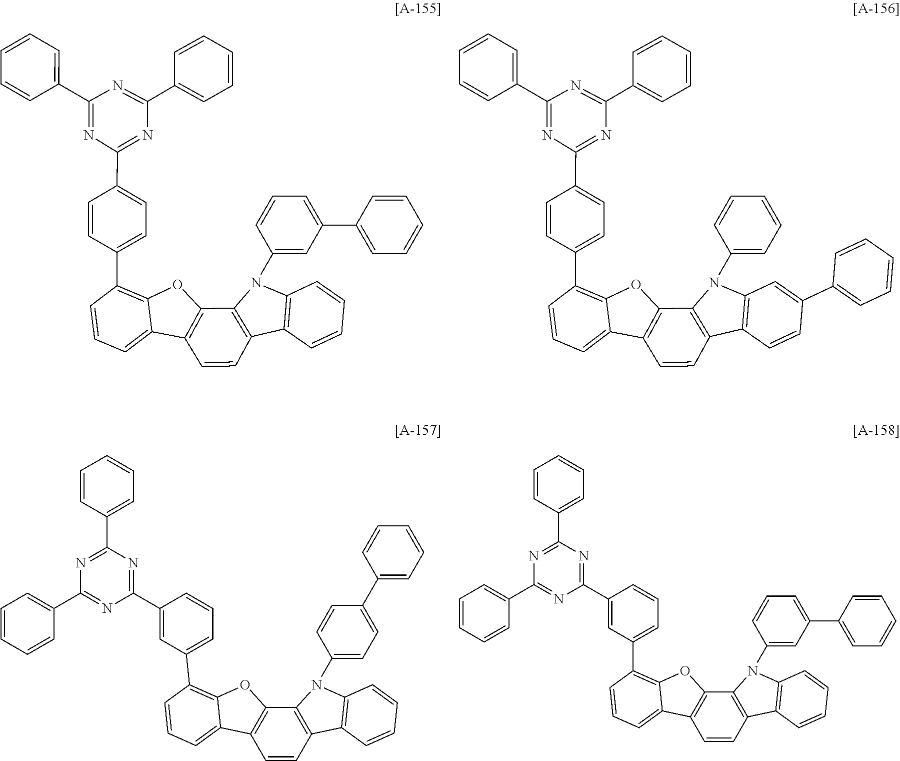 [SMILES: c1ccc(-c2cccc(-n3c4ccccc4c4ccc5c6cccc(-c7ccc(-c8nc(-c9ccccc9)nc(-c9ccccc9)n8)cc7)c6oc5c43)c2)cc1 ; c1ccc(-c2ccc3c4ccc5c6cccc(-c7ccc(-c8nc(-c9ccccc9)nc(-c9ccccc9)n8)cc7)c6oc5c4n(-c4ccccc4)c3c2)cc1 ; c1ccc(-c2ccc(-n3c4ccccc4c4ccc5c6cccc(-c7cccc(-c8nc(-c9ccccc9)nc(-c9ccccc9)n8)c7)c6oc5c43)cc2)cc1 ; c1ccc(-c2cccc(-n3c4ccccc4c4ccc5c6cccc(-c7cccc(-c8nc(-c9ccccc9)nc(-c9ccccc9)n8)c7)c6oc5c43)c2)cc1]

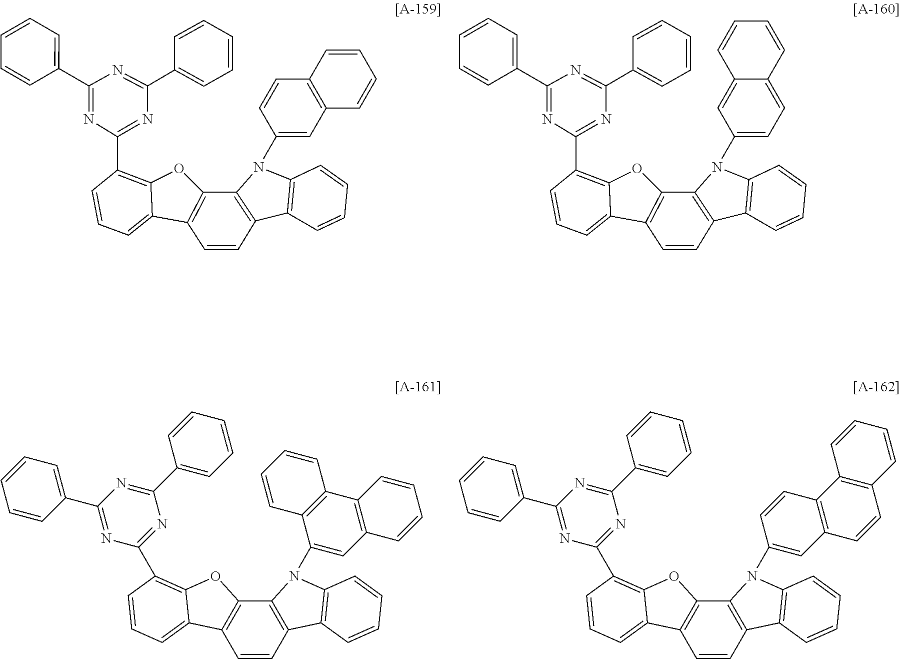

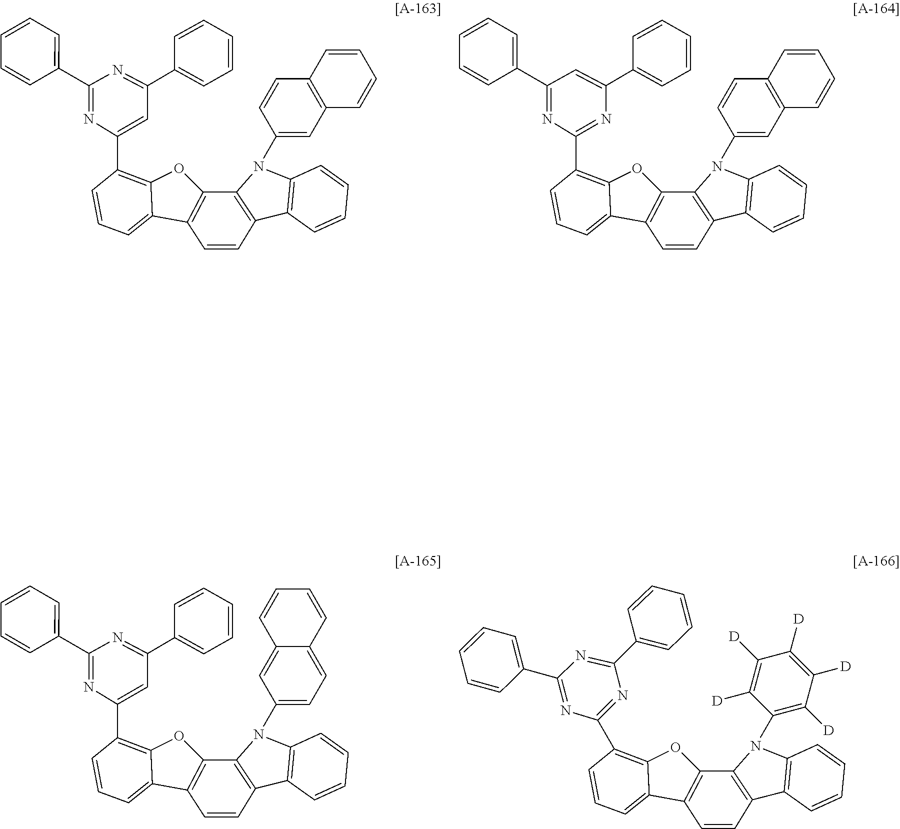 [SMILES: c1ccc(-c2cc(-c3cccc4c3oc3c4ccc4c5ccccc5n(-c5ccc6ccccc6c5)c43)nc(-c3ccccc3)n2)cc1 ; c1ccc(-c2cc(-c3ccccc3)nc(-c3cccc4c3oc3c4ccc4c5ccccc5n(-c5ccc6ccccc6c5)c43)n2)cc1 ; c1ccc(-c2cc(-c3cccc4c3oc3c4ccc4c5ccccc5n(-c5ccc6ccccc6c5)c43)nc(-c3ccccc3)n2)cc1 ; [2H]c1c([2H])c([2H])c(-n2c3ccccc3c3ccc4c5cccc(-c6nc(-c7ccccc7)nc(-c7ccccc7)n6)c5oc4c32)c([2H])c1[2H]]

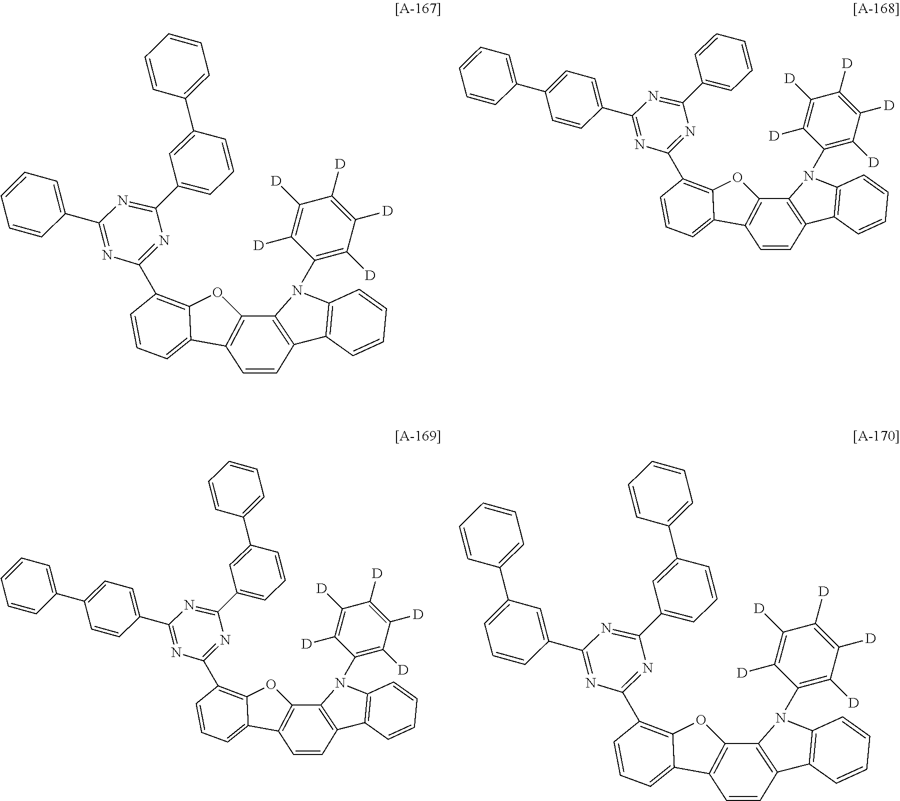 [SMILES: [2H]c1c([2H])c([2H])c(-n2c3ccccc3c3ccc4c5cccc(-c6nc(-c7ccccc7)nc(-c7cccc(-c8ccccc8)c7)n6)c5oc4c32)c([2H])c1[2H] ; [2H]c1c([2H])c([2H])c(-n2c3ccccc3c3ccc4c5cccc(-c6nc(-c7ccccc7)nc(-c7ccc(-c8ccccc8)cc7)n6)c5oc4c32)c([2H])c1[2H] ; [2H]c1c([2H])c([2H])c(-n2c3ccccc3c3ccc4c5cccc(-c6nc(-c7ccc(-c8ccccc8)cc7)nc(-c7cccc(-c8ccccc8)c7)n6)c5oc4c32)c([2H])c1[2H] ; [2H]c1c([2H])c([2H])c(-n2c3ccccc3c3ccc4c5cccc(-c6nc(-c7cccc(-c8ccccc8)c7)nc(-c7cccc(-c8ccccc8)c7)n6)c5oc4c32)c([2H])c1[2H]]

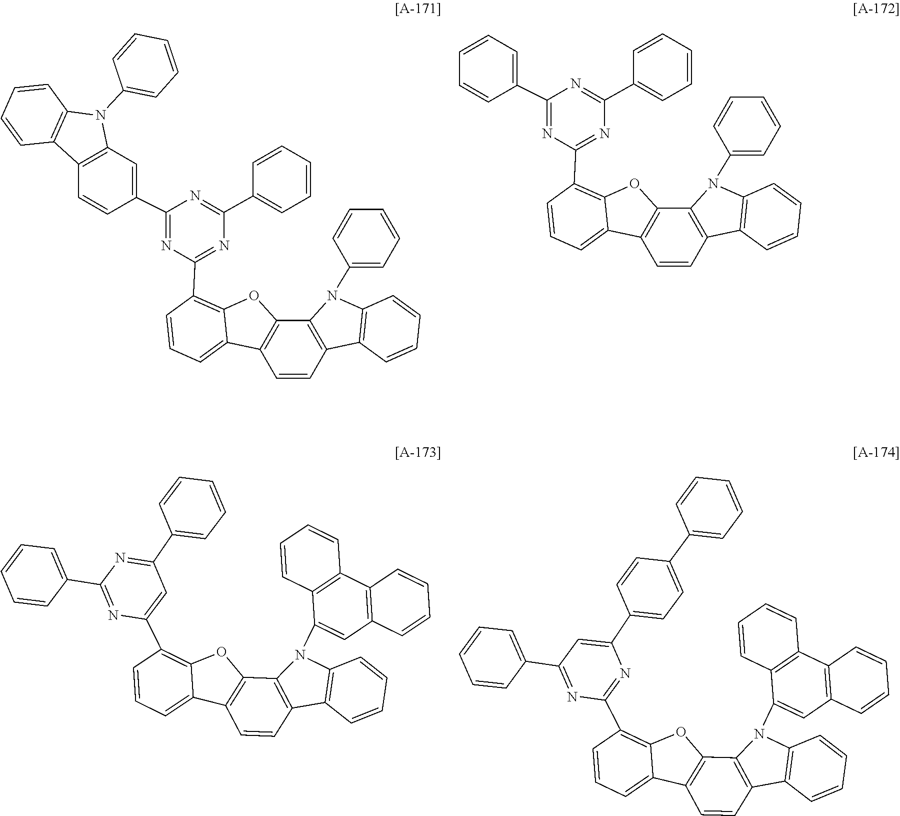 [SMILES: c1ccc(-c2nc(-c3ccc4c5ccccc5n(-c5ccccc5)c4c3)nc(-c3cccc4c3oc3c4ccc4c5ccccc5n(-c5ccccc5)c43)n2)cc1 ; c1ccc(-c2nc(-c3ccccc3)nc(-c3cccc4c3oc3c4ccc4c5ccccc5n(-c5ccccc5)c43)n2)cc1 ; c1ccc(-c2cc(-c3cccc4c3oc3c4ccc4c5ccccc5n(-c5cc6ccccc6c6ccccc56)c43)nc(-c3ccccc3)n2)cc1 ; c1ccc(-c2ccc(-c3cc(-c4ccccc4)nc(-c4cccc5c4oc4c5ccc5c6ccccc6n(-c6cc7ccccc7c7ccccc67)c54)n3)cc2)cc1]

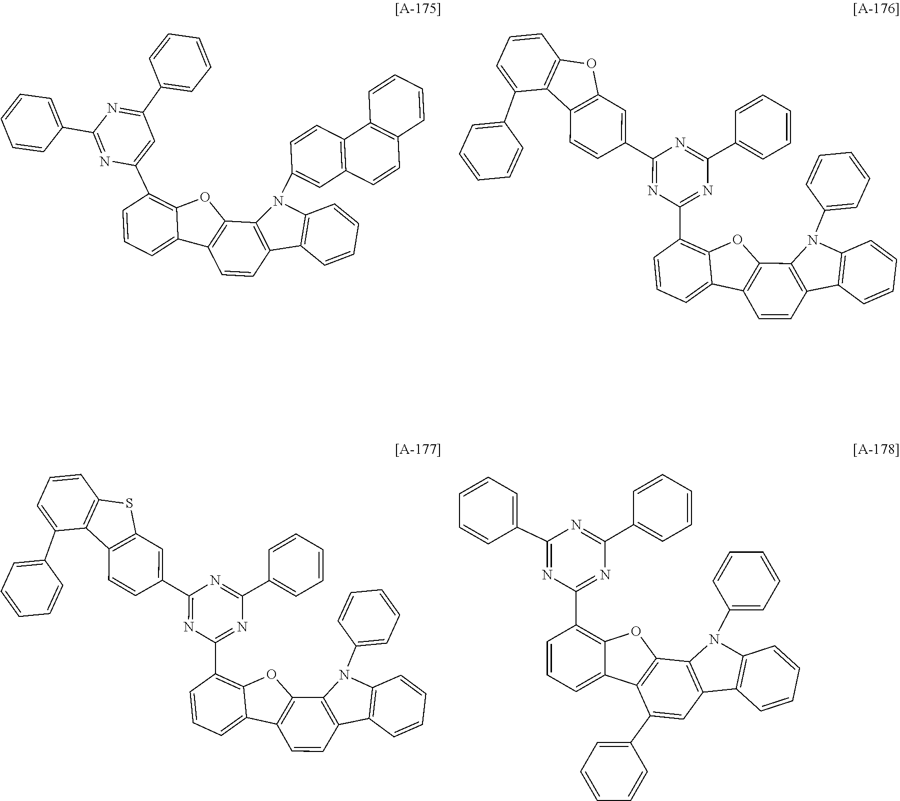 [SMILES: c1ccc(-c2cc(-c3cccc4c3oc3c4ccc4c5ccccc5n(-c5ccc6c(ccc7ccccc76)c5)c43)nc(-c3ccccc3)n2)cc1 ; c1ccc(-c2nc(-c3ccc4c(c3)oc3cccc(-c5ccccc5)c34)nc(-c3cccc4c3oc3c4ccc4c5ccccc5n(-c5ccccc5)c43)n2)cc1 ; c1ccc(-c2nc(-c3ccc4c(c3)sc3cccc(-c5ccccc5)c34)nc(-c3cccc4c3oc3c4ccc4c5ccccc5n(-c5ccccc5)c43)n2)cc1 ; c1ccc(-c2nc(-c3ccccc3)nc(-c3cccc4c3oc3c4c(-c4ccccc4)cc4c5ccccc5n(-c5ccccc5)c43)n2)cc1]

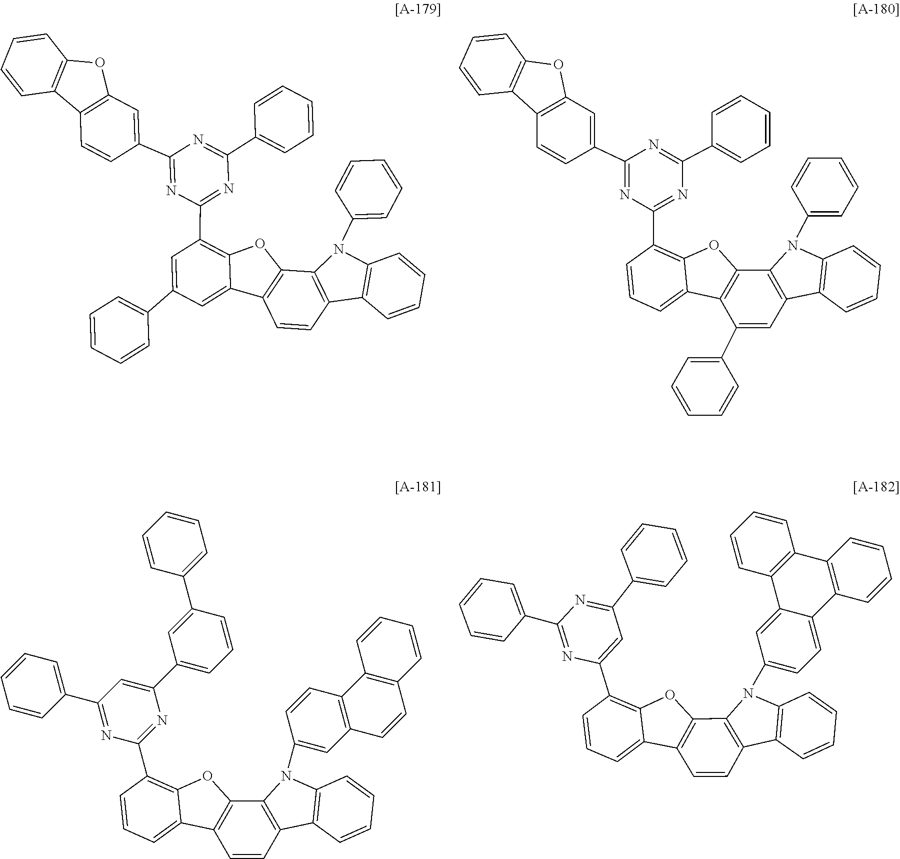 [SMILES: c1ccc(-c2cc(-c3nc(-c4ccccc4)nc(-c4ccc5c(c4)oc4ccccc45)n3)c3oc4c(ccc5c6ccccc6n(-c6ccccc6)c54)c3c2)cc1 ; c1ccc(-c2nc(-c3ccc4c(c3)oc3ccccc34)nc(-c3cccc4c3oc3c4c(-c4ccccc4)cc4c5ccccc5n(-c5ccccc5)c43)n2)cc1 ; c1ccc(-c2cccc(-c3cc(-c4ccccc4)nc(-c4cccc5c4oc4c5ccc5c6ccccc6n(-c6ccc7c(ccc8ccccc87)c6)c54)n3)c2)cc1 ; c1ccc(-c2cc(-c3cccc4c3oc3c4ccc4c5ccccc5n(-c5ccc6c7ccccc7c7ccccc7c6c5)c43)nc(-c3ccccc3)n2)cc1]

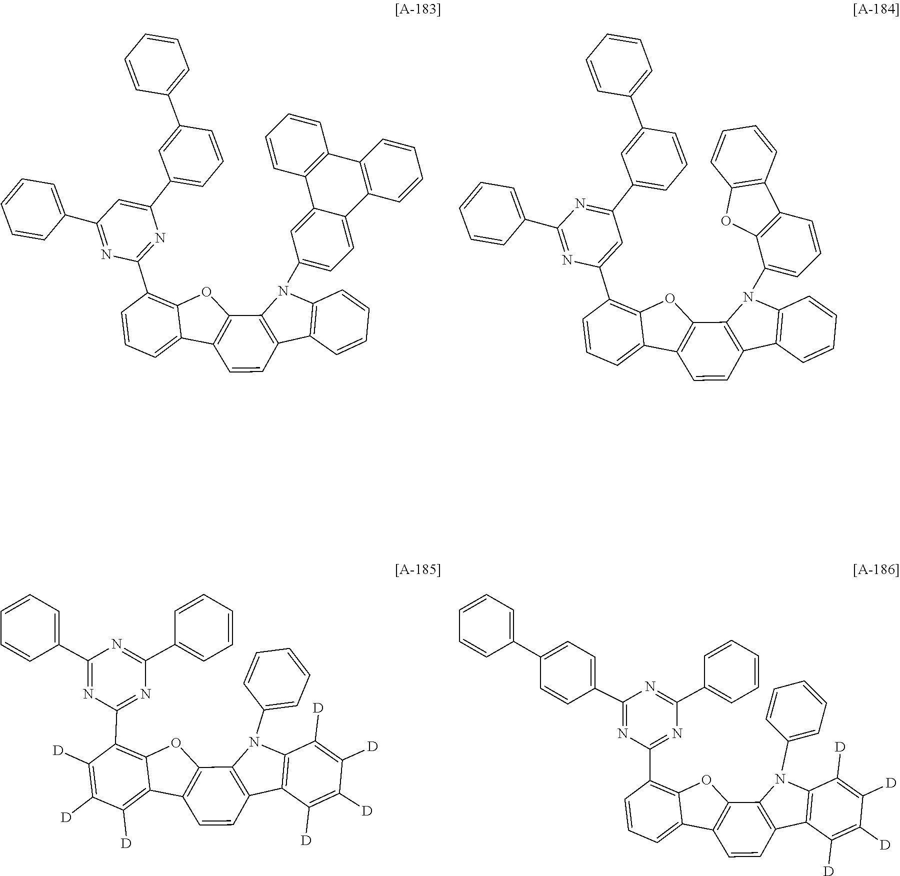 [SMILES: c1ccc(-c2cccc(-c3cc(-c4ccccc4)nc(-c4cccc5c4oc4c5ccc5c6ccccc6n(-c6ccc7c8ccccc8c8ccccc8c7c6)c54)n3)c2)cc1 ; c1ccc(-c2cccc(-c3cc(-c4cccc5c4oc4c5ccc5c6ccccc6n(-c6cccc7c6oc6ccccc67)c54)nc(-c4ccccc4)n3)c2)cc1 ; [2H]c1c([2H])c([2H])c2c(oc3c2ccc2c4c([2H])c([2H])c([2H])c([2H])c4n(-c4ccccc4)c23)c1-c1nc(-c2ccccc2)nc(-c2ccccc2)n1 ; [2H]c1c([2H])c([2H])c2c(c1[2H])c1ccc3c4cccc(-c5nc(-c6ccccc6)nc(-c6ccc(-c7ccccc7)cc6)n5)c4oc3c1n2-c1ccccc1]

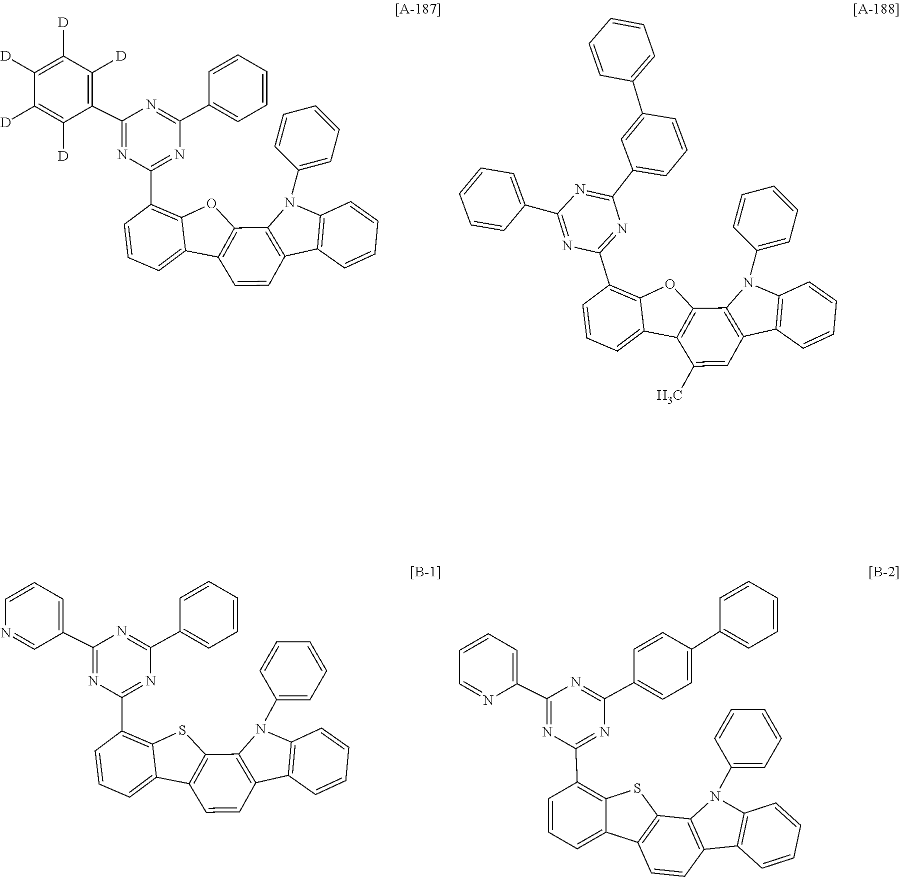 [SMILES: [2H]c1c([2H])c([2H])c(-c2nc(-c3ccccc3)nc(-c3cccc4c3oc3c4ccc4c5ccccc5n(-c5ccccc5)c43)n2)c([2H])c1[2H] ; Cc1cc2c3ccccc3n(-c3ccccc3)c2c2oc3c(-c4nc(-c5ccccc5)nc(-c5cccc(-c6ccccc6)c5)n4)cccc3c12 ; c1ccc(-c2nc(-c3cccnc3)nc(-c3cccc4c3sc3c4ccc4c5ccccc5n(-c5ccccc5)c43)n2)cc1 ; c1ccc(-c2ccc(-c3nc(-c4ccccn4)nc(-c4cccc5c4sc4c5ccc5c6ccccc6n(-c6ccccc6)c54)n3)cc2)cc1]

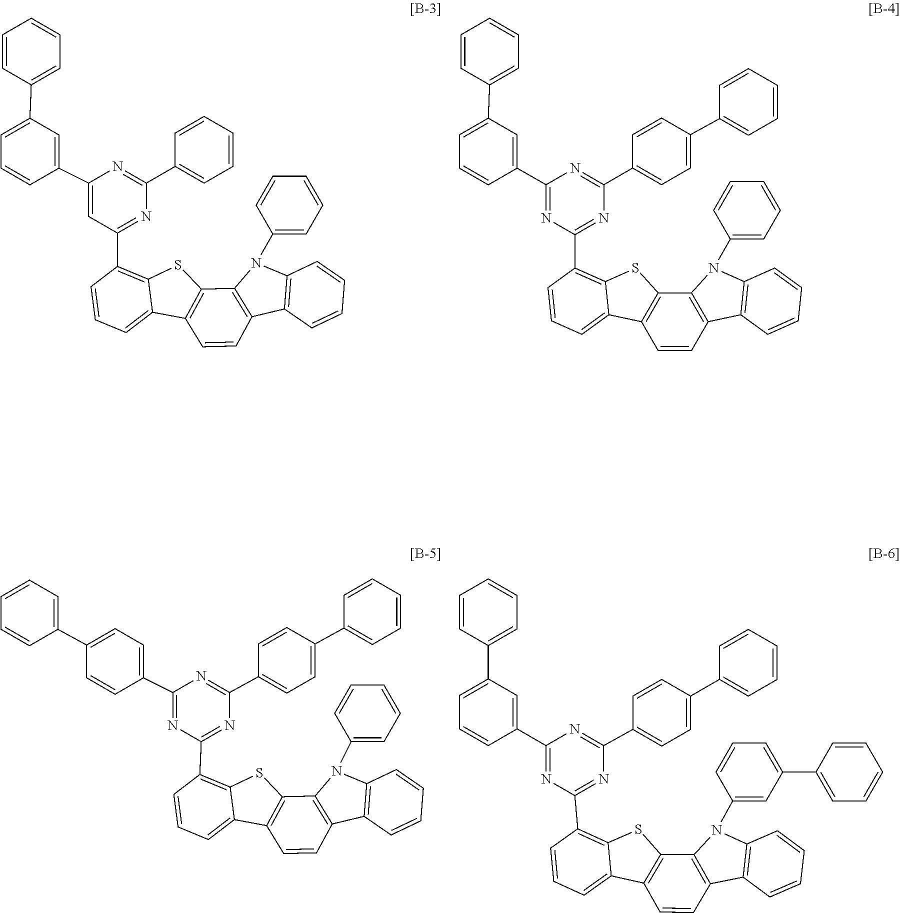 [SMILES: c1ccc(-c2cccc(-c3cc(-c4cccc5c4sc4c5ccc5c6ccccc6n(-c6ccccc6)c54)nc(-c4ccccc4)n3)c2)cc1 ; c1ccc(-c2ccc(-c3nc(-c4cccc(-c5ccccc5)c4)nc(-c4cccc5c4sc4c5ccc5c6ccccc6n(-c6ccccc6)c54)n3)cc2)cc1 ; c1ccc(-c2ccc(-c3nc(-c4ccc(-c5ccccc5)cc4)nc(-c4cccc5c4sc4c5ccc5c6ccccc6n(-c6ccccc6)c54)n3)cc2)cc1 ; c1ccc(-c2ccc(-c3nc(-c4cccc(-c5ccccc5)c4)nc(-c4cccc5c4sc4c5ccc5c6ccccc6n(-c6cccc(-c7ccccc7)c6)c54)n3)cc2)cc1]

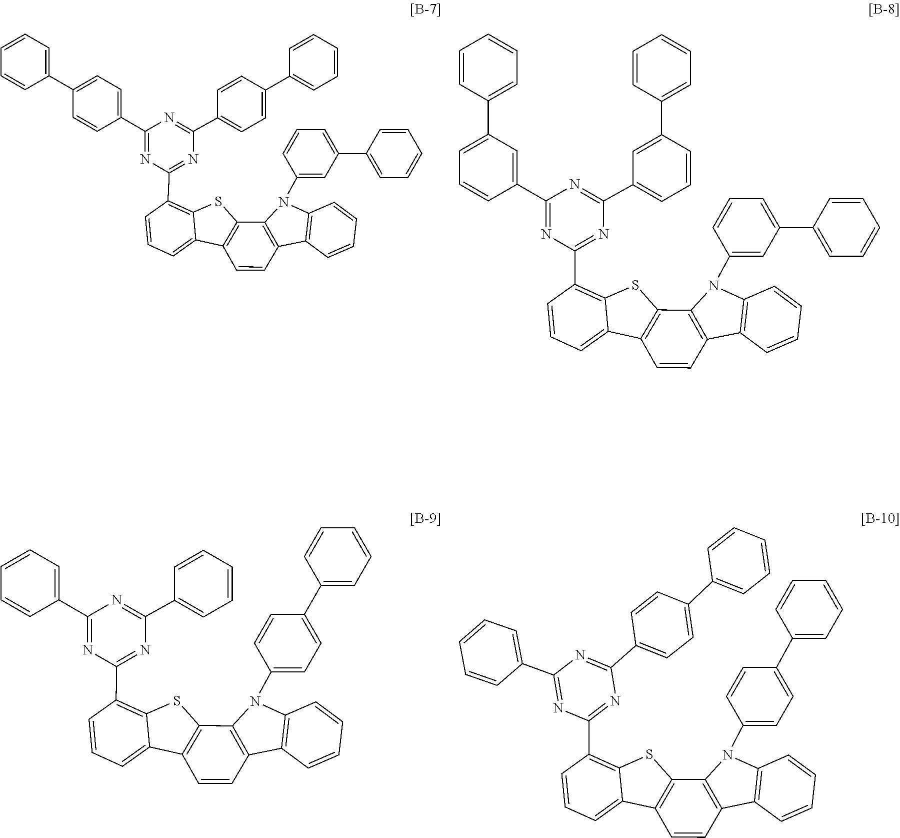

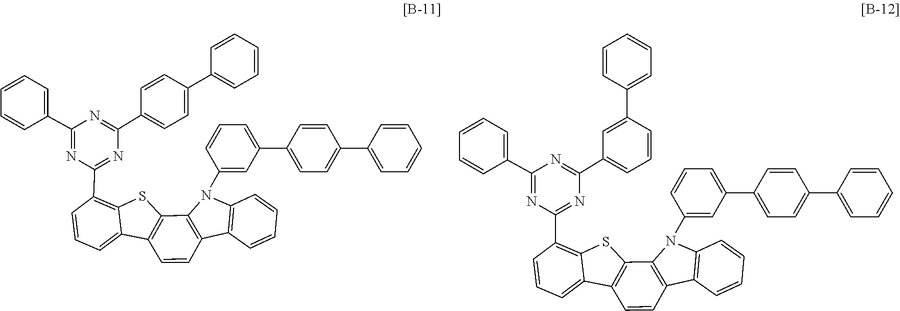 [SMILES: c1ccc(-c2ccc(-c3cccc(-n4c5ccccc5c5ccc6c7cccc(-c8nc(-c9ccccc9)nc(-c9ccc(-c%10ccccc%10)cc9)n8)c7sc6c54)c3)cc2)cc1 ; c1ccc(-c2ccc(-c3cccc(-n4c5ccccc5c5ccc6c7cccc(-c8nc(-c9ccccc9)nc(-c9cccc(-c%10ccccc%10)c9)n8)c7sc6c54)c3)cc2)cc1]

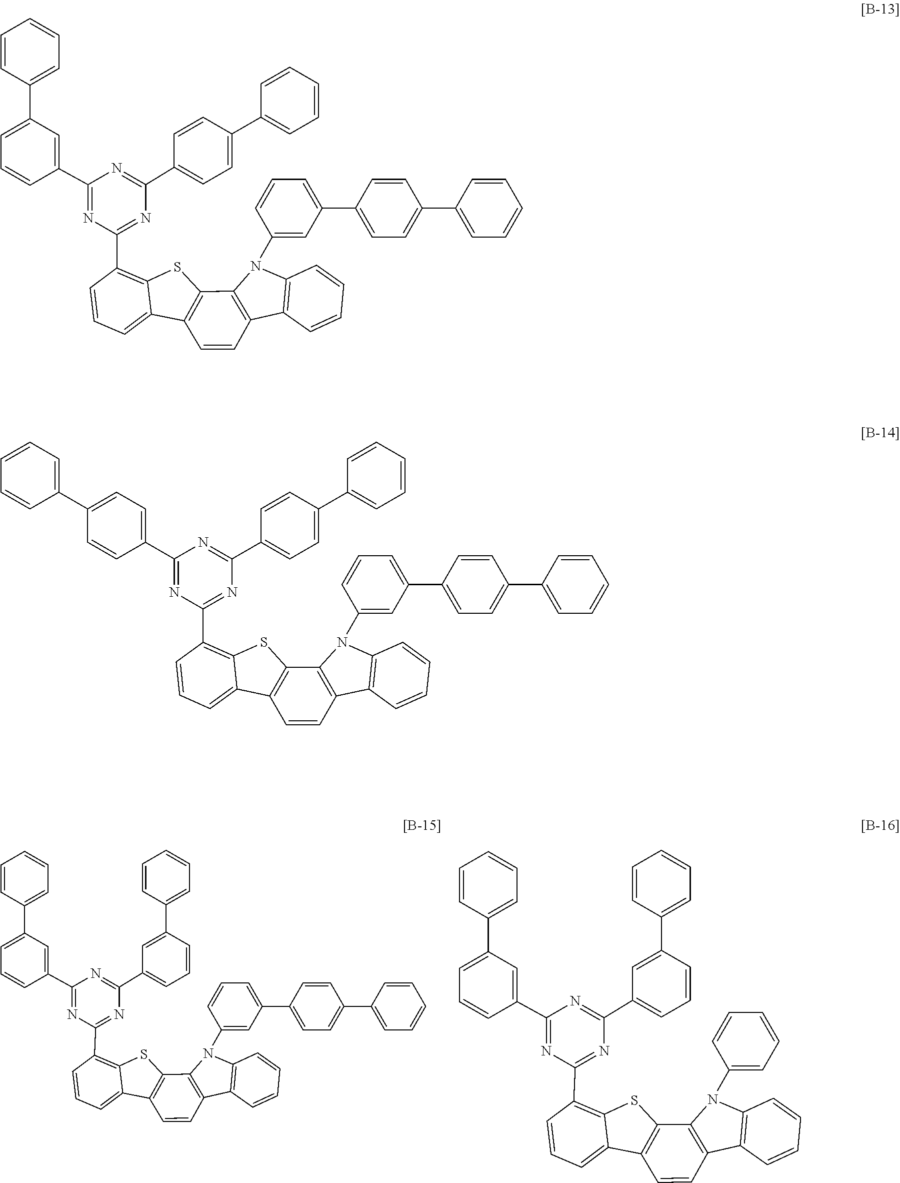

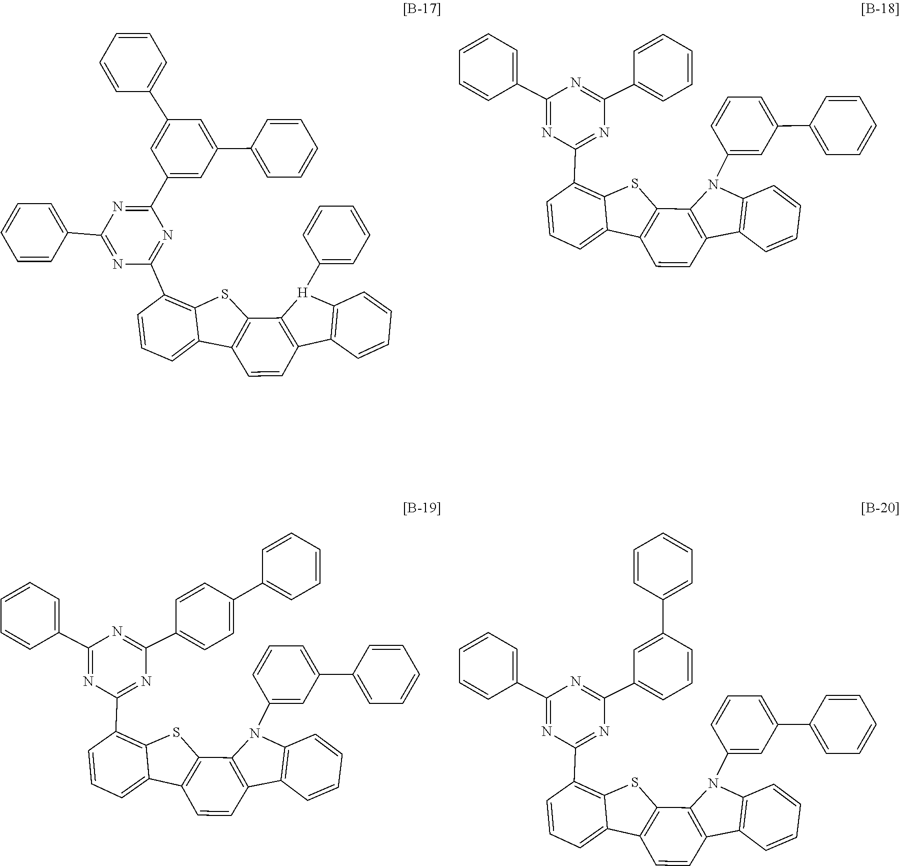

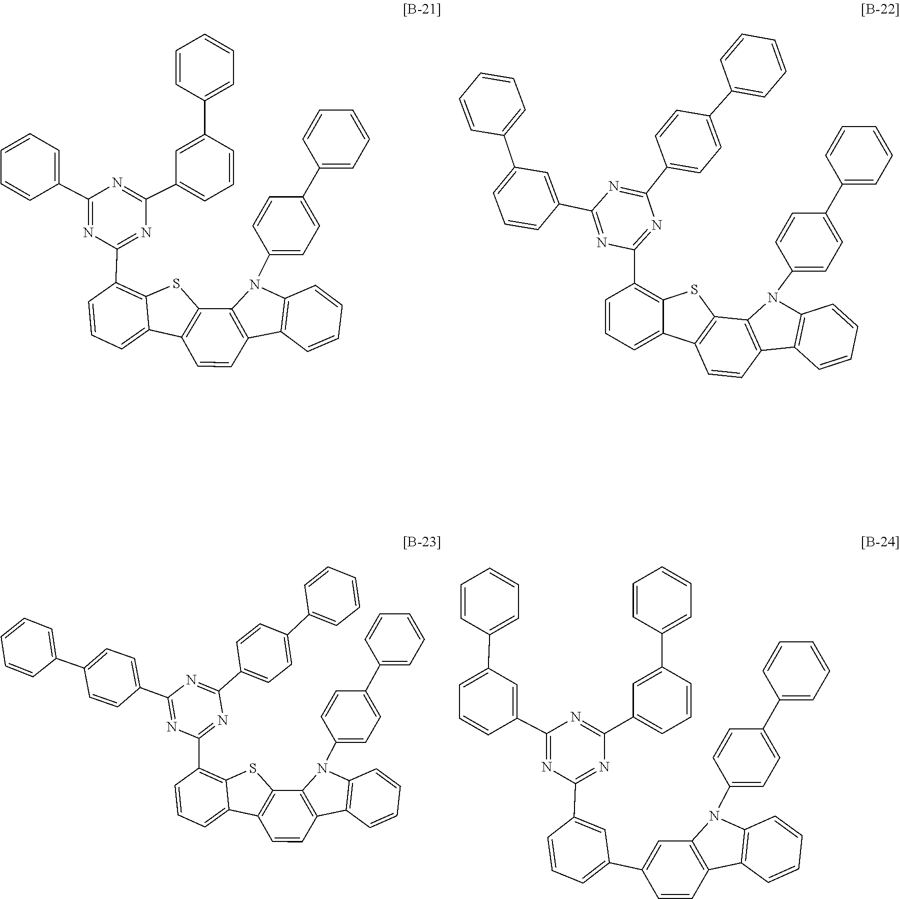

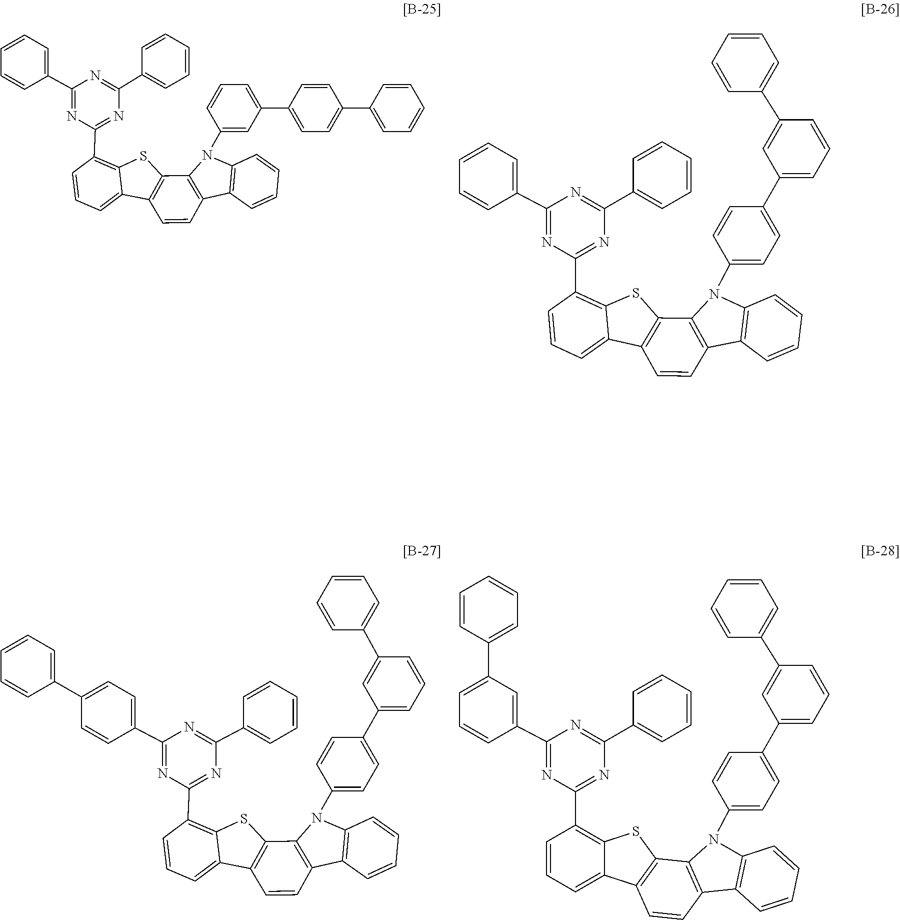

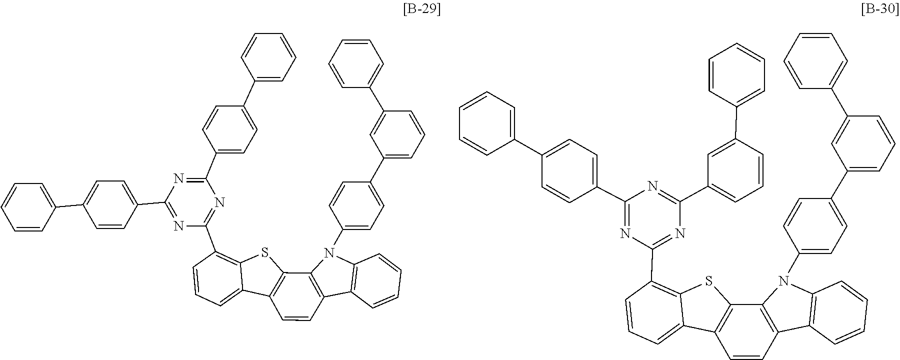

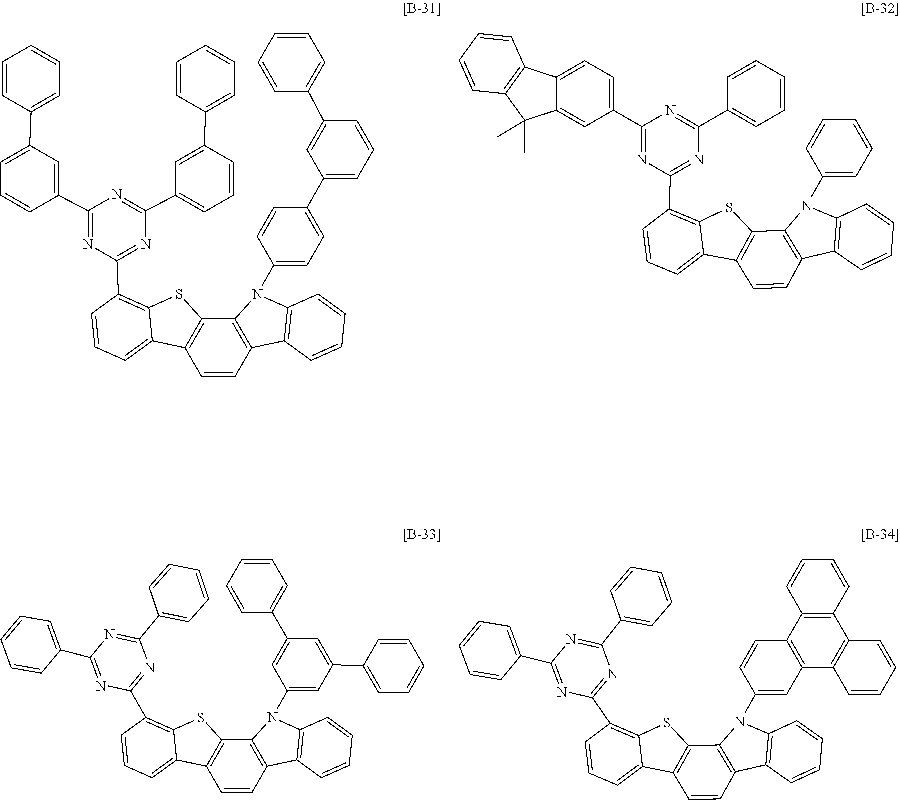

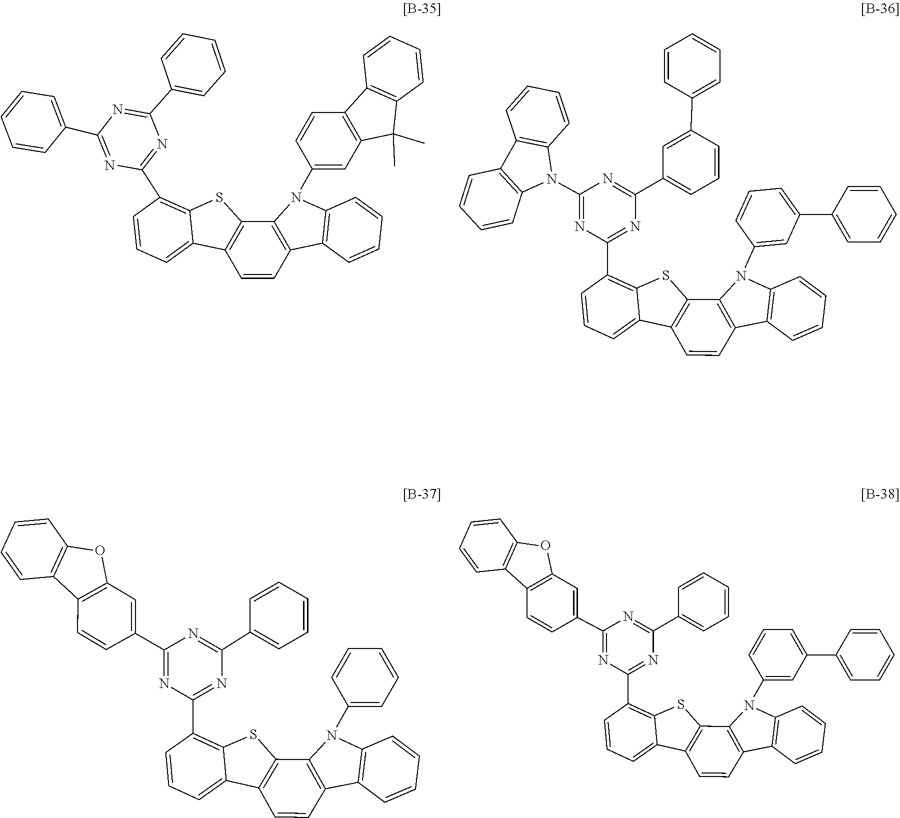 [SMILES: CC1(C)c2ccccc2-c2ccc(-n3c4ccccc4c4ccc5c6cccc(-c7nc(-c8ccccc8)nc(-c8ccccc8)n7)c6sc5c43)cc21 ; c1ccc(-c2cccc(-c3nc(-c4cccc5c4sc4c5ccc5c6ccccc6n(-c6cccc(-c7ccccc7)c6)c54)nc(-n4c5ccccc5c5ccccc54)n3)c2)cc1 ; c1ccc(-c2nc(-c3ccc4c(c3)oc3ccccc34)nc(-c3cccc4c3sc3c4ccc4c5ccccc5n(-c5ccccc5)c43)n2)cc1 ; c1ccc(-c2cccc(-n3c4ccccc4c4ccc5c6cccc(-c7nc(-c8ccccc8)nc(-c8ccc9c(c8)oc8ccccc89)n7)c6sc5c43)c2)cc1]

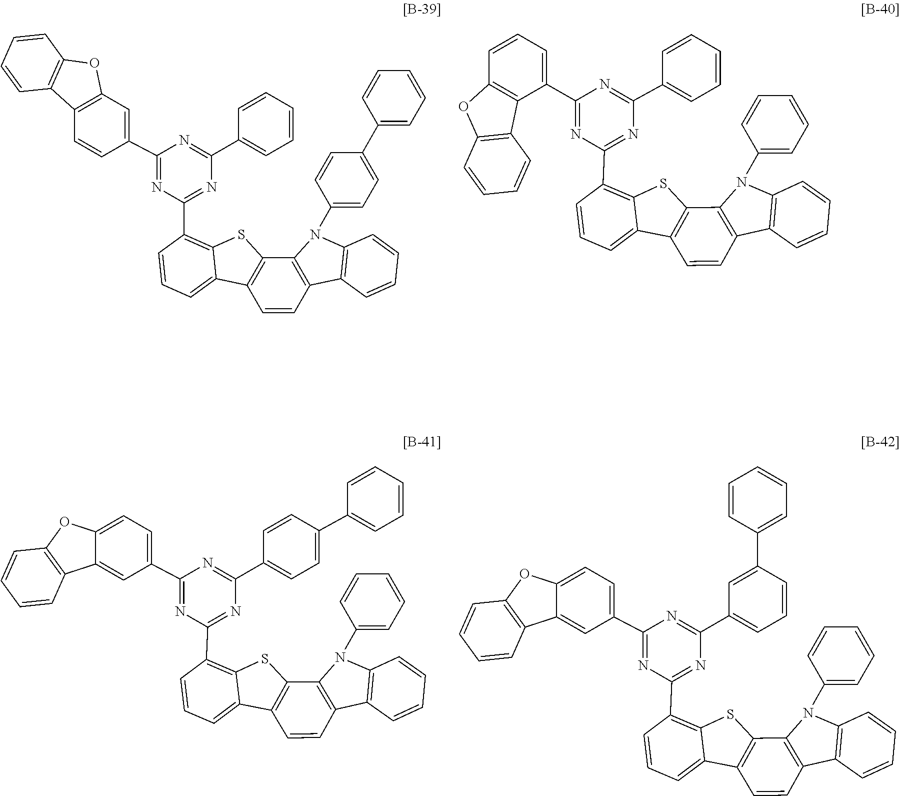

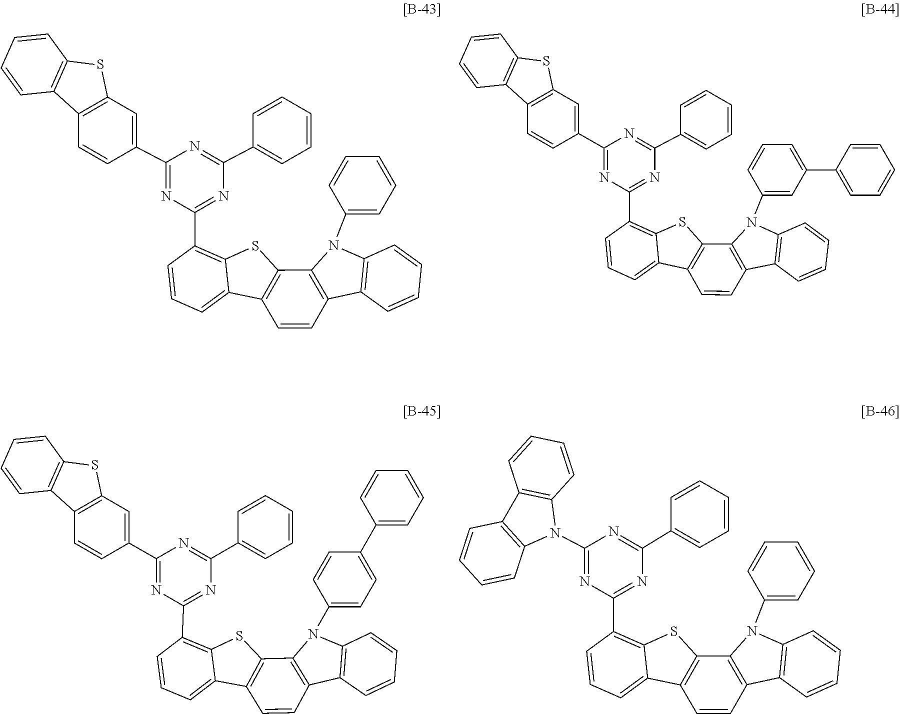 [SMILES: c1ccc(-c2nc(-c3ccc4c(c3)sc3ccccc34)nc(-c3cccc4c3sc3c4ccc4c5ccccc5n(-c5ccccc5)c43)n2)cc1 ; c1ccc(-c2cccc(-n3c4ccccc4c4ccc5c6cccc(-c7nc(-c8ccccc8)nc(-c8ccc9c(c8)sc8ccccc89)n7)c6sc5c43)c2)cc1 ; c1ccc(-c2ccc(-n3c4ccccc4c4ccc5c6cccc(-c7nc(-c8ccccc8)nc(-c8ccc9c(c8)sc8ccccc89)n7)c6sc5c43)cc2)cc1 ; c1ccc(-c2nc(-c3cccc4c3sc3c4ccc4c5ccccc5n(-c5ccccc5)c43)nc(-n3c4ccccc4c4ccccc43)n2)cc1]

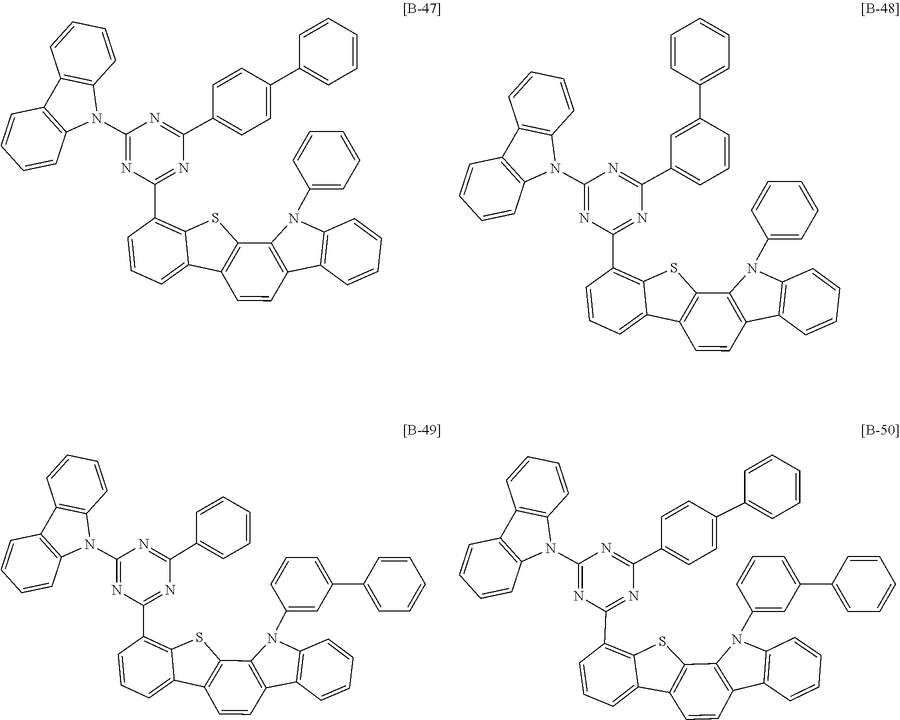

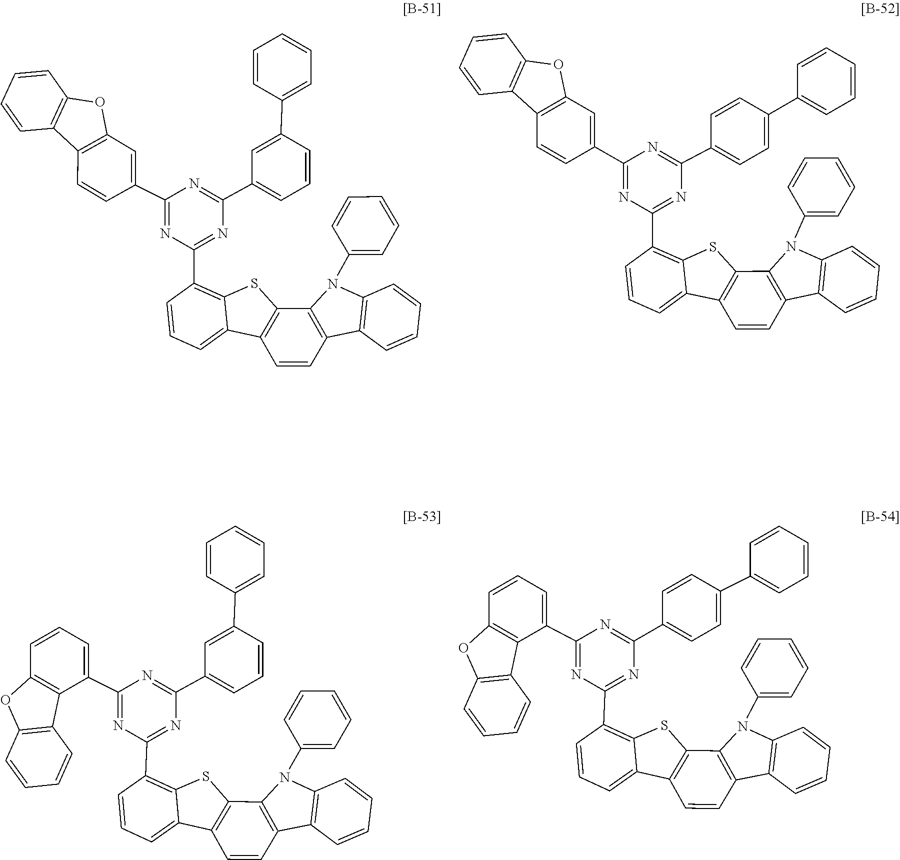 [SMILES: c1ccc(-c2cccc(-c3nc(-c4ccc5c(c4)oc4ccccc45)nc(-c4cccc5c4sc4c5ccc5c6ccccc6n(-c6ccccc6)c54)n3)c2)cc1 ; c1ccc(-c2ccc(-c3nc(-c4ccc5c(c4)oc4ccccc45)nc(-c4cccc5c4sc4c5ccc5c6ccccc6n(-c6ccccc6)c54)n3)cc2)cc1 ; c1ccc(-c2cccc(-c3nc(-c4cccc5c4sc4c5ccc5c6ccccc6n(-c6ccccc6)c54)nc(-c4cccc5oc6ccccc6c45)n3)c2)cc1 ; c1ccc(-c2ccc(-c3nc(-c4cccc5c4sc4c5ccc5c6ccccc6n(-c6ccccc6)c54)nc(-c4cccc5oc6ccccc6c45)n3)cc2)cc1]

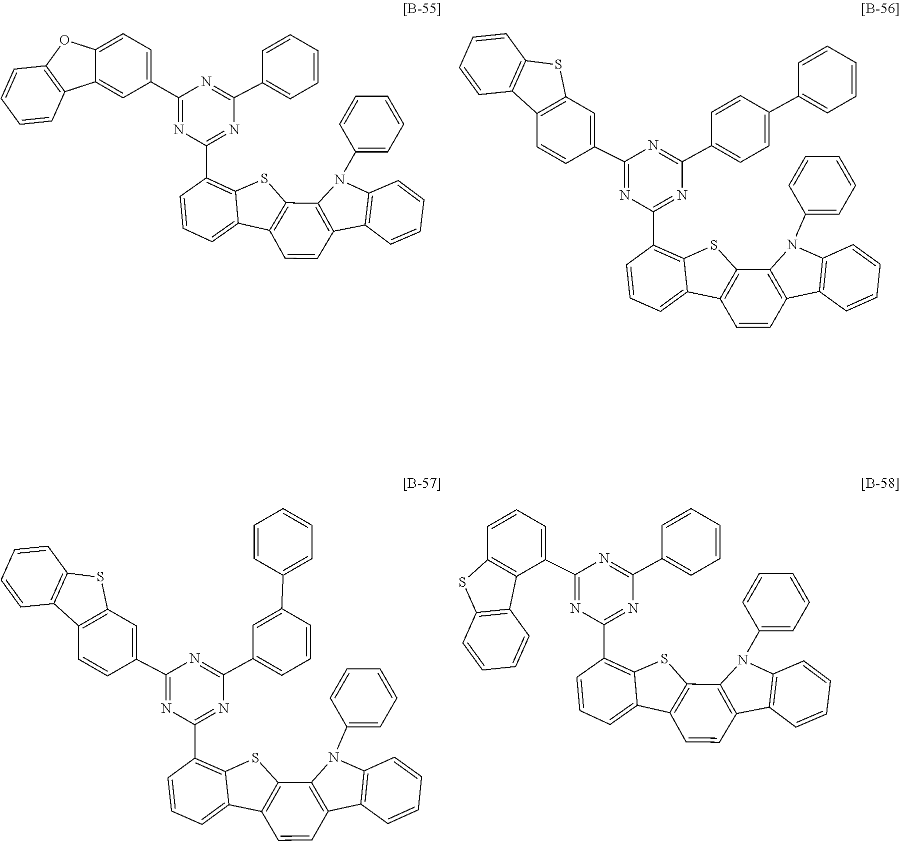 [SMILES: c1ccc(-c2nc(-c3ccc4oc5ccccc5c4c3)nc(-c3cccc4c3sc3c4ccc4c5ccccc5n(-c5ccccc5)c43)n2)cc1 ; c1ccc(-c2ccc(-c3nc(-c4ccc5c(c4)sc4ccccc45)nc(-c4cccc5c4sc4c5ccc5c6ccccc6n(-c6ccccc6)c54)n3)cc2)cc1 ; c1ccc(-c2cccc(-c3nc(-c4ccc5c(c4)sc4ccccc45)nc(-c4cccc5c4sc4c5ccc5c6ccccc6n(-c6ccccc6)c54)n3)c2)cc1 ; c1ccc(-c2nc(-c3cccc4c3sc3c4ccc4c5ccccc5n(-c5ccccc5)c43)nc(-c3cccc4sc5ccccc5c34)n2)cc1]

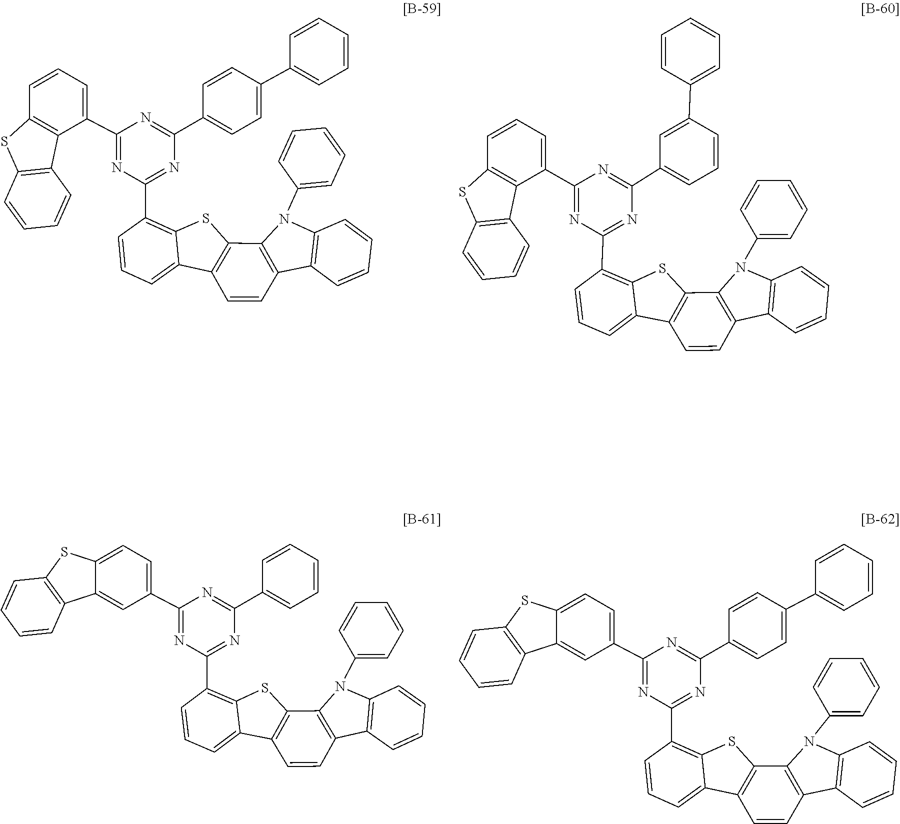 [SMILES: c1ccc(-c2ccc(-c3nc(-c4cccc5c4sc4c5ccc5c6ccccc6n(-c6ccccc6)c54)nc(-c4cccc5sc6ccccc6c45)n3)cc2)cc1 ; c1ccc(-c2cccc(-c3nc(-c4cccc5c4sc4c5ccc5c6ccccc6n(-c6ccccc6)c54)nc(-c4cccc5sc6ccccc6c45)n3)c2)cc1 ; c1ccc(-c2nc(-c3ccc4sc5ccccc5c4c3)nc(-c3cccc4c3sc3c4ccc4c5ccccc5n(-c5ccccc5)c43)n2)cc1 ; c1ccc(-c2ccc(-c3nc(-c4ccc5sc6ccccc6c5c4)nc(-c4cccc5c4sc4c5ccc5c6ccccc6n(-c6ccccc6)c54)n3)cc2)cc1]

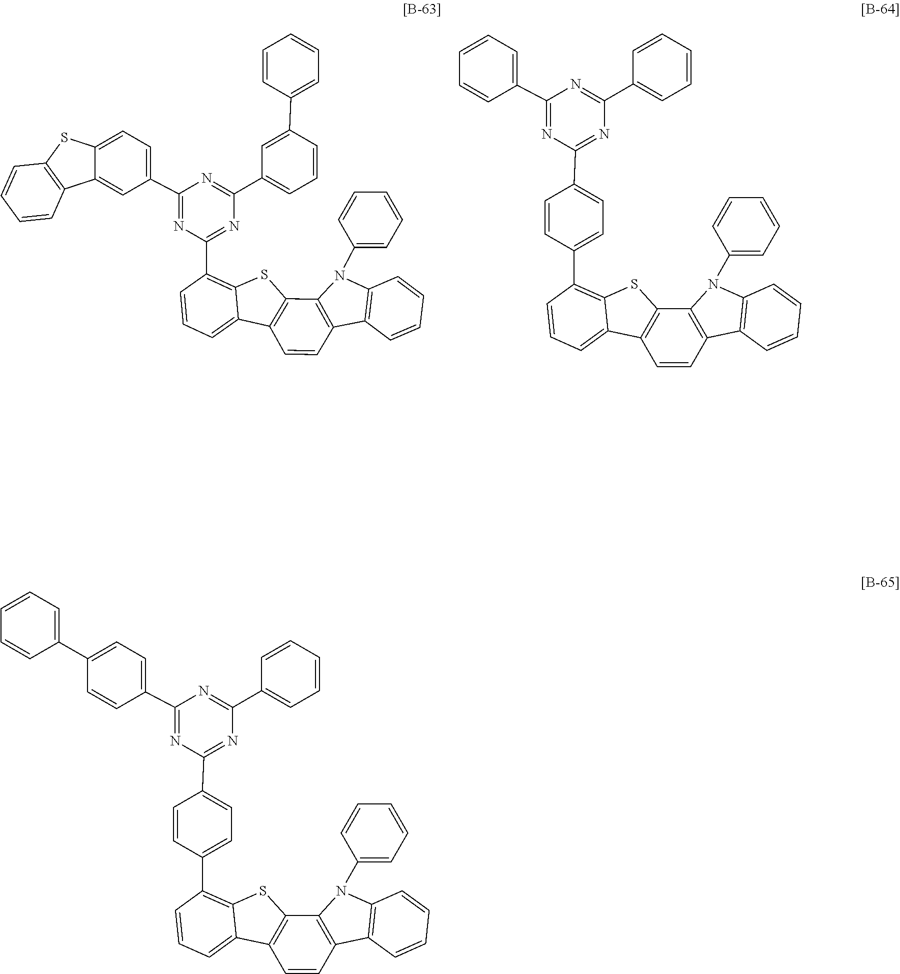 [SMILES: c1ccc(-c2cccc(-c3nc(-c4ccc5sc6ccccc6c5c4)nc(-c4cccc5c4sc4c5ccc5c6ccccc6n(-c6ccccc6)c54)n3)c2)cc1 ; c1ccc(-c2nc(-c3ccccc3)nc(-c3ccc(-c4cccc5c4sc4c5ccc5c6ccccc6n(-c6ccccc6)c54)cc3)n2)cc1 ; c1ccc(-c2ccc(-c3nc(-c4ccccc4)nc(-c4ccc(-c5cccc6c5sc5c6ccc6c7ccccc7n(-c7ccccc7)c65)cc4)n3)cc2)cc1]

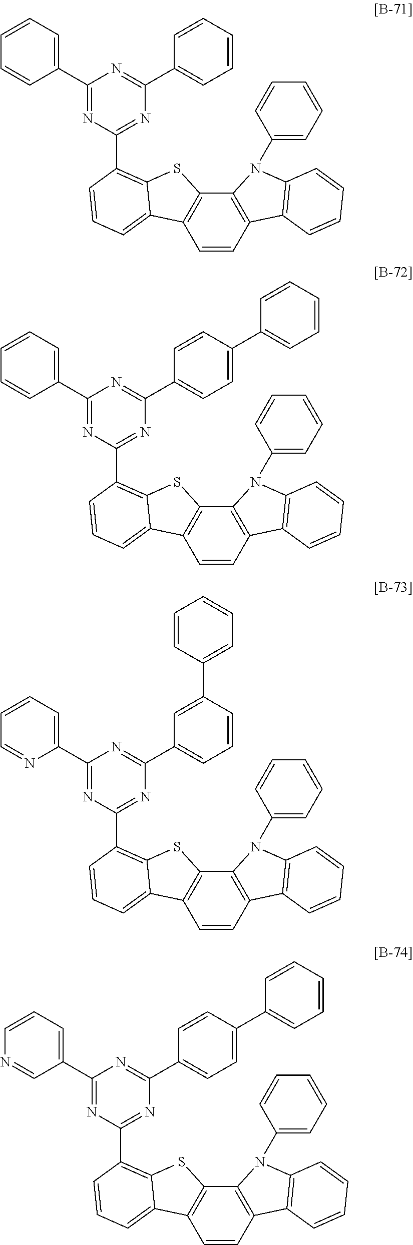

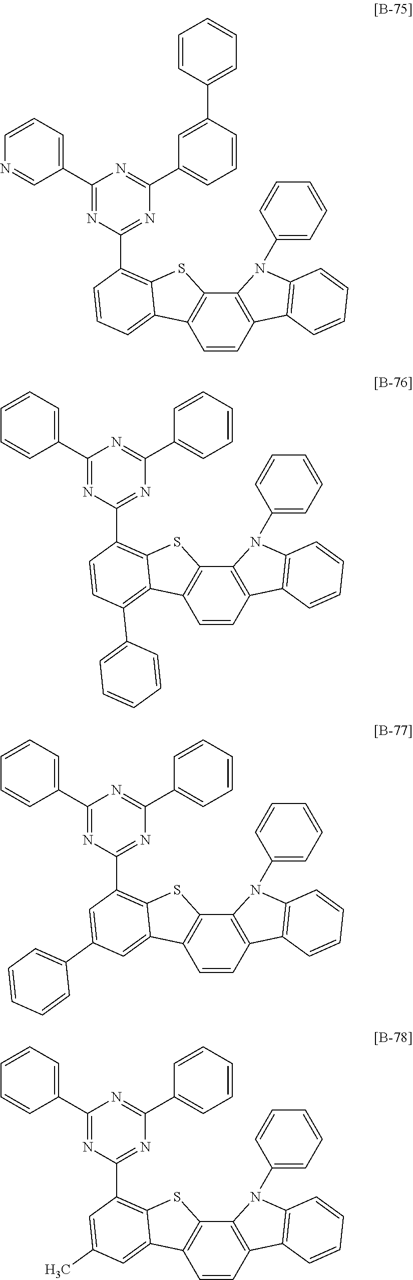

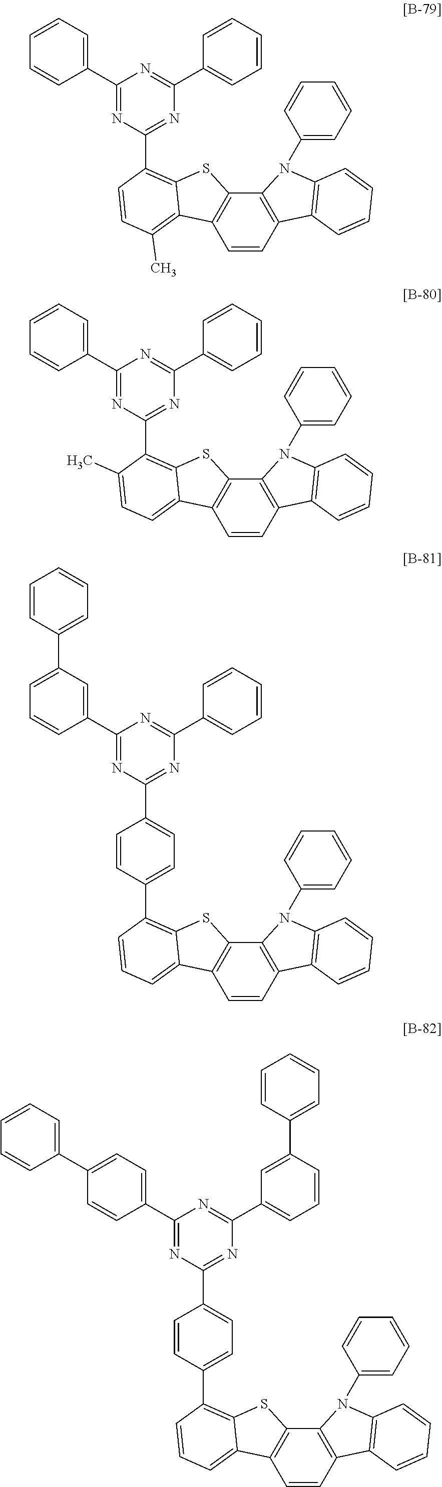 [SMILES: Cc1ccc(-c2nc(-c3ccccc3)nc(-c3ccccc3)n2)c2sc3c(ccc4c5ccccc5n(-c5ccccc5)c43)c12 ; Cc1ccc2c(sc3c2ccc2c4ccccc4n(-c4ccccc4)c23)c1-c1nc(-c2ccccc2)nc(-c2ccccc2)n1 ; c1ccc(-c2cccc(-c3nc(-c4ccccc4)nc(-c4ccc(-c5cccc6c5sc5c6ccc6c7ccccc7n(-c7ccccc7)c65)cc4)n3)c2)cc1 ; c1ccc(-c2ccc(-c3nc(-c4ccc(-c5cccc6c5sc5c6ccc6c7ccccc7n(-c7ccccc7)c65)cc4)nc(-c4cccc(-c5ccccc5)c4)n3)cc2)cc1]

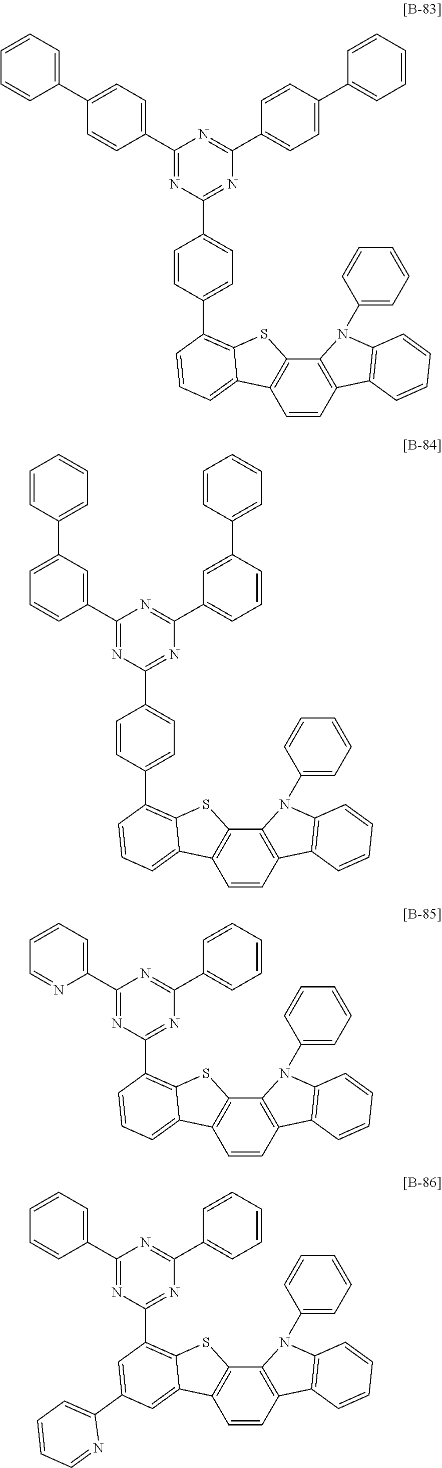

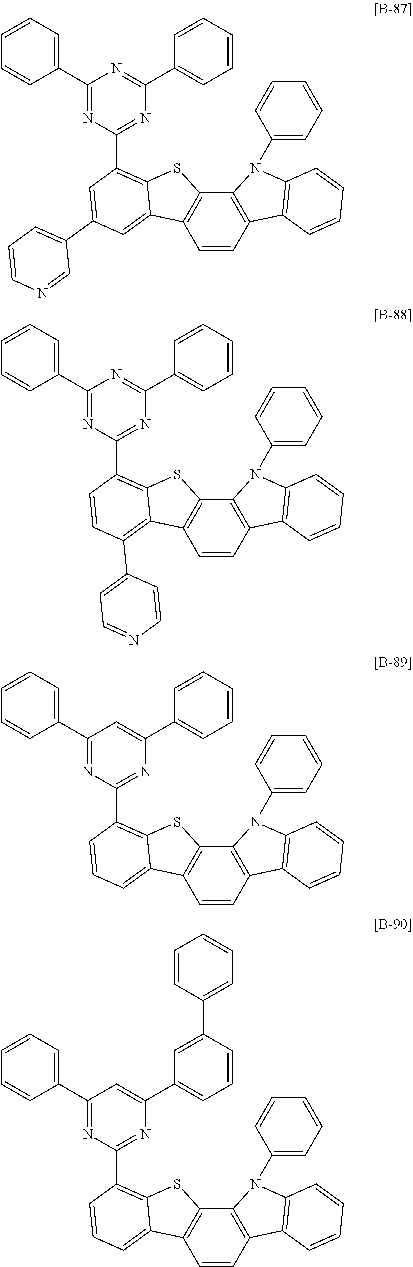

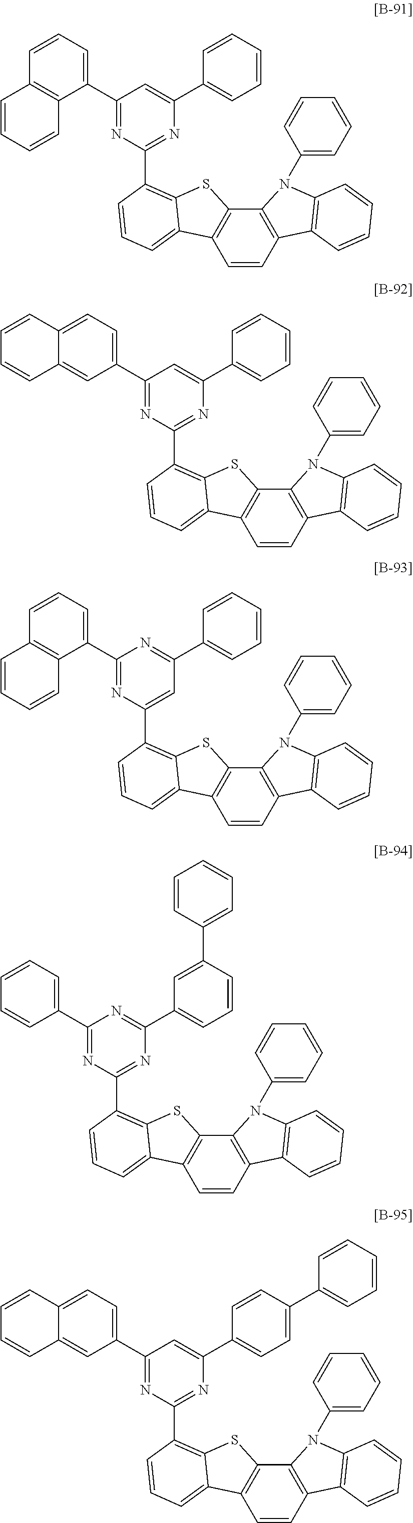

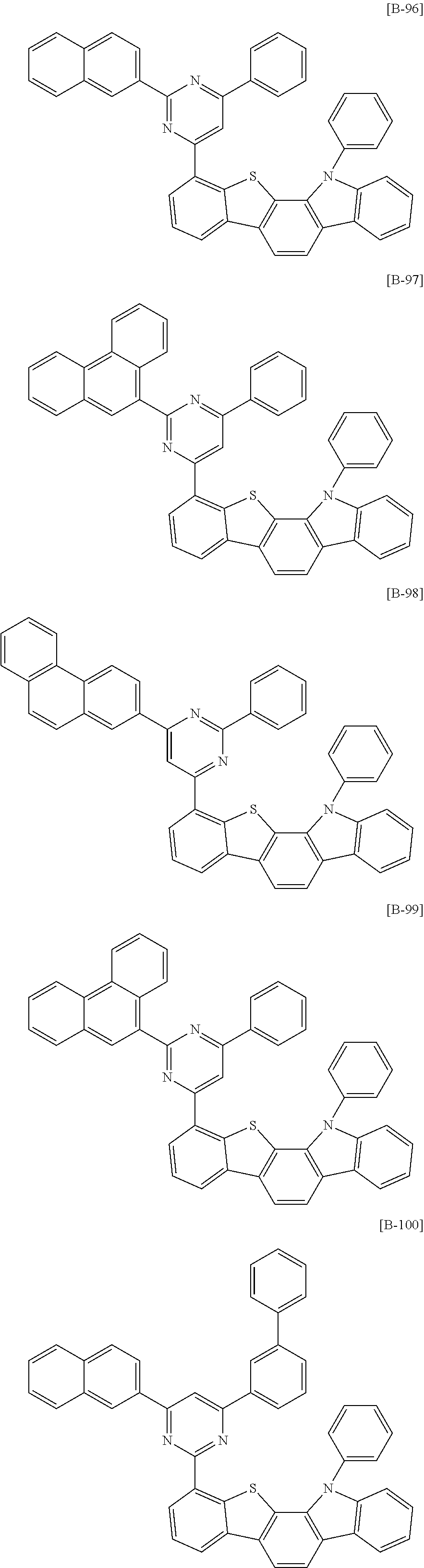

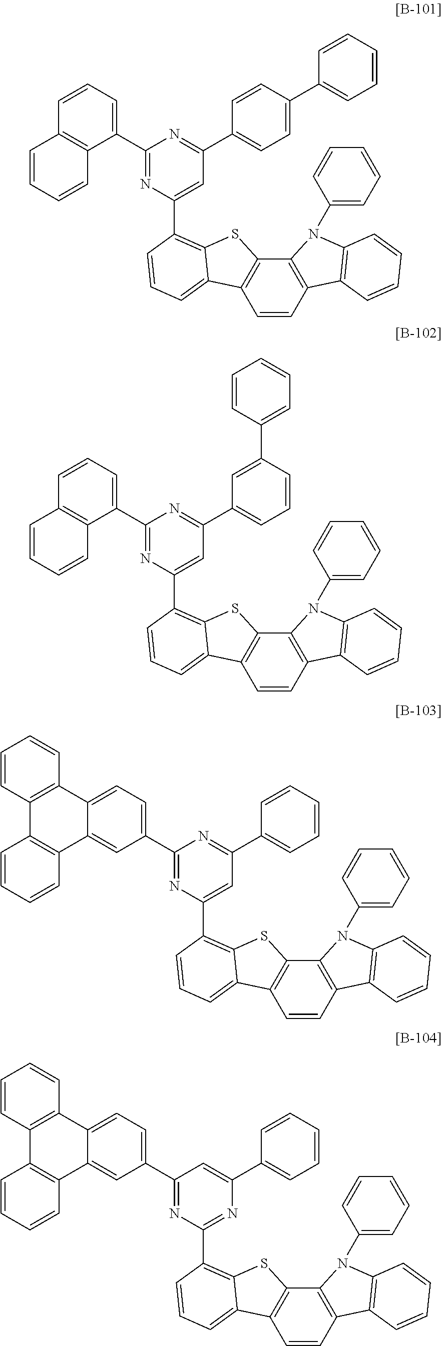

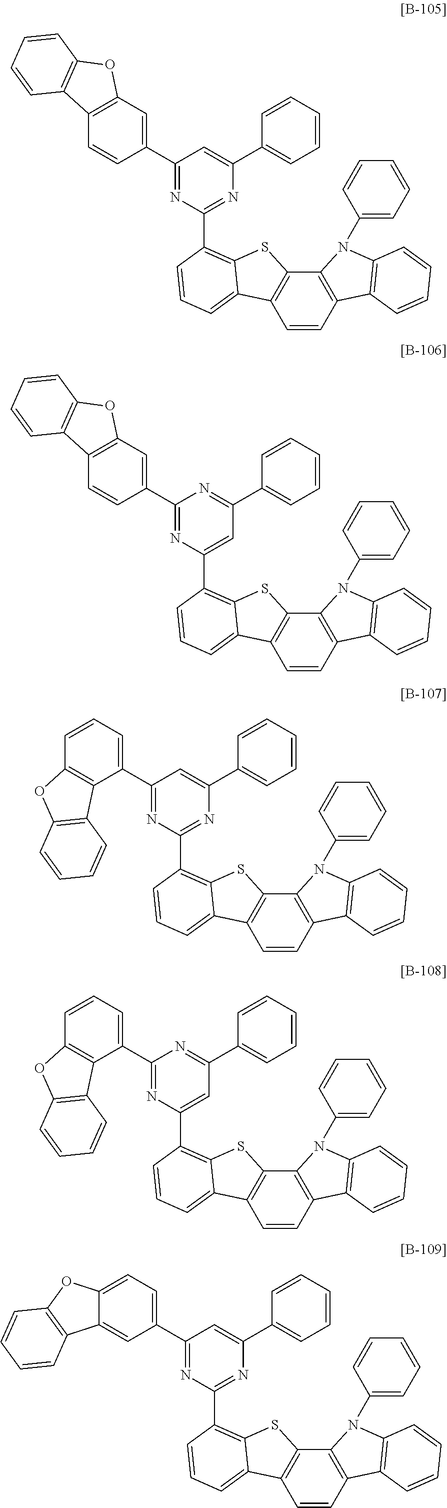

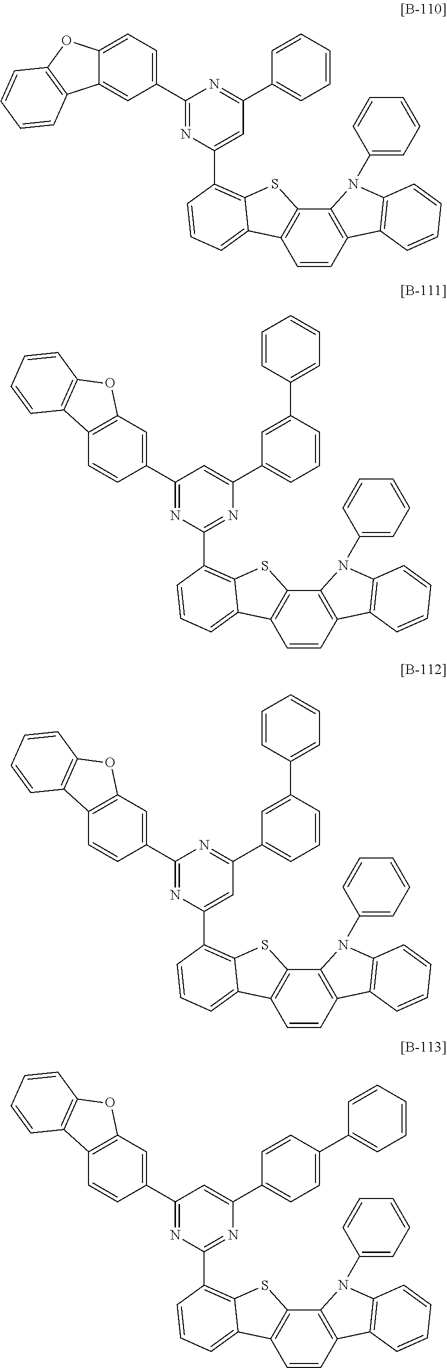 [SMILES: c1ccc(-c2cc(-c3cccc4c3sc3c4ccc4c5ccccc5n(-c5ccccc5)c43)nc(-c3ccc4oc5ccccc5c4c3)n2)cc1 ; c1ccc(-c2cccc(-c3cc(-c4ccc5c(c4)oc4ccccc45)nc(-c4cccc5c4sc4c5ccc5c6ccccc6n(-c6ccccc6)c54)n3)c2)cc1 ; c1ccc(-c2cccc(-c3cc(-c4cccc5c4sc4c5ccc5c6ccccc6n(-c6ccccc6)c54)nc(-c4ccc5c(c4)oc4ccccc45)n3)c2)cc1 ; c1ccc(-c2ccc(-c3cc(-c4ccc5c(c4)oc4ccccc45)nc(-c4cccc5c4sc4c5ccc5c6ccccc6n(-c6ccccc6)c54)n3)cc2)cc1]

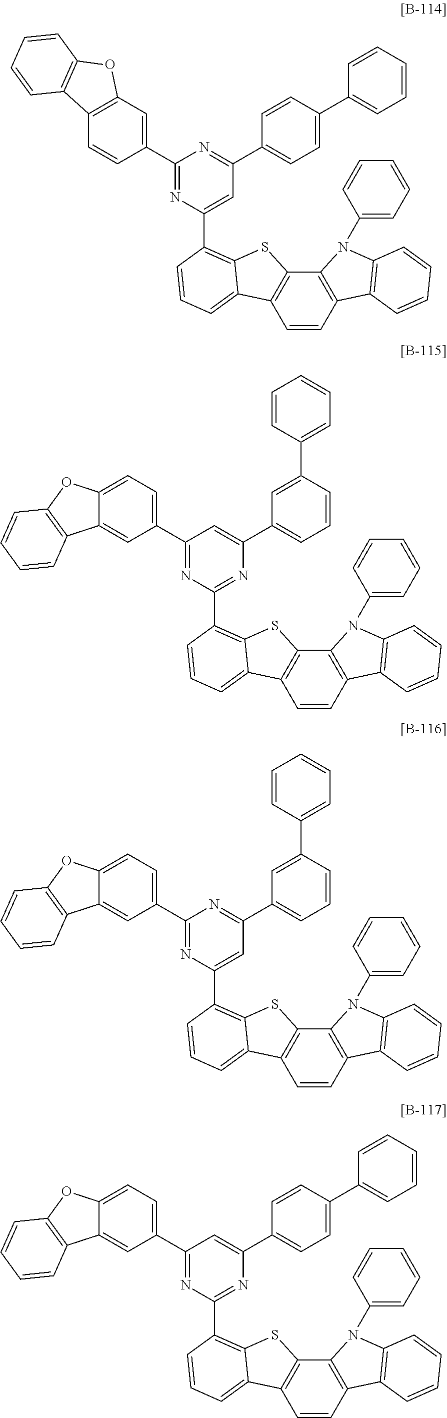

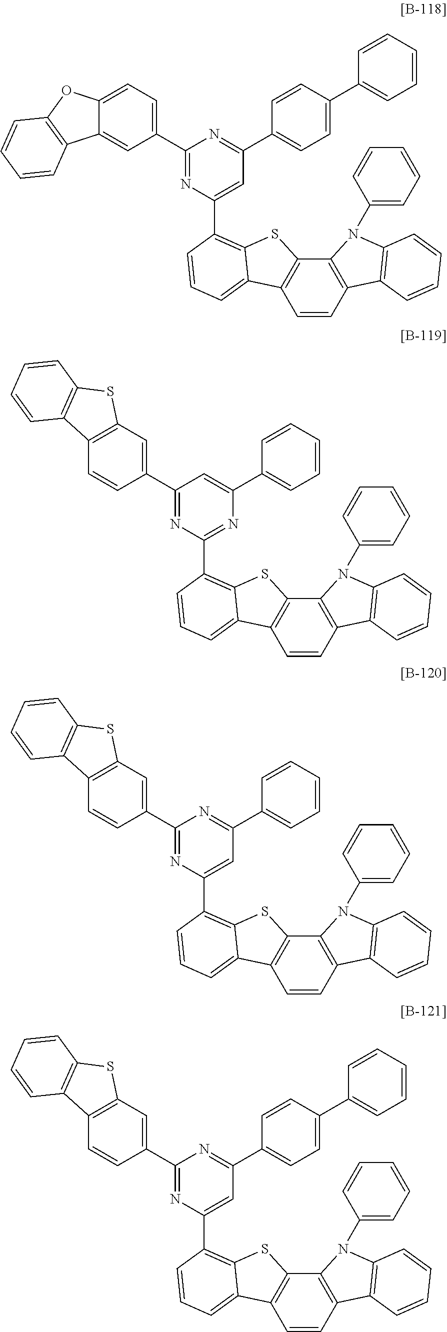 [SMILES: c1ccc(-c2ccc(-c3cc(-c4cccc5c4sc4c5ccc5c6ccccc6n(-c6ccccc6)c54)nc(-c4ccc5oc6ccccc6c5c4)n3)cc2)cc1 ; c1ccc(-c2cc(-c3ccc4c(c3)sc3ccccc34)nc(-c3cccc4c3sc3c4ccc4c5ccccc5n(-c5ccccc5)c43)n2)cc1 ; c1ccc(-c2cc(-c3cccc4c3sc3c4ccc4c5ccccc5n(-c5ccccc5)c43)nc(-c3ccc4c(c3)sc3ccccc34)n2)cc1 ; c1ccc(-c2ccc(-c3cc(-c4cccc5c4sc4c5ccc5c6ccccc6n(-c6ccccc6)c54)nc(-c4ccc5c(c4)sc4ccccc45)n3)cc2)cc1]

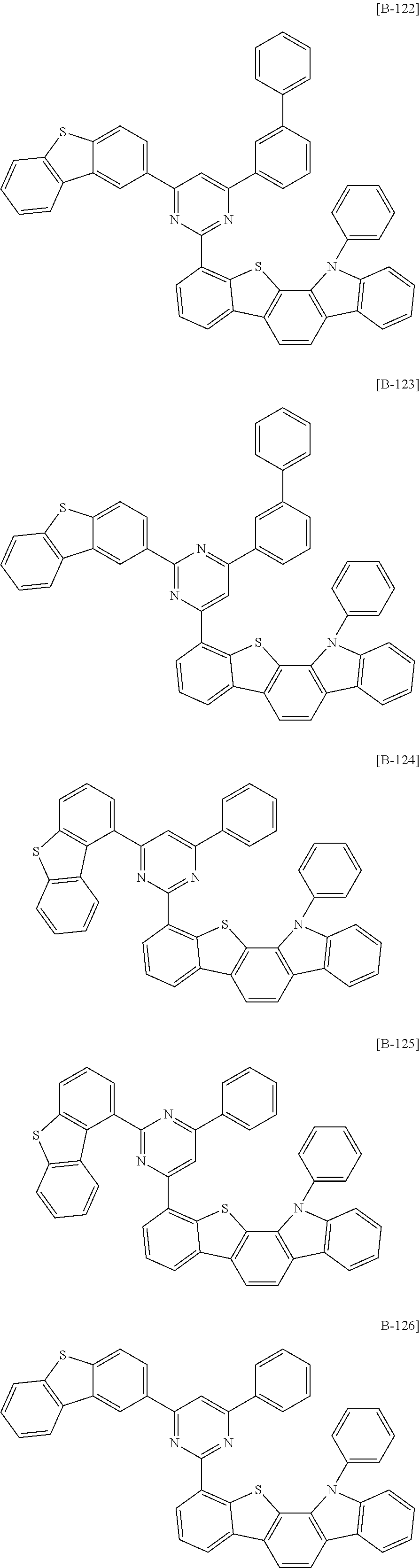 [SMILES: c1ccc(-c2cccc(-c3cc(-c4ccc5sc6ccccc6c5c4)nc(-c4cccc5c4sc4c5ccc5c6ccccc6n(-c6ccccc6)c54)n3)c2)cc1 ; c1ccc(-c2cccc(-c3cc(-c4cccc5c4sc4c5ccc5c6ccccc6n(-c6ccccc6)c54)nc(-c4ccc5sc6ccccc6c5c4)n3)c2)cc1 ; c1ccc(-c2cc(-c3cccc4sc5ccccc5c34)nc(-c3cccc4c3sc3c4ccc4c5ccccc5n(-c5ccccc5)c43)n2)cc1 ; c1ccc(-c2cc(-c3cccc4c3sc3c4ccc4c5ccccc5n(-c5ccccc5)c43)nc(-c3cccc4sc5ccccc5c34)n2)cc1 ; c1ccc(-c2cc(-c3ccc4sc5ccccc5c4c3)nc(-c3cccc4c3sc3c4ccc4c5ccccc5n(-c5ccccc5)c43)n2)cc1]

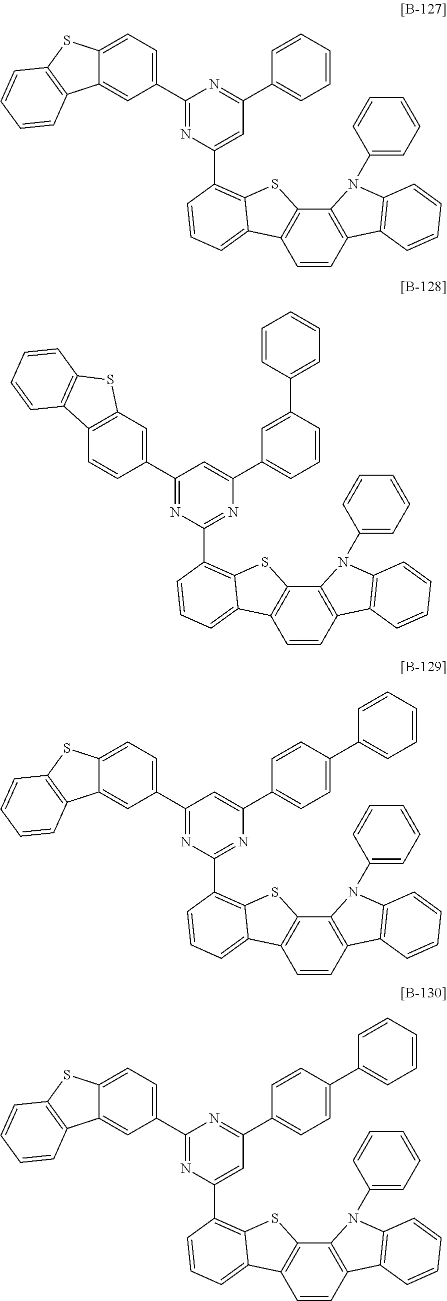

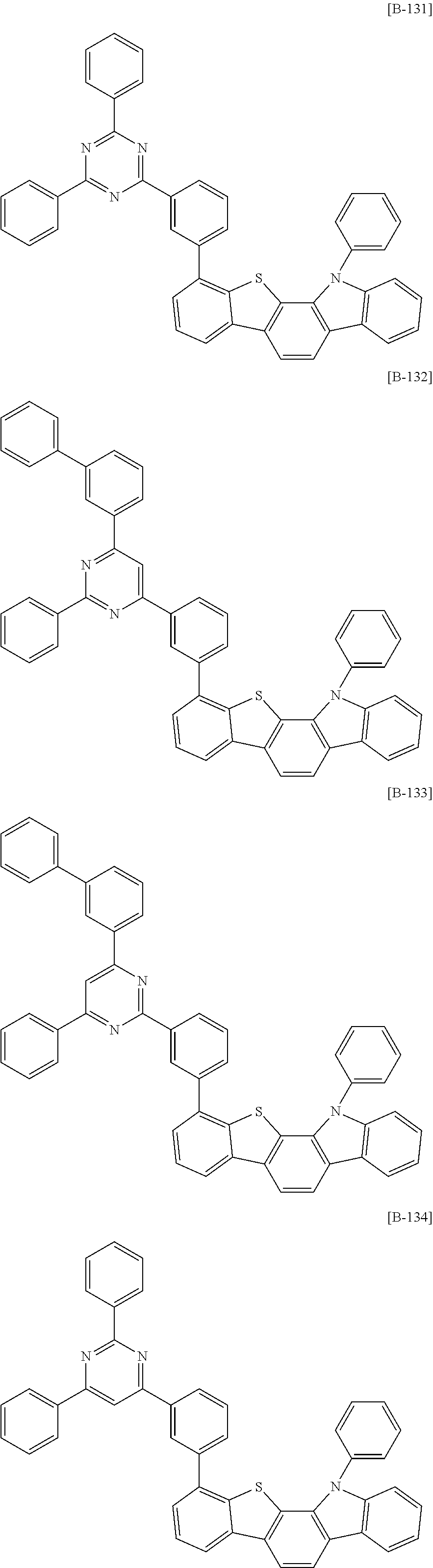 [SMILES: c1ccc(-c2nc(-c3ccccc3)nc(-c3cccc(-c4cccc5c4sc4c5ccc5c6ccccc6n(-c6ccccc6)c54)c3)n2)cc1 ; c1ccc(-c2cccc(-c3cc(-c4cccc(-c5cccc6c5sc5c6ccc6c7ccccc7n(-c7ccccc7)c65)c4)nc(-c4ccccc4)n3)c2)cc1 ; c1ccc(-c2cccc(-c3cc(-c4ccccc4)nc(-c4cccc(-c5cccc6c5sc5c6ccc6c7ccccc7n(-c7ccccc7)c65)c4)n3)c2)cc1 ; c1ccc(-c2cc(-c3cccc(-c4cccc5c4sc4c5ccc5c6ccccc6n(-c6ccccc6)c54)c3)nc(-c3ccccc3)n2)cc1]

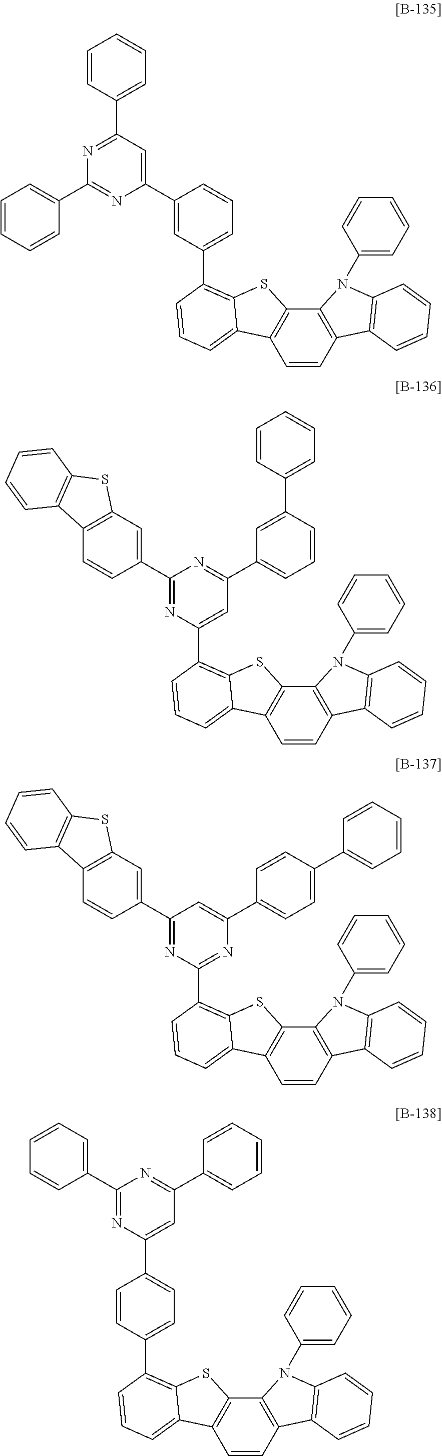

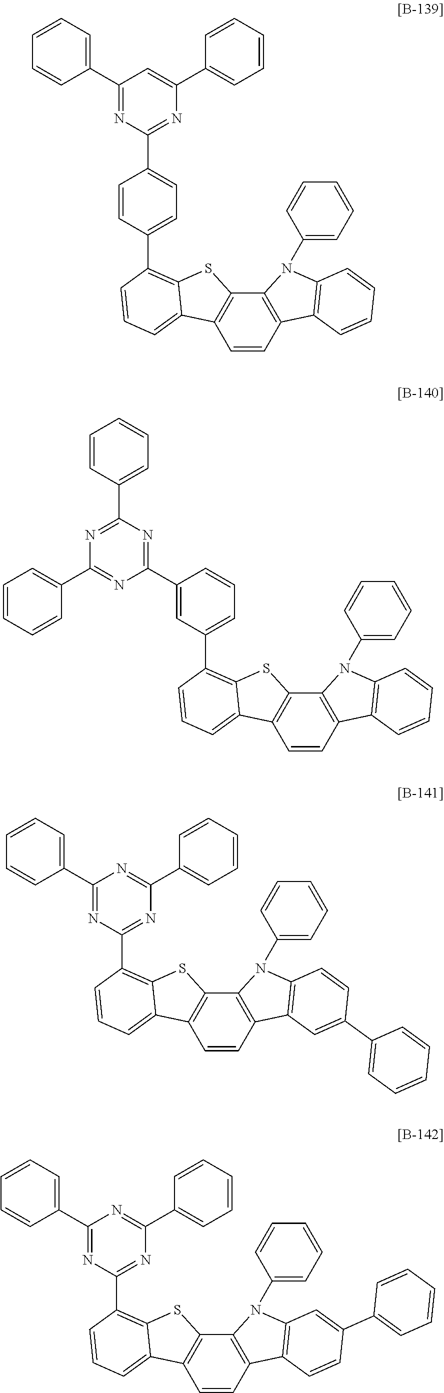 [SMILES: c1ccc(-c2cc(-c3ccccc3)nc(-c3ccc(-c4cccc5c4sc4c5ccc5c6ccccc6n(-c6ccccc6)c54)cc3)n2)cc1 ; c1ccc(-c2nc(-c3ccccc3)nc(-c3cccc(-c4cccc5c4sc4c5ccc5c6ccccc6n(-c6ccccc6)c54)c3)n2)cc1 ; c1ccc(-c2ccc3c(c2)c2ccc4c5cccc(-c6nc(-c7ccccc7)nc(-c7ccccc7)n6)c5sc4c2n3-c2ccccc2)cc1 ; c1ccc(-c2ccc3c4ccc5c6cccc(-c7nc(-c8ccccc8)nc(-c8ccccc8)n7)c6sc5c4n(-c4ccccc4)c3c2)cc1]

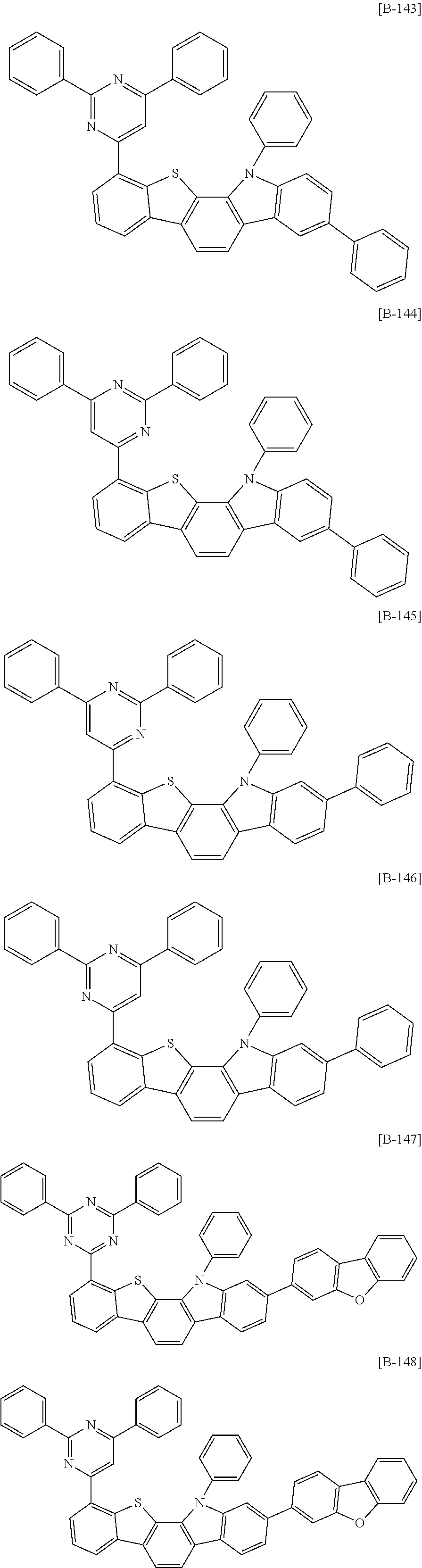

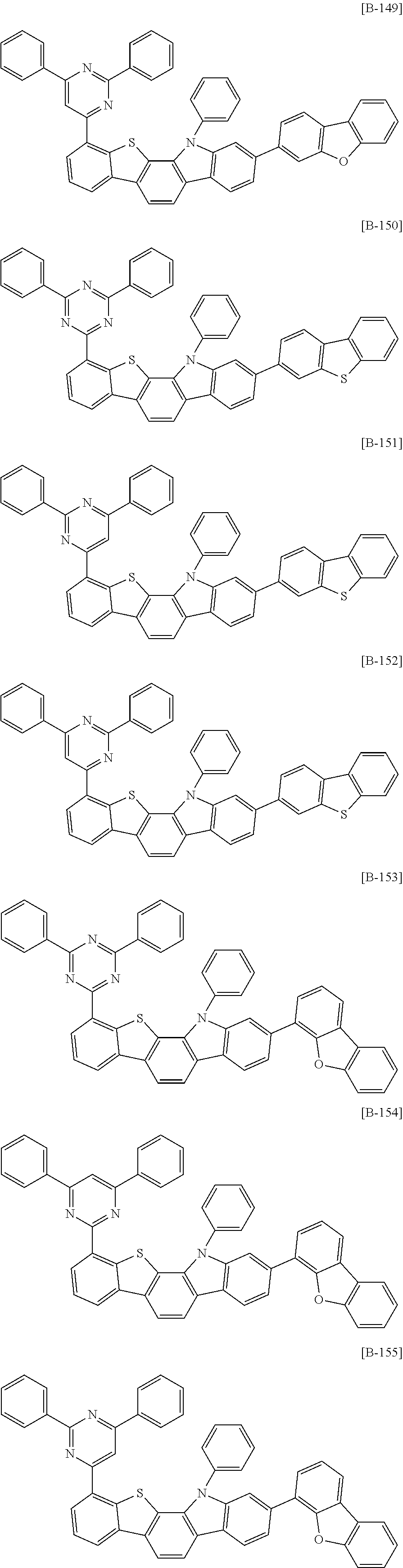

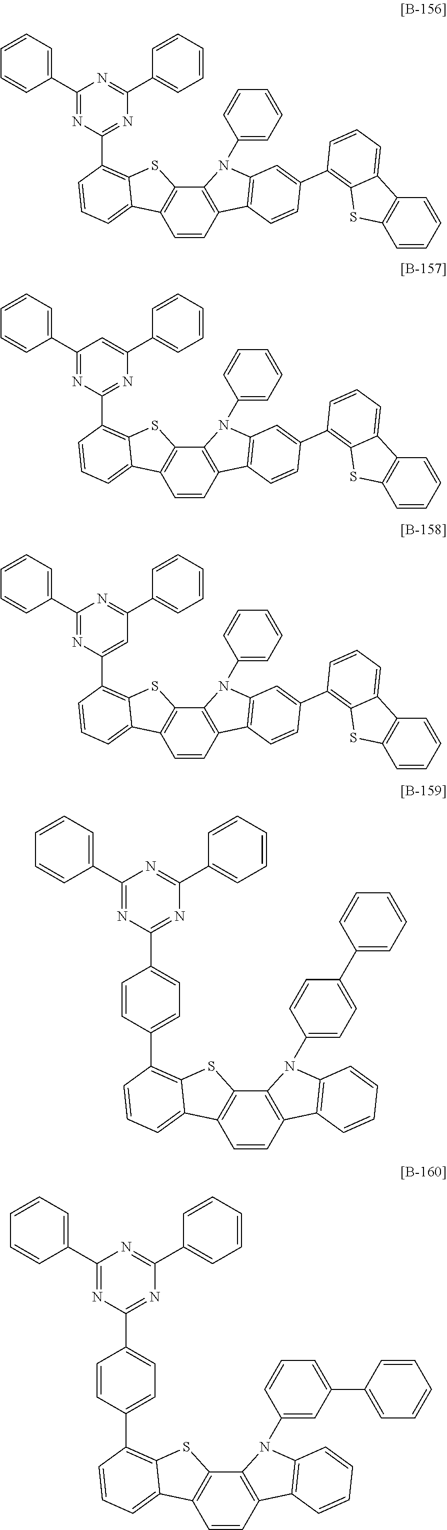 [SMILES: c1ccc(-c2nc(-c3ccccc3)nc(-c3cccc4c3sc3c4ccc4c5ccc(-c6cccc7c6sc6ccccc67)cc5n(-c5ccccc5)c43)n2)cc1 ; c1ccc(-c2cc(-c3ccccc3)nc(-c3cccc4c3sc3c4ccc4c5ccc(-c6cccc7c6sc6ccccc67)cc5n(-c5ccccc5)c43)n2)cc1 ; c1ccc(-c2cc(-c3cccc4c3sc3c4ccc4c5ccc(-c6cccc7c6sc6ccccc67)cc5n(-c5ccccc5)c43)nc(-c3ccccc3)n2)cc1 ; c1ccc(-c2ccc(-n3c4ccccc4c4ccc5c6cccc(-c7ccc(-c8nc(-c9ccccc9)nc(-c9ccccc9)n8)cc7)c6sc5c43)cc2)cc1 ; c1ccc(-c2cccc(-n3c4ccccc4c4ccc5c6cccc(-c7ccc(-c8nc(-c9ccccc9)nc(-c9ccccc9)n8)cc7)c6sc5c43)c2)cc1]

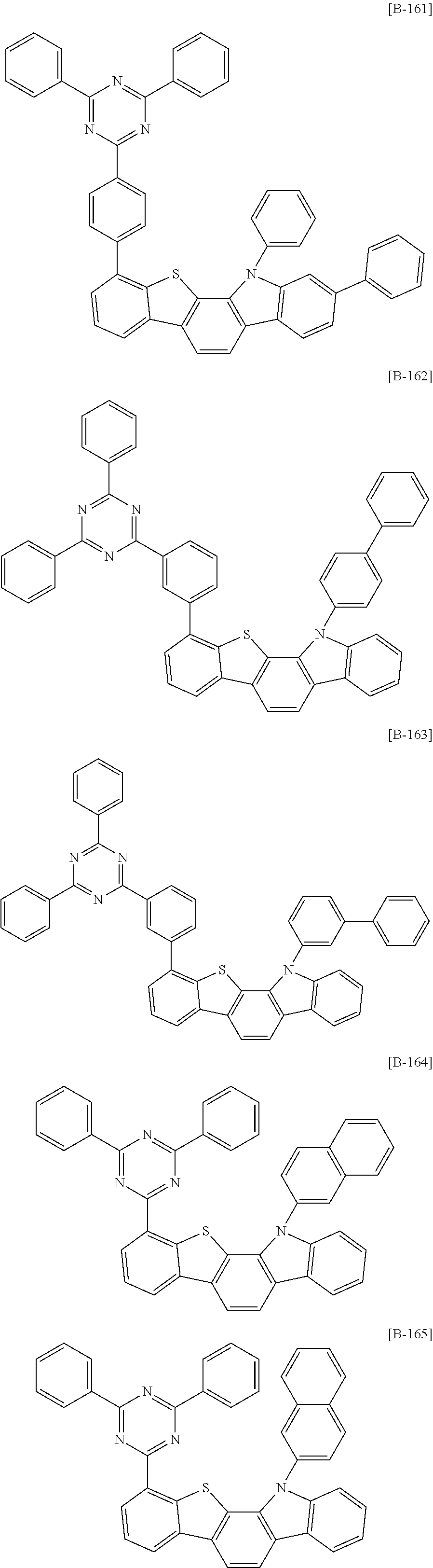

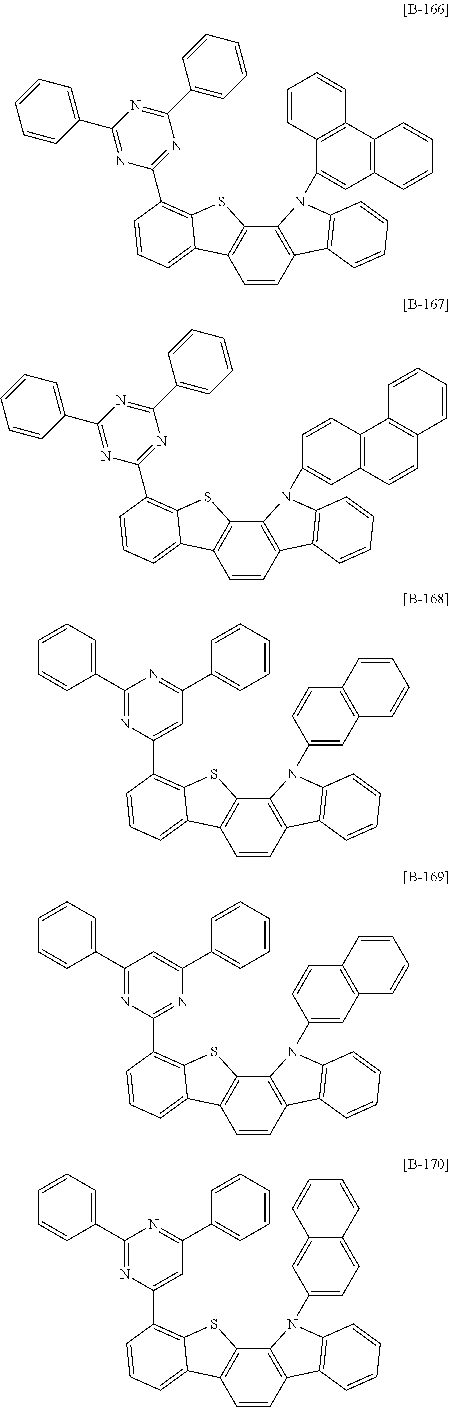

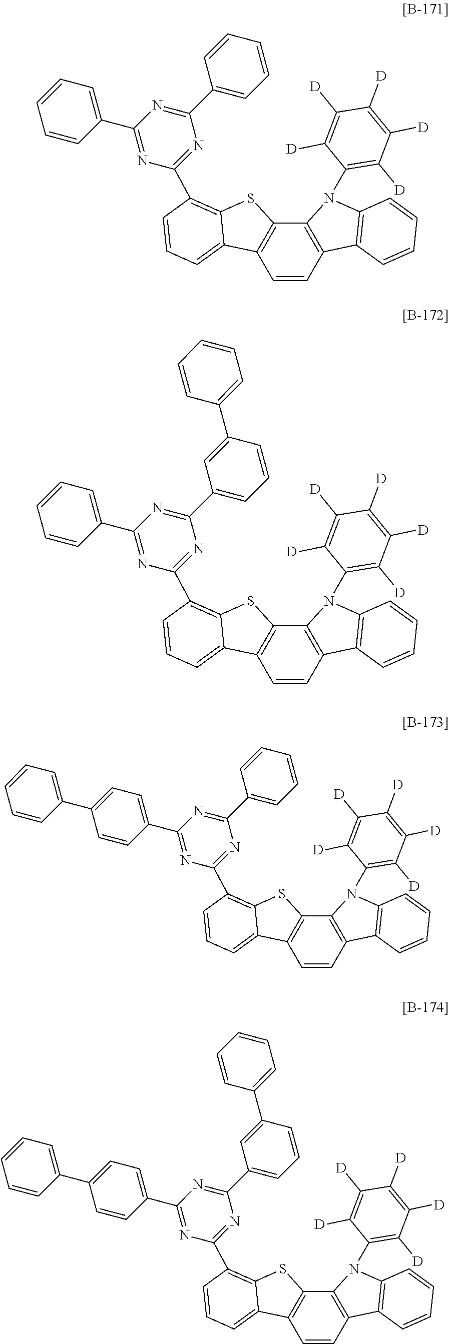 [SMILES: [2H]c1c([2H])c([2H])c(-n2c3ccccc3c3ccc4c5cccc(-c6nc(-c7ccccc7)nc(-c7ccccc7)n6)c5sc4c32)c([2H])c1[2H] ; [2H]c1c([2H])c([2H])c(-n2c3ccccc3c3ccc4c5cccc(-c6nc(-c7ccccc7)nc(-c7cccc(-c8ccccc8)c7)n6)c5sc4c32)c([2H])c1[2H] ; [2H]c1c([2H])c([2H])c(-n2c3ccccc3c3ccc4c5cccc(-c6nc(-c7ccccc7)nc(-c7ccc(-c8ccccc8)cc7)n6)c5sc4c32)c([2H])c1[2H] ; [2H]c1c([2H])c([2H])c(-n2c3ccccc3c3ccc4c5cccc(-c6nc(-c7ccc(-c8ccccc8)cc7)nc(-c7cccc(-c8ccccc8)c7)n6)c5sc4c32)c([2H])c1[2H]]

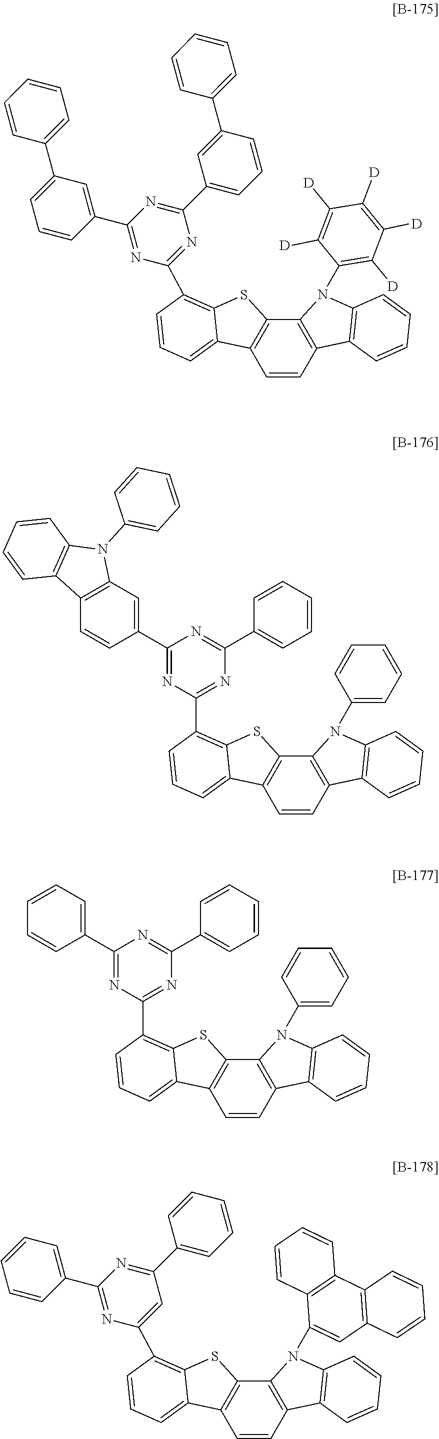 [SMILES: [2H]c1c([2H])c([2H])c(-n2c3ccccc3c3ccc4c5cccc(-c6nc(-c7cccc(-c8ccccc8)c7)nc(-c7cccc(-c8ccccc8)c7)n6)c5sc4c32)c([2H])c1[2H] ; c1ccc(-c2nc(-c3ccc4c5ccccc5n(-c5ccccc5)c4c3)nc(-c3cccc4c3sc3c4ccc4c5ccccc5n(-c5ccccc5)c43)n2)cc1 ; c1ccc(-c2nc(-c3ccccc3)nc(-c3cccc4c3sc3c4ccc4c5ccccc5n(-c5ccccc5)c43)n2)cc1 ; c1ccc(-c2cc(-c3cccc4c3sc3c4ccc4c5ccccc5n(-c5cc6ccccc6c6ccccc56)c43)nc(-c3ccccc3)n2)cc1]

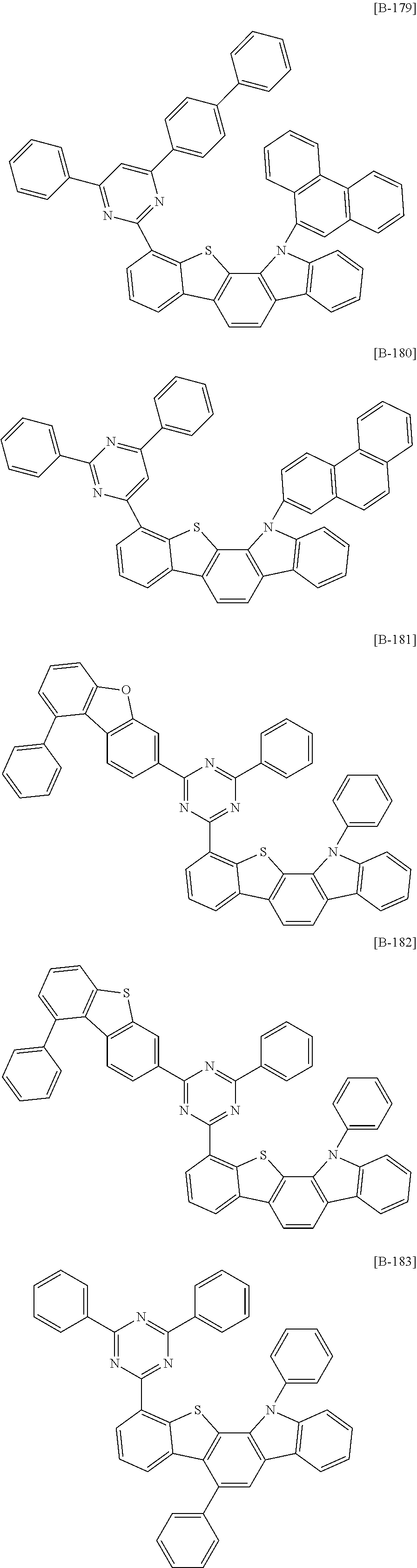 [SMILES: c1ccc(-c2ccc(-c3cc(-c4ccccc4)nc(-c4cccc5c4sc4c5ccc5c6ccccc6n(-c6cc7ccccc7c7ccccc67)c54)n3)cc2)cc1 ; c1ccc(-c2cc(-c3cccc4c3sc3c4ccc4c5ccccc5n(-c5ccc6c(ccc7ccccc76)c5)c43)nc(-c3ccccc3)n2)cc1 ; c1ccc(-c2nc(-c3ccc4c(c3)oc3cccc(-c5ccccc5)c34)nc(-c3cccc4c3sc3c4ccc4c5ccccc5n(-c5ccccc5)c43)n2)cc1 ; c1ccc(-c2nc(-c3ccc4c(c3)sc3cccc(-c5ccccc5)c34)nc(-c3cccc4c3sc3c4ccc4c5ccccc5n(-c5ccccc5)c43)n2)cc1 ; c1ccc(-c2nc(-c3ccccc3)nc(-c3cccc4c3sc3c4c(-c4ccccc4)cc4c5ccccc5n(-c5ccccc5)c43)n2)cc1]

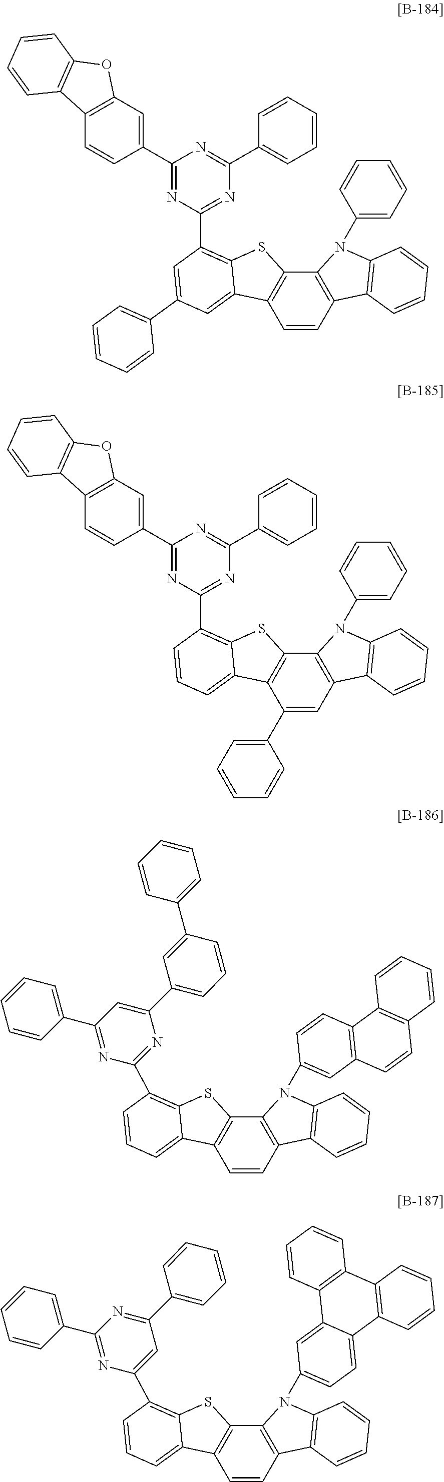 [SMILES: c1ccc(-c2cc(-c3nc(-c4ccccc4)nc(-c4ccc5c(c4)oc4ccccc45)n3)c3sc4c(ccc5c6ccccc6n(-c6ccccc6)c54)c3c2)cc1 ; c1ccc(-c2nc(-c3ccc4c(c3)oc3ccccc34)nc(-c3cccc4c3sc3c4c(-c4ccccc4)cc4c5ccccc5n(-c5ccccc5)c43)n2)cc1 ; c1ccc(-c2cccc(-c3cc(-c4ccccc4)nc(-c4cccc5c4sc4c5ccc5c6ccccc6n(-c6ccc7c(ccc8ccccc87)c6)c54)n3)c2)cc1 ; c1ccc(-c2cc(-c3cccc4c3sc3c4ccc4c5ccccc5n(-c5ccc6c7ccccc7c7ccccc7c6c5)c43)nc(-c3ccccc3)n2)cc1]

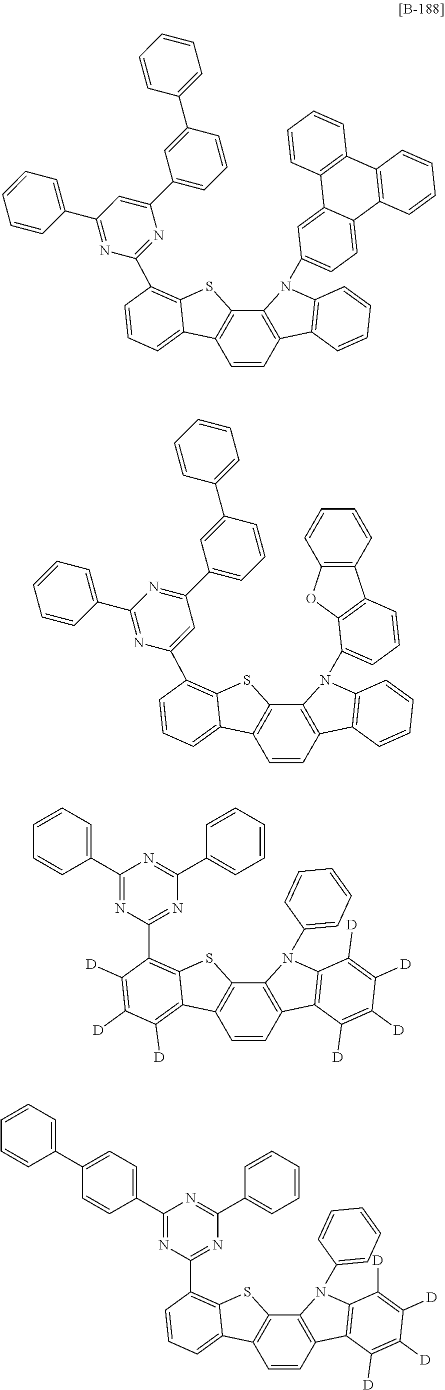 [SMILES: c1ccc(-c2cccc(-c3cc(-c4ccccc4)nc(-c4cccc5c4sc4c5ccc5c6ccccc6n(-c6ccc7c8ccccc8c8ccccc8c7c6)c54)n3)c2)cc1 ; c1ccc(-c2cccc(-c3cc(-c4cccc5c4sc4c5ccc5c6ccccc6n(-c6cccc7c6oc6ccccc67)c54)nc(-c4ccccc4)n3)c2)cc1 ; [2H]c1c([2H])c([2H])c2c(sc3c2ccc2c4c([2H])c([2H])c([2H])c([2H])c4n(-c4ccccc4)c23)c1-c1nc(-c2ccccc2)nc(-c2ccccc2)n1 ; [2H]c1c([2H])c([2H])c2c(c1[2H])c1ccc3c4cccc(-c5nc(-c6ccccc6)nc(-c6ccc(-c7ccccc7)cc6)n5)c4sc3c1n2-c1ccccc1]

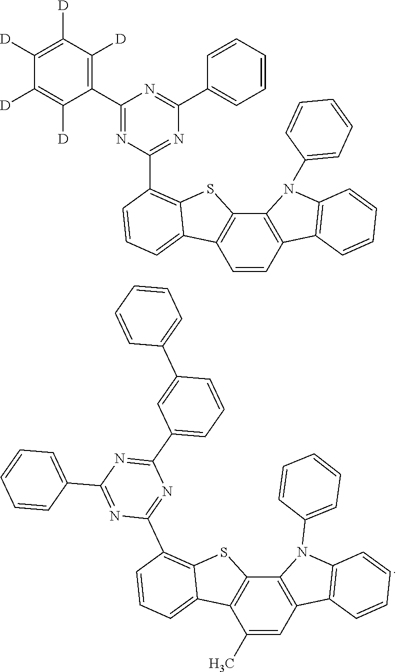

7. The composition of claim 1, wherein the second compound is represented by Chemical Formula 2-8: 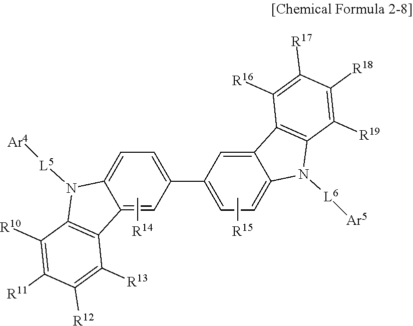
wherein, in Chemical Formula 2-8,
Ar⁴, Ar⁵, L⁵, L⁶, and R¹⁰ to R¹⁹ are the same as defined in claim 1.

8. The composition of claim 7, wherein
L⁵ and L⁶ of Chemical Formula 2-8 are each independently a single bond, a substituted or unsubstituted phenylene group, or a substituted or unsubstituted biphenylene group,
Ar⁴ and Ar⁵ are each independently a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, a substituted or unsubstituted fluorenyl group, or a substituted or unsubstituted pyridinyl group, and
R¹⁰ to R¹⁹ are each independently hydrogen, deuterium, a substituted or unsubstituted C1 to C10 alkyl group, a substituted or unsubstituted C6 to C12 aryl group, a substituted or unsubstituted dibenzofuranyl group, or a substituted or unsubstituted dibenzothiophenyl group.

9. The composition of claim 7, wherein *-L⁵-Ar⁴ and *-L⁶-Ar⁵ of Chemical Formula 2-8 are each independently selected from substituted or unsubstituted groups of Group II: 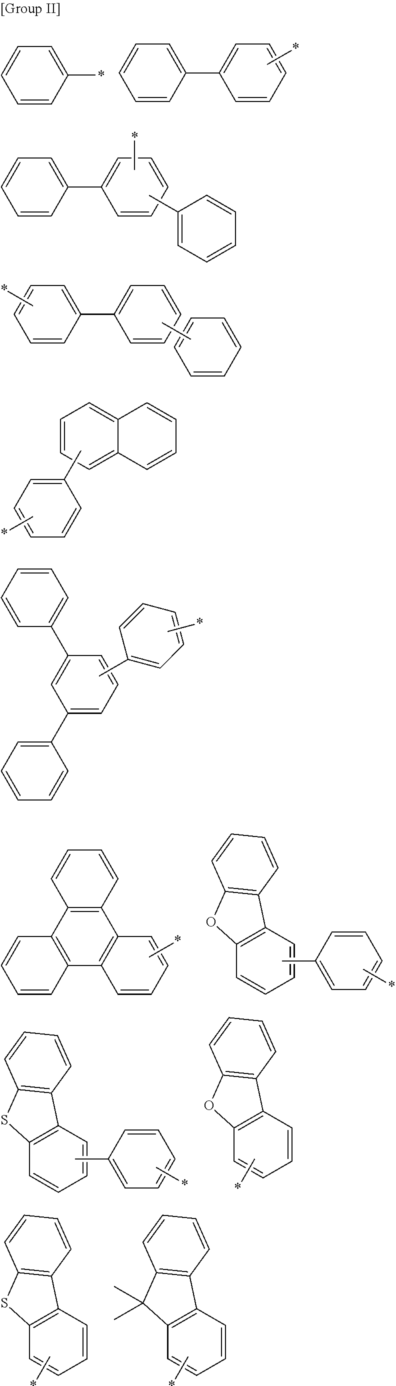 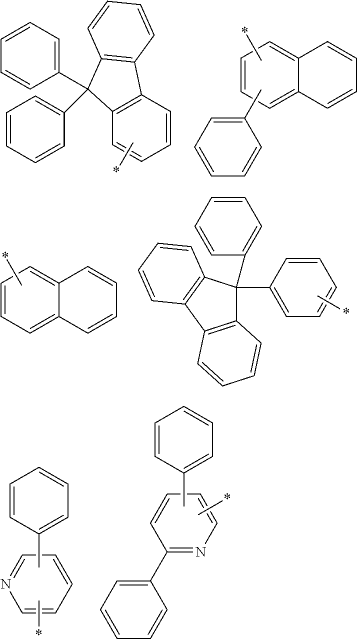
wherein, in Group II, “substituted” means substitution with deuterium, a fluoro group, a C1 to C5 alkyl group, or a C6 to C12 aryl group, and
* is a linking point.

10. The composition of claim 7, wherein
*-L⁵-Ar⁴ and *-L⁶-Ar⁵ of Chemical Formula 2-8 are each independently selected from substituted or unsubstituted groups of Group II-1: 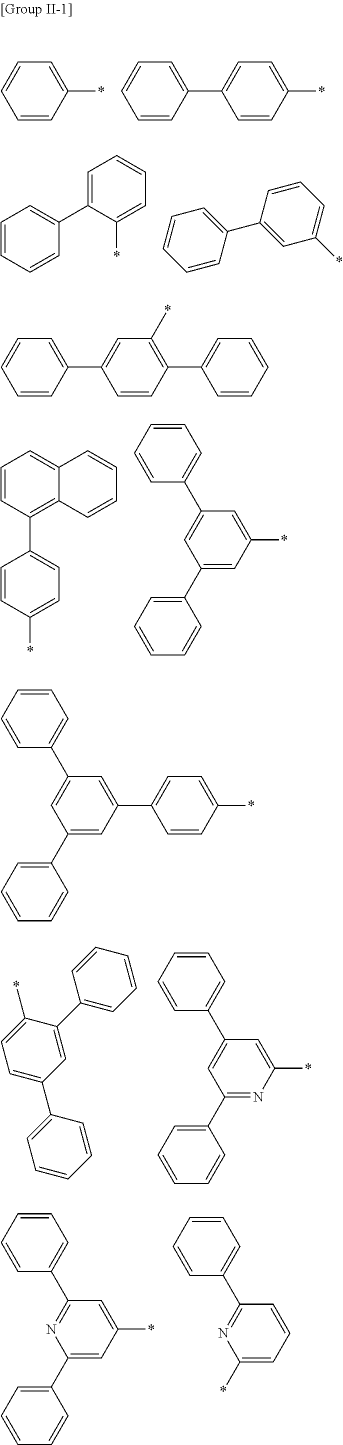 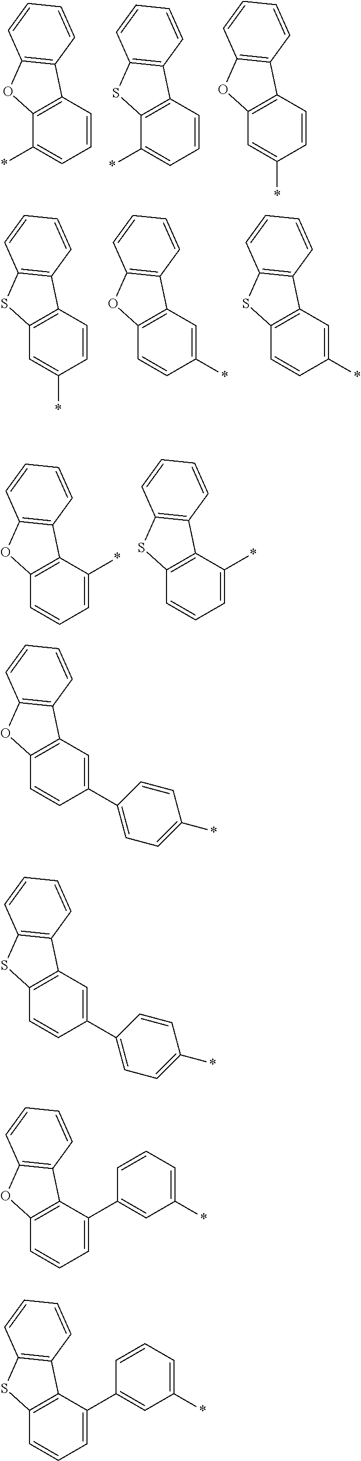 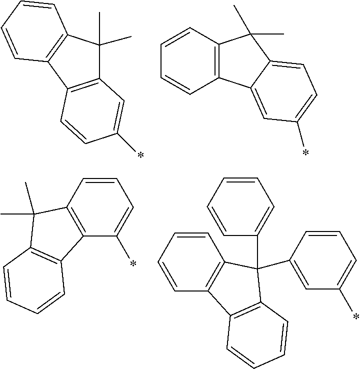
wherein, in Group II-1, “‘substituted” means substitution with deuterium, a fluoro group, a C1 to C5 alkyl group, or a C6 to C12 aryl group, and
* is a linking point.

11. The composition of claim 1, wherein
the first compound is one of compounds of Group 1-1, and
the second compound is one of compounds of Group 2-1:

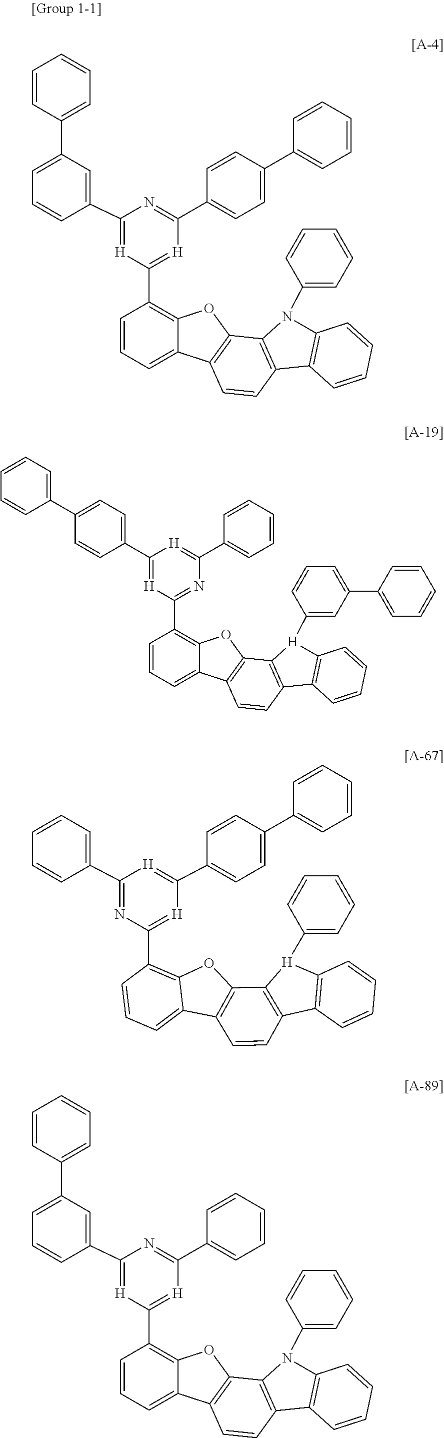

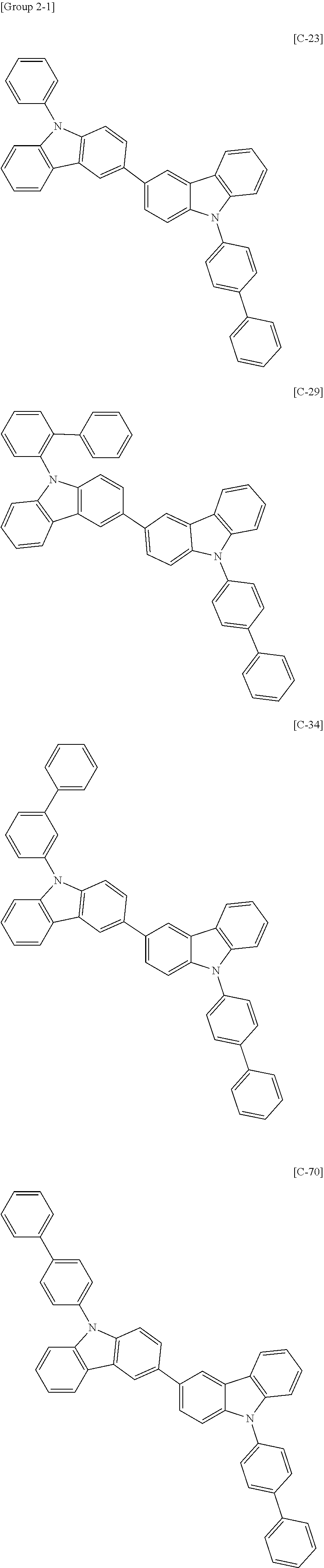 [SMILES: c1ccc(-c2ccc(-n3c4ccccc4c4cc(-c5ccc6c(c5)c5ccccc5n6-c5ccccc5)ccc43)cc2)cc1 ; c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

12. An organic optoelectronic device, comprising:
an anode and a cathode facing each other; and
at least one organic layer between the anode and the cathode,
wherein the at least one organic layer includes a light emitting layer, and
the light emitting layer includes the composition for an organic optoelectronic device of claim 1.

13. The organic optoelectronic device of claim 12, wherein the composition for an organic optoelectronic device is included as a phosphorescent host of the light emitting layer.

14. The organic optoelectronic device of claim 12, wherein the composition for an organic optoelectronic device includes the first compound and the second compound in a weight ratio of 20:80 to 50:50.

15. A display device comprising the organic optoelectronic device of claim 12.
